SEQUENCE LISTING (6 sequences)

SEQ ID NO: 1 - protein, 121 aa, Artificial
  Humanized heavy chain variable region
QVQLVQSGAEVKKPGASVKVSCKASGYTFTNYWIHWVRQAPGQGLEWMGVIDPSDTYPNYNKKFKGRVTMTTDTSTSTAYMELRSLRSDDTAVYYCARNGDSDYYSGMDYWGQGTTVTVSS

SEQ ID NO: 2 - protein, 107 aa, Artificial
  Humanized 2B6 light chain variable region - Hu2B6VL-1
EIVLTQSPDFQSVTPKEKVTITCRTSQSIGTNIHWYQQKPDQSPKLLIKNVSESISGVPSRFSGSGSGTDFTLTINSLEAEDAATYYCQQSNTWPFTFGGGTKVEIK

SEQ ID NO: 3 - protein, 107 aa, Artificial
  Humanized 2B6 light chain variable region - Hu2B6VL-2
EIVLTQSPDFQSVTPKEKVTITCRTSQSIGTNIHWYQQKPDQSPKLLIKYVSESISGVPSRFSGSGSGTDFTLTINSLEAEDAATYYCQQSNTWPFTFGGGTKVEIK

SEQ ID NO: 4 - protein, 107 aa, Artificial
  Humanized 2B6 light chain variable region - Hu2B6VL-3
EIVLTQSPDFQSVTPKEKVTITCRTSQSIGTNIHWYQQKPDQSPKLLIKYASESISGVPSRFSGSGSGTDFTLTINSLEAEDAATYYCQQSNTWPFTFGGGTKVEIK

SEQ ID NO: 5 - protein, 115 aa, Mus sp.
  MISC_FEATURE (1)..(115): mouse 3H7 Heavy Chain Variable Region
EVKFEESGGGLVQPGGSMKLSCAASGFTFSDAWMDWVRQGPEKGLEWVAEIRNKANNLATYYAESVKGRFTIPRDDSKSSVYLHMNSLRAEDTGIYYCYSPFAYWGQGTLVTVSA

SEQ ID NO: 6 - protein, 107 aa, mus sp.
  MISC_FEATURE (1)..(107): mouse 3H7 Light Chain Variable Region
DIQMTQSPSSLSASLGERVSLTCRASQEISGYLSWLQQKPDGTIRRLIYAASTLDSGVPKRFSGSWSGSDYSLTISSLESEDFADYYCLQYVSYPYTFGGGTKLEIK

(19) United States
(12) Patent Application Publication
(10) Pub. No.: US 2009/0098124 A1
(43) Pub. Date: Apr. 16, 2009

(54) IDENTIFICATION AND ENGINEERING OF ANTIBODIES WITH VARIANT HEAVY CHAINS AND METHODS OF USING SAME

(75) Inventor: Jeffrey B. Stavenhagen, Brookville, MD (US)

Correspondence Address: EDELL, SHAPIRO & FINNAN, LLC, 1901 RESEARCH BLVD., SUITE 400, ROCKVILLE, MD, 20850-3164, US

(73) Assignee: MACROGENICS, INC., Rockville, MD (US)

(21) Appl. No.: 12/205,471

(22) Filed: Sep. 5, 2008

Related U.S. Application Data
(63) Continuation of application No. PCT/US2007/063548, filed on Mar. 8, 2007.
(60) Provisional application No. 60/781,564, filed on Mar. 10, 2006.

(51) Int. Cl. A61K 39/395; C07K 16/18; C12N 15/11

(52) U.S. Cl. 424/136.1; 530/387.3; 536/23.53

(57) ABSTRACT

The present invention relates to molecules, particularly polypeptides, more particularly immunoglobulins (e.g., antibodies), comprising a variant heavy chain, which variant heavy chain comprises constant domains from more than one IgG isotype. The variant heavy chain of the invention may further comprise at least one amino acid modification relative to the parental heavy chain, such that the Fc region of said variant heavy chain binds an FcγR with an altered affinity relative to a comparable molecule comprising the wild-type heavy cahin. The molecules of the invention are particularly useful in preventing, treating, or ameliorating one or more symptoms associated with a disease, disorder, or infection. The molecules of the invention are particularly useful for the treatment or prevention of a disease or disorder where an enhanced efficacy of effector cell function (e.g., ADCC) mediated by FcγR is desired, e.g., cancer, infectious disease, and in enhancing the therapeutic efficacy of therapeutic antibodies the effect of which is mediated by ADCC.

### 1. CROSS-REFERENCE TO RELATED APPLICATIONS

This application claims priority to United States Patent Application Serial No. PCT/US2007/063548 (pending; filed on Mar. 8, 2007), and to 60/781,564 (presently lapsed; filed on Mar. 10, 2006), each of which applications is herein incorporated by reference in its entirety.

### 2. FIELD OF THE INVENTION

The present invention relates to molecules, particularly polypeptides, more particularly immunoglobulins (e.g., antibodies), comprising a variant heavy chain, which variant heavy chain comprises domains or regions, e.g., constant domains, a hinge region or an Fc region, from two or more IgG isotypes. The invention also encompasses molecules comprising a variant heavy chain, wherein said domains or regions thereof further comprise at least one amino acid modification relative to the wild-type domains or regions, such that the Fc region of said variant heavy chain binds an FcγR with an altered affinity relative to a comparable molecule comprising the wild-type heavy chain. The molecules of the invention are particularly useful in preventing, treating, or ameliorating one or more symptoms associated with a disease, disorder, or infection wherein a modification of antibody response, e.g., a modification of effector cell function mediated by antibody-FcγR interaction, is desired. The molecules of the invention also have particular use in enhancing the therapeutic efficacy of antibodies the effect of which is mediated by antibody-FcγR interaction.

### 3. BACKGROUND OF THE INVENTION

3.1 Fc Receptors and Their Roles in the Immune System

The interaction of antibody-antigen complexes with cells of the immune system results in a wide array of responses, ranging from effector functions such as antibody-dependent cytotoxicity, mast cell degranulation, and phagocytosis to immunomodulatory signals such as regulating lymphocyte proliferation and antibody secretion. All these interactions are initiated through the binding of the Fc domain of antibodies or immune complexes to specialized cell surface receptors on hematopoietic cells. The diversity of cellular responses triggered by antibodies and immune complexes results from the structural heterogeneity of Fc receptors and antibody isotypes. Fc receptors share structurally related ligand binding domains which presumably mediate intracellular signaling.

The Fe receptors, members of the immunoglobulin gene superfamily of proteins, are surface glycoproteins that can bind the Fe portion of immunoglobulin molecules. Each member of the family recognizes immunoglobulins of one or more isotypes through a recognition domain on the α chain of the Fe receptor. Fe receptors are defined by their specificity for immunoglobulin subtypes. Fe receptors for IgG are referred to as FcγR, for IgE as FεR, and for IgA as FcαR. Different accessory cells bear Fe receptors for antibodies of different isotype, and the isotype of the antibody determines which accessory cells will be engaged in a given response (reviewed by Ravetch J. V. et al 1991, Annu. Rev. Immunol. 9: 457-92; Gerber J. S. et al 2001 Microbes and Infection, 3: 131-139; Billadeau D. D. et al. 2002, The Journal of Clinical Investigation, 2(109): 161-1681; Ravetch J. V. et al. 2000, Science, 290: 84-89; Ravetch J. V. et al., 2001 Annu. Rev. Immunol. 19:275-90; Ravetch J. V. 1994, Cell, 78(4): 553-60). The different Fe receptors, the cells that express them, and their isotype specificity is summarized in Table 1 (adapted from Immunobiology: The Immune System in Health and Disease, 4^{th} ed. 1999, Elsevier Science Ltd/Garland Publishing, New York).

Fcγ Receptors

Each member of this family is an integral membrane glycoprotein, possessing extracellular domains related to a C2-set of immunoglobulin-related domains, a single membrane spanning domain and an intracytoplasmic domain of variable length. There are three known FcγRs, designated FcγRII(CD64), FcγRII(CD32), and FcγRIII(CD16). The three receptors are encoded by distinct genes; however, the extensive homology between the three family members suggest they arose from a common progenitor perhaps by gene duplication.

FcγRII(CD32)

FcγRII proteins are 40 KDa integral membrane glycoproteins which bind only the complexed IgG due to a low affinity for monomeric Ig (10⁶ M⁻¹). This receptor is the most widely expressed FcγR, present on all hematopoietic cells, including monocytes, macrophages, B cells, NK cells, neutrophils, mast cells, and platelets. FcγRII has only two immunoglobulin-like regions in its immunoglobulin binding chain and hence a much lower affinity for IgG than FcγRI. There are three human FcγRII genes (FcγRII-A, FcγRII-B, FcγRII-C), all of which bind IgG in aggregates or immune complexes.

Distinct differences within the cytoplasmic domains of FcγRII-A and FcγRII-B create two functionally heterogenous responses to receptor ligation. The fundamental difference is that the A isoform initiates intracellular signaling leading to cell activation such as phagocytosis and respiratory burst, whereas the β isoform initiates inhibitory signals, e.g., inhibiting B-cell activation.

IgG Subclasses

Four distinct subclasses of human IgG have been identified, designated IgG1, IgG2, IgG3 and IgG4. The subclasses are more than 95% homologous in the amino acid sequence of their heavy chain constant domains (“Fc domains”), with the majority of the differences found in the amino acid composition and structure of their respective hinge regions. The hinge region of the antibody determines the flexibility of the molecule and the resulting structure of the antigen-antibody complex, both of which are important in triggering effector functions such as receptor binding, complement activation and antibody dependent cellular cytotoxicity (“ADCC”). Discovery of minor variants in the amino-acid sequences of the heavy chains of the subclasses has also lead to the identification of multiple IgG allotypes. Four IgG1 allotypes, one IgG2 allotype and thirteen IgG3 allotypes have been identified; no IgG4 heavy chain allotypes have been discovered. Consistent with the variability in structure, the IgG subclasses exhibit differences in their physical properties such as susceptibility to proteolytic enzymes and receptor affinity. For example, IgG3 has an extended hinge region relative to the other IgG subclasses (at 62 amino acids, at least 4 times that of the other subclasses), which is thought to account for its greater susceptability to cleavage by cellular enzymes, e.g., plasmin, trypsin, pepsin, and its relatively reduced serum half-life (about one third of that of the other subclasses). IgG subclasses also exhibit marked differences in both FcγR affinity and functionality. IgG1 and IgG3 bind to all receptors, whereas IgG2 and IgG4 effectively bind to one receptor each. IgG2 binds only to one of the two allotypes of FcγRII-A, FcγRIIA-H131, and IgG4 binds to only FcγRI, although at a 10 times lower affinity than either IgG1 or IgG3. Thus, antibody effector functions dependent on Fc region-receptor binding, e.g., ADCC, will vary dependent on the specific IgG and FcγR. Additionally, unlike IgG1 and IgG3, IgG2 and IgG4 have only limited ability to bind C1q and therefore only poorly activate, if at all, the complement cascade.

Signaling through FcγRs

Both activating and inhibitory signals are transduced through the FcγRs following ligation. These diametrically opposing functions result from structural differences among the different receptor isoforms. Two distinct domains within the cytoplasmic signaling domains of the receptor called immunoreceptor tyrosine based activation motifs (ITAMs) or immunoreceptor tyrosine based inhibitory motifs (ITIMS) account for the different responses. The recruitment of different cytoplasmic enzymes to these structures dictates the outcome of the FcγR-mediated cellular responses. ITAM-containing FcγR complexes include FcγRI, FcγRIIA, FcγRIIIA, whereas ITIM-containing complexes only include FcγRIIB.

Human neutrophils express the FcγRIIA gene. FcγRIIA clustering via immune complexes or specific antibody cross-linking serves to aggregate ITAMs along with receptor-associated kinases which facilitate ITAM phosphorylation. ITAM phosphorylation serves as a docking site for Syk kinase, activation of which results in activation of downstream substrates (e.g., PI₃K). Cellular activation leads to release of proinflammatory mediators.

The FcγRIIB gene is expressed on B lymphocytes; its extracellular domain is 96% identical to FcγRIIA and binds IgG complexes in an indistinguishable manner. The presence of an ITIM in the cytoplasmic domain of FcγRIIB defines this inhibitory subclass of FcγR. Recently the molecular basis of this inhibition was established. When colligated along with an activating FcγR, the ITIM in FcγRIIB becomes phosphorylated and attracts the SH2 domain of the inosital polyphosphate 5′-phosphatase (SHIP), which hydrolyzes phosphoinositol messengers released as a consequence of ITAM-containing FcγR-mediated tyrosine kinase activation, consequently preventing the influx of intracellular Ca⁺⁺. Thus crosslinking of FcγRIIB dampens the activating response to FcγR ligation and inhibits cellular responsiveness. B cell activation, B cell proliferation and antibody secretion is thus aborted.

TABLE 1
Receptors for the Fc Regions of Immunoglobulin Isotypes

| | FcγRI | FcγRII-A | FcγRII-B2 | FcγRII-B1 | FcγRIII | | FcαRI |
|---|---|---|---|---|---|---|---|
| Receptor | (CD64) | (CD32) | (CD32) | (CD32) | (CD16) | FcεRI | (CD89) |
| Binding | IgG1 | IgG1 | IgG1 | IgG1 | IgG1 | IgE | IgA1, IgA2 |
| | 10⁸ M⁻¹ | 2 × 10⁶ M⁻¹ | 2 × 10⁶ M⁻¹ | 2 × 10⁶ M⁻¹ | 5 × 10⁵ M⁻¹ | 1010 M⁻¹ | 10⁷ M⁻¹ |
| Cell Type | Macrophages | Macrophages | Macrophages | B cells | NK cells | Mast cells | Macrophages |
| | Neutrophils | Neutrophils | Neutrophils | Mast cells | Eosinophil | Eosinophil | Neutrophils |
| | Eosinophils | Eosinophils | Eosinophils | | Macrophages | Basophils | Eosinophils |
| | Dendritic cells | Dendritic cells | | | Neutrophils | | |
| | | Platelets | | | Mast Cells | | |
| | | Langerhan cells | | | | | |
| Effect of | Uptake | Uptake | Uptake | No uptake | Induction of | Secretion of | Uptake |
| Ligation | Stimulation | Granule release | Inhibition of | Inhibition of | Killing | granules | Induction of |
| | Activation of | | Stimulation | Stimulation | | | killing |
| | respiratory burst | | | | | | |
| | Induction of | | | | | | |
| | killing | | | | | | |

3.2 Diseases of Relevance

3.2.1 Cancer

A neoplasm, or tumor, is a neoplastic mass resulting from abnormal uncontrolled cell growth which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell, 1976, Basic Pathology, 2d Ed., W.B. Saunders Co., Philadelphia, pp. 68-122). Cancer can arise in many sites of the body and behave differently depending upon its origin. Cancerous cells destroy the part of the body in which they originate and then spread to other part(s) of the body where they start new growth and cause more destruction.

More than 1.2 million Americans develop cancer each year. Cancer is the second leading case of death in the United States and if current trends continue, cancer is expected to be the leading cause of the death by the year 2010. Lung and prostate cancer are the top cancer killers for men in the United States. Lung and breast cancer are the top cancer killers for women in the United States. One in two men in the United States will be diagnosed with cancer at some time during his lifetime. One in three women in the United States will be diagnosed with cancer at some time during her lifetime.

A cure for cancer has yet to be found. Current treatment options, such as surgery, chemotherapy and radiation treatment, are oftentimes either ineffective or present serious side effects.

Cancer Therapy

Currently, cancer therapy may involve surgery, chemotherapy, hormonal therapy and/or radiation treatment to eradicate neoplastic cells in a patient (See, for example, Stockdale, 1998, “Principles of Cancer Patient Management”, in Scientific American: Medicine, vol. 3, Rubenstein and Federman, eds., Chapter 12, Section IV). Recently, cancer therapy could also involve biological therapy or immunotherapy. All of these approaches pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and although can be effective, is often used to prevent or delay recurrence of cancer after other treatments have removed the majority of the cancer cells. Biological therapies/immunotherapies are limited in number and may produce side effects such as rashes or swellings, flu-like symptoms, including fever, chills and fatigue, digestive tract problems or allergic reactions.

With respect to chemotherapy, there are a variety of chemotherapeutic agents available for treatment of cancer. A significant majority of cancer chemotherapeutics act by inhibiting DNA synthesis, either directly, or indirectly by inhibiting the biosynthesis of the deoxyribonucleotide triphosphate precursors, to prevent DNA replication and concomitant cell division (See, for example, Gilman et al., Goodman and Gilman's: The Pharmacological Basis of Therapeutics, Eighth Ed. (Pergamom Press, New York, 1990)). These agents, which include alkylating agents, such as nitrosourea, anti-metabolites, such as methotrexate and hydroxyurea, and other agents, such as etoposides, campathecins, bleomycin, doxorubicin, daunorubicin, etc., although not necessarily cell cycle specific, kill cells during S phase because of their effect on DNA replication. Other agents, specifically colchicine and the vinca alkaloids, such as vinblastine and vincristine, interfere with microtubule assembly resulting in mitotic arrest. Chemotherapy protocols generally involve administration of a combination of chemotherapeutic agents to increase the efficacy of treatment.

Despite the availability of a variety of chemotherapeutic agents, chemotherapy has many drawbacks (See, for example, Stockdale, 1998, “Principles Of Cancer Patient Management” in Scientific American Medicine, vol. 3, Rubenstein and Federman, eds., ch. 12, sect. 10). Almost all chemotherapeutic agents are toxic, and chemotherapy causes significant, and often dangerous, side effects, including severe nausea, bone marrow depression, immunosuppression, etc. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even those agents that act by mechanisms different from the mechanisms of action of the drugs used in the specific treatment; this phenomenon is termed pleiotropic drug or multidrug resistance. Thus, because of drug resistance, many cancers prove refractory to standard chemotherapeutic treatment protocols.

There is a significant need for alternative cancer treatments, particularly for treatment of cancer that has proved refractory to standard cancer treatments, such as surgery, radiation therapy, chemotherapy, and hormonal therapy. A promising alternative is immunotherapy, in which cancer cells are specifically targeted by cancer antigen-specific antibodies. Major efforts have been directed at harnessing the specificity of the immune response, for example, hybridoma technology has enabled the development of tumor selective monoclonal antibodies (See Green M. C. et al., 2000 Cancer Treat Rev., 26: 269-286; Weiner L M, 1999 Semin Oncol. 26(suppl. 14):43-51), and in the past few years, the Food and Drug Administration has approved the first MAbs for cancer therapy: Rituxin (anti-CD20) for non-Hodgkin's Lymphoma and HERCEPTIN® [anti-(c-erb-2/HER-2)] for metastatic breast cancer (Suzanne A. Eccles, 2001, Breast Cancer Res., 3: 86-90). However, the potency of antibody effector function, e.g., to mediate ADCC, is an obstacle to such treatment. Methods to improve the efficacy of such immunotherapy are thus needed.

3.2.2 Inflammatory Diseases and Autoimmune Diseases

Inflammation is a process by which the body's white blood cells and chemicals protect our bodies from infection by foreign substances, such as bacteria and viruses. It is usually characterized by pain, swelling, warmth and redness of the affected area. Chemicals known as cytokines and prostaglandins control this process, and are released in an ordered and self-limiting cascade into the blood or affected tissues. This release of chemicals increases the blood flow to the area of injury or infection, and may result in the redness and warmth. Some of the chemicals cause a leak of fluid into the tissues, resulting in swelling. This protective process may stimulate nerves and cause pain. These changes, when occurring for a limited period in the relevant area, work to the benefit of the body.

In autoimmune and/or inflammatory disorders, the immune system triggers an inflammatory response when there are no foreign substances to fight and the body's normally protective immune system causes damage to its own tissues by mistakenly attacking self. There are many different autoimmune disorders which affect the body in different ways. For example, the brain is affected in individuals with multiple sclerosis, the gut is affected in individuals with Crohn's disease, and the synovium, bone and cartilage of various joints are affected in individuals with rheumatoid arthritis. As autoimmune disorders progress destruction of one or more types of body tissues, abnormal growth of an organ, or changes in organ function may result. The autoimmune disorder may affect only one organ or tissue type or may affect multiple organs and tissues. Organs and tissues commonly affected by autoimmune disorders include red blood cells, blood vessels, connective tissues, endocrine glands (e.g., the thyroid or pancreas), muscles, joints, and skin. Examples of autoimmune disorders include, but are not limited to, Hashimoto's thyroiditis, pernicious anemia, Addison's disease, type 1 diabetes, rheumatoid arthritis, systemic lupus erythematosus, dermatomyositis, Sjogren's syndrome, dermatomyositis, lupus erythematosus, multiple sclerosis, autoimmune inner ear disease myasthenia gravis, Reiter's syndrome, Graves disease, autoimmune hepatitis, familial adenomatous polyposis and ulcerative colitis.

Rheumatoid arthritis (RA) and juvenile rheumatoid arthritis are types of inflammatory arthritis. Arthritis is a general term that describes inflammation in joints. Some, but not all, types of arthritis are the result of misdirected inflammation. Besides rheumatoid arthritis, other types of arthritis associated with inflammation include the following: psoriatic arthritis, Reiter's syndrome, ankylosing spondylitis arthritis, and gouty arthritis. Rheumatoid arthritis is a type of chronic arthritis that occurs in joints on both sides of the body (such as both hands, wrists or knees). This symmetry helps distinguish rheumatoid arthritis from other types of arthritis. In addition to affecting the joints, rheumatoid arthritis may occasionally affect the skin, eyes, lungs, heart, blood or nerves.

Rheumatoid arthritis affects about 1% of the world's population and is potentially disabling. There are approximately 2.9 million incidences of rheumatoid arthritis in the United States. Two to three times more women are affected than men. The typical age that rheumatoid arthritis occurs is between 25 and 50. Juvenile rheumatoid arthritis affects 71,000 young Americans (aged eighteen and under), affecting six times as many girls as boys.

Rheumatoid arthritis is an autoimmune disorder where the body's immune system improperly identifies the synovial membranes that secrete the lubricating fluid in the joints as foreign. Inflammation results, and the cartilage and tissues in and around the joints are damaged or destroyed. In severe cases, this inflammation extends to other joint tissues and surrounding cartilage, where it may erode or destroy bone and cartilage and lead to joint deformities. The body replaces damaged tissue with scar tissue, causing the normal spaces within the joints to become narrow and the bones to fuse together. Rheumatoid arthritis creates stiffness, swelling, fatigue, anemia, weight loss, fever, and often, crippling pain. Some common symptoms of rheumatoid arthritis include joint stiffness upon awakening that lasts an hour or longer; swelling in a specific finger or wrist joints; swelling in the soft tissue around the joints; and swelling on both sides of the joint. Swelling can occur with or without pain, and can worsen progressively or remain the same for years before progressing.

The diagnosis of rheumatoid arthritis is based on a combination of factors, including: the specific location and symmetry of painful joints, the presence of joint stiffness in the morning, the presence of bumps and nodules under the skin (rheumatoid nodules), results of X-ray tests that suggest rheumatoid arthritis, and/or positive results of a blood test called the rheumatoid factor. Many, but not all, people with rheumatoid arthritis have the rheumatoid-factor antibody in their blood. The rheumatoid factor may be present in people who do not have rheumatoid arthritis. Other diseases can also cause the rheumatoid factor to be produced in the blood. That is why the diagnosis of rheumatoid arthritis is based on a combination of several factors and not just the presence of the rheumatoid factor in the blood.

The typical course of the disease is one of persistent but fluctuating joint symptoms, and after about 10 years, 90% of sufferers will show structural damage to bone and cartilage. A small percentage will have a short illness that clears up completely, and another small percentage will have very severe disease with many joint deformities, and occasionally other manifestations of the disease. The inflammatory process causes erosion or destruction of bone and cartilage in the joints. In rheumatoid arthritis, there is an autoimmune cycle of persistent antigen presentation, T-cell stimulation, cytokine secretion, synovial cell activation, and joint destruction. The disease has a major impact on both the individual and society, causing significant pain, impaired function and disability, as well as costing millions of dollars in healthcare expenses and lost wages. (See, for example, the NIH website and the NIAID website).

Currently available therapy for arthritis focuses on reducing inflammation of the joints with anti-inflammatory or immunosuppressive medications. The first line of treatment of any arthritis is usually anti-inflammatories, such as aspirin, ibuprofen and Cox-2 inhibitors such as celecoxib and rofecoxib. “Second line drugs” include gold, methotrexate and steroids. Although these are well-established treatments for arthritis, very few patients remit on these lines of treatment alone. Recent advances in the understanding of the pathogenesis of rheumatoid arthritis have led to the use of methotrexate in combination with antibodies to cytokines or recombinant soluble receptors. For example, recombinant soluble receptors for tumor necrosis factor (TNF)-α have been used in combination with methotrexate in the treatment of arthritis. However, only about 50% of the patients treated with a combination of methotrexate and anti-TNF-α agents such as recombinant soluble receptors for TNF-α show clinically significant improvement. Many patients remain refractory despite treatment. Difficult treatment issues still remain for patients with rheumatoid arthritis. Many current treatments have a high incidence of side effects or cannot completely prevent disease progression. So far, no treatment is ideal, and there is no cure. Novel therapeutics are needed that more effectively treat rheumatoid arthritis and other autoimmune disorders.

3.2.3 Infectious Diseases

Infectious agents that cause disease fall into five groups: viruses, bacteria, fungi, protozoa, and helminths (worms). The remarkable variety of these pathogens has caused the natural selection of two crucial features of adaptive immunity. First, the advantage of being able to recognize a wide range of different pathogens has driven the development of receptors on B and T cells of equal or greater diversity. Second, the distinct habitats and life cycles of pathogens have to be countered by a range of distinct effector mechanisms. The characteristic features of each pathogen are its mode of transmission, its mechanism of replication, its pathogenesis or the means by which it causes disease, and the response it elicits.

The record of human suffering and death caused by smallpox, cholera, typhus, dysentery, malaria, etc. establishes the eminence of the infectious diseases. Despite the outstanding successes in control afforded by improved sanitation, immunization, and antimicrobial therapy, the infectious diseases continue to be a common and significant problem of modern medicine. The most common disease of mankind, the common cold, is an infectious disease, as is the feared modern disease AIDS. Some chronic neurological diseases that were thought formerly to be degenerative diseases have proven to be infectious. There is little doubt that the future will continue to reveal the infectious diseases as major medical problems.

An enormous number of human and animal diseases result from virulent and opportunistic infections from any of the above mentioned infectious agents (see Belshe (Ed.) 1984 Textbook of Human Virology, PSG Publishing, Littleton, Mass.).

One category of infectious diseases are viral infections for example. Viral diseases of a wide array of tissues, including the respiratory tract, CNS, skin, genitourinary tract, eyes, ears, immune system, gastrointestinal tract, and musculoskeletal system, affect a vast number of humans of all ages (see Table 328-2 In: Wyngaarden and Smith, 1988, Cecil Textbook of Medicine, 18^{th} Ed., W.B. Saunders Co., Philadelphia, pp. 1750-1753). Although considerable effort has been invested in the design of effective anti-viral therapies, viral infections continue to threaten the lives of millions of people worldwide. In general, attempts to develop anti-viral drugs have focused on several stages of viral life cycle (See e.g., Mitsuya et al., 1991, FASEB J. 5:2369-2381, discussing HIV). However, a common drawback associated with using of many current anti-viral drugs is their deleterious side effects, such as toxicity to the host or resistance by certain viral strains.

### 4. SUMMARY OF THE INVENTION

The present invention relates to modifications of antibody functionality, e.g., effector function, in immunoglobulins with Fe regions from IgG isotypes IgG2, IgG3 or IgG4. Such modifications are effected, in part, by modification of the heavy chain, such that the Fe region thereof exhibits altered affinities for FcγR receptors (e.g., activating FcγRs, inhibitory FcγRs). In vivo animal modeling and clinical experiments indicate that the Fe region and Fc-FcγR interactions may play an essential role in determining the outcome of monoclonal antibody therapy. Current approaches to optimize therapeutic antibody functionality (e.g., antibody-dependent cell mediated cytotoxicity (ADCC), complement dependent cytotoxicity (CDC) activity) have focused on amino acid modification or modification of the glycosylation state of the native Fe region. In contrast, the present invention is based, in part, on the modification of antibody functionality through the creation of a variant heavy chain by combining heavy chain domains or regions (e.g., CH domains, hinge region, Fe region) from two or more IgG isotypes. Independent selection of these domains or regions from the varying IgG isotypes allows the combination of their disparate in vivo properties, e.g., altered complement fixation or serum half-life, into a single molecule. The domains or regions comprising the variant heavy chain may be altered by amino acid modification relative to the wild type domain or region to further refine the resulting effector function of the molecule of the invention.

The invention relates to molecules, preferably polypeptides, and more preferably immunoglobulins (e.g., antibodies), comprising a variant heavy chain, wherein said variant heavy chain comprises domains or regions from two or more IgG isotypes. In certain embodiments, the invention relates to molecules comprising CH1 and hinge domains of an IgG1 and an Fe region of IgG2, IgG3 or IgG4. The invention further encompasses molecules comprising variant heavy chains having domains or regions from IgG2, IgG3 or IgG4, and one or more amino acid modifications (e.g., substitutions, but also including insertions or deletions) in one or more regions, which modifications alter, e.g., increase or decrease, the affinity of the Fe region of said variant heavy chain for an FcγR. Preferably, said one or more amino acid modifications increase the affinity of the Fe region of said variant heavy chain for FcγRIIIA and/or FcγRIIA. In a preferred embodiment, the molecules of the invention further specifically bind FcγRIIB (via the Fe region) with a lower affinity than a comparable molecule (i.e., having the same amino acid sequence as the molecule of the invention except for the one or more amino acid modifications in the heavy chain) comprising the wild-type heavy chain and/or Fe region binds FcγRIIB. In some embodiments, the invention encompasses molecules with variant heavy chains having the Fe region of IgG2, IgG3 or IgG4 and one or more amino acid modifications, which modifications increase or enhance the affinity of the Fc region of said variant heavy chain for FcγRIIIA and/or FcγRIIA and/or FcγRIIB relative to a comparable molecule with a wild type heavy chain having an Fc region of the same isotype. In other embodiments, the invention encompasses molecules with variant heavy chains having the Fc region of IgG2, IgG3 or IgG4, and one or more amino acid modifications, which modifications increase the affinity of the Fc region of said variant heavy chain for FcγRIIIA and/or FcγRIIA but do not alter the affinity of the Fc region of said variant heavy chain for FcγRIIB relative to a comparable molecule with a wild type heavy chain and/or Fc region of the same isotype. A preferred embodiment is a variant heavy chain comprising an Fc region of IgG2, IgG3 or IgG4 that has enhanced affinity for FcγRIIIA and FcγRIIA but reduced affinity for FcγRIIB relative to a comparable molecule with a wild type heavy chain and/or Fc region of the same isotype.

The heavy chain variants of the present invention may be combined with other modifications to the domains or regions thereof, e.g., Fc region, including but not limited to modifications that alter effector function. The invention encompasses combining a heavy chain variant of the invention with other heavy chain modifications to provide additive, synergistic, or novel properties in antibodies or Fc fusions. Preferably, the heavy chain variants of the invention enhance the phenotype of the modification with which they are combined. For example, if a heavy chain variant of the invention is combined with a mutant known to bind FcγRIIIA with a higher affinity than a comparable molecule comprising a wild type heavy chain region; the combination with a mutant of the invention results in a greater fold enhancement in FcγRIIIA affinity.

The molecules of the invention comprising IgG2, IgG3 or IgG4 Fc domains may be further modified as disclosed in Duncan et al., 1988, Nature 332:563-564; Lund et al., 1991, J. Immunol. 147:2657-2662; Lund et al., 1992, Mol Immunol 29:53-59; Alegre et al., 1994, Transplantation 57:1537-1543; Hutchins et al., 1995, Proc Natl. Acad Sci USA 92:11980-11984; Jefferis et al., 1995, Immunol Lett. 44:111-117; Lund et al., 1995, Faseb J 9:115-119; Jefferis et al., 1996, Immunol Lett 54:101-104; Lund et al., 1996, J Immunol 157:49634969; Armour et al., 1999, Eur J Immunol 29:2613-2624; Idusogie et al., 2000, J Immunol 164:41784184; Reddy et al., 2000, J Immunol 164:1925-1933; Xu et al., 2000, Cell Immunol 200:16-26; Idusogie et al., 2001, J Immunol 166:2571-2575; Shields et al., 2001, J Biol Chem 276:6591-6604; Jefferis et al., 2002, Immunol Lett 82:57-65; Presta et al., 2002, Biochem Soc Trans 30:487-490); U.S. Pat. No. 5,624,821; U.S. Pat. No. 5,885,573; U.S. Pat. No. 6,194,551; PCT WO 00/42072; PCT WO 99/58572; each of which is incorporated herein by reference in its entirety.

The invention encompasses molecules that are homodimers or heterodimers of heavy chains or regions thereof, e.g., Fc regions. Heterodimers comprising heavy chains or Fc regions refer to molecules where the two heavy chains or Fc regions have the same or different sequences. In some embodiments, in the heterodimeric molecules comprising variant heavy chains and/or Fc regions, each chain has one or more different modifications from the other chain. In other embodiments, in the heterodimeric molecules comprising variant heavy chains and/or Fc regions, one heavy chain contains a wild-type region and the other heavy chain comprises one or more modifications. Methods of engineering heterodimeric molecules are known in the art and encompassed within the invention.

In some embodiments, the invention encompasses molecules comprising a variant heavy chain which contains an Fc region of IgG2, IgG3 or IgG4, having at least one amino acid modification relative to a wild type heavy chain containing an Fc region of the same isotype, which Fc region of said variant heavy chain does not bind any FcγR or binds with a reduced affinity, relative to a comparable molecule comprising the wild-type heavy chain containing the Fc region of the same isotype and/or Fc region, as determined by standard assays (e.g., in vitro assays) known to one skilled in the art. In a specific embodiment, the invention encompasses molecules comprising a variant heavy chain having the Fc region from IgG2, IgG3 or IgG4, wherein said variant heavy chain comprises at least one amino acid modification relative to a wild type heavy chain having an Fc region of the same isotype, which Fc region of the variant heavy chain binds one FcγR, wherein said FcγR is FcγIIIA. In another specific embodiment, the invention encompasses molecules comprising a variant heavy chain having the Fc region of IgG2, IgG3 or IgG4, wherein said variant heavy chain comprises at least one amino acid modification relative to a wild type heavy chain having an Fc region of the same isotype, which Fc region of the variant heavy chain binds only one FcγR, wherein said FcγR is FcγRIIA. In yet another embodiment, the invention encompasses molecules comprising a variant heavy chain having the Fc region of IgG2, IgG3 or IgG4, wherein said variant heavy chain comprises at least one amino acid modification relative to a wild type heavy chain having an Fc region of the same isotype, which Fc region of the variant heavy chain binds only one FcγR, wherein said FcγR is FcγRIIB.

The affinities and binding properties of the molecules of the invention for an FcγR are initially determined using in vitro assays (biochemical or immunological based assays) known in the art for determining heavy chain-antibody receptor interactions, in particular, Fc-FcγR interactions, i.e., specific binding of an Fe region to an FcγR, including but not limited to ELISA assay, surface plasmon resonance assay, immunoprecipitation assays (See Section 5.2). Preferably, the binding properties of the molecules of the invention are also characterized by in vitro functional assays for determining one or more FcγR mediator effector cell functions (See Section 5.3). In most preferred embodiments, the molecules of the invention have similar binding properties in in vivo models (such as those described and disclosed herein) as those in in vitro based assays. However, the present invention does not exclude molecules of the invention that do not exhibit the desired phenotype in in vitro based assays but do exhibit the desired phenotype in vivo.

In certain embodiments, the invention encompasses a molecule comprising a variant heavy chain having the Fe region of IgG2, IgG3 or IgG4, wherein said variant heavy chain comprises at least one amino acid modification relative to a wild type heavy chain having an Fe region of the same isotype, which Fe region of said variant heavy chain specifically binds FcγRIIIA with a greater affinity than a comparable molecule comprising the wild-type heavy chain an/or Fe region binds FcγRIIIA, provided that said when said variant heavy chain comprises the CH1 domain and hinge region of IgG1 and Fe region of IgG2, said variant heavy chain does not solely have a substitution at position 233 with glutamic acid, at position 234 with leucine, at position 235 with leucine and an insertion at position 237 with glycine; or a substitution at position 234 with leucine, at position 235 with leucine, and an insertion at position 237 with glycine. The amino acid positions recited herein are numbered according to the EU index as set forth in Kabat et al., Sequence of Proteins of Immunological Interest, 5^{th} Ed. Public Health Service, NH1, MD (1991), expressly incorporated herein by reference.

In a preferred specific embodiment, the invention encompasses a molecule comprising a variant heavy chain having the Fe region of IgG2, IgG3 or IgG4, wherein said variant heavy chain comprises at least one amino acid modification relative to a wild type heavy chain having an Fe region of the same isotype, such that said molecule has an altered affinity for an FcγR, provided that said variant heavy chain does not soley have or does not solely comprise a substitution or modification at positions that make a direct contact with FcγR based on crystallographic and structural analysis of Fc-FcγR interactions such as those disclosed by Sondermann et al., (2000 Nature, 406: 267-273, which is incorporated herein by reference in its entirety). Examples of positions within the heavy chain that make a direct contact with FcγR are amino acids 234-239 (hinge region), amino acids 265-269 (B/C loop), amino acids 297-299 (C′/E loop), and amino acids 327-332 (F/G) loop. In some embodiments, the molecules of the invention comprising variant heavy chains comprise modification of at least one residue that does not make a direct contact with an FcγR based on structural and crystallographic analysis, e.g., is not within the Fc-FcγR binding site.

In a specific embodiment, molecules of the invention comprise a variant heavy chain having the Fc region of IgG2, IgG3 or IgG4, wherein said variant heavy chain comprises at least one amino acid modification (e.g., substitutions) relative to a wild type heavy chain having an Fc region of the same isotype, which modifications increase the affinity of the variant heavy chain for FcγRIIIA and/or FcγRIIA by at least 2-fold, relative to a comparable molecule comprising a wild-type heavy chain having an Fc region of the same isotype. In certain embodiments, molecules of the invention comprise a variant heavy chain having the Fc region of IgG2, IgG3 or IgG4, wherein said variant heavy chain comprises at least one amino acid modification (e.g., substitutions) relative to a wild type heavy chain having an Fc region of the same isotype, which modifications increase the affinity of the variant heavy chain for FcγRIIIA and/or FcγRIIA by greater than 2-fold, at least 4-fold, at least 5-fold, at least 6-fold, at least 8-fold, or at least 10-fold relative to a comparable molecule comprising a wild-type heavy chain having an Fc region of the same isotype. In other embodiments of the invention, molecules of the invention comprising a variant heavy chain having the Fc region of IgG2, IgG3 or IgG4 specifically bind FcγRIIIA and/or FcγRIIA with at least 65%, at least 75%, at least 85%, at least 95%, at least 100%, at least 150%, at least 200% greater affinity relative to a molecule comprising a wild-type heavy chain having an Fc region of the same isotype. Such measurements are preferably in vitro assays.

The invention encompasses molecules with altered affinities for the activating and/or inhibitory Fcγ receptors. In particular, the invention contemplates molecules with variant heavy chains having the Fc region of IgG2, IgG3 or IgG4, comprising one amino acid modifications, which modifications increase the affinity of the Fc regions of the variant heavy chain for FcγRIIB but decrease the affinity of the Fc regions of the variant heavy chain for FcγRIIIA and/or FcγRIIA, relative to a comparable molecule with a wild-type heavy chain. In other embodiments, the invention encompasses molecules with variant heavy chains having the Fc region of IgG2, IgG3 or IgG4, comprising one or more amino acid modifications, which modifications decrease the affinity of the Fc regions of the variant heavy chain for FcγRIIB and also decrease the affinity of the Fc regions of the variant heavy chains for FcγRIIIA and/or FcγRIIA relative to a comparable molecule with a wild-type heavy chain. In yet other embodiments, the invention encompasses molecules with variant heavy chains having the Fc region of IgG2, IgG3 or IgG4, comprising one or more amino acid modifications, which modifications increase the affinity of the Fc region of the variant heavy chain for FcγRIIB and also increase the affinity of the Fc region of the variant heavy chains for FcγRIIIA and/or FcγRIIA relative to a comparable molecule with a wild-type heavy chain. In yet other embodiments, the invention encompasses molecules with variant heavy chains having the Fc region of IgG2, IgG3 or IgG4, comprising one or more amino acid modifications, which modifications decrease the affinity of the Fc region of the variant heavy chain for FcγRIIIA and/or FcγRIIA but do not alter the affinity of the Fc region of the variant heavy chain for FcγRIIB relative to a comparable molecule with a wild-type heavy chain. In yet other embodiments, the invention encompasses molecules with variant heavy chains having the Fc region of IgG2, IgG3 or IgG4, comprising one or more amino acid modifications, which modifications increase the affinity of the Fc region of the variant heavy chain for FcγRIIIA and/or FcγRIIA but reduce the affinity of the variant Fc region for FcγRIB relative to a comparable molecule with a wild-type Fc region.

In most preferred embodiments, the molecules of the invention with altered affinities for activating and/or inhibitory receptors having variant heavy chains containing the Fc region of IgG2, IgG3 or IgG4, have one or more amino acid modifications, wherein said one or more amino acid modification is a substitution at position 288 with asaparagine, at position 330 with serine and at position 396 with leucine (MgFc10) (See Tables 6 & 7); or a substitution at position 334 with glutamic acid, at position 359 with asparagine, and at position 366 with serine (MgFc13); or a substitution at position 316 with aspartic acid, at position 378 with valine, and at position 399 with glutamic acid (MgFc27); or a substitution at position 392 with threonine, and at position 396 with leucine (MgFc38); or a substitution at position 221 with glutamic acid, at position 270 with glutamic acid, at position 308 with alanine, at position 311 with histidine, at position 396 with leucine, and at position 402 with aspartic acid (MgFc42); or a substitution at position 240 with alanine, and at position 396 with leucine (MgFc52); or a substitution at position 410 with histidine, and at position 396 with leucine (MgFc53); or a substitution at position 243 with leucine, at position 305 with isoleucine, at position 378 with aspartic acid, at position 404 with serine, and at position 396 with leucine (MgFc54); or a substitution at position 255 with isoleucine, and at position 396 with leucine (MgFc55); or a substitution at position 370 with glutamic acid and at position 396 with leucine (MgFc59); or a substitution at position 243 with leucine, at position 292 with proline, at position 300 with leucine, at position 305 with isoleucine, and at position 396 with leucine (MgFc88); or a substitution at position 243 with leucine, at position 292 with proline, at position 300 with leucine, and at position 396 with leucine (MgFc88A); or a substitution at position 243 with leucine, at position 292 with proline, and at position 300 with leucine (MgFc155).

One aspect of the invention provides a method for cloning mutations originally identified in the context of an IgG1 heavy chain or an IgG1 Fc region into molecules comprising heavy chains harboring having the Fc region of IgG2, IgG3 or IgG4. In certain embodiments, the original mutations were identified in in vitro studies as conferring on the variant IgG1 heavy chain or variant IgG1 Fc region a desirable binding property (e.g., the ability to mediate binding to FcγRIIIA with a greater affinity than a comparable polypeptide comprising a wild-type heavy chain or Fc region).

In preferred embodiments, the molecules of the invention are screened or charactered using one or more biochemical based assays, preferably in a high throughput manner. The one or more biochemical assays can be any assay known in the art for identifying heavy chain-receptor interactions, and in particular, Fc-FcγR interations (i.e., specific binding of an Fc region to an FcγR) including, but not limited to, an ELISA assay, surface plasmon resonance assays, immunoprecipitation assay, affinity chromatography, or equilibrium dialysis. In some embodiments, the molecules of the invention comprising variant heavy chains having the Fc region of IgG2, IgG3 or IgG4, and exhibiting altered FcγR affinities (e.g., enhanced FcγRIIIA affinity) are characterized or screened using one or more biochemical based assays described herein in combination with one or more functional assays, preferably in a high throughput manner. The functional based assays can be any assay known in the art for characterizing one or more FcγR mediated effector cell function such as those described herein in Section 5.3. Non-limiting examples of effector cell functions that can be used in accordance with the methods of the invention, include but are not limited to, antibody-dependent cell mediated cytotoxicity (ADCC), antibody-dependent phagocytosis, phagocytosis, opsonization, opsonophagocytosis, cell binding, rosetting, C1q binding, and complement dependent cell mediated cytotoxicity. In some embodiments, the molecules of the invention are screened or characterized using biochemical based assays in combination or in parallel with one or more functional based assays, preferably in a high throughput manner.

A preferred method for measuring the heavy chain-FcγR interaction in accordance with the invention is an assay developed by the inventors that allows detection and quantitation of the Fc-FcγR interaction despite the inherently weak affinity of the receptor for its ligand, e.g., in the micromolar range for FcγRIIB and FcγRIIIA. The method involves the formation of an FcγR complex (e.g., FcγRIIIA, FcγRIIB) that has an improved avidity for the Fc region, relative to an uncomplexed FcγR. The method comprises: (i) producing a fusion protein, such that a 15 amino acid AVITAG sequence operably linked to the soluble region of FcγR; (ii) biotinylating the protein produced using an E. coli BirA enzyme; (iii) mixing the biotinylated protein produced with streptaividn-phycoerythrin in an appropriate molar ratio, such that a tetrameric FcγR complex is formed. Such methods are described in detail in International Application WO04/063351 and U.S. Patent Application Publications 2005/0037000 and 2005/0064514, concurrent applications of the inventors, each of which is incorporated by reference herein in its entirety.

In a preferred embodiment of the invention, molecules comprising a variant heavy chain having the Fc region of IgG2, IgG3 or IgG4 bind the tetrameric FcγR complexes with at least an 8-fold higher affinity than they bind the monomeric uncomplexed FcγR. The binding of molecules comprising a variant heavy chain having the Fc region of IgG2, IgG3 or IgG4 to the tetrameric FcγR complexes may be determined using standard techniques known to those skilled in the art, such as for example, fluorescence activated cell sorting (FACS), radioimmunoassays, ELISA assays, etc.

The invention encompasses the use of the immune complexes formed according to the methods described above for determining the functionality of molecules comprising molecules comprising a variant heavy chain having the Fc region of IgG2, IgG3 or IgG4 in cell-based or cell-free assays.

In a specific embodiment, the invention provides modified immunoglobulins comprising a variant heavy chains or portions thereof having the Fc region of IgG2, IgG3, or IgG4, which immunoglobulins have enhanced affinity for FcγRIIIA and/or FcγRIIA. In certain embodiments, the invention encompasses immunoglobulins that comprise domains or regions from two or more IgG isotypes. Such immunoglobulins also include molecules that naturally contain FcγR binding regions (e.g., FcγRIIIA and/or FcγRIIB binding regions), or immunoglobulin derivatives that have been engineered to contain an FcγR binding region (e.g., FcγRIIIA and/or FcγRIIB binding regions). The modified immunoglobulins of the invention include any immunoglobulin molecule that binds, preferably, immunospecifically, i.e., competes off non-specific binding as determined by immunoassays well known in the art for assaying specific antigen-antibody binding, an antigen and contains an FcγR binding region (e.g., a FcγRIIIA and/or FcγRIIB binding region). Such antibodies include, but are not limited to, polyclonal, monoclonal, bi-specific, multi-specific, human, humanized, chimeric antibodies, single chain antibodies, Fab fragments, F(ab′)₂ fragments, disulfide-linked Fvs, and fragments containing either a VL or VH domain or even a complementary determining region (CDR) that specifically binds an antigen, in certain cases, engineered to contain or fused to an FcγR binding region.

In certain embodiment, the invention encompasses immunoglobulins comprising a variant heavy chain having the Fc region of IgG2, IgG3, or IgG4, which immunoglobulins exhibit enhanced affinity for FcγRIIIA and/or FcγRIIA such that the immunoglobulin has an enhanced effector function, e.g., antibody dependent cell mediated cytotoxicity. The effector function of the molecules of the invention can be assayed using any assay described herein or known to those skilled in the art. In some embodiments, immunoglobulins comprising a variant heavy chain having the Fc region of IgG2, IgG3 or IgG4, and an enhanced affinity for FcγRIIIA and/or FcγRIIA also have an enhanced ADCC activity relative to wild-type immunoglobulin comprising the wild type heavy chain and/or the Fc region of the same isotype by at least 2-fold, at least 4-fold, at least 8-fold, at least 10-fold, at least 50-fold, or at least 100-fold.

In a specific embodiment, the invention encompasses a molecule comprising a variant heavy chain having the Fc region of IgG2, IgG3 or IgG4, wherein said variant heavy chain comprises at least one amino acid modification relative to the wild-type heavy chain containing an Fc region of the same isotype such that the molecule has an enhanced effector activity, provided said one or more amino acid modifications includes substitutions at one or more positions. In a specific embodiment, the variant heavy chain comprises a leucine at position 247, a lysine at position 421, or a glutamic acid at position 270. In other specific embodiments, the variant heavy chain comprises a leucine at position 247, a lysine at position 421 and a glutamic acid at position 270 (MgFc31/60); a threonine at position 392, a leucine at position 396, a glutamic acid at position 270, and a leucine at position 243 (MgFc38/60/F243L); a histidine at position 419, a leucine at position 396, and a glutamic acid at position 270 (MGFc51/60); an alanine at position 240, a leucine at position 396, and a glutamic acid at position 270 (MGFc52/60); a histidine at position 419, a leucine at position 396, a glutamic acid at position 270, and a leucine at position 243 (MGFc51/60/F243L); a lysine at position 255 and a leucine at position 396 (MgFc55); a lysine at position 255, a leucine at position 396, and a glutamic acid at position 270 (MGFc55/60); a lysine at position 255, a leucine at position 396, a glutamic acid at position 270, and a lysine at position 300 (MGFc55/60/Y300L); a lysine at position 255, a leucine at position 396, a glutamic acid at position 270, and a leucine at position 243 (MgFc55/60/F243L); a lysine at position 255, a leucine at position 396, a glutamic acid at position 270, and a glycine at position 292 (MgFc55/60/R292G); a glutamic acid at position 370, a leucine at position 396, and a glutamic acid at position 270 (MGFc59/60); a glutamic acid at position 270, an aspartic acid at position 316, and a glycine at position 416 (MgFc71); a leucine at position 243, a proline at position 292, an isoleucine at position 305, and a leucine at position 396 (MGFc74/P396L); a leucine at position 243, a glutamic acid at position 270, a glycine at position 292, and a leucine at position 396; a leucine at position 243, a lysine at position 255, a glutamic acid at position 270, and a leucine at position 396; or a glutamine at position 297.

The invention encompasses engineering human or humanized therapeutic antibodies (e.g., tumor specific monoclonal antibodies) by substituting or replacing one or more regions/domains of the native heavy chain with one or more corresponding regions/domains of a heterologous IgG isotype and by modifying one or more amino acid residues of the resultant heavy chain (e.g., substitution, insertion, deletion), which modifications modulate the affinity of the therapeutic antibody for an FcγR activating receptor and/or an FcγR inhibitory receptor. In one embodiment, the invention relates to engineering human or humanized therapeutic antibodies (e.g., tumor specific monoclonal antibodies) by substituting or replacing one or more regions/domains of the native heavy chain with one or more corresponding regions/domains of a heterologous IgG isotype and by modifying one or more amino acid residues of the resultant heavy chain (e.g., substitution, insertion, deletion), which modifications increase the affinity of the Fc region of said variant heavy chain for FcγRIIIA and/or FcγRIIA. In another embodiment, the invention relates to engineering human or humanized therapeutic antibodies (e.g., tumor specific monoclonal antibodies) by substituting or replacing one or more regions/domains of the native heavy chain with one or more corresponding regions/domains of a heterologous IgG isotype and by modifying one or more amino acid residues of the resultant heavy chain (e.g., substitution, insertion, deletion), which modification increases the affinity of the Fc region of said variant heavy chain for FcγRIIIA and/or FcγRIIA and further decreases the affinity of the Fc region for FcγRIIB. The engineered therapeutic antibodies may further have an enhanced effector function, e.g., enhanced ADCC activity, phagocytosis activity, etc., as determined by standard assays known to those skilled in the art.

In a specific embodiment, the invention encompasses engineering a humanized monoclonal antibody specific for Her2/neu protooncogene (e.g., Ab4D5 humanized antibody as disclosed in Carter et al., 1992, Proc. Natl. Acad. Sci. USA 89:4285-9) by substituting or replacing one or more regions/domains of the native heavy chain with one or more corresponding regions/domains of a heterologous IgG isotype and by modifying one or more amino acid residues of the resultant heavy chain (e.g., substitution, insertion, deletion,) which modification increases the affinity of the Fc region of the heavy chain for FcγRIIIA and/or FcγRIIA. In another specific embodiment, modification of the humanized Her2/neu monoclonal antibody may also decrease the affinity of the Fc region of the heavy chain for FcγRIIB. In yet another specific embodiment, the engineered humanized monoclonal antibodies specific for Her2/neu may further have an enhanced effector function as determined by standard assays known in the art and disclosed and exemplified herein.

In another specific embodiment, the invention encompasses engineering a mouse human chimeric anti-CD20 monoclonal antibody, 2H7 by substituting or replacing one or more regions/domains of the native heavy chain with one or more corresponding regions/domains of a heterologous IgG isotype and by modifying one or more amino acid residues of the resultant heavy chain (e.g., substitution, insertion, deletion), which modification increases the affinity of the Fc region of the variant heavy chain for FcγRIIIA and/or FcγRIIA. In another specific embodiment, modification of the anti-CD20 monoclonal antibody, 2H7 may also further decrease the affinity of the Fc region of the variant heavy chain for FcγRIIB. In yet another specific embodiment, the engineered anti-CD20 monoclonal antibody, 2H7 may further have an enhanced effector function as determined by standard assays known in the art and disclosed and exemplified herein.

In another specific embodiment, the invention encompasses engineering an anti-FcγRIIB antibody including but not limited to any of the antibodies disclosed in U.S. Provisional Application No. 60/403,266 filed on Aug. 12, 2002 and U.S. application Ser. No. 10/643,857 filed on Aug. 14, 2003, having Attorney Docket No. 011183-010-999, by substituting or replacing one or more regions/domains of the native heavy chain with one or more corresponding regions/domains of a heterologous IgG isotype and by modifying one or more amino acid residues of the resultant heavy chain (e.g., substitution, insertion, deletion), which modification increases the affinity of the Fc region for FcγRIIIA and/or FcγRIIA. Examples of anti-FcγRIIB antibodies that may be engineered in accordance with the methods of the invention are 2B6 monoclonal antibody having ATCC accession number PTA-4591 and 3H7 having ATCC accession number PTA-4592 (deposited at ATCC, 10801 University Boulevard, Manassas, Va. 02209-2011, which are incorporated herein by reference. In another specific embodiment, modification of the anti-FcγRIIB antibody may also further decrease the affinity of the Fc region of the variant heavy chain for FcγRIIB. In yet another specific embodiment, the engineered anti-FcγRIIB antibody may further have an enhanced effector function as determined by standard assays known in the art and disclosed and exemplified herein. In a specific embodiment, the 2B6 monoclonal antibody engineered in accordance with the invention comprises a modification at position 334 with glutamic acid, at position 359 with asparagine, and at position 366 with serine (MgFc13); or a substitution at position 316 with aspartic acid, at position 378 with valine, and at position 399 with glutamic acid (MgFc27); or a substitution at position 243 with isoleucine, at position 379 with leucine, and at position 420 with valine (MgFc29); or a substitution at position 392 with threonine and at position 396 with leucine (MgFc38); or a substitution at position 221 with glutamic acid, at position 270 with glutamic acid, at position 308 with alanine, at position 311 with histidine, at position 396 with leucine, and at position 402 with aspartic (MgFc42); or a substitution at position 410 with histidine, and at position 396 with leucine (MgFc53); or a substitution at position 243 with leucine, at position 305 with isoleucine, at position 378 with aspartic acid, at position 404 with serine, and at position 396 with leucine (MgFc54); or a substitution at position 255 with isoleucine, and at position 396 with leucine (MgFc55); or a substitution at position 370 with glutamic acid, and at position 396 with leucine (MgFc59); or a substitution at position 243 with leucine, at position 292 with proline, at position 300 with leucine, at position 305 with isoleucine, and at position 396 with leucine (MgFc88); or a substitution at position 243 with leucine, at position 292 with proline, at position 300 with leucine, and at position 396 with leucine (MgFc88A); or a substitution at position 243 with leucine, at position 292 with proline, and at position 300 with leucine (MgFc155).

The present invention also includes polynucleotides that encode a molecule of the invention, including polypeptides and antibodies, identified by the methods of the invention. The polynucleotides encoding the molecules of the invention may be obtained, and the nucleotide sequence of the polynucleotides determined, by any method known in the art.

The invention relates to an isolated nucleic acid encoding a molecule of the invention. The invention also provides a vector comprising said nucleic acid. The invention further provides host cells containing the vectors or polynucleotides of the invention.

The invention further provides methods for the production of the molecules of the invention. The molecules of the invention, including polypeptides and antibodies, can be produced by any method known to those skilled in the art, in particular, by recombinant expression. In a specific embodiment, the invention relates to a method for recombinantly producing a molecule of the invention, said method comprising: (i) culturing in a medium a host cell comprising a nucleic acid encoding said molecule, under conditions suitable for the expression of said molecule; and (ii) recovery of said molecule from said medium.

The molecules identified in accordance with the methods of the invention are useful in preventing, treating, or ameliorating one or more symptoms associated with a disease, disorder, or infection. The molecules of the invention are particularly useful for the treatment or prevention of a disease or disorder where an enhanced efficacy of effector cell function (e.g., ADCC) mediated by FcγR is desired, e.g., cancer, infectious disease, and in enhancing the therapeutic efficacy of therapeutic antibodies the effect of which is mediated by ADCC.

In one embodiment, the invention encompasses a method of treating cancer in a patient having a cancer characterized by a cancer antigen, said method comprising administering a therapeutically effective amount of a therapeutic antibody that binds the cancer antigen, which antibody has been engineered in accordance with the methods of the invention. In a specific embodiment, the invention encompasses a method for treating cancer in a patient having a cancer characterized by a cancer antigen, said method comprising administering a therapeutically effective amount of a therapeutic antibody that specifically binds said cancer antigen, said therapeutic antibody comprising a variant heavy chain having the Fc region of IgG2, IgG3 or IgG4, wherein said variant heavy chain comprises at least one amino acid modification relative to a wild-type heavy chain having the Fc region of the same isotype, such that said therapeutic antibody specifically binds FcγRIIIA with a greater affinity than the therapeutic antibody comprising the wild-type heavy chain binds FcγRIIIA. In another specific embodiment, the invention encompasses a method for treating cancer in a patient having a cancer characterized by a cancer antigen, said method comprising administering a therapeutically effective amount of a therapeutic antibody that specifically binds a cancer antigen, said therapeutic antibody comprising a variant heavy chain having the Fc region of IgG2, IgG3 or IgG4, wherein said variant heavy chain comprises at least one amino acid modification relative to a wild-type heavy chain having the Fc region of the same isotype, such that said therapeutic antibody specifically binds FcγRIIIA with a greater affinity than a therapeutic antibody comprising the wild-type heavy chain having the Fc region of the same isotype binds FcγRIIIA, and said therapeutic antibody further specifically binds FcγRIIB with a lower affinity than a therapeutic antibody comprising the wild-type heavy chain having an Fc region of the same isotype binds FcγRIIB. The invention encompasses a method for treating cancer in a patient characterized by a cancer antigen, said method comprising administering a therapeutically effective amount of a therapeutic antibody that specifically binds said cancer antigen and said therapeutic antibody comprises a variant heavy chain having the Fc region of IgG2, IgG3 or IgG4, wherein said variant heavy chain comprises at least one amino acid modification relative to a wild-type heavy chain having the Fc region of the same isotype, such that the antibody has an enhanced ADCC activity.

The invention encompasses a method of treating an autoimmune disorder and/or inflammatory disorder in a patient in need thereof, said method comprising administering to said patient a therapeutically effective amount of a molecule comprising a variant heavy chain, wherein said molecule binds an immune complex (e.g., an antigen/antibody complex) and said variant heavy chain has the Fc region of IgG2, IgG3 or IgG4, wherein said variant heavy chain comprises at least one amino acid modification relative to a wild-type heavy chain having an Fc region of the same isotype, such that said molecule specifically binds FcγRIIB with a greater affinity than a comparable molecule comprising the wild type heavy chain having an Fc region of the same isotype, and said molecule further specifically binds FcγRIIIA with a lower affinity than a comparable molecule comprising the wild type heavy chain having the Fc region of the same isotype. The invention encompasses a method of treating an autoimmune disorder and/or inflammatory disorder further comprising administering one or more additional prophylactic or therapeutic agents, e.g., immunomodulatory agents, anti-inflammatory agents, used for the treatment and/or prevention of such diseases.

The invention also encompasses methods for treating or preventing an infectious disease in a subject comprising administering a therapeutically or prophylactically effective amount of one or more molecules of the invention that bind an infectious agent or cellular receptor therefor. Infectious diseases that can be treated or prevented by the molecules of the invention are caused by infectious agents including but not limited to viruses, bacteria, fungi, protozae, and viruses.

According to one aspect of the invention, molecules of the invention comprising variant heavy chains having the Fc region of IgG2, IgG3 or IgG4 have an enhanced antibody effector function towards an infectious agent, e.g., a pathogenic protein, relative to a comparable molecule comprising a wild-type heavy chain having an Fc region of the same isotype. In a specific embodiment, molecules of the invention enhance the efficacy of treatment of an infectious disease by enhancing phagocytosis and/or opsonization of the infectious agent causing the infectious disease. In another specific embodiment, molecules of the invention enhance the efficacy of treatment of an infectious disease by enhancing ADCC of infected cells causing the infectious disease.

In some embodiments, the molecules of the invention may be administered in combination with a therapeutically or prophylactically effective amount of one or additional therapeutic agents known to those skilled in the art for the treatment and/or prevention of an infectious disease. The invention contemplates the use of the molecules of the invention in combination with antibiotics known to those skilled in the art for the treatment and or prevention of an infectious disease.

The invention provides pharmaceutical compositions comprising a molecule of the invention, or portion thereof, e.g., a polypeptide comprising a variant heavy chain having the Fc region of IgG2, IgG3 or IgG4; an immunoglobulin comprising a variant heavy chain having the Fc region of IgG2, IgG3 or IgG4; a therapeutic antibody engineered in accordance with the invention, and a pharmaceutically acceptable carrier. The invention additionally provides pharmaceutical compositions further comprising one or more additional therapeutic agents, including but not limited to anti-cancer agents, anti-inflammatory agents, immunomodulatory agents.

### 4.1 DEFINITIONS

As used herein, the term “heavy chain” is used to define the heavy chain of an IgG antibody. In an intact, native IgG, the heavy chain comprises the immunoglobulin domains VH, CH1, CH2 and CH3. Throughout the present specification, the numbering of the residues in an IgG heavy chain is that of the EU index as in Kabat et al., Sequences of Proteins of Immunological Interest, 5^{th} Ed. Public Health Service, NH1, MD (1991), expressly incorporated herein by references. The “EU index as in Kabat” refers to the numbering of the human IgG1 EU antibody. Examples of the amino acid sequences containing human IgG1 CH1, CH2 and CH3 domains are shown in FIG. 1A to FIG. 1C as described, infra. FIGS. 1A to 1C also set forth amino acid sequences of the CH1, hinge, CH2 and CH3 domains of the heavy chains of IgG2, IgG3 and IgG4. The amino acid sequences of IgG2, IgG3 and IgG4 isotypes are aligned with the IgG1 sequence by placing the first and last cysteine residues of the respective hinge regions, which form the inter-heavy chain S—S bonds, in the same positions.

The CH1 domain of a human IgG1 is generally defined as stretching from amino acid 118 to amino acid 215 according to the numbering system of Kabat. An example of the amino acid sequence of the human IgG1 CH1 domain is shown in FIG. 1A (amino acid residues in FIG. 1A are numbered according to the Kabat system). FIG. 1A also provides examples of the amino acid sequences of the CH1 domains of IgG isotypes IgG2, IgG3 and IgG4.

The “hinge region” is generally defined as stretching from Glu216 to Pro230 of human IgG1. An example of the amino acid sequence of the human IgG1 hinge region is shown in FIG. 1B (amino acid residues in FIG. 1B are numbered according to the Kabat system). Hinge regions of other IgG isotypes may be aligned with the IgG1 sequence by placing the first and last cysteine residues forming inter-heavy chain S—S binds in the same positions as shown in FIG. 1B.

As used herein, the term “Fc region” is used to define a C-terminal region of an IgG heavy chain. An example of the amino acid sequence containing the human IgG1 is shown in FIG. 1C. Although boundaries may vary slightly, as numbered according to the Kabat system, the Fc domain extends from amino acid 231 to amino acid 447 (amino acid residues in FIG. 1C are numbered according to the Kabat system). FIG. 1C also provides examples of the amino acid sequences of the Fc regions of IgG isotypes IgG2, IgG3, and IgG4.

The Fc region of an IgG comprises two constant domains, CH2 and CH3. The CH2 domain of a human IgG Fc region usually extends from amino acids 231 to amino acid 341 according to the numbering system of Kabat (FIG. 1C). The CH3 domain of a human IgG Fc region usually extends from amino acids 342 to 447 according to the numbering system of Kabat (FIG. 1C). The CH2 domain of a human IgG Fc region (also referred to as “Cγ2” domain) is unique in that it is not closely paired with another domain. Rather, two N-linked branched carbohydrate chains are interposed between the two CH2 domains of an intact native IgG.

As used herein, the terms “antibody” and “antibodies” refer to monoclonal antibodies, multispecific antibodies, human antibodies, humanized antibodies, synthetic antibodies, chimeric antibodies, polyclonal antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

As used herein, the term “derivative” in the context of polypeptides or proteins refers to a polypeptide or protein that comprises an amino acid sequence which has been altered by the introduction of amino acid residue substitutions, deletions or additions. The term “derivative” as used herein also refers to a polypeptide or protein which has been modified, i.e., by the covalent attachment of any type of molecule to the polypeptide or protein. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative polypeptide or protein may be produced by chemical modifications using techniques known to those of skill in the art, including, but not limited to specific chemical cleavage, acetylation, formylation, metabolic synthesis of tunicamycin, etc. Further, a derivative polypeptide or protein derivative possesses a similar or identical function as the polypeptide or protein from which it was derived.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated.

As used herein, the terms “disorder” and “disease” are used interchangeably to refer to a condition in a subject. In particular, the term “autoimmune disease” is used interchangeably with the term “autoimmune disorder” to refer to a condition in a subject characterized by cellular, tissue and/or organ injury caused by an immunologic reaction of the subject to its own cells, tissues and/or organs. The term “inflammatory disease” is used interchangeably with the term “inflammatory disorder” to refer to a condition in a subject characterized by inflammation, preferably chronic inflammation. Autoimmune disorders may or may not be associated with inflammation. Moreover, inflammation may or may not be caused by an autoimmune disorder. Thus, certain disorders may be characterized as both autoimmune and inflammatory disorders.

As used herein, the term “cancer” refers to a neoplasm or tumor resulting from abnormal uncontrolled growth of cells. As used herein, cancer explicitly includes, leukemias and lymphomas. In some embodiments, cancer refers to a benign tumor, which has remained localized. In other embodiments, cancer refers to a malignant tumor, which has invaded and destroyed neighboring body structures and spread to distant sites. In some embodiments, the cancer is associated with a specific cancer antigen.

As used herein, the term “immunomodulatory agent” and variations thereof refer to an agent that modulates a host's immune system. In certain embodiments, an immunomodulatory agent is an immunosuppressant agent. In certain other embodiments, an immunomodulatory agent is an immunostimulatory agent. Immunomodatory agents include, but are not limited to, small molecules, peptides, polypeptides, fusion proteins, antibodies, inorganic molecules, mimetic agents, and organic molecules.

As used herein, the term “epitope” refers to a fragment of a polypeptide or protein or a non-protein molecule having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays. Antigenic epitopes need not necessarily be immunogenic.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide. In a specific embodiment, a fragment of a polypeptide retains at least one function of the polypeptide.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., delay or minimize the spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the terms “prophylactic agent” and “prophylactic agents” refer to any agent(s) which can be used in the prevention of a disorder, or prevention of recurrence or spread of a disorder. A prophylactically effective amount may refer to the amount of prophylactic agent sufficient to prevent the recurrence or spread of hyperproliferative disease, particularly cancer, or the occurrence of such in a patient, including but not limited to those predisposed to hyperproliferative disease, for example those genetically predisposed to cancer or previously exposed to carcinogens. A prophylactically effective amount may also refer to the amount of the prophylactic agent that provides a prophylactic benefit in the prevention of disease. Further, a prophylactically effective amount with respect to a prophylactic agent of the invention means that amount of prophylactic agent alone, or in combination with other agents, that provides a prophylactic benefit in the prevention of disease.

As used herein, the terms “prevent”, “preventing” and “prevention” refer to the prevention of the recurrence or onset of one or more symptoms of a disorder in a subject as result of the administration of a prophylactic or therapeutic agent.

As used herein, the term “in combination” refers to the use of more than one prophylactic and/or therapeutic agents. The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

“Effector function” as used herein is meant a biochemical event that results from the interaction of an antibody Fc region with an Fc receptor or ligand. Effector functions include but are not limited to antibody dependent cell mediated cytotoxicity (ADCC), antibody dependent cell mediated phagocytosis (ADCP), and complement dependent cytotoxicity (CDC). Effector functions include both those that operate after the binding of an antigen and those that operate independent of antigen binding.

“Effector cell” as used herein is meant a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and may be from any organism including but not limited to humans, mice, rats, rabbits, and monkeys.

“Fe ligand” as used herein is meant a molecule, preferably a polypeptide, from any organism that binds to the Fe region of an antibody to form an Fc-ligand complex. Fe ligands include but are not limited to FcγRs, FcγRs, FcγRs, FcRn, C1q, C3, staphylococcal protein A, streptococcal protein G, and viral FcγR. Fe ligands may include undiscovered molecules that bind Fe.

### 5. BRIEF DESCRIPTION OF THE DRAWINGS

FIGS. 1A-1C Amino Acid Sequence of Human IgG CH1, Hinge and Fc Regions

FIGS. 1A-1C provides the amino acid sequences of human IgG1, IgG2, IgG3 and IgG4 CH1 (FIG. 1A), hinge (FIG. 1B) and Fe (FIG. 1C) domains. The amino acid residues shown in the figure are numbered according to the numbering system of Kabat. Isotype sequences are aligned with the IgG1 sequence by placing the first and last cysteine residues of the respective hinge regions, which form the inter-heavy chain S—S bonds, in the same positions. For FIG. 1C, residues in the CH2 domain are indicated by +, while residues in the CH3 domain are indicated by ˜.

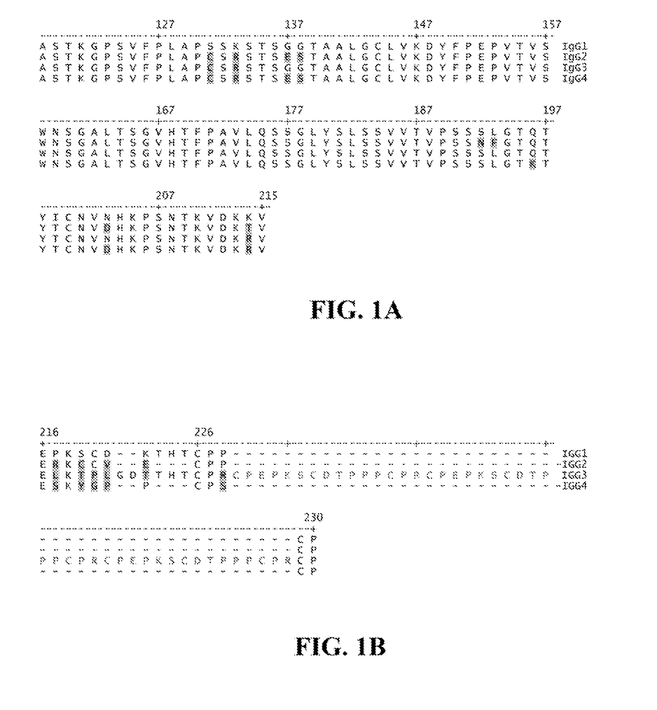

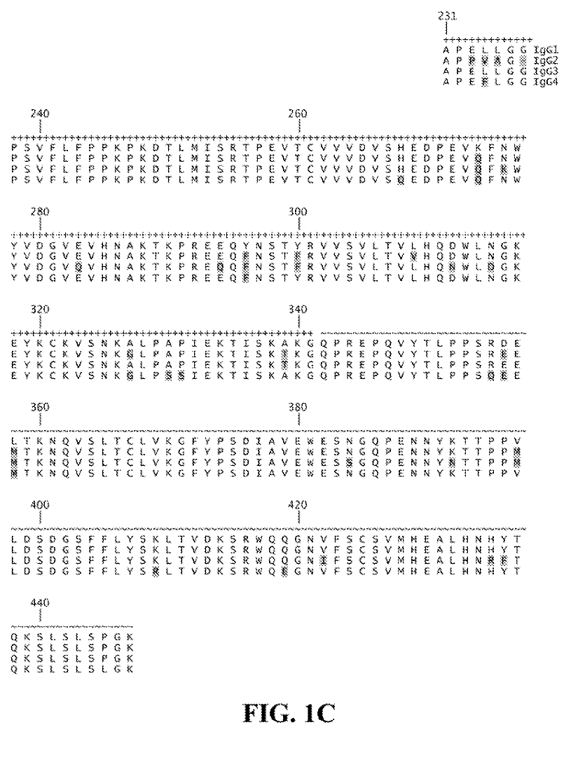

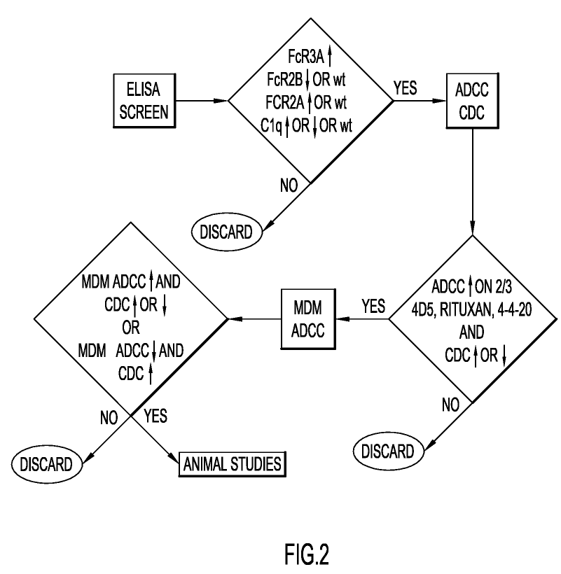

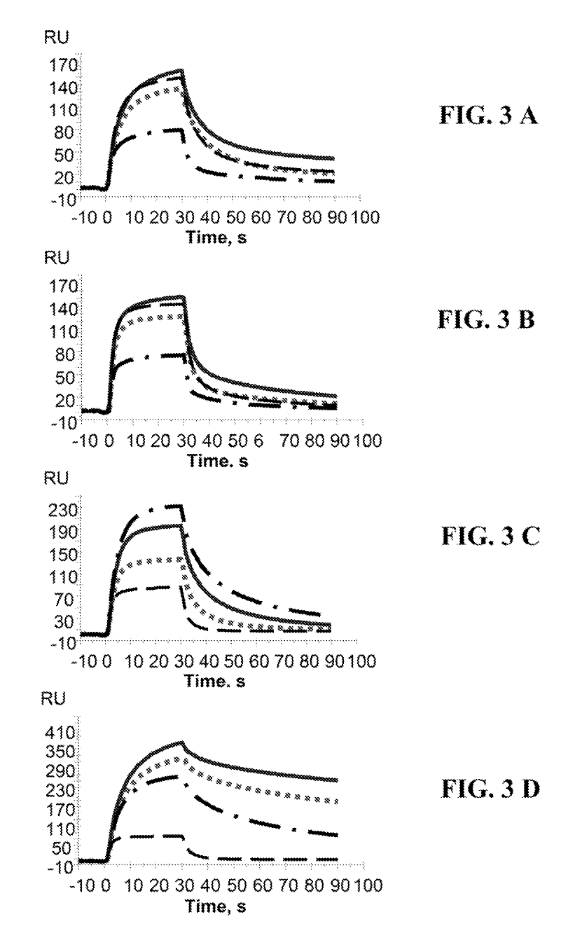

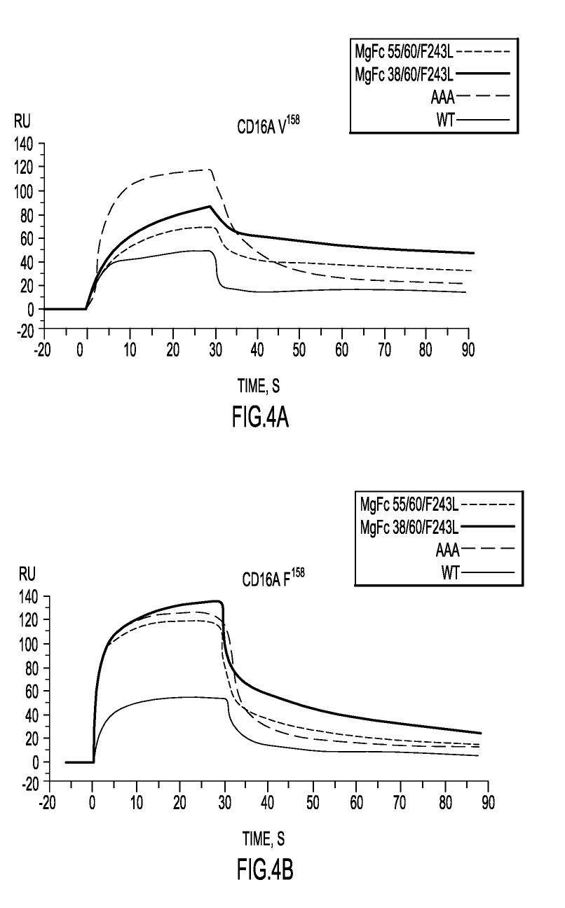

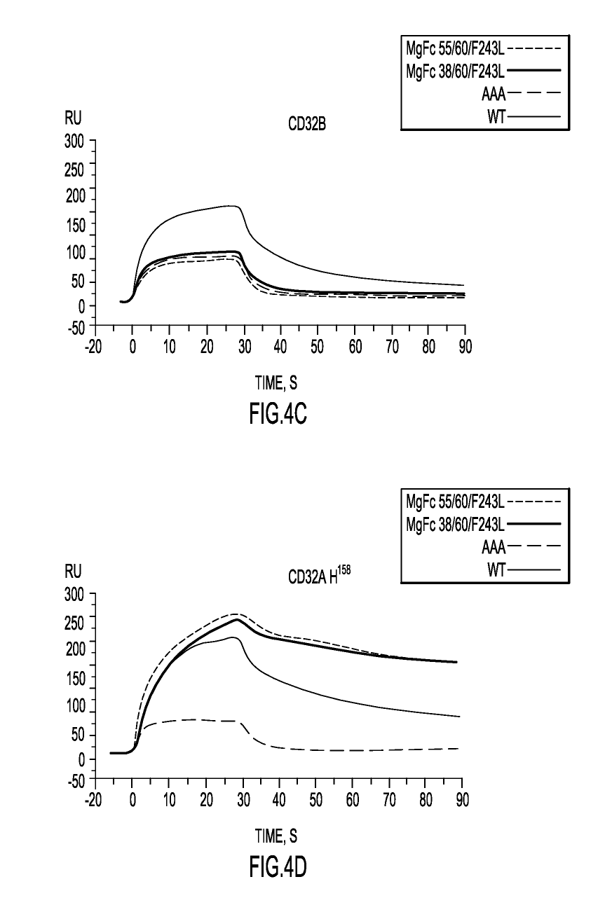

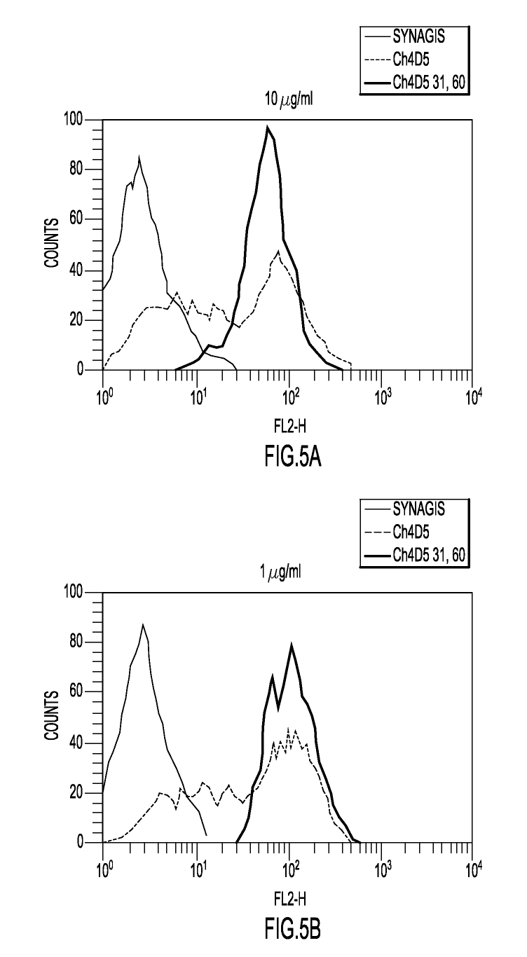

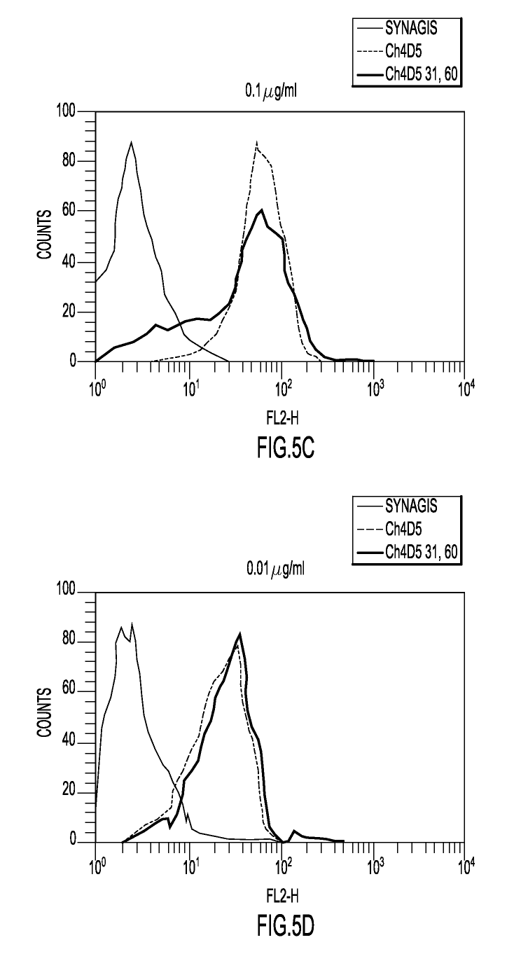

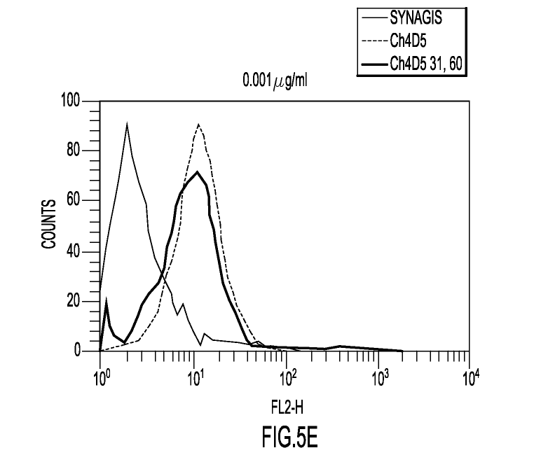

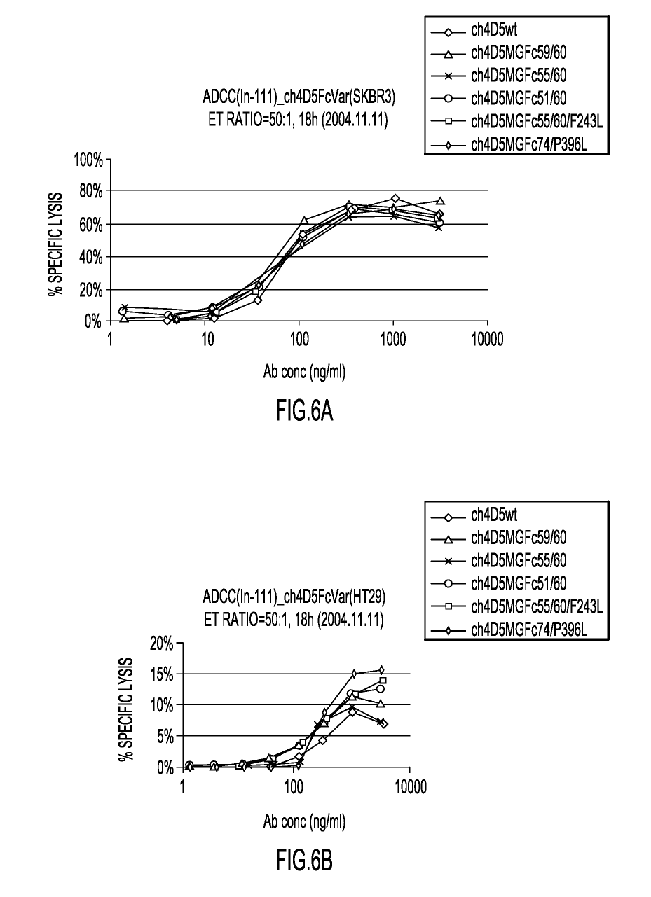

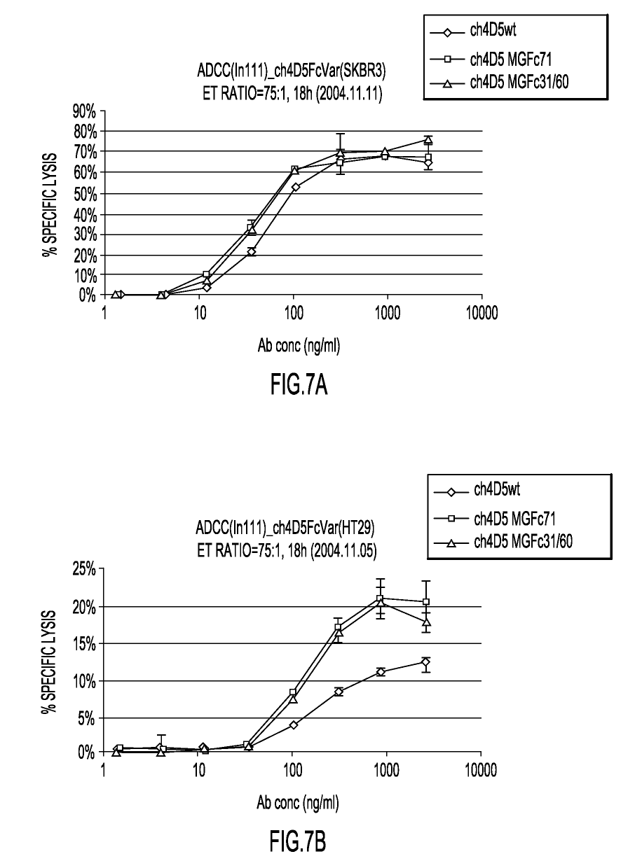

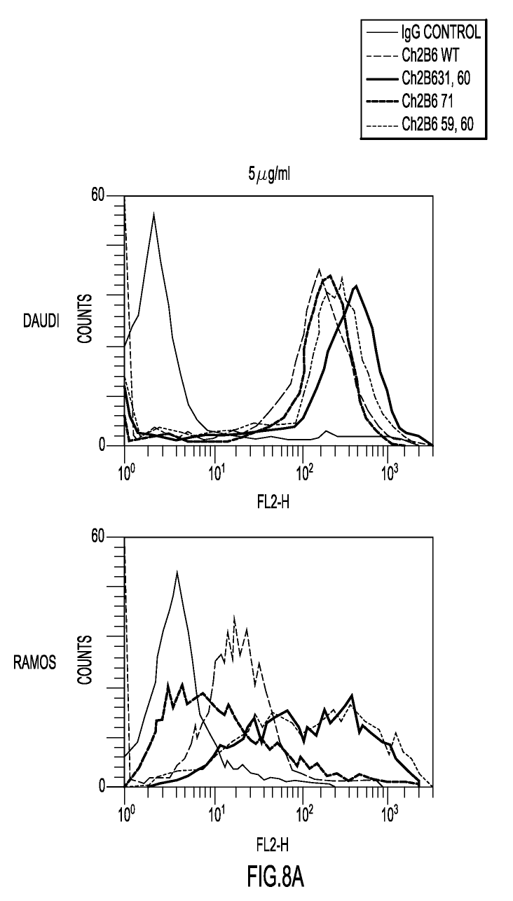

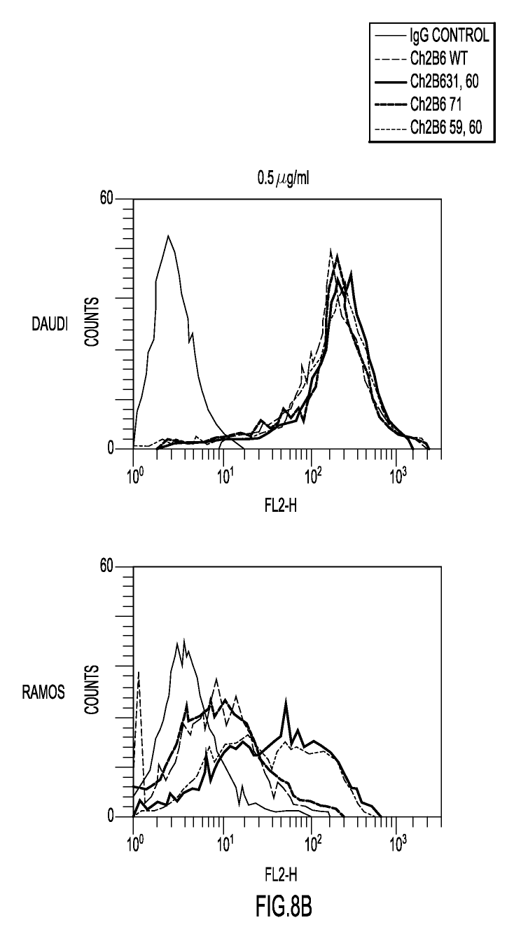

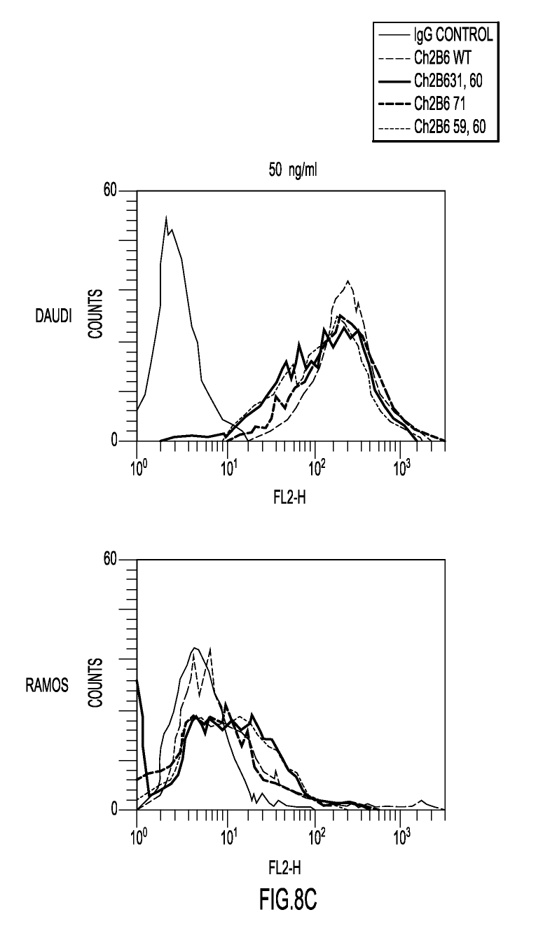

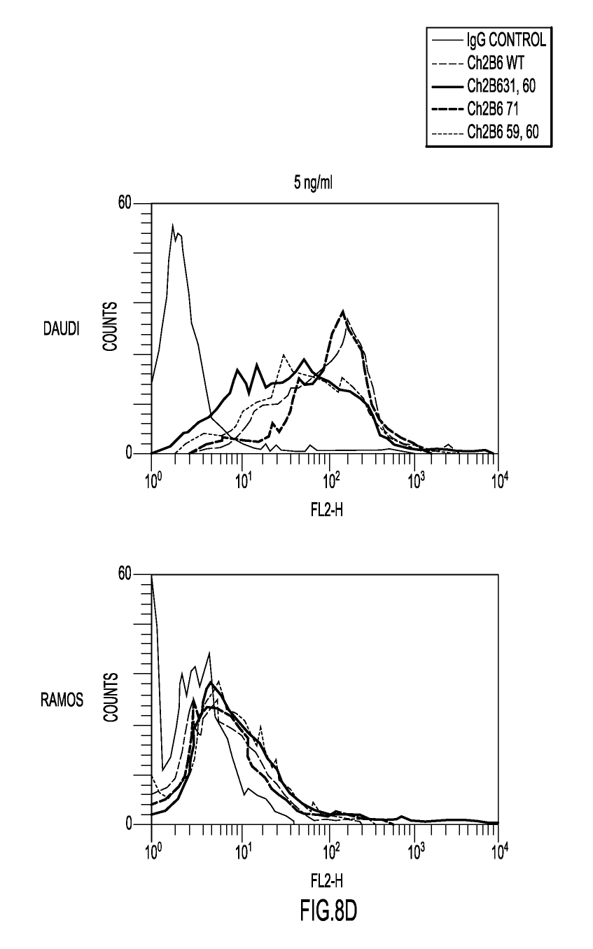

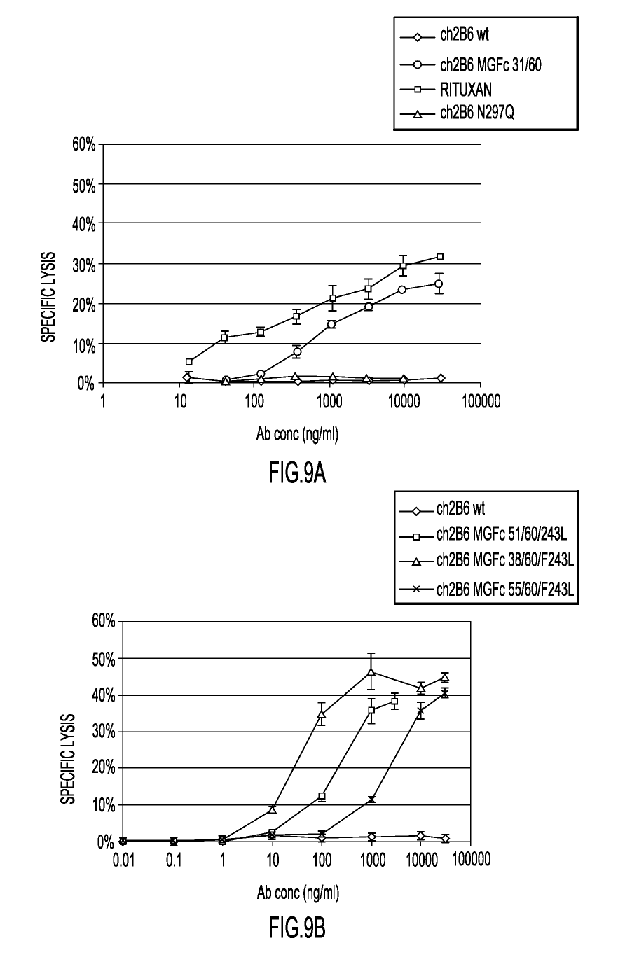

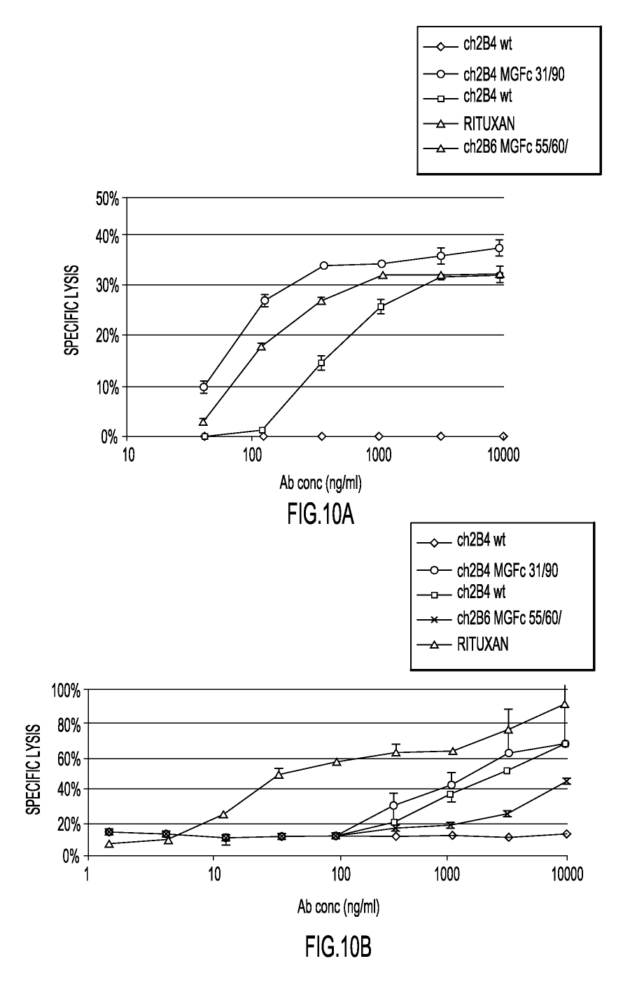

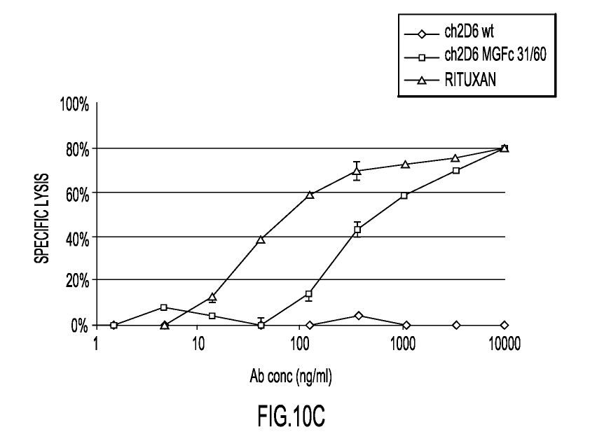

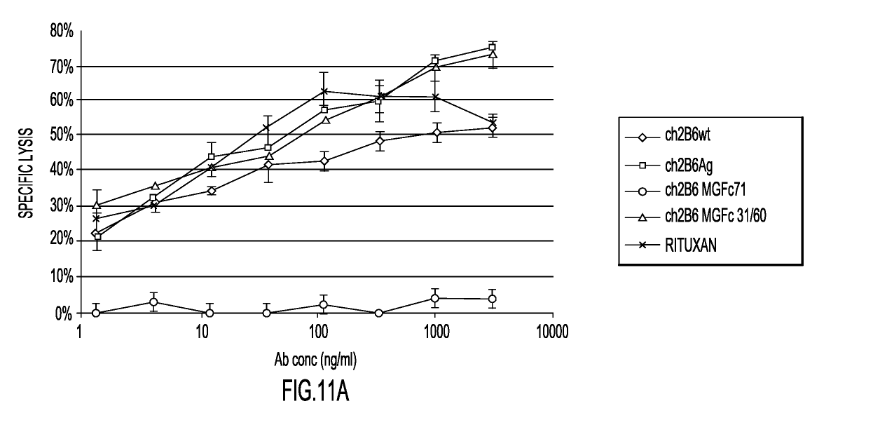

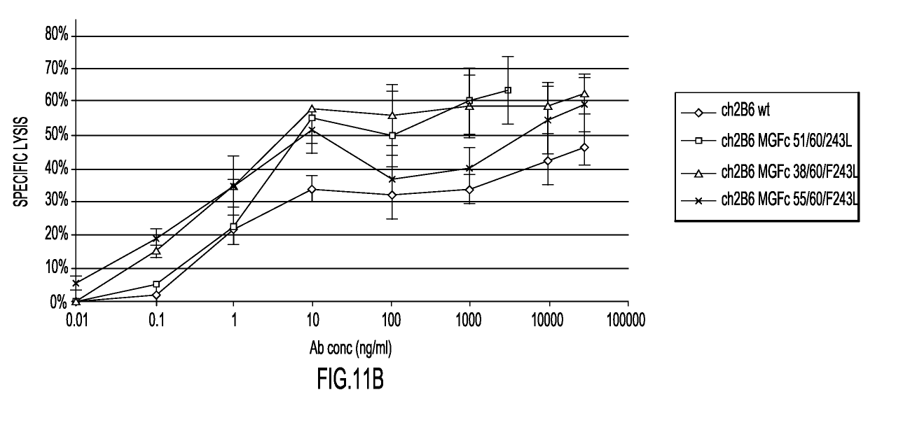

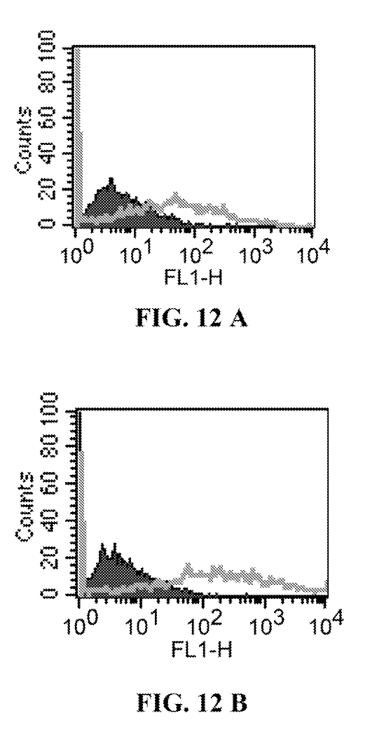

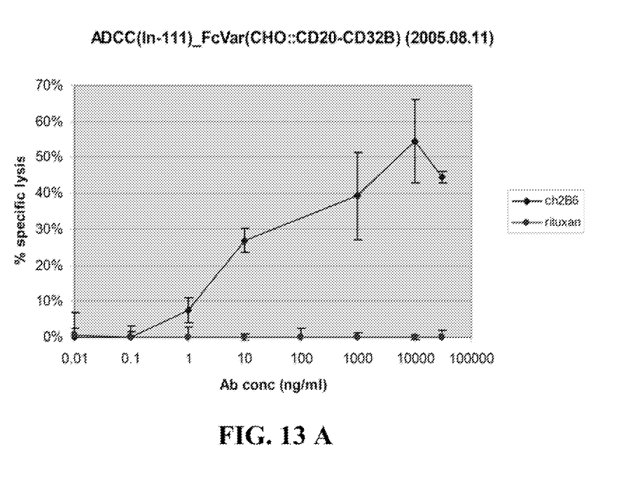

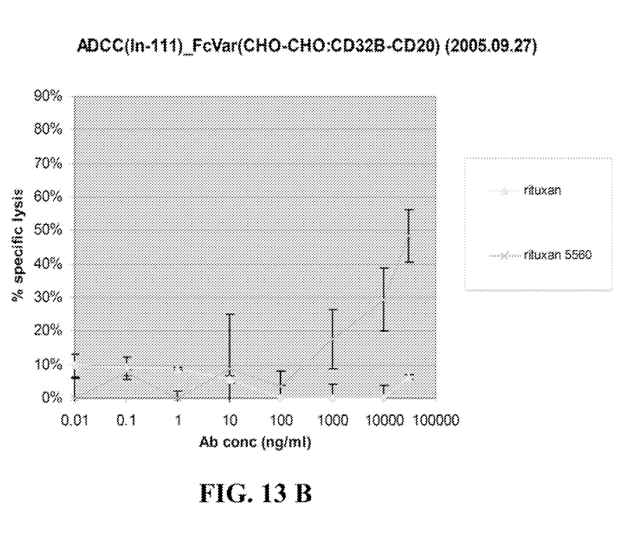

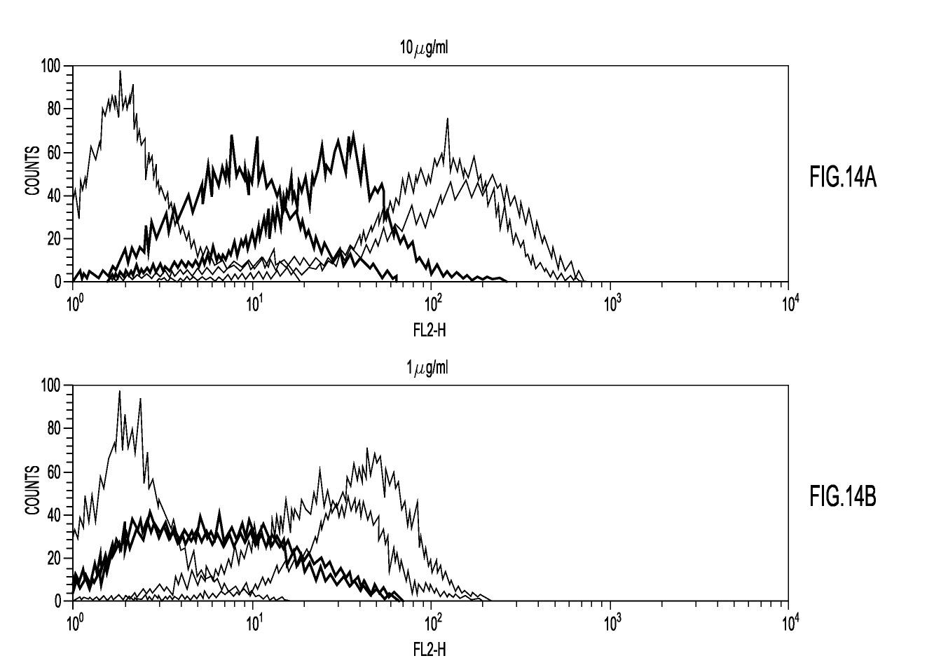

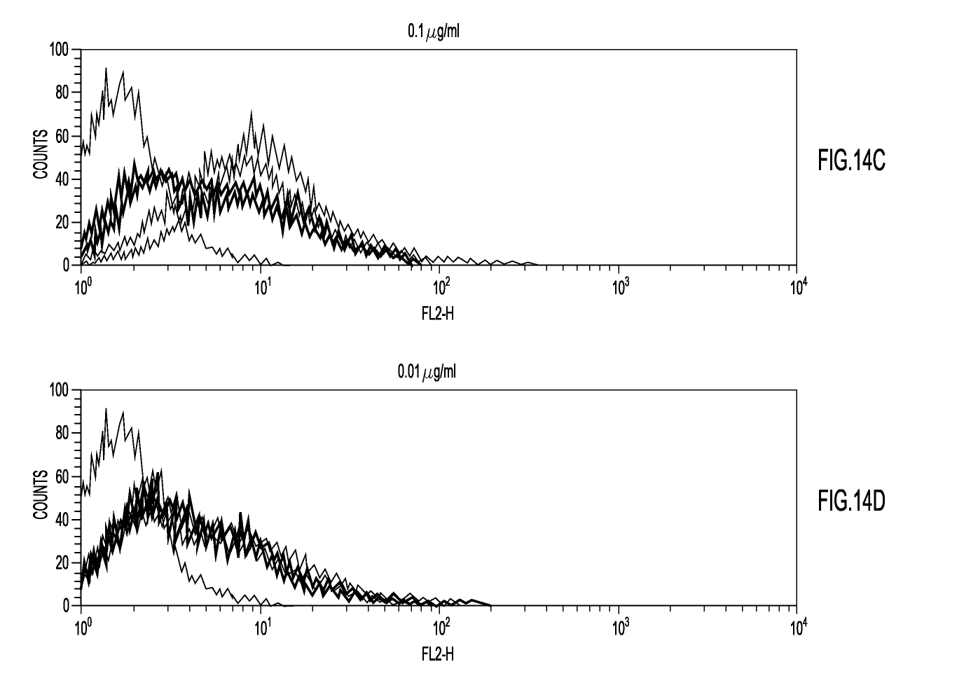

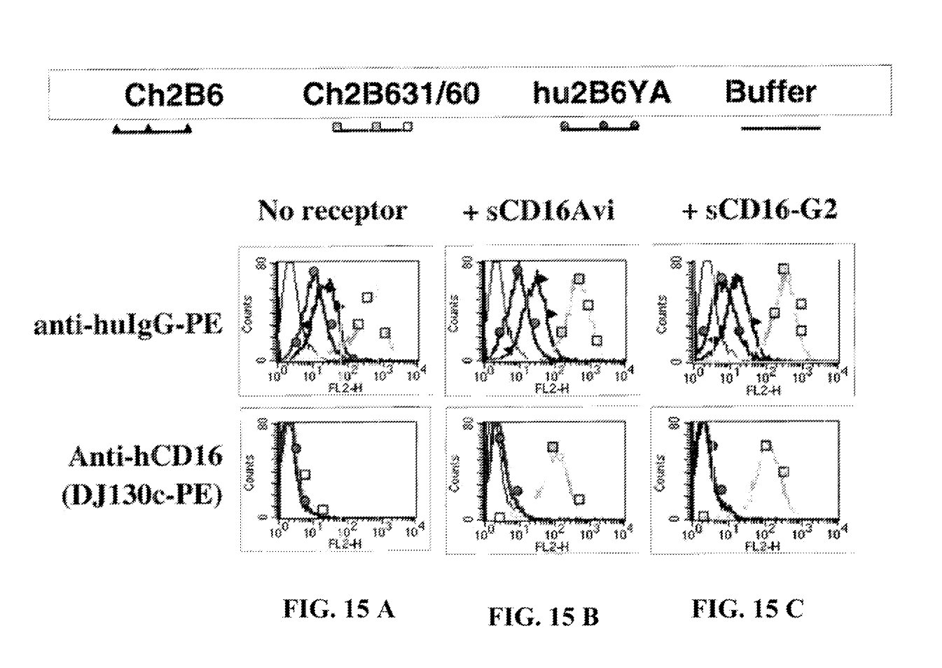

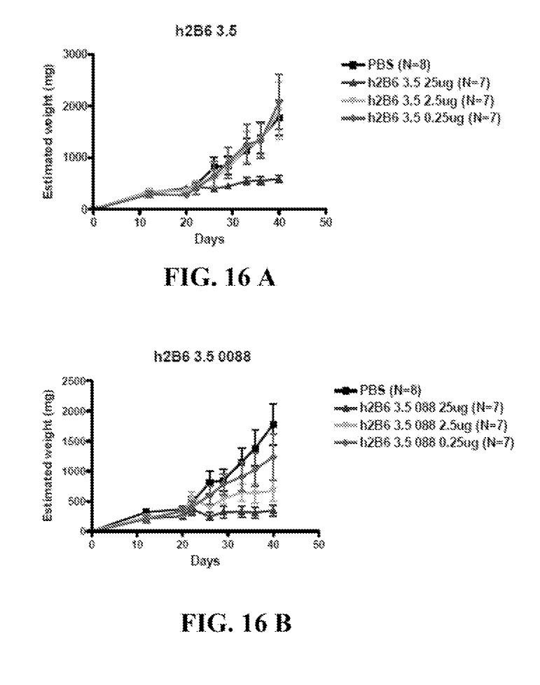

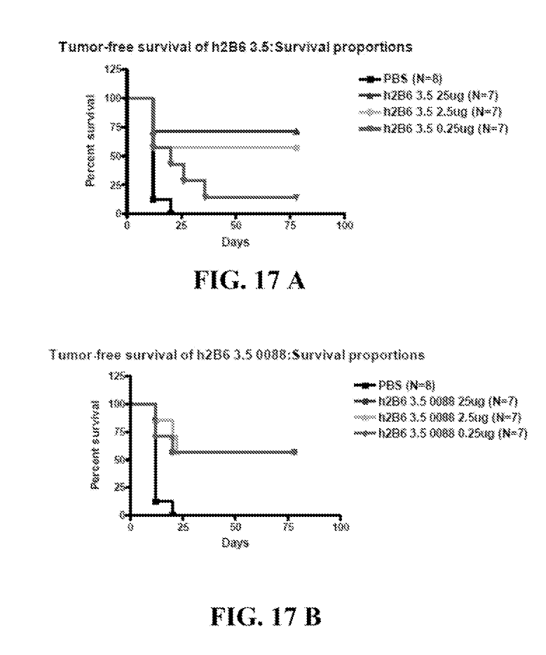

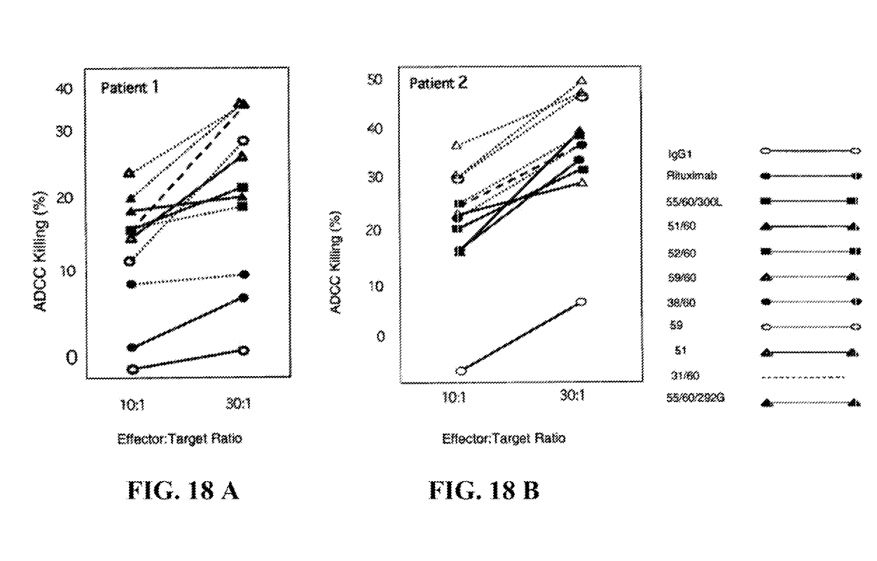

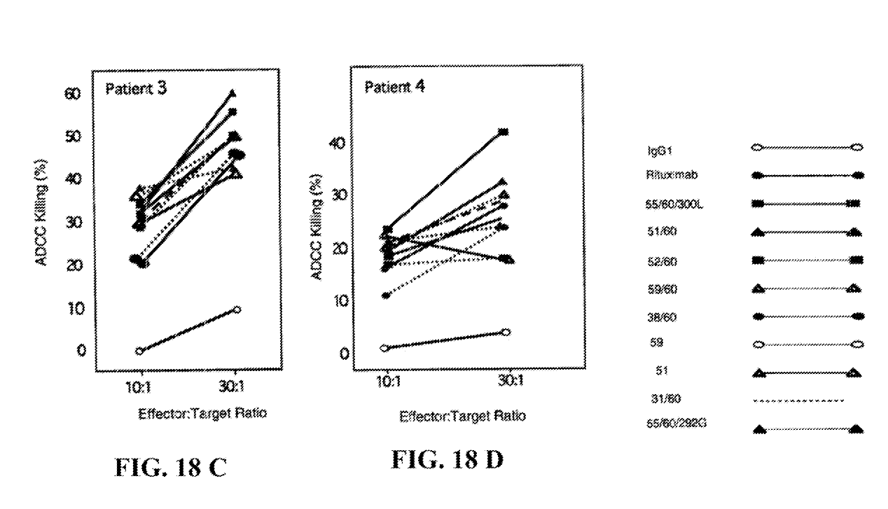

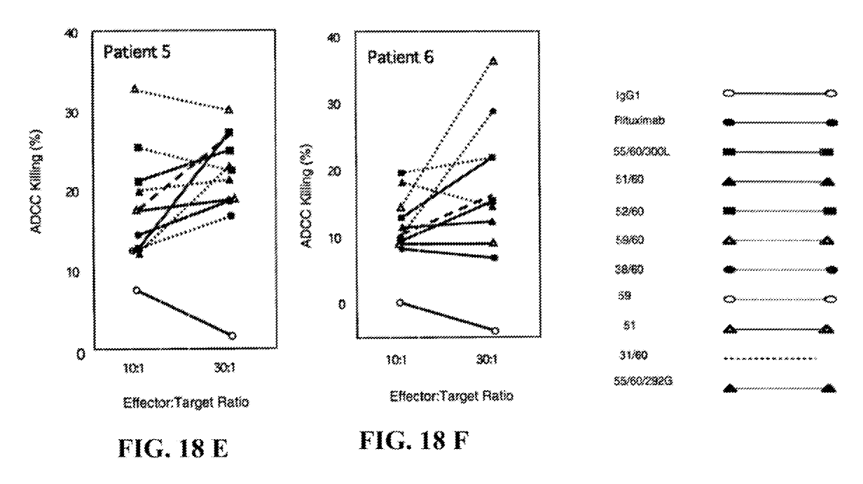

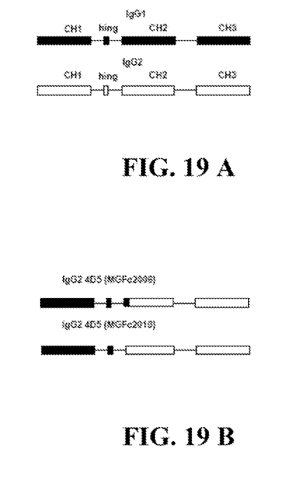

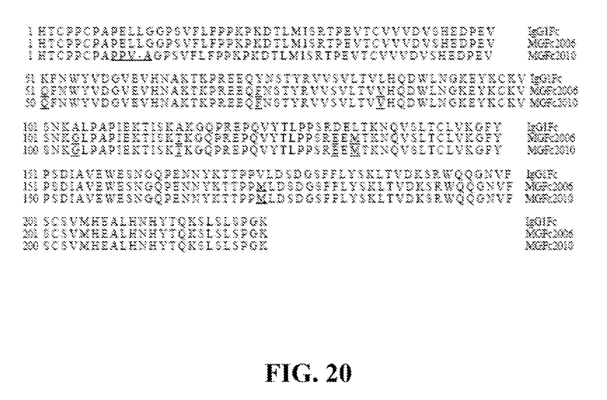

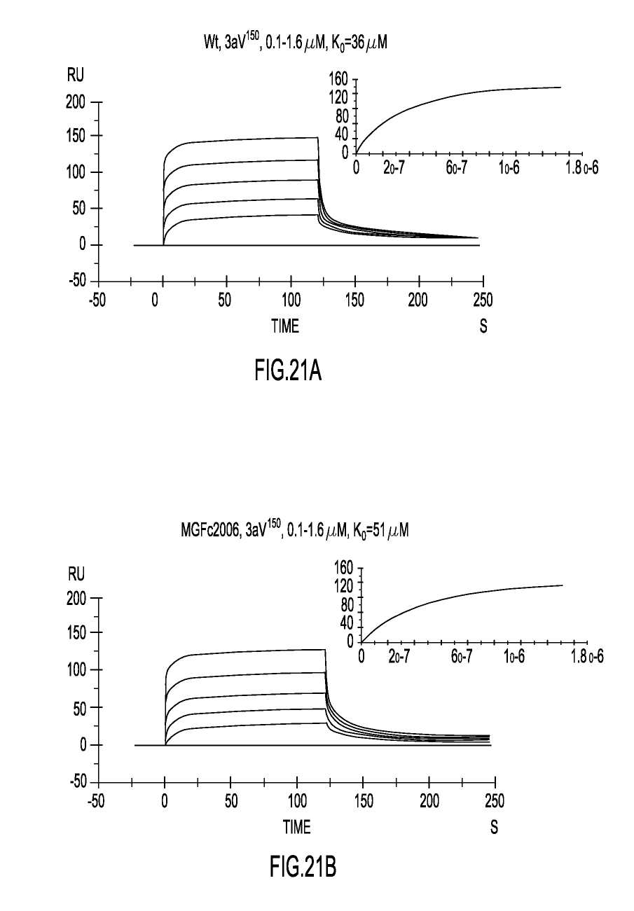

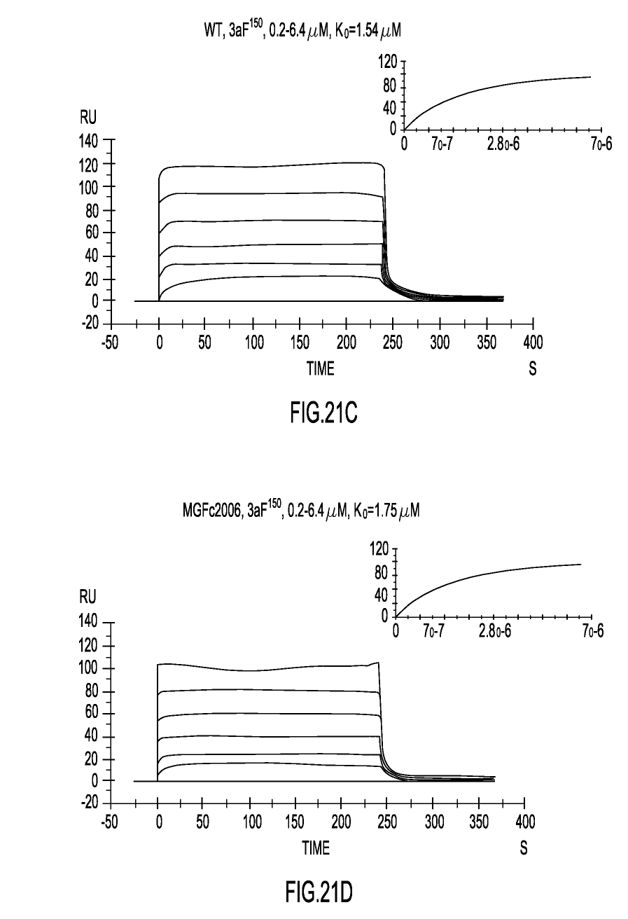

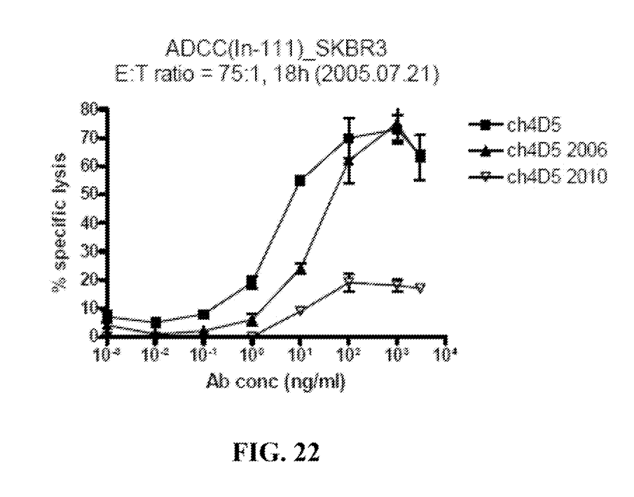

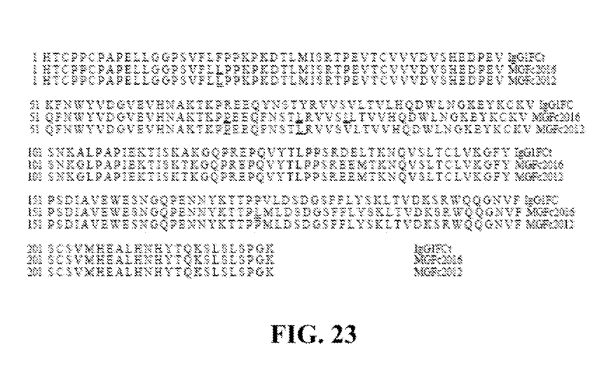

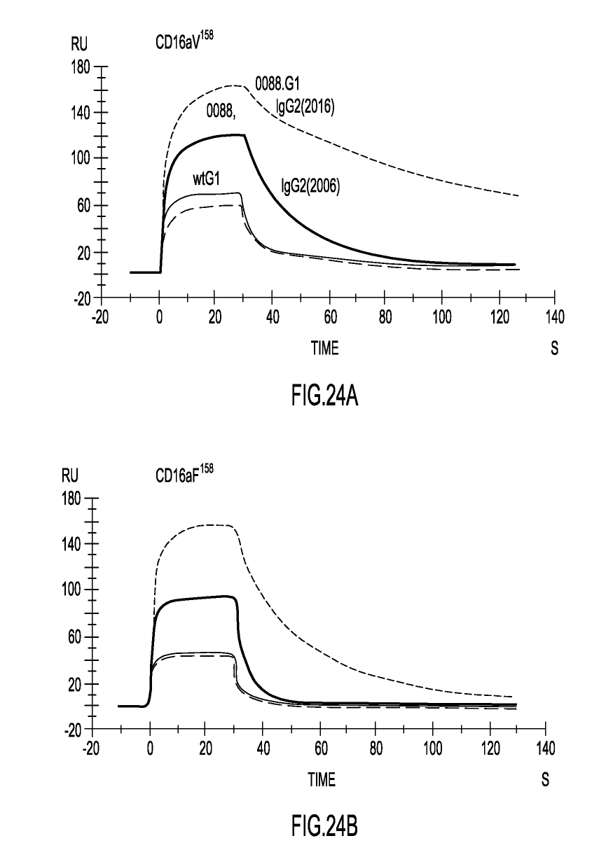

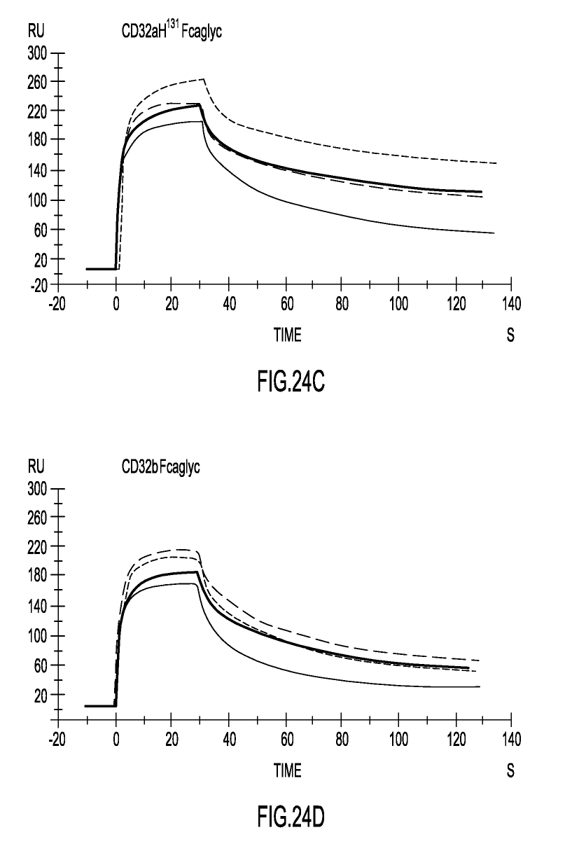

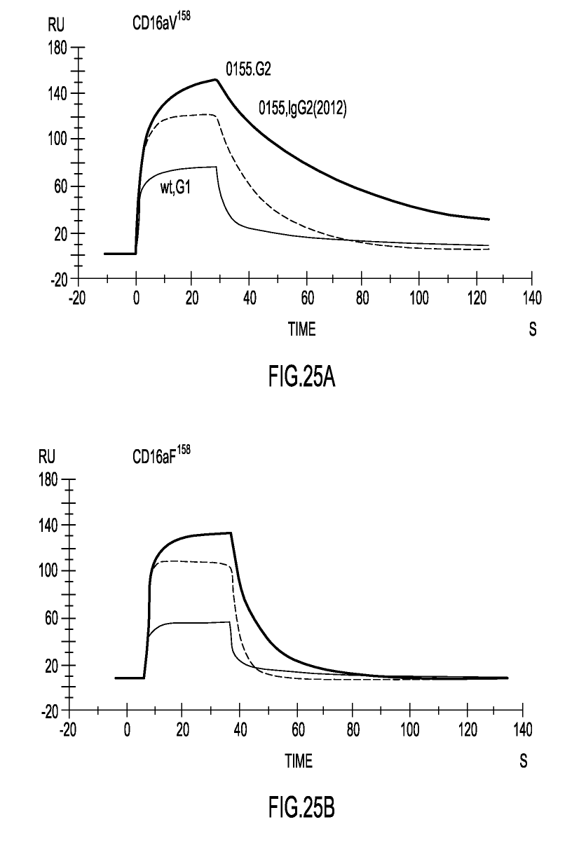

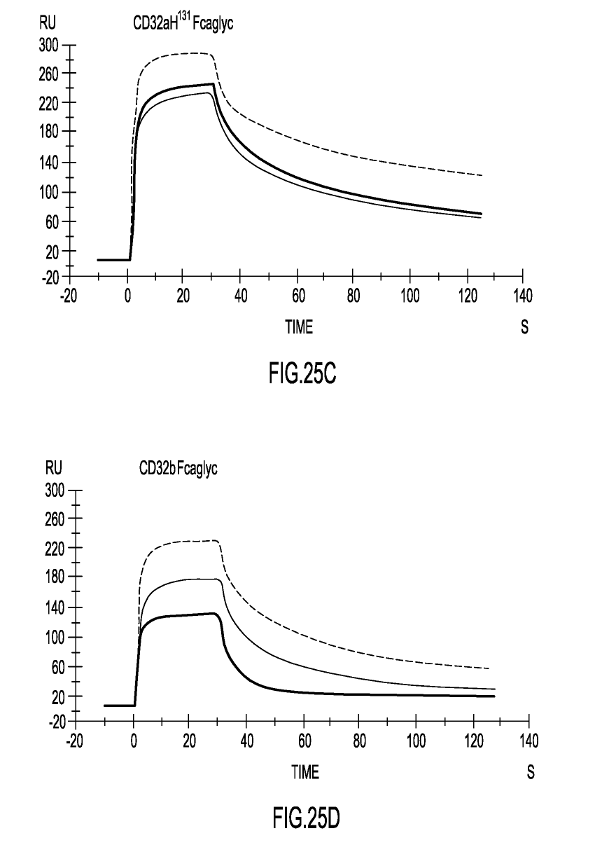

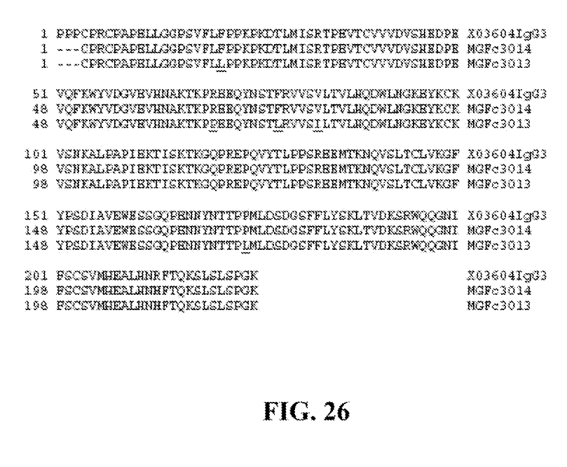

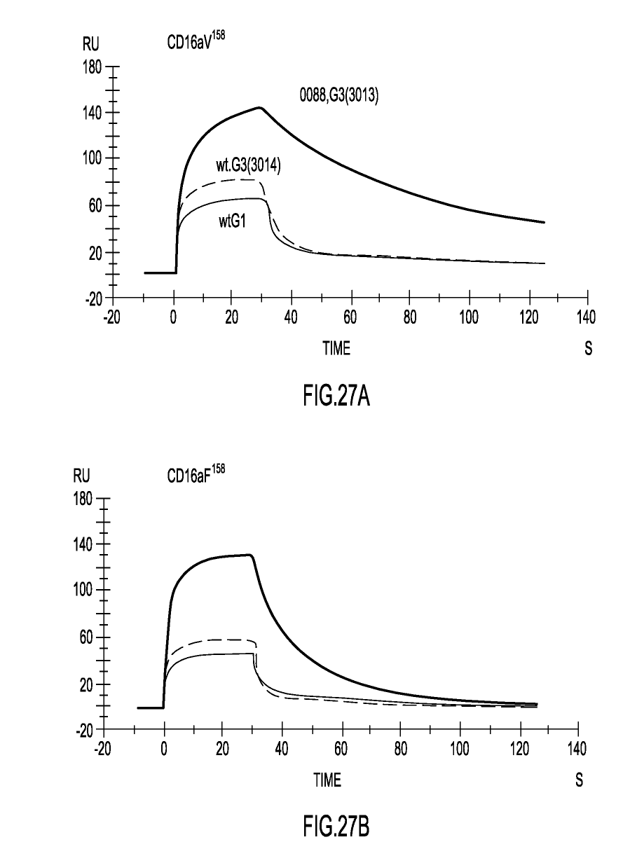

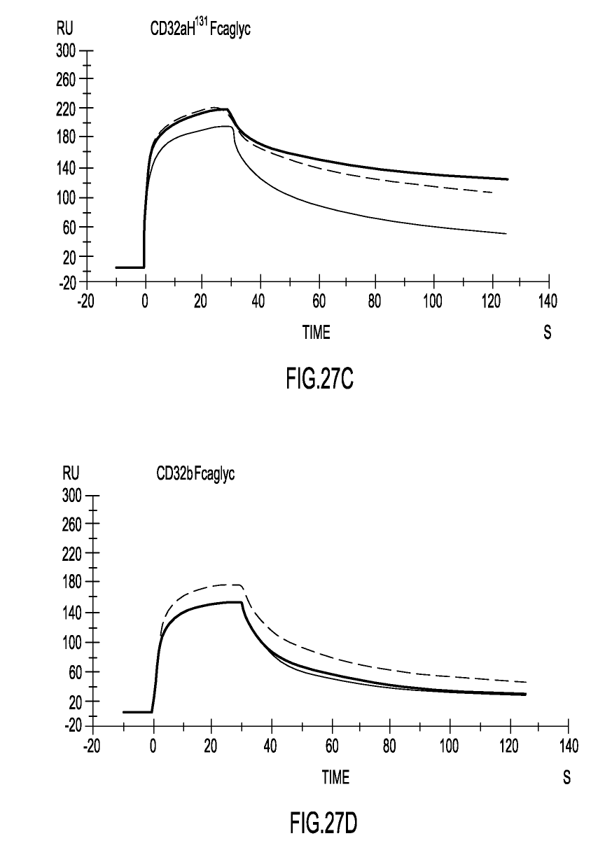

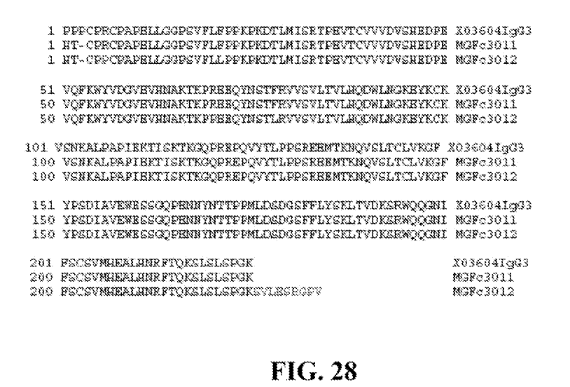

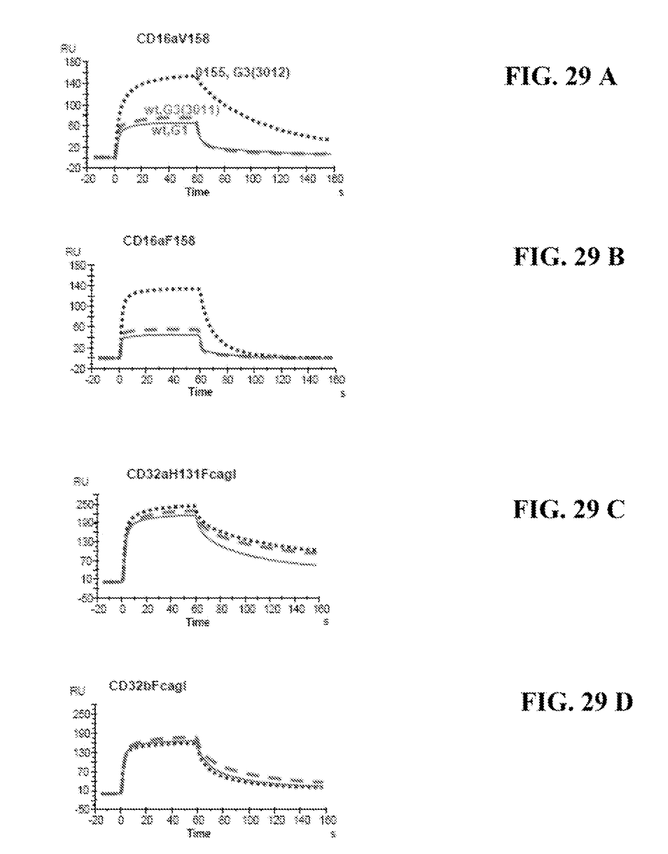

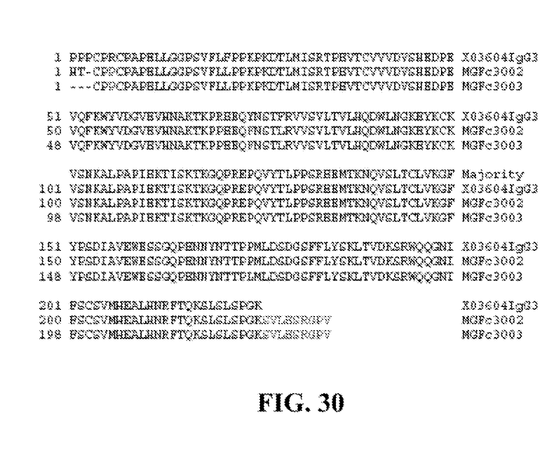

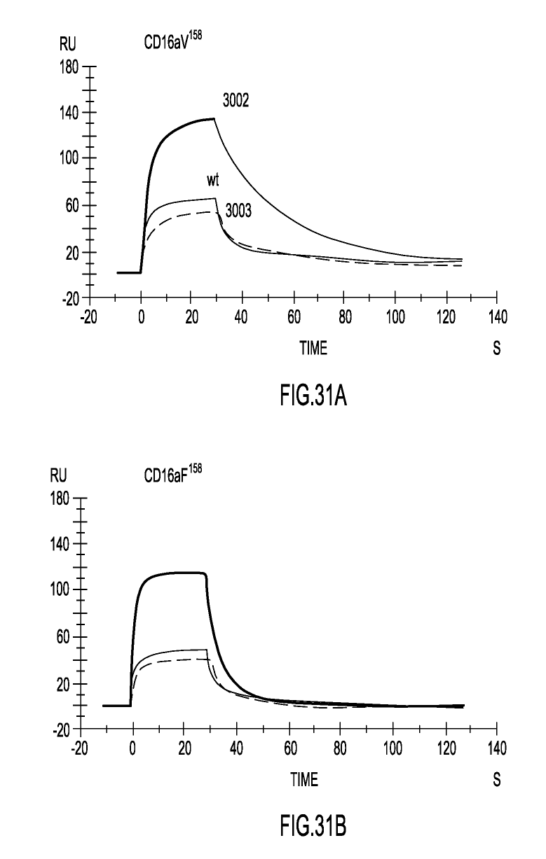

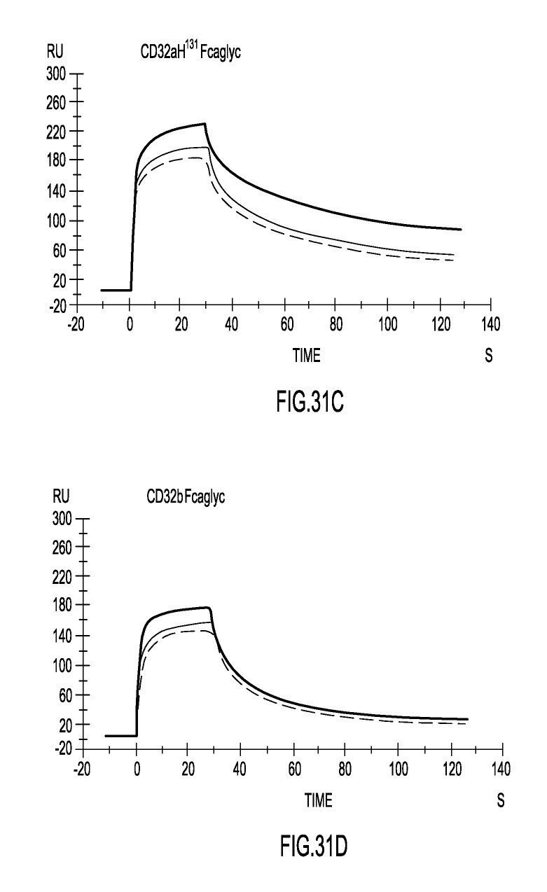

### FIG. 2 Decision Tree for Selection of Fc Mutants

FIG. 2 shows an exemplary protocol for selecting Fe mutants.

### FIGS. 3A-3D FCγR Binding to 4D5 Mutant Antibody, Triple Mutation

FIGS. 3A-3D show sensogram of real time binding of 4D5 mutants to FcγRIII3A (CD16Z V¹⁵⁸, FIG. 3A, and CD16A F¹⁵⁸, FIG. 3B), FcγRIIB (CD32B, FIG. 3C) and FcγRIIA (CD32A H¹³¹, FIG. 3D). Mutants depicted are MgFc31/60 (P247L; N421K; D270E), MgFc71 (D270E; G316D; R416G) and AAA (E333A; K334A; S298A). The binding of wild-type 4D5 is also provided.

### FIGS. 4A-4D FCγR Binding to 4D5 Mutant Antibody, Quadruple Mutation

FIGS. 4A-4D show sensogram of real time binding of 4D5 mutants to FcγRIII3A (CD16Z V¹⁵⁸, FIG. 4A, and CD16A F¹⁵⁸, FIG. 4B), FcγRIIB (CD32B, FIG. 4C) and FcγRIIA (CD32A H¹³¹, FIG. 4D). Mutants depicted are MgFc55/60/F243L (R255L; P396L; D270E; F243L), MgFc38/60/F243L (K392T; P396L; D270E; F243L) and AAA (E333A; K334A; S298A). The binding of wild-type 4D5 is also provided.

### FIGS. 5A-5E Binding of 4D5 Variant 31/60 to HT29 Cells

FACS analysis was used to characterize the binding of monoclonal anti-HER2/neu antibody ch4D5, variant 31/60 (P247L; N421K; D270E), to HT29 cells (low expression of HER2/neu). Incubation with primary antibody was at 10 μg/ml (FIG. 5A), 1 μg/ml (FIG. 5B), 0.1 μg/ml (FIG. 5C), 0.001 μg/ml (FIG. 5D), or 0.001 μg/ml (FIG. 5E). Wild-type ch4D5 and Synagis were used as controls. PE-conjugated polyclonal F(ab)₂ goat anti-humanFCγR was used as the secondary antibody.

FIGS. 6A-6B ADCC Activity of Mutants in the Anti-HER2/neu Antibody, ch4D5

CH4D5 antibodies containing mutant Fc regions were assessed for their ADCC activity and compared to the ADCC activity of wild type ch4D5. SKBR3 (high expression of HER2/neu) and HT29 (low expression of HER2/neu) cells lines were used as targets (FIGS. 6A and 6B, respectively). Effector to target ratio (E:T ratio) was 50:1 with 18 h incubation. Mutants analyzed were MGFc59/60 (K370E; P396L; D270E), MGFc55/60 (R255L; P396L; D270E), MGFc51/60 (Q419H; P396L; D270E), MGFc55/60/F243L (R255L; P396L; D270E; F243L); MGFc74/P396L (F243L; R292P; V305I; P396L).

FIGS. 7A-7B ADCC Activity of Mutants in the Anti-HER2/neu Antibody, ch4D5

Ch4D5 antibodies containing mutant Fc regions were assessed for their ADCC activity and compared to the ADCC activity of wild type ch4D5. SKBR3 (high expression of HER2/neu) and HT29 (low expression of HER2/neu) cells lines were used as targets (FIGS. 7A and 7B, respectively). Effector to target ratio (E:T ratio) was 75:1 with 18 h incubation. Mutants analyzed were MgFc31/60 (P247L; N421K; D270E) and MgFc71 (D270E; G316D; R416G).

FIG. 8 Binding of Mutants in the Monoclonal Anti-CD32B Antibody ch2B6 to Daudi Cells and Ramos Cells

FACS analysis was used to characterize the binding of monoclonal anti-CD32B antibody ch2B6 variant 31/60 (P247L; N421K; D270E), variant 71 (D270E; G316D; R416G) and variant 59/60 (K370E; P396L; D270E) to either Daudi cells (high expression of CD32B) or Ramos cells (low expression of CD32B). Incubation with primary antibody was at 5 μg/ml (A), 0.5 μg/ml (B), 50 ng/ml (C), or 5 ng/ml (D). Wild-type ch2B6 and IgG (SYNAGIS) were used as controls. PE-conjugated polyclonal F(ab)₂ goat anti-humanFCγR was used as the secondary antibody.

FIGS. 9A-9B ADCC Activity of Mutants in the Anti-CD32B Antibody, ch2B6

Ch2B6 antibodies containing mutant Fe regions were assessed for their ADCC activity and compared to the ADCC activity of wild type 2B6. The Ramos cell line (low expression of CD32B) was used as target. Effector to target ratio (E:T ratio) was 75:1 with 18 h incubation. Mutants analyzed were variant 31/60 (P247L; N421K; D270E) and ch2B6 N297Q (aglycoslyated Fe, no FcR binding) (FIG. 9A); and MGFc51/60/F243L (Q419H; P396L; D270E; F243L); MGFc55/60/F243L (R255L; P396L; D270E; F243L) and MGFc38/60/F243L (K392T; P396L; D270E; F243L) (FIG. 9B). Wild-type ch2B6 or RITUXAN® were used as controls.

FIGS. 10A-10C CDC Activity of Mutants in the Anti-CD32B Antibody, ch2B6

Ch2B6 antibodies containing mutant Fe regions were assessed for their CDC activity and compared to the CDC activity of wild type ch2B6. BL41 (a Burkitt's lymphoma cell line) (FIGS. 10A and 10B) and Ramos (low expression of CD32B) (FIG. 10C) cells lines were used as targets. Effector to target ratio (E:T ratio) was 75:1 with 18 h incubation. Mutants analyzed were MgFc31/60 (P247L; N421K; D270E) and, MGFc55/60/Y300L (R255L; P396L; D270E; Y300L) (FIG. 10A); MgFc71 (D270E; G316D; R416G), MGFc51/60/F243L (Q419H; P396L; D270E; F243L), and MGFc55/60/F243L (R255L; P396L; D270E; F243L) (FIG. 10B); and MgFc31/60 (P247L; N421K; D270E) (FIG. 10C). Wild-type ch2B6, wild-type humanized ch2B6 (hu2B6 wt) or RITUXAN® were used as controls.

FIGS. 11A-11B ADCC Activity of Mutants in the Anti-CD32B Antibody, ch2B6

Ch2B6 antibodies containing mutant Fe regions were assessed for their ADCC activity and compared to the ADCC activity of wild type ch2B6. The Daudi cell line (high expression of CD32B) was used as target. Effector to target ratio (E:T ratio) was 75:1 with 18 h incubation. Mutants analyzed were MgFc31/60 (P247L; N421K; D270E), ch2B6 Ag (297Q; aglycoslyated Fe, no FcR binding) and MgFc71 (D270E; G316D; R416G) (FIG. 11A); and MGFc55/60/F243L (R255L; P396L; D270E; F243L), MGFc51/60/F243L (Q419H; P396L; D270E; F243L) and MGFc38/60/F243L (K392T; P396L; D270E; F243L) (FIG. 11B). Wild-type ch2B6, RITUXAN® or were used as controls.

FIGS. 12A-12B FACS Analysis of the Binding of the Anti-CD32B Antibody, ch2B6, and the Anti-CD20 Antibody, RITUXAN™, to a Transgenic CHO Cell Line.

Cho cells were engineered to express both recombinant CD32B and recombinant CD20 on the cell surface. Following incubation and amplification in selective media, cells were analyzed by FACS. Cells were incubated in either FITC-conjugated wild-type 2B6 (FIG. 12A) or FITC-conjugated RITUXAN® (FIG. 12B).

### FIGS. 13A-13B ADCC Activity of Mutants in the Anti-CD20 Antibody, RITUXAN™

RITUXAN® antibodies containing mutant Fc regions were assessed for their ADCC activity and compared to the ADCC activity of wild type RITUXAN® and ch2B6. A Cho cell line engineered to express both CD32B and CD20 was used as target. Effector to target ratio (E:T ratio) was 75:1 with 18 h incubation. FIG. 13A shows the ADCC activity of wild type ch2B6 and RITUXAN®. FIG. 13B shows a comparison of the ADCC activity of wild type RITUXAN® and RITUXAN® comprising mutation variant MGFc55/60 (R255L; P396L; D270E).

FIGS. 14A-14D Comparison of Binding Affinity and Kinetic Characteristics of ch2B6 Mutants

FACS analysis was used to characterize the binding of mutant ch2B6 antibodies to Ramos cells (low expression of CD32B). Data were compared to a BIAcore analysis of the k_{off} for the same variant antibodies. Mutants analyzed were MgFc55 (R255L; P396L), MgFc55/60 (R255L; P396L; D270E) and MgFc55/60/F243L (R255L; P396L; D270E; F243L). Wild-type ch2B6 was used as control. Incubation with primary antibody was at 10 μg/ml (FIG. 14A), 1 μg/ml (FIG. 14B), 0.1 ng/ml (FIG. 14C), or 0.01 ng/ml (FIG. 14D). PE-conjugated polyclonal F(ab)₂ goat anti-humanFCγR was used as the secondary antibody.

FIGS. 15A-15C Binding of Activating Receptor CD16A to Ramos Cells Opsonized with Mutant ch2B6 Antibody

FACS analysis was used to characterize the binding of activating receptor CD16A to Ramos cells opsonized with mutant ch2B631/60 antibody (P247L; N421K; D270E). Opsonization with wild-type ch2B6, hu2B6YA (humanized 2B6 with YA substitution at positions 50,51 of antibody light-chain—eliminates glycosylation at position 50 of the light-chain protein), or antibody-free buffer was used as a control. PE-conjugated polyclonal F(ab)₂ goat anti-humanFCγR was used as the secondary antibody. FIG. 15A (no receptor; anti-huIgG-PE, anti-hCD16); FIG. 15B (sCD16Avi; anti-huIgG-PE, anti-hCD16); FIG. 15C (sCD16-G2; anti-huIgG-PE, anti-hCD16).

FIGS. 16A-16B. Estimated Tumor Weight in Mice Treated with Wild-Type or Fc Mutant h2B6

Balb/c nude mice were inoculated subcutaneously with Daudi cells and administered 25 μg, 2.5 μg or 0.25 μg weekly doses of either wild-type h2B6 (FIG. 16A) or h2B6 harboring Fc mutant MGFc 0088 (F243L, R292P, Y300L, V305I, P396L) (FIG. 16B). Mice administered buffer alone were used as control. Tumor weight was calculated based on the estimated volume of the subcutaneous tumor according to the formula (width²×length)/2.

FIGS. 17A-17B. Survival in Tumor Bearing Mice Treated with Wild-Type or Fc Mutant h2B6

Nude mice were inoculated with Daudi cells and administered 25 μg, 2.5 μg or 0.25 μg weekly doses of either wild-type h2B6 (FIG. 17A) or h2B6 harboring Fc mutant MGFc 0088 (F243L, R292P, Y300L, V305I, P396L) (FIG. 17B). Mice administered buffer alone were used as control.

FIGS. 18A-18F ADCC Activity of Modified Rituximab Antibodies in Human Patients Treated with Rituximab

Rituximab antibodies containing mutant Fc regions were assessed for their ADCC activity and compared to the ADCC activity of wild type rituximab. Patient derived cells (FIGS. 18A-18F) were used as target. Effector to target ratio (E:T ratio) was 30:1 and 10:1. Mutants analyzed were MGFc55/60/300L (R255L; P396L; D270E; Y300L); MGFc51/60 (Q419H; P396L; D270E); MGFc52/60 (V240A; P396L; D270E); MGFc59/60 (K370E; P396L; D270E); MGFc38/60 (K392T; P396L; D270E); MGFc59 (K370E; P396L); MGFc51 (Q419H; P396L); MGFc31/60 (P247L; N421K; D270E); MGFc55/292G (R255L; P396L; D270E; R292G).

### FIGS. 19A-19B Schematic Representation of Heavy Chain Variants of the Invention

FIG. 19A. Representation of wild-type IgG1 and IgG2 heavy chains. FIG. 19B. Representation of heavy chain variant IgG2 MgFc2006, which comprises CH1, hinge and upper amino terminal CH2 domains from IgG1 and the remainder of the Fc region from IgG2. Representation of heavy chain variant MgFc2010, which comprises CH1 and hinge domains of IgG1 and the Fc region of IgG2.

### FIG. 20 Alignment of Fc Regions of Wild-Type IgG1, MgFc2006 and MgFc2010

FIG. 20 shows the alignment of the Fe regions of wild-type IgG1, MgFc2006 and MgFc2010. MgFc2006 and MgFc2011 use the Fe region of IgG2 as a backbone. The amino acid residues of IgG1 shown in the figure, 1-224, correspond to amino acid residues 223 to 447 of the IgG heavy chain according to the numbering system of Kabat. IgG1 amino acids 1-8 (corresponding to IgG1 amino acid residues 223-230 according to the numbering system of Kabat) are the carboxy terminal portions of the IgG1 hinge region. The sequences of MgFc2006 and MgFc2010 have been aligned to the IgG1 sequence by aligning the cysteine residues of the corresponding hinge regions.

### FIGS. 21A-21D FCγR Binding to Variant MgFc2006

Sensogram of real time binding of wild-type and variants MgFc2006 to FcγRIIIA V¹⁵⁸ (FIGS. 21A and 21B, respectively) and to FcγRIIIA F¹⁵⁸ (FIGS. 21C and 21D, respectively).

FIG. 22 ADCC Activity of Variants MgFc2006 and MgFc2010 in ch4D5

Ch4d5 antibodies containing variant heavy chins were assessed for their ADCC activity and compared to the ADCC activity of wild type 4D5. SKBR lymphoma cells were used as target. Effector to target ratio (E:T ratio) was 75:1 with 18 h incubation.

### FIG. 23 Alignment of Fc Regions of Wild-Type IgG1, MgFc2016 AND MgFc2022

FIG. 23 shows the alignment of the Fe regions of wild-type IgG1, MgFc2016 and MgFc2012. MgFc2006 and MgFc2010 use the Fe region of IgG2 as a backbone. The amino acid residues of IgG1 shown in the figure, 1-224, correspond to amino acid residues 223 to 447 of the IgG heavy chain according to the numbering system of Kabat. IgG1 amino acids 1-8 (corresponding to IgG1 amino acid residues 223-230 according to the numbering system of Kabat) are the carboxy terminal portions of the IgG1 hinge region. The sequences of MgFc2016 and MgFc2012 have been aligned to the IgG1 sequence by aligning the cysteine residues of the corresponding hinge regions.

### FIGS. 24A-24D FCγR Binding to Variant MgFc2016

Sensogram of real time binding of the Fe regions wild-type IgG1 (solid thin line), wild type IgG2 (long-dashed line), IgG1 variant MgFc0088 (short-dashed line) and IgG2 MgFc2016 (thick solid line) FcγRIIIA V¹⁵⁸ (FIG. 24A), FcγRIIIA F¹⁵⁸ (FIG. 24B), FcγRIIB H¹³¹ (FIG. 24C) and FcγRIIB (FIG. 24D). Variant MgFc2016 in the context of MgFc2006 corresponds to MgFc0088 in the context of wild-type IgG1.

### FIGS. 25A-25D FCγR Binding to Variant MgFc2012

Sensogram of real time binding of the Fc regions wild-type IgG1 (solid thin line), IgG1 variant MgFc0155 (solid thick line) and IgG2 MgFc2012 (dashed line) FcγRIIIA V¹⁵⁸ (FIG. 25A), FcγRIIIA F¹⁵⁸ (FIG. 25B), FcγRIIB H¹³¹ (FIG. 25C) and FcγRIIB (FIG. 25D). Variant MgFc2012 in the context of MgFc2006 corresponds to MgFc0155 in the context of wild-type IgG1.

### FIG. 26 Alignment of Fc Regions of Wild-Type IgG3, MgFc3013 and MgFc3014

FIG. 26 shows the alignment of the Fe regions of wild-type IgG3, MgFc3013 and MgFc3014. MgFc3013 and MgFc3014 use the Fe region of IgG3 as a backbone. The amino acid residues of IgG3 shown in the figure, 1-225, correspond to the carboxy terminal hinge region and Fe region of wild-type IgG3. Amino acids 1-8 of the wild type IgG3 sequence have been aligned similarly to FIGS. 20 and 23. The sequences of MgFc3013 and MgFc3014 have been aligned to the IgG3 sequence by aligning the cysteine residues of the corresponding hinge regions.

### FIGS. 27A-27D FCγR Binding to Variant MgFc3013 and MgFc3014.

Sensogram of real time binding of the Fc regions wild-type IgG1 (solid thin line), IgG3 variant MgFc3013 (solid thick line) and IgG2 MgFc3013 (dashed line) FcγRIIIA V¹⁵⁸ (FIG. 27A), FcγRIIIA F¹⁵⁸ (FIG. 27B), FcγRIIB H¹³¹ (FIG. 27C) and FcγRIIB (FIG. 27D).

### FIG. 28 Alignment of Fc Regions of Wild-Type IgG3, MgFc3011 and MgFc3012

FIG. 28 shows the alignment of the Fe regions of wild-type IgG3, MgFc301 and MgFc3012. MgFc3011 and MgFc3012 use the Fe region of IgG3 as a backbone. The amino acid residues of IgG3 shown in the figure, 1-225, correspond to the carboxy terminal hinge region and Fe region of wild-type IgG3. Amino acids 1-8 of the wild type IgG3 sequence have been aligned similarly to FIGS. 20 and 23. The sequences of MgFc3011 and MgFc3012 have been aligned to the IgG3 sequence by aligning the cysteine residues of the corresponding hinge regions.

### FIGS. 29A-29D FCγR Binding to Variant MgFc3011 and MgFc3012.

Sensogram of real time binding of the Fc regions wild-type IgG1 (solid thin line), IgG3 variant MgFc3011 (long-dashed line) and IgG2 MgFc3012 (short dashed line) FcγRIIIA V¹⁵⁸ (FIG. 29A), FcγRIIIA F¹⁵⁸ (FIG. 29B), FcγRIIB H¹³¹ (FIG. 29C) and FcγRIIB (FIG. 29D). MgFc3011 in the context of IgG3 corresponds to a wild type IgG3 Fc region. MgFc3012 in the context of IgG3 corresponds to MgFc0155 in the context of IgG1.

### FIG. 30 Alignment of Fc Regions of Wild-Type IgG3 (Allotype Y296F), MgFc3002 and MgFc3003

FIG. 30 shows the alignment of the Fc regions of wild-type IgG3 F²⁹⁶, MgFc3002 and MgFc3003. MgFc3002 and MgFc3003 use the Fe region of IgG3 F²⁹⁶ as a backbone. The amino acid residues of IgG3 shown in the figure, 1-225, correspond to the carboxy terminal hinge region and Fe region of wild-type IgG3 F296. Amino acids 1-8 of the wild type IgG3 F296 sequence have been aligned similarly to FIGS. 20 and 23. The sequences of MgFc3002 and MgFc3003 have been aligned to the IgG3 F296 sequence by aligning the cysteine residues of the corresponding hinge regions. MgFc3002 in the contect of IgG3 F²⁹⁶ corresponds to MgFc0155 in the context of IgG1. MgFc3003 in the contect of IgG3 F296 corresponds to MgFc0088a in the context of IgG1.

### FIGS. 31A-31D FCγR Binding to Variant MgFc3011 and MgFc3012.

Sensogram of real time binding of the Fc regions wild-type IgG1 (solid thin line), IgG3 variant MgFc3002 (solid thick line) and IgG2 MgFc3003 (dashed line) FcγRIIIA V¹⁵⁸ (FIG. 31A), FcγRIIIA F¹⁵⁸ (FIG. 31B), FcγRIIB H¹³¹ (FIG. 31C) and FcγRIIB (FIG. 31D). MgFc3002 in the contect of IgG3 F²⁹⁶ corresponds to MgFc0055 in the context of IgG1. MgFc3003 in the contect of IgG3 F²⁹⁶ corresponds to MgFc0088a in the context of IgG1.

### 6. DESCRIPTION OF THE PREFERRED EMBODIMENTS

The present invention relates to molecules, preferably polypeptides, and more preferably immunoglobulins (e.g., antibodies), comprising a variant heavy chain, wherein said variant heavy chain comprises domains or regions from two or more IgG isotypes. In certain embodiments the invention relates to molecules comprising CH1 and hinge domains of an IgG1 and an Fe region of IgG2, IgG3 or IgG4. The invention further encompasses molecules comprising variant heavy chains having domains or regions from IgG2, IgG3 or IgG4, and one or more amino acid modifications (e.g., substitutions, but also including insertions or deletions) in one or more regions, which modifications alter, e.g., increase or decrease, the affinity of the Fe region of said variant heavy chain for an FcγR. In some embodiments, the invention comprises modifications to the Fe region of the variant heavy chain including but not limited to any of the modifications disclosed in U.S. Pat. No. 7,355,008; U.S. Provisional Application Ser. No. 60/439,498 filed Jan. 9, 2003; U.S. Provisional Application Ser. No. 60/456,041 filed Mar. 19, 2003; U.S. Provisional Application Ser. No. 60/514,549 filed Oct. 23, 2003; PCT Publication WO 2006/088494; U.S. Provisional Application Ser. No. 60/587,251 filed Jul. 12, 2004; PCT Publication WO2006/113665; U.S. Provisional Application Ser. No. 60/636,056 filed Dec. 13, 2004; U.S. Provisional Application Ser. No. 60/626,510 filed Nov. 10, 2004; and U.S. Provisional Application 60/707,419 filed Aug. 10, 2005. Each of the above mentioned applications is incorporated herein by reference in its entirety. In some embodiments, the invention provides molecules comprising a variant heavy chain which contains an Fe region of IgG2, IgG3 or IgG4, having at least one amino acid modification relative to a wild type heavy chain containing an Fe region of the same isotype, which Fe region of the variant heavy chain binds FcγRIIIA with a greater affinity, relative to a comparable molecule, i.e., being the same as said molecule comprising a heavy chain with the Fe region of IgG2, IgG3 or IgG4, but not having the one or more amino acid modifications, as determined by methods known to one skilled in the art for determining heavy chain-antibody receptor interactions, in particular Fc-FcγR interactions, and methods disclosed herein, for example, an ELISA assay or a surface plasmon resonance assay. In yet other embodiments, the invention encompasses molecules comprising a variant heavy chain which contains an Fe region of IgG2, IgG3 or IgG4, having at least one amino acid modification relative to a wild type heavy chain containing an Fe region of the same isotype, which Fe region of the variant heavy chain binds FcγRIIIA with a reduced affinity relative to a comparable molecule comprising the wild-type Fe region. In a preferred embodiment, the molecules of the invention further specifically bind FcγRIIB (via the Fe region) with a lower affinity than a comparable molecule comprising the wild-type heavy chain having the Fe region of the same isotype binds FcγRIIB. In some embodiments, the invention encompasses molecules comprising a variant heavy chain which contains an Fe region of IgG2, IgG3 or IgG4, having at least one amino acid modification relative to a wild type heavy chain containing an Fe region of the same isotype, which Fe region of the variant heavy chain binds FcγRIIIA and FcγRIIB with a greater affinity, relative to a comparable molecule comprising the wild-type heavy chain with an Fe region of the same isotype. In other embodiments, the invention encompasses molecules comprising a variant heavy chain which contains an Fe region of IgG2, IgG3 or IgG4, having at least one amino acid modification relative to a wild type heavy chain containing an Fc region of the same isotype, which Fc region of the variant heavy chain binds FcγRIIB with a greater affinity, relative to a comparable molecule comprising the wild-type heavy chain having an Fc region of the same isotype. In other embodiments, the invention encompasses molecules comprising a variant heavy chain which contains an Fc region of IgG2, IgG3 or IgG4, having at least one amino acid modification relative to a wild type heavy chain containing an Fc region of the same isotype, which Fc region of the variant heavy chain binds FcγRIIB with a reduced affinity, relative to a comparable molecule comprising the wild-type heavy chain having an Fc region of the same isotype.

The invention encompasses the use of the amino acid modifications disclosed herein or known in the art in the context of a heavy chain containing the domains or regions from two or more IgG isotypes. As disclosed herein, amino acid modification of the Fc region can profoundly affect immunoglobulin binding and/or effector function activity. However, these alterations in functional characteristics can be further refined and/or manipulated when implemented in the context of selected IgG isotypes. Similarly, the native characteristics of the isotype may be manipulated by the one or more amino acid modifications. The multiple IgG isotypes (i.e., IgG1, IgG2, IgG3 and IgG4) have differing physical and functional properties including serum half-life, complement fixation, FcγR binding affinities and effector function activities (e.g. ADCC, CDC). In preferred embodiments, the amino acid modification and IgG region are independently selected based on their respective, separate binding and/or effector function activities in order to engineer a variant heavy chain with desired characteristics. In most embodiments, said amino acid modifications and IgG regions have been separately assayed for binding and/or effector function activity as described herein or known in the art in an the context of an IgG1. In certain embodiments, said amino acid modification and IgG region display similar functionality, e.g., increased affinity for FcγRIIA, when separately assayed for FcγR binding or effector function in the context of a wild-type heavy chain and/or Fc region. The combination of said amino acid modification and selected IgG region then act additively or, more preferably, synergistically to modify said functionality in the variant heavy chain of the invention relative to a wild-type heavy chain having corresponding region(s) of the same isotype. In other embodiments, said amino acid modification and IgG region display opposite functionalities, e.g., increased and decreased, respectively, affinity for FcγRIIA, when separately assayed for FcγR binding or effector function in the context of a wild-type heavy chain and/or Fc region as described herein or known in the art; the combination of said “opposite” amino acid modification and selected IgG region then act to selectively temper or reduce a specific functionality in the variant heavy chain of the invention relative to a wild-type heavy chain having corresponding region(s) of the same isotype. Alternatively, the invention encompasses variant heavy chains comprising combinations of amino acid modifications known in the art and/or described herein and selected IgG regions that exhibit novel properties, which properties were not detectable when said modifications and/or regions were independently assayed as described herein.

The functional characteristics of the multiple IgG isotypes, and domains thereof, are well known in the art. The amino acid sequences of IgG1, IgG2, IgG3 and IgG4 are presented in FIG. X. Selection and/or combinations of two or more domains from specific IgG isotypes for use in the variant heavy chain of the invention may be based on any known parameter of the parent isotypes including affinity to FcγR (Table X; Flesch and Neppert, 1999, J. Clin. Lab. Anal. 14:141-156; Chappel et al., 1993, J. Biol. Chem. 33:25124-25131; Chappel et al., 1991, Proc. Natl. Acad. Sci. USA 88:9036-9040, each of which is hereby incorporated by reference in its entirety). For example, use of regions or domains from IgG isotypes the exhibit limited or no binding to FcγRIIB, e.g., IgG2 or IgG4, may find particular use where a variant heavy chain is desired to be engineered to maximize binding to an activating receptor and minimize binding to an inhibitory receptor. Similarly, use of regions or domains from IgG isotypes known to preferentially bind C1q or FcγRIIIA, e.g., IgG3 (Brüggemann et al., 1987, J. Exp. Med. 166:1351-1361), may be combined with amino acid modifications known in the art to enhance ADCC, see Table 8, to engineer a variant heavy chain such that effector function activity, e.g., complement activation or ADCC, is maximized.

TABLE 2
General characteristics of IgG binding to FcγR, adapted from
Flesch and Neppert, 1999, J. Clin. Lab. Anal. 14: 141-156

| | Estimated | |
|---|---|---|
| | Affinity for IgG | |
| Receptor | (M⁻¹) | Relative Affinity |
| FcγRI | 10⁸-10⁹ | IgG3 > IgG1 >> IgG4 |
| | | no-binding: IgG2 |
| FcγRIIA R^{131A} | <10⁷ | IgG3 > IgG1 |
| | | no-binding: IgG2, IgG4 |
| FcγRIIA H^{131A} | <10⁷ | IgG3 > IgG1 > IgG2 |
| | | no-binding: IgG4 |
| FcγRIIB^{A} | <10⁷ | IgG3 > IgG1 > IgG4 |
| | | no-binding: IgG2 |
| FcγRIII | <10⁷ | IgG3 = IgG1 |
| | | no-binding: IgG2, IgG4 |

^{A}binds only complexed IgG

The invention also encompasses the use of the amino acid modifications disclosed herein or known in the art in the context of a heavy chain containing the domains or regions from two or more IgG isotypes, to introduce known IgG polymorphisms in the novel context of the variant heavy chain of the invention. Polymorphisms found in the Fc regions of differing IgG isotypes has been suggested to underlie their differences in eliciting specific effector function activities (Kim et al., 2001, J. Mol. Evol. 53:1-9, hereby incorporated by reference in its entirety). Use of known polymorphisms in the context of the variant heavy chain of the invention may therefore effect modulation of specific interactions with select effector cell populations.

In some embodiments, the invention encompasses molecules comprising a variant heavy chain which contains an Fc region of IgG2, IgG3 or IgG4, having at least one amino acid modification relative to a wild type heavy chain containing an Fc region of the same isotype, which Fc region of the variant heavy chain does not show a detectable binding to any FcγR (e.g., does not bind FcγRIIA, FcγRIIB, or FcγRIIIA, as determined by, for example, an ELISA assay), relative to a comparable molecule comprising the wild-type Fc region having an Fc region of the same isotype.

In a specific embodiment, the invention encompasses molecules comprising a variant heavy chain which contains an Fc region of IgG2, IgG3 or IgG4, having at least one amino acid modification relative to a wild type heavy chain containing an Fc region of the same isotype, which Fc region of the variant heavy chain only binds one FcγR, wherein said FcγR is FcγIIIA. In another specific embodiment, the invention encompasses molecules comprising a variant heavy chain which contains an Fc region of IgG2, IgG3 or IgG4, having at least one amino acid modification relative to a wild type heavy chain containing an Fc region of the same isotype, which Fc region of the variant heavy chain only binds one FcγR, wherein said FcγR is FcγRIIA. In yet another embodiment, the invention encompasses molecules comprising a variant heavy chain which contains an Fc region of IgG2, IgG3 or IgG4, having at least one amino acid modification relative to a wild type heavy chain containing an Fc region of the same isotype, which Fc region of the variant heavy chain only binds one FcγR, wherein said FcγR is FcγRIIB. The invention particularly relates to the modification of human or humanized therapeutic antibodies (e.g., tumor specific anti-angiogenic or anti-inflammatory monoclonal antibodies) for enhancing the efficacy of therapeutic antibodies by enhancing, for example, the effector function of the therapeutic antibodies, e.g., enhancing ADCC.

The affinities and binding properties of the molecules of the invention for an FcγR are initially determined using in vitro assays (biochemical or immunological based assays) known in the art for determining Fc-FcγR interactions, i.e., specific binding of an Fc region to an FcγR including but not limited to ELISA assay, surface plasmon resonance assay, immunoprecipitation assays (See Section 5.2). Preferably, the binding properties of the molecules of the invention are also characterized by in vitro functional assays for determining one or more FcγR mediator effector cell functions (See Section 5.3). In most preferred embodiments, the molecules of the invention have similar binding properties in in vivo models (such as those described and disclosed herein) as those in in vitro based assays However, the present invention does not exclude molecules of the invention that do not exhibit the desired phenotype in in vitro based assays but do exhibit the desired phenotype in vivo.

In some embodiments, the molecules of the invention comprising a variant heavy chain comprise at least one amino acid modification in the CH3 domain of the Fc region, which is defined as extending from amino acids 342-447. In other embodiments, the molecules of the invention comprising a variant heavy chain comprise at least one amino acid modification in the CH2 domain of the Fc region, which is defined as extending from amino acids 231-341. In other embodiments, the molecules of the invention comprising a variant heavy chain comprise at least one amino acid modification in the CH1 domain of the Fc region, which is defined as extending from amino acids 118-215. In some embodiments, the molecules of the invention comprise at least two amino acid modifications, wherein each modification is in a separate region of the variant heavy chain, e.g., one modification is in the CH3 region and one modification is in the CH2 region, one modification is in the CH3 region and one modification is in the CH1 region or one modification is in the CH2 region and one modification is in the CH1 region. The invention further encompasses amino acid modification in the hinge region. Molecules of the invention with one or more amino acid modifications in the CH1, CH2 and/or CH3 domains have altered affinities for an FcγR as determined using methods described herein or known to one skilled in the art.

In particularly preferred embodiments, the invention encompasses molecules comprising a variant heavy chain wherein said variant has an increased binding to FcγRIIA (CD32A) and/or an increased ADCC activity, as measured using methods known to one skilled in the art and exemplified herein. The ADCC assays used in accordance with the methods of the invention may be NK dependent or macrophage dependent.

The heavy chain variants of the present invention may be combined with other known heavy chain modifications, in particular modifications to the Fc region, including but not limited to modifications which alter effector function and modifications which alter FcγR binding affinity. In a particular embodiment, an heavy variant of the invention comprising a first amino acid modification in the CH1 domain, CH2 domain, CH3 domain or the hinge region may be combined with a second heavy chain modification such that the second heavy chain modification is not in the same domain as the first so that the first modification confers an additive, synergistic or novel property on the second modification. In some embodiments, the heavy chain variants of the invention do not have any amino acid modification in the CH1 domain. In other embodiments, the heavy chain variants of the invention do not have any amino acid modification in the CH2 domain.

The heavy chain variants of the present invention may be combined with any modifications in the art such as those disclosed in Table 3 below.

TABLE 3
Substitution(s)

| | | | |
|---|---|---|---|
| V264A | V264L | V264I | F241W |
| F241L | F243W | F243L | F241L/F243L/V262I/V264I |
| F241L/V262I | F243L/V2641 | F241W/F243W | F241W/F243W/V262A/V264A |
| L328M | L328E | L328F | F243L/V262I/V264W |
| I332E | L328M/I332E | P244H | F241Y/F243Y/V262T/V264T |
| P245A | P247V | V264I/I332E | F241E/F243R/V262E/V264R |
| W313F | P247G | S239E/I332E | F241E/F243Q/V262T/V264E |
| S298A | S298A/I332E | S239Q/I332E | F241R/F243Q/V262T/V264R |
| S239E | D265G | D265N | F241E/F243Y/V262T/V264R |
| S239E/D265G | S239E/D265N | S239E/D265Q | P244H/P245A/P247V |
| Y296E | Y296Q | S298T | F241E/F243R/V262E/V264R/I332E |
| S298N | T299I | A327S | F241E/F243Q/V262T/V264E/I332E |
| S267Q/A327S | A327N | S267L/A327S | F241R/F243Q/V262T/V264R/I332E |
| A327L | P329F | A330L | F241E/F243Y/V262T/V264R/I332E |
| A330Y | I332D | N297S | S298A/E333A/K334A |
| N297D | N297S/I332E | N297D/I332E | D265Y/N297D/I332E |
| N297E/I332E | L328I/I332E | L328Q/I332E | D265Y/N297D/T299L/I332E |
| I332N | I332Q | V264T | D265F/N297E/I332E |
| V264F | V240I | V263I | S239D/I332D |
| V266I | T299A | T299S | S239D/I332E |
| T299V | N325Q | N325L | S239E/V264I/I332E |
| S239D | S239N | S239F | S239Q/V264I/I332E |
| S239D/I332Q | S239E/I332D | S239E/I332N | S239E/V264I/A330Y/I332E |
| S239N/I332D | S239N/I332E | S239N/I332N | S239N/I332Q |
| S239Q/I332D | S239Q/I332N | S239Q/I332Q | K326E |
| N325T | N325V | N325H | L328D/I332E |
| L328E/I332E | L328N/I332E | L328Q/I332E | L328V/I332E |
| L328T/I332E | L328H/I332E | L328I/I332E | L328A |
| I332T | I332H | I332Y | I332A |
| N325I | S239D/I332N | S239E/I332Q | S239E/V264I/S298A/A330Y/I332E |
| T256A | K290A | D312A | S239D/N297D/I332E |
| *K326A | S298A | E333A | S239E/N297D/I332E |
| K334A | E430A | T359A | S239D/D265V/N297D/I332E |
| K360A | E430A | K320M | S239D/D265I/N297D/I332E |
| K326S | K326N | K326D | S239D/D265L/N297D/I332E |
| K326E | K334Q | K334E | S239D/D265F/N297D/I332E |
| K334H | K334V | K334L | S239D/D265Y/N297D/I332E |
| K334M | A330K | T335K | S239D/D265H/N297D/I332E |
| A339T | E333A/K334A | T256A/S298A | S239D/D265T/N297D/I332E |
| S298A/E333A | T256A | K290A | T256A/D280A/S298A/T307A |
| K326A | R255A | E258A | S298A/E333A/K334A/S298A/K334A |
| S267A | E272A | N276A | S267A/E258A/D280A/R255A |
| D280A | E283A | H285A | V264I/N297D/I332E |
| N286A | P331A | S337A | Y296D/N297D/I332E |
| H268A | E272A | E430A | Y296E/N297D/I332E |
| A330K | R301M | H268N | Y296N/N297D/I332E |
| H268S | E272Q | N286Q | Y296Q/N297D/I332E |
| N286S | N286D | K290S | Y296H/N297D/I332E |
| K320M | K320Q | K320E | Y296T/N297D/I332E |
| T335E | K320R | K322E | N297D/T299V/I332E |
| K326S | K326D | K326E | N297D/T299I/I332E |
| A330K | S267A/E258A | S267A/R255A | N297D/T299L/I332E |
| S267A/D280A | S267A/E272A | S267A/E293A | N297D/T299F/I332E |
| P238A | D265A | E269A | N297D/T299H/I332E |
| D270A | N297A | P329A | N297D/T299E/I332E |
| A327Q | S239A | E294A | N297D/A330Y/I332E |
| Q295A | V303A | K246A | N297D/S298A/A330Y/I332E |
| I253A | T260A | K274A | S239D/A330Y/I332E |
| V282A | K288A | Q311A | S239N/A330Y/I332E |
| K317A | E318A | K338A | S239D/A330L/I332E |
| K340A | Q342A | R344A | S239N/A330L/I332E |
| E345A | Q347A | R355A | V264I/S298A/I332E |
| E356A | M358A | K360A | S239D/S298A/I332E |
| N361A | Q362A | Y373A | S239N/S298A/I332E |
| S375A | D376A | E380A | S239D/V264I/I332E |
| E382A | S383A | D413A | S239D/V264I/S298A/I332E |
| N384A | Q386A | E388A | S239D/V264I/A330L/I332E |
| K414A | N389A | N390A | S440A |
| Y391A | K392A | L398A | S442A |
| S400A | D401A | S415A | S444A |
| R416A | Q418A | Q419A | K447A |
| N421A | V422A | S424A | K246M |
| E430A | H433A | N434A | K248M |
| H435A | Y436A | T437A | A330Q |
| Q438A | K439A | Y391F | K338M |
| K340M | A378Q | Y300F | |

In other embodiments, the heavy chain variants of the present invention may be combined with any of the known heavy chain modifications in the art such as those disclosed in Tables 4 A and B below.

TABLE 4A

| Starting | Position | Position | Position | Position | Position |
|---|---|---|---|---|---|
| Variant | 300 | 298 | 296 | 295 | 294 |
| Y3001 + → | — | S298N, S298V, | Y296P, Y296F, | Q295K, Q295L, | E294N, |
| | | S298D, S298P, | or N276Q. | or Q295A. | E294A, |
| | | S298A, S298G, | | | E294Q, or |
| | | S298T, or | | | E294D. |
| | | S298L. | | | |
| Y300L + → | — | S298N, S298V, | Y296P, Y296F, | Q295K, Q295L, | E294N, |
| | | S298D, S298P, | or N276Q. | or Q295A. | E294A, |
| | | S298A, S298G, | | | E294Q, or |
| | | S298T, or | | | E294D. |
| | | S298L. | | | |
| S298N + → | Y3001, Y300L, | — | Y296P, Y296F, | Q295K, Q295L, | E294N, |
| | or Y300F. | | or N276Q. | or Q295A. | E294A, |
| | | | | | E294Q, or |
| | | | | | E294D. |
| S298V + → | Y3001, Y300L, | — | Y296P, Y296F, | Q295K, Q295L, | E294N, |
| | or Y300F. | | or N276Q. | or Q295A. | E294A, |
| | | | | | E294Q, or |
| | | | | | E294D. |
| S298D + → | Y3001, Y300L, | — | Y296P, Y296F, | Q295K, Q295L, | E294N, |
| | or Y300F. | | or N276Q. | or Q295A. | E294A, |
| | | | | | E294Q, or |
| | | | | | E294D. |
| S298P + → | Y3001, Y300L, | — | Y296P, Y296F, | Q295K, Q295L, | E294N, |
| | or Y300F. | | or N276Q. | or Q295A. | E294A, |
| | | | | | E294Q, or |
| | | | | | E294D. |
| Y296P + → | Y3001, Y300L, | S298N, S298V, | — | Q295K, Q295L, | E294N, |
| | or Y300F. | S298D, S298P, | | or Q295A. | E294A, |
| | | S298A, S298G, | | | E294Q, or |
| | | S298T, or | | | E294D. |
| | | S298L. | | | |
| Q295K + → | Y3001, Y300L, | S298N, S298V, | Y296P, Y296F, | — | E294N, |
| | or Y300F. | S298D, S298P, | or N276Q. | | E294A, |
| | | S298A, S298G, | | | E294Q, or |
| | | S298T, or | | | E294D. |
| | | S298L. | | | |
| Q295L + → | Y3001, Y300L, | S298N, S298V, | Y296P, Y296F, | — | E294N, |
| | or Y300F. | S298D, S298P, | or N276Q. | | E294A, |
| | | S298A, S298G, | | | E294Q, or |
| | | S298T, or | | | E294D. |
| | | S298L. | | | |
| E294N + → | Y3001, Y300L, | S298N, S298V, | Y296P, Y296F, | Q295K, Q295L, | — |
| | or Y300F. | S298D, S298P, | or N276Q. | or Q295A. | |
| | | S298A, S298G, | | | |
| | | S298T, or | | | |
| | | S298L. | | | |

** Note that table uses EU numbering as in Kabat.

TABLE 4B

| | Position | Position | Position | Position | Position |
|---|---|---|---|---|---|
| Starting Variant | 334 | 333 | 324 | 286 | 276 |
| Y3001 + → | K334A, K334R, K334Q, | E33A, | S324A, | N286Q, | N276Q, |
| | K334N, K334S, K334E, | E333Q, | S324N, | N286S, | N276A, |
| | K334D, K334M, K334Y, | E333N, | S324Q, | N286A, | or |
| | K334W, K334H, K334V, | E333S, | S324K, or | or | N276K. |
| | or K334L. | E333K, | S324E. | N286D. | |
| | | E333R, | | | |
| | | E333D, or | | | |
| | | E333G. | | | |
| Y300L + → | K334A, K334R, K334Q, | E33A, | S324A, | N286Q, | N276Q, |
| | K334N, K334S, K334E, | E333Q, | S324N, | N286S, | N276A, |
| | K334D, K334M, K334Y, | E333N, | S324Q, | N286A, | or |
| | K334W, K334H, K334V, | E333S, | S324K, or | or | N276K. |
| | or K334L. | E333K, | S324E. | N286D. | |
| | | E333R, | | | |
| | | E333D, or | | | |
| | | E333G. | | | |
| S298N + → | K334A, K334R, K334Q, | E33A, | S324A, | N286Q, | N276Q, |
| | K334N, K334S, K334E, | E333Q, | S324N, | N286S, | N276A, |
| | K334D, K334M, K334Y, | E333N, | S324Q, | N286A, | or |
| | K334W, K334H, K334V, | E333S, | S324K, or | or | N276K. |
| | or K334L. | E333K, | S324E. | N286D. | |
| | | E333R, | | | |
| | | E333D, or | | | |
| | | E333G. | | | |
| S298V + → | K334A, K334R, K334Q, | E33A, | S324A, | N286Q, | N276Q, |
| | K334N, K334S, K334E, | E333Q, | S324N, | N286S, | N276A, |
| | K334D, K334M, K334Y, | E333N, | S324Q, | N286A, | or |
| | K334W, K334H, K334V, | E333S, | S324K, or | or | N276K. |
| | or K334L. | E333K, | S324E. | N286D. | |
| | | E333R, | | | |
| | | E333D, or | | | |
| | | E333G. | | | |
| S298D + → | K334A, K334R, K334Q, | E33A, | S324A, | N286Q, | N276Q, |
| | K334N, K334S, K334E, | E333Q, | S324N, | N286S, | N276A, |
| | K334D, K334M, K334Y, | E333N, | S324Q, | N286A, | or |
| | K334W, K334H, K334V, | E333S, | S324K, or | or | N276K. |
| | or K334L. | E333K, | S324E. | N286D. | |
| | | E333R, | | | |
| | | E333D, or | | | |
| | | E333G. | | | |
| S298P + → | K334A, K334R, K334Q, | E33A, | S324A, | N286Q, | N276Q, |
| | K334N, K334S, K334E, | E333Q, | S324N, | N286S, | N276A, |
| | K334D, K334M, K334Y, | E333N, | S324Q, | N286A, | or |
| | K334W, K334H, K334V, | E333S, | S324K, or | or | N276K. |
| | or K334L. | E333K, | S324E. | N286D. | |
| | | E333R, | | | |
| | | E333D, or | | | |
| | | E333G. | | | |
| Y296P + → | K334A, K334R, K334Q, | E33A, | S324A, | N286Q, | N276Q, |
| | K334N, K334S, K334E, | E333Q, | S324N, | N286S, | N276A, |
| | K334D, K334M, K334Y, | E333N, | S324Q, | N286A, | or |
| | K334W, K334H, K334V, | E333S, | S324K, or | or | N276K. |
| | or K334L. | E333K, | S324E. | N286D. | |
| | | E333R, | | | |
| | | E333D, or | | | |
| | | E333G. | | | |
| Q295K + → | K334A, K334R, K334Q, | E33A, | S324A, | N286Q, | N276Q, |
| | K334N, K334S, K334E, | E333Q, | S324N, | N286S, | N276A, |
| | K334D, K334M, K334Y, | E333N, | S324Q, | N286A, | or |
| | K334W, K334H, K334V, | E333S, | S324K, or | or | N276K. |
| | or K334L. | E333K, | S324E. | N286D. | |
| | | E333R, | | | |
| | | E333D, or | | | |
| | | E333G. | | | |
| Q295L + → | K334A, K334R, K334Q, | E33A, | S324A, | N286Q, | N276Q, |
| | K334N, K334S, K334E, | E333Q, | S324N, | N286S, | N276A, |
| | K334D, K334M, K334Y, | E333N, | S324Q, | N286A, | or |
| | K334W, K334H, K334V, | E333S, | S324K, or | or | N276K. |
| | or K334L. | E333K, | S324E. | N286D. | |
| | | E333R, | | | |
| | | E333D, or | | | |
| | | E333G. | | | |
| E294N + → | K334A, K334R, K334Q, | E33A, | S324A, | N286Q, | N276Q, |
| | K334N, K334S, K334E, | E333Q, | S324N, | N286S, | N276A, |
| | K334D, K334M, K334Y, | E333N, | S324Q, | N286A, | or |
| | K334W, K334H, K334V, | E333S, | S324K, or | or | N276K. |
| | or K334L. | E333K, | S324E. | N286D. | |
| | | E333R, | | | |
| | | E333D, or | | | |
| | | E333G. | | | |

** Note that table uses EU numbering as in Kabat.

In a preferred specific embodiment, the invention encompasses a molecule comprising a variant heavy chain which contains an Fc region of IgG2, IgG3 or IgG4, having at least one amino acid modification relative to a wild type heavy chain containing an Fc region of the same isotype, such that said molecule has an altered affinity for an FcγR, provided that said variant heavy chain does not have a substitution at positions that make a direct contact with FcγR based on crystallographic and structural analysis of Fc-FcγR interactions such as those disclosed by Sondermann et al., 2000 (Nature, 406: 267-273 which is incorporated herein by reference in its entirety). Examples of positions within the Fc region of the heavy chain that make a direct contact with FcγR are amino acids 234-239 (hinge region), amino acids 265-269 (B/C loop), amino acids 297-299 (C′/E loop), and amino acids 327-332 (F/G) loop. In some embodiments, the molecules of the invention comprising variant heavy chains comprise modification of at least one residue that makes a direct contact with an FcγR based on structural and crystallographic analysis.

The FcγR interacting domain maps to the lower hinge region and select sites within the CH2 and CH3 domains of the IgG heavy chain. Amino acid residues flanking the actual contact positions and amino acid residues in the CH3 domain play a role in IgG/FcγR interactions as indicated by mutagenesis studies and studies using small peptide inhibitors, respectively (Sondermann et al., 2000 Nature, 406: 267-273; Diesenhofer et al., 1981, Biochemistry, 20: 2361-2370; Shields et al., 2001, J. Biol. Chem. 276: 6591-6604; each of which is incorporated herein by reference in its entirety). Direct contact as used herein refers to those amino acids that are within at least 1 Å, at least 2, or at least 3 angstroms of each other or within 1 Å, 1.2 Å, 1.5 Å, 1.7 Å or 2 Å Van Der Waals radius. An exemplary list of previously identified sites on the Fc that effect binding of Fc interacting proteins is listed in the Table 5 below. In some embodiments, the invention encompasses heavy chain variants that do not have any modifications at the sites listed below. In other embodiments, the invention encompasses heavy chain variants comprising amino acid modifications at one or more sites listed below in combination with other modifications disclosed herein such that such modification has a synergistic or additive effect on the property of the mutant.

TABLE 5
PREVIOUSLY IDENTIFIED SITES IN THE HEAVY CHAIN
Fc REGION THAT EFFECT BINDING OF Fc INTERACTING
PROTEINS.

| FcR-Fc | Domain | residue | FcRI | FcRII | FcRIII | C1q | FcRn |
|---|---|---|---|---|---|---|---|
| | CH2 | 233 | C | C | C | | C |
| A, B | CH2 | 234 | C | C | C | G | C |
| A, B | CH2 | 235 | C | C | C | G | C |
| A, B | CH2 | 236 | C | C | C | | C |
| A, B | CH2 | 237 | | | | | |
| A, B | CH2 | 238 | D | | | | |
| A, B | CH2 | 239 | | | C | | |
| | CH2 | 241 | D | | | | |
| | CH2 | 243 | D | | | | |
| | CH2 | 246 | D | | | | |
| | CH2 | 250 | | | | | E |
| | CH2 | 254 | | | | | C |
| | CH2 | 255 | | C | | | |
| | CH2 | 256 | | C | C | | |
| | CH2 | 258 | | C | | | |
| B | CH2 | 265 | C | C | C | F | C |
| B | CH2 | 267 | | C | | | |
| | CH2 | 268 | | C | C | | |
| B | CH2 | 269 | | | C | | |
| | CH2 | 270 | | C | C | F | |
| | CH2 | 272 | | C | | | |
| | CH2 | 276 | | C | | | |
| | CH2 | 285 | | C | | | |
| | CH2 | 286 | | C | | | |
| | CH2 | 288 | | | | | C |
| | CH2 | 290 | | C | C | | |
| | CH2 | 292 | | C | | | |
| | CH2 | 293 | | | C | | |
| | CH2 | 295 | | C | C | | |
| | CH2 | 296 | | | C | | |
| B | CH2 | 297 | X | X | X | X | |
| B | CH2 | 298 | | | | | |
| B | CH2 | 299 | | | | | |
| | CH2 | 301 | D | C | C | | |
| | CH2 | 311 | | | | | C |
| | CH2 | 312 | | | | | C |
| | CH2 | 315 | | C | | | |
| | CH2 | 317 | | | | | C |
| | CH2 | 322 | | C | C | F | |
| | CH2 | 326 | | C | | F | |
| A, B | CH2 | 327 | D, C | C | C | | |
| A | CH2 | 328 | | | | | |
| A | CH2 | 329 | D, C | C | C | F | |
| A | CH2 | 330 | | | | | |
| | CH2 | 331 | | C | | F | |
| A | CH2 | 332 | | | | | |
| | CH2 | 333 | | | C | F | |
| | CH2 | 334 | | | C | | |
| | CH2 | 337 | | C | | | |
| | CH2 | 338 | | | C | | |
| | CH3 | 339 | | | C | | |
| | CH3 | 360 | | | | | C |
| | CH3 | 362 | | | | | C |
| | CH3 | 376 | | | C | | |
| | CH3 | 378 | | C | | | |
| | CH3 | 380 | | | | | C |
| | CH3 | 382 | | | | | C |
| | CH3 | 414 | | C | | | |
| | CH3 | 415 | | | | | C |
| | CH3 | 424 | | | | | C |
| | CH3 | 428 | | | | | E |
| | CH3 | 430 | | C | | | |
| | CH3 | 433 | | | | | C |
| | CH3 | 434 | | | | | C |
| | CH3 | 435 | | | | | C |
| | CH3 | 436 | | | | | C |

Table 5 lists sites within the heavy chain Fe region that have previously been identified to be important for the Fc-FcR interaction. Columns labeled FcR-Fc identifies the Fe chain contacted by the FcR. Letters identify the reference in which the data was cited. C is Shields et al., 2001, J. Biol. Chem. 276: 6591-6604; D is Jefferis et al., 1995, Immunol. Lett. 44: 111-7; E is Hinton et al; 2004, J. Biol. Chem. 279(8): 6213-6; F is Idusogie et al., 2000, J. Immunol. 164: 4178-4184; each of which is incorporated herein by reference in its entirety.

In another preferred embodiment, the invention encompasses a molecule comprising a variant heavy chain having an Fe region of IgG2, IgG3 or IgG4, having at least one amino acid modification relative to a wild type heavy chain containing an Fe region of the same isotype, such that said molecule binds an FcγR with an altered affinity relative to a molecule comprising a wild-type Fe region. In certain embodiments, the molecules of the invention with altered affinities for FcγRs having variant heavy chains, comprise one or more amino acid modifications, wherein said one or more amino acid modification is a substitution at position 288 with asparagine, at position 330 with serine and at position 396 with leucine (MgFc10)(See Table 6); or a substitution at position 334 with glutamic acid, at position 359 with asparagine, and at position 366 with serine (MgFc13); or a substitution at position 316 with aspartic acid, at position 378 with valine, and at position 399 with glutamic acid (MgFc27); or a substitution at position 247 with leucine, and a substitution at position 421 with lysine (MgFc31); or a substitution at position 392 with threonine, and at position 396 with leucine (MgFc38); or a substitution at position 221 with glutamic acid, at position 270 with glutamic acid, at position 308 with alanine, at position 311 with histidine, at position 396 with leucine, and at position 402 with aspartic acid (MgFc42); or a substitution at position 419 with histidine, and a substitution at position 396 with leucine (MgFc51); or a substitution at position 240 with alanine, and at position 396 with leucine (MgFc52); or a substitution at position 410 with histidine, and at position 396 with leucine (MgFc53); or a substitution at position 243 with leucine, at position 305 with isoleucine, at position 378 with aspartic acid, at position 404 with serine, and at position 396 with leucine (MgFc54); or a substitution at position 255 with isoleucine, and at position 396 with leucine (MgFc55); or a substitution at position 370 with glutamic acid and at position 396 with leucine (MgFc59); or a substitution at position 243 with leucine, at position 292 with proline, at position 300 with leucine, at position 305 with isoleucine, and at position 396 with leucine (MgFc88); or a substitution at position 243 with leucine, at position 292 with proline, at position 300 with leucine, and at position 396 with leucine (MgFc88A); or a substitution at position 243 with leucine, at position 292 with proline, and at position 300 with leucine (MgFc155); or a substitution at position 435 with histidine; or a substitution at position 270 with glutamic acid; or a combination of the foregoing. In a specific embodiment, the invention encompasses a molecule comprising a variant Fc region wherein said variant Fc region comprises a substitution at position 396 with leucine, at position 270 with glutamic acid and at position 243 with leucine. In another specific embodiment the molecule further comprises one or more amino acid modification such as those disclosed herein.

In some embodiments, the invention encompasses molecules comprising a variant heavy which contains the Fc region of IgG2, IgG3 or IgG4 and which has an amino acid modification at one or more of the following positions: 119, 125, 132, 133, 141, 142, 147, 149, 162, 166, 185, 192, 202, 205, 210, 214, 215, 216, 217, 218, 219, 221, 222, 223, 224, 225, 227, 229, 231, 232, 233, 235, 240, 241, 242, 243, 244, 246, 247, 248, 250, 251, 252, 253, 254, 255, 256, 258, 261, 262, 263, 268, 269, 270, 272, 274, 275, 276, 279, 280, 281, 282, 284, 287, 288, 289, 290, 291, 292, 293, 295, 298, 301, 303, 304, 305, 306, 307, 308, 309, 310, 311, 312, 313, 315, 316, 317, 318, 319, 320, 323, 326, 327, 328, 330, 333, 334, 335, 337, 339, 340, 343, 344, 345, 347, 348, 352, 353, 354, 355, 358, 359, 360, 361, 362, 365, 366, 367, 369, 370, 371, 372, 375, 377, 378, 379, 380, 381, 382, 383, 384, 385, 386, 387, 388, 389, 390, 392, 393, 394, 395, 396, 397, 398, 399, 400, 401, 402, 404, 406, 407, 408, 409, 410, 411, 412, 414, 415, 416, 417, 419, 420, 421, 422, 423, 424, 427, 428, 431, 433, 435, 436, 438, 440, 441, 442, 443, 446, 447. Preferably such mutations result in molecules that have an altered affinity for an FcγR and/or have an altered effector cell mediated function as determined using methods disclosed and exemplified herein and/or known to one skilled in the art.

The invention encompasses molecules comprising variant heavy chains having the Fc region of IgG2, IgG3 or IgG4 and consisting of or comprising any of the mutations listed in the table below in Table 6.

TABLE 6
EXEMPLARY MUTATIONS

| SINGLE SITE MUTANTS | DOUBLE SITE MUTANTS |
|---|---|
| K392R | Q347H/A339V |
| N315I | S415I/L251F |
| S132I | K290E/L142P |
| P396L | G285E/P247H |
| P396H | K409R/S166N |
| A162V | E334A/K334A |
| R292L | R292L/K334E |
| T359N | K288N/A330S |
| T366S | R255L/E318K |
| V379L | F243L/E318K |
| K288N | V279L/P395S |
| A330S | K246T/Y319F |
| F243L | F243I/V379L |
| E318K | K288M/K334E |
| V379M | K334E/E308D |
| S219Y | E233D/K334E |
| V282M | K246T/P396H |
| D401V | H268D/E318D |
| K222N | K246I/K334N |
| K334I | K320E/K326E |
| K334E | S375C/P396L |
| I377F | K288N/K326N |
| P247L | P247L/N421K |
| F372Y | S298N/W381R |
| K326E | R255Q/K326E |
| H224L | V284A/F372L |
| F275Y | T394M/V397M |
| L398V | P247L/E389G |
| K334N | K290T/G371D |
| S400P | P247L/L398Q |
| S407I | P247L/I377F |
| F372Y | K326E/G385E |
| T366N | S298N/S407R |
| K414N | E258D/N384K |
| M352L | F241L/E258G |
| T225S | K370N/S440N |
| I377N | K317N/F423-DELETED |
| K248M | P227S/K290E |
| R292G | K334E/E380D |
| S298N | P291S/P353Q |
| D270E | V240I/V281M |
| E233G | P232S/S304G |
| R292P | P247L/L406F |
| | D399E/M428L |
| | L251F/F372L |
| | D399E/G402D |
| | D399E/M428L |
| | K392T/P396L |
| | H268N/P396L |
| | K326I/P396L |
| | H268D/P396L |
| | K210M/P396L |
| | L358P/P396L |
| | K334N/P396L |
| | V379M/P396L |
| | P227S/P396L |
| | P217S/P396L |
| | Q419H/P396L |
| | K370E/P396L |
| | L242F/P396L |
| | R255L/P396L |
| | V240A/P396L |
| | T250A/P396L |
| | P247S/P396L |
| | L410H/P396L |
| | Q419L/P396L |
| | V427A/P396L |
| | E258D/P396L |
| | N384K/P396L |
| | V323I/P396L |
| | P244H/P396L |
| | V305L/P396L |
| | S400F/P396L |
| | V303I/P396L |
| | A330V/Q419H |
| | V263Q/E272D |
| | K326E/A330T |
| | F243L/R292P |
| | F243L/P396L |

In yet other embodiments, the invention encompasses molecules comprising variant heavy chains which contain the Fc regions of IgG2, IgG3 or IgG4 and which have more than two amino acid modifications. A non-limiting example of such variants is listed in the table below (Table 7). The invention also encompasses molecules comprising mutations listed in Table 6 and further comprising one or more amino acid modifications such as those disclosed herein.

TABLE 7
EXEMPLARY COMBINATION VARIANTS

| | |
|---|---|
| D399E/R292L/V185M | P247L/N421K/D270E |
| R301C/M252L/S192T | R292P/V305I |
| P291S/K288E/H268L/A141V | R292P/V305I/F243L |
| S408I/V215I/V125L | V284M/R292L/K370N |
| G385E/P247H | R292P/V305I/P396L |
| V348M/K334N/F275I/Y202M/K147T | F243L/R292P/Y300L |
| H310Y/T289A/Y407V/E258D | F243L/Y300L/V305I/P396L |
| R292L/P396L/T359N | F243L/R292P/V305I/P396L |
| F275I/K334N/V348M | F243L/R292P/Y300L/V305I/P396L |
| F243L/R255L/E318K | R255L/P396L/D270E/Y300L |
| K334E/T359N/T366S | R255L/P396L/D270E/R292G |
| T256S/V305I/K334E/N390S | F243L/D270E/K392N/P396L |
| T335N/K370E/A378V/T394M/S424L | F243L/R255L/D270E/P296L |
| K334E/T359N/T366S/Q386R | K334E/E380D/G446V |
| K288N/A330S/P396L | V303I/V369F/M428L |
| P244H/L358M/V379M/N384K/V397M | K246E/V284M/V308A |
| P217S/A378V/S408R | E293V/Q295E/A327T |
| P247L/I253N/K334N | Y319F/P352L/P396L |
| D312E/K327N/I378S | K290T/N390I/P396L |
| D280E/S354F/A431D/L441I | K288R/T307A/K344E/P396L |
| K218R/G281D/G385R | V273I/K326E/L328I/P396L |
| P247L/A330T/S440G | K326I/S408N/P396L |
| T355N/P387S/H435Q | K261N/K210M/P396L |
| P247L/A431V/S442F | F243L/V305I/A378D/F404S/P396L |
| P343S, P353L, S375I, S383N | K290E/V369A/T393A/P396L |
| E216D, E345K, S375I | K210N/K222I/K320M/P396L |
| K288N, A330S, P396L | P217S/V305I/I309L/N390H/P396L |
| K222N, T335N, K370E, A378V, T394M | K246N/Q419R/P396L |
| G316D, A378V, D399E | P217A/T359A/P396L |
| N315I, V379M, T394M | V215I/K290V/P396L |
| K326Q, K334E, T359N, T366S | F275L/Q362H/N384K/P396L |
| A378V, N390I, V422I | A330V/H433Q/V427M |
| V282E, V369I, L406F | V263Q/E272D/Q419H |
| V397M, T411A, S415N | N276Y/T393N/W417R |
| T223I, T256S, L406F | V282L/A330V/H433Y/T436R |
| L235P, V382M, S304G, V305I, V323I | V284M/S298N/K334E/R355W |
| P247L, W313R, E388G | A330V/G427M/K438R |
| F243I/V379L/G420V | S219T/T225K/D270E/K360R |
| A231V/Q386H/V412M | K222E/V263Q/S298N |
| T215P/K274N/A287G/K334N/L365V/P396L | E233G/P247S/L306P |
| P244A/K326I/C367R/S375I/K447T | S219T/T225K/D270E |
| R301H/K340E/D399E | S254T/A330V/N361D/P243L |
| C229Y/A287T/V379M/P396L/L443V | V284M/S298N/K334E/R355W/ |
| | R416T |
| E269K/K290N/Q311R/H433Y | D270E/G316D/R416G |
| E216D/K334R/S375I | K392T/P396L/D270E |
| T335N/P387S/H435Q | R255L/P396L/D270E |
| K246I/Q362H/K370E | V240A/P396L/D270E |
| Q419H/P396L/D270E | K370E/P396L/D270E |

D221Y/M252I/A330G/A339T, T359N, V422I, H433L
D221E/D270E/V308A/Q311H/P396L/G402D
S383N/N384K/T256N/V262L/K218E/R214I/K205E/F149Y/K133M

In specific embodiments, the variant heavy chain has a leucine at position 247, a lysine at position 421 and a glutamic acid at position 270 (MgFc31/60); a threonine at position 392, a leucine at position 396, a glutamic acid at position 270, and a leucine at position 243 (MgFc38/60/F243L); a histidine at position 419, a leucine at position 396, and a glutamic acid at position 270 (MGFc51/60); a histidine at position 419, a leucine at position 396, a glutamic acid at position 270, and a leucine at position 243 (MGFc51/60/F243L); an alanine at position 240, a leucine at position 396, and a glutamic acid at position 270 (MGFc52/60); a lysine at position 255 and a leucine at position 396 (MgFc55); a lysine at position 255, a leucine at position 396, and a glutamic acid at position 270 (MGFc55/60); a lysine at position 255, a leucine at position 396, a glutamic acid at position 270, and a lysine at position 300 (MGFc55/60/Y300L); a lysine at position 255, a leucine at position 396, a glutamic acid at position 270, and a glycine at position 292 (MGFc55/60/R292G); a lysine at position 255, a leucine at position 396, a glutamic acid at position 270, and a leucine at position 243 (MgFc55/60/F243L); a glutamic acid at position 370, a leucine at position 396, and a glutamic acid at position 270 (MGFc59/60); a glutamic acid at position 270, an aspartic acid at position 316, and a glycine at position 416 (MgFc71); a leucine at position 243, a proline at position 292, an isoleucine at position 305, and a leucine at position 396 (MGFc74/P396L); a leucine at position 243, a glutamic acid at position 270, an asparagine at position 392 and a leucine at position 396; or a leucine at position 243, a leucine at position 255, a glutamic acid at position 270 and a leucine at position 396; a glutamine at position 297, or any combination of the individual substitutions.

In some embodiments, the molecules, preferably the immunoglobulins of the invention further comprise one or more glycosylation sites, so that one or more carbohydrate moieties are covalently attached to the molecule. Preferably, the antibodies of the invention with one or more glycosylation sites and/or one or more modifications in the heavy chain have an enhanced antibody mediated effector function, e.g., enhanced ADCC activity compared to a parent and/or wild-type antibody. In some embodiments, the invention further comprises antibodies comprising one or more modifications of amino acids that are directly or indirectly known to interact with a carbohydrate moiety of the antibody, including but not limited to amino acids at positions 241, 243, 244, 245, 245, 249, 256, 258, 260, 262, 264, 265, 296, 299, and 301. Amino acids that directly or indirectly interact with a carbohydrate moiety of an antibody are known in the art, see, e.g., Jefferis et al., 1995 Immunology Letters, 44: 111-7, which is incorporated herein by reference in its entirety.

In another embodiment, the invention encompasses antibodies that have been modified by introducing one or more glycosylation sites into one or more sites of the antibodies, preferably without altering the functionality of the antibody, e.g., binding activity to FcγR. Glycosylation sites may be introduced into the variable and/or constant region of the antibodies of the invention. As used herein, “glycosylation sites” include any specific amino acid sequence in an antibody to which an oligosaccharide (i.e., carbohydrates containing two or more simple sugars linked together) will specifically and covalently attach. Oligosaccharide side chains are typically linked to the backbone of an antibody via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. The antibodies of the invention may comprise one or more glycosylation sites, including N-linked and O-linked glycosylation sites. Any glycosylation site for N-linked or O-linked glycosylation known in the art may be used in accordance with the instant invention. An exemplary N-linked glycosylation site that is useful in accordance with the methods of the present invention, is the amino acid sequence: Asn-X-Thr/Ser, wherein X may be any amino acid and Thr/Ser indicates a threonine or a serine. Such a site or sites may be introduced into an antibody of the invention using methods well known in the art to which this invention pertains. See, for example, “In Vitro Mutagenesis,” Recombinant DNA: A Short Course, J. D. Watson, et al. W.H. Freeman and Company, New York, 1983, chapter 8, pp. 106-116, which is incorporated herein by reference in its entirety. An exemplary method for introducing a glycosylation site into an antibody of the invention may comprise: modifying or mutating an amino acid sequence of the antibody so that the desired Asn-X-Thr/Ser sequence is obtained.

In some embodiments, the invention encompasses methods of modifying the carbohydrate content of an antibody of the invention by adding or deleting a glycosylation site. Methods for modifying the carbohydrate content of antibodies are well known in the art and encompassed within the invention, see, e.g., U.S. Pat. No. 6,218,149; EP 0 359 096 B1; U.S. Publication No. US 2002/0028486; WO 03/035835; U.S. Publication No. 2003/0115614; U.S. Pat. No. 6,218,149; U.S. Pat. No. 6,472,511; all of which are incorporated herein by reference in their entirety. In other embodiments, the invention encompasses methods of modifying the carbohydrate content of an antibody of the invention by deleting one or more endogenous carbohydrate moieties of the antibody. In a specific embodiment, the invention encompasses shifting the glycosylation site of the Fc region of an antibody, by modifying positions adjacent to 297. In a specific embodiment, the invention encompasses modifying position 296 so that position 296 and not position 297 is glycosylated.

6.1 Polypeptides and Antibodies with Variant Heavy Chains

The present invention is based, in part, on the modification of the human IgG heavy chain functionality both by combining heavy chain domains or regions (e.g., CH domains, hinge region, Fc region) from two or more IgG isotypes and by one or more amino acid modifications (e.g., substitutions, but also including insertions or deletions) in one or more regions, which modifications alter, e.g., increase or decrease, the affinity of the Fc region of said variant heavy chain for an FcγR. Accordingly, the invention relates to molecules, preferably polypeptides, and more preferably immunoglobulins (e.g., antibodies), comprising a variant heavy chain containing domains or regions from two or more IgG isotypes, and having one or more amino acid modifications (e.g., substitutions, but also including insertions or deletions) in one or more regions, which modifications alter the affinity of the Fc region of the variant heavy chain for an FcR.

It will be appreciated by one skilled in the art that aside from amino acid substitutions, the present invention contemplates other modifications of the heavy chain amino acid sequence in order to generate an heavy chain variant with one or more altered properties, e.g., altered effector function. The invention contemplates deletion of one or more amino acid residues in one or more domains of the heavy chain in order to reduce binding to an FcγR. Preferably, no more than 5, no more than 10, no more than 20, no more than 30, no more than 50 Fc region residues will be deleted according to this embodiment of the invention. The variant heavy chain herein comprising one or more amino acid deletions will preferably retain at least about 80%, and preferably at least about 90%, and most preferably at least about 95%, of the wild type Fc region. In some embodiments, one or more properties of the molecules are maintained such as for example, non-immunogenicity, FcγRIIIA binding, FcγRIIA binding, or a combination of these properties.

In alternate embodiments, the invention encompasses amino acid insertion to generate the heavy chain variants, which variants have altered properties including altered effector function. In one specific embodiment, the invention encompasses introducing at least one amino acid residue, for example one to two amino acid residues and preferably no more than 10 amino acid residues adjacent to one or more of the heavy chain positions identified herein. In alternate embodiments, the invention further encompasses introducing at least one amino acid residue, for example one to two amino acid residues and preferably no more than 10 amino acid residues adjacent to one or more of the heavy chain positions known in the art as impacting FcγR interaction and/or binding.

The invention further encompasses incorporation of unnatural amino acids to generate the heavy chain variants of the invention. Such methods are known to those skilled in the art such as those using the natural biosynthetic machinery to allow incorporation of unnatural amino acids into proteins, see, e.g., Wang et al., 2002 Chem. Comm. 1:1-11; Wang et al., 2001, Science, 292: 498-500; van Hest et al., 2001. Chem. Comm. 19: 1897-1904, each of which is incorporated herein by reference in its entirety. Alternative strategies focus on the enzymes responsible for the biosynthesis of amino acyl-tRNA, see, e.g., Tang et al., 2001, J. Am. Chem. 123(44): 11089-11090; Kiick et al., 2001, FEBS Lett. 505(3): 465; each of which is incorporated herein by reference in its entirety.

The affinities and binding properties of the molecules of the invention for an FcγR are initially determined using in vitro assays (biochemical or immunological based assays) known in the art for determining heavy chain-antibody receptor interactions, in particular Fc-FcγR interactions, i.e., specific binding of an Fe region to an FcγR including but not limited to ELISA assay, surface plasmon resonance assay, immunoprecipitation assays (See Section 5.2). Preferably, the binding properties of the molecules of the invention are also characterized by in vitro functional assays for determining one or more FcγR mediator effector cell functions (See Section 5.3). In most preferred embodiments, the molecules of the invention have similar binding properties in in vivo models (such as those described and disclosed herein) as those in in vitro based assays. However, the present invention does not exclude molecules of the invention that do not exhibit the desired phenotype in in vitro based assays but do exhibit the desired phenotype in vivo. A representative flow chart of the screening and characterization of molecules of the invention is described in FIG. 2.

The invention encompasses molecules comprising a variant heavy chain having the Fe region of IgG2, IgG3 or IgG4 that binds with a greater affinity to one or more FcγRs relative to a wild type heavy chain having an Fe region of the same isotype. Such molecules preferably mediate effector function more effectively as discussed infra. In other embodiments, the invention encompasses molecules comprising a variant heavy chain having the Fe region of IgG2, IgG3 or IgG4 that bind with a weaker affinity to one or more FcγRs relative to a wild type heavy chain having an Fe region of the same isotype. Reduction or elimination of effector function is desirable in certain cases for example in the case of antibodies whose mechanism of action involves blocking or antagonism but not killing of the cells bearing a target antigen. Reduction or elimination of effector function would be desirable in cases of autoimmune disease where one would block FcγR activating receptors in effector cells (This type of function would be present in the host cells). In general increased effector function would be directed to tumor and foreign cells.

The heavy chain variants of the present invention may be combined with other heavy chain modifications, including but not limited to modifications that alter effector function. The invention encompasses combining a heavy chain variant of the invention with other heavy chain modifications to provide additive, synergistic, or novel properties in antibodies or Fe fusions. Preferably the heavy chain variants of the invention enhance the phenotype of the modification with which they are combined. For example, if an heavy chain variant of the invention is combined with a mutant known to bind FcγRIIIA with a higher affinity than a comparable molecule comprising a wild type heavy chain having an Fe region of the same isotype; the combination with a mutant of the invention results in a greater fold enhancement in FcγRIIIA affinity.

In one embodiment, the heavy variants of the present invention may be combined with other known heavy chain variants such as those disclosed in Duncan et al., 1988, Nature 332:563-564; Lund et al., 1991, J. Immunol. 147:2657-2662; Lund et al., 1992, Mol Immunol 29:53-59; Alegre et al., 1994, Transplantation 57:1537-1543; Hutchins et al., 1995, Proc Natl. Acad Sci USA 92:11980-11984; Jefferis et al., 1995, Immunol Lett. 44:111-117; Lund et al., 1995, Faseb J 9:115-119; Jefferis et al., 1996, Immunol Lett 54: 101-104; Lund et al., 1996, J Immunol 157:49634969; Armour et al., 1999, Eur J Immunol 29:2613-2624; Idusogie et al., 2000, J Immunol 164:41784184; Reddy et al., 2000, J Immunol 164:1925-1933; Xu et al., 2000, Cell Immunol 200:16-26; Idusogie et al., 2001, J Immunol 166:2571-2575; Shields et al., 2001, J Biol Chem 276:6591-6604; Jefferis et al., 2002, Immunol Lett 82:57-65; Presta et al., 2002, Biochem Soc Trans 30:487-490); U.S. Pat. No. 5,624,821; U.S. Pat. No. 5,885,573; U.S. Pat. No. 6,194,551; PCT WO 00/42072; PCT WO 99/58572; each of which is incorporated herein by reference in its entirety.

In some embodiments, the heavy chain variants of the present invention are incorporated into an antibody or Fc fusion that comprises one or more engineered glycoforms, i.e., a carbohydrate composition that is covalently attached to a molecule comprising a heavy chain or region thereof, wherein said carbohydrate composition differs chemically from that of a parent molecule comprising a heavy chain or region thereof. Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example DI N-acetylglucosaminyltransferase III (GnTIII), by expressing a molecule comprising a heavy chain or region thereof in various organisms or cell lines from various organisms, or by modifying carbohydrate(s) after the molecule comprising the heavy chain or region thereof has been expressed. Methods for generating engineered glycoforms are known in the art, and include but are not limited to those described in Umana et al., 1999, Nat. Biotechnol 17:176-180; Davies et al., 20017 Biotechnol Bioeng 74:288-294; Shields et al., 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland); each of which is incorporated herein by reference in its entirety. See, e.g., WO 00061739; EA01229125; US 20030115614; Okazaki et al., 2004, JMB, 336: 1239-49 each of which is incorporated herein by reference in its entirety.

The heavy chain variants of the present invention may be optimized for a variety of properties. Properties that may be optimized include but are not limited to enhanced or reduced affinity for an FcγR, enhanced or reduced effector function. In a preferred embodiment, the heavy chain variants of the present invention are optimized to possess enhanced affinity for a human activating FcγR, preferably FcγR, FcγRIIA, FcγRIIc, FcγRIIIA, and FcγRIIIB, most preferably FcγRIIIA. In an alternate preferred embodiment, the Fc variants are optimized to possess reduced affinity for the human inhibitory receptor FcγRIIB. These preferred embodiments are anticipated to provide antibodies and Fc fusions with enhanced therapeutic properties in humans, for example enhanced effector function and greater anti-cancer potency as described and exemplified herein. These preferred embodiments are anticipated to provide antibodies and Fc fusions with enhanced tumor elimination in mouse xenograft tumor models.

In an alternate embodiment the heavy chain variants of the present invention are optimized to have reduced affinity for a human FcγR, including but not limited to FcγRI, FcγRIIA, FcγRIIB, FcγRIIc, FcγRIIIA, and FcγRIIIB. These embodiments are anticipated to provide antibodies and Fc fusions with enhanced therapeutic properties in humans, for example reduced effector function and reduced toxicity.

In alternate embodiments the heavy chain variants of the present invention possess enhanced or reduced affinity for FcγRs from non-human organisms, including but not limited to mice, rats, rabbits, and monkeys. Heavy chain variants that are optimized for binding to a non-human FcγR may find use in experimentation. For example, mouse models are available for a variety of diseases that enable testing of properties such as efficacy, toxicity, and pharmacokinetics for a given drug candidate. As is known in the art, cancer cells can be grafted or injected into mice to mimic a human cancer, a process referred to as xenografting. Testing of antibodies or Fc fusions that comprise heavy chain variants, or portions thereof, that are optimized for one or more mouse FcγRs, may provide valuable information with regard to the efficacy of the antibody or Fc fusion, its mechanism of action, and the like.

While it is preferred to alter binding to an FcγR, the instant invention further contemplates heavy chain variants with altered binding affinity to the neonatal receptor (FcRn). Although not intending to be bound by a particular mechanism of action, the heavy chain variants with improved affinity for FcRn are anticipated to have longer serum half-lives, and such molecules will have useful applications in methods of treating mammals where long half-life of the administered polypeptide is desired, e.g., to treat a chronic disease or disorder. Although not intending to be bound by a particular mechanism of action, heavy chain variants with decreased FcRn binding affinity, on the contrary, are expected to have shorter half-lives, and such molecules may, for example, be administered to a mammal where a shortened circulation time may be advantageous, e.g., for in vivo diagnostic imaging or for polypeptides which have toxic side effects when left circulating in the blood stream for extended periods. Fc region variants with decreased FcRn binding affinity are anticipated to be less likely to cross the placenta, and thus may be utilized in the treatment of diseases or disorders in pregnant women.

In other embodiments, these variants may be combined with other known heavy chain modifications with altered FcRn affinity such as those disclosed in International Publication Nos. WO 98/23289; and WO 97/34631; and U.S. Pat. No. 6,277,375, each of which is incorporated herein by reference in its entirety.

The invention encompasses any other method known in the art for generating antibodies having an increased half-life in vivo, for example, by introducing one or more amino acid modifications (i.e., substitutions, insertions or deletions) into an IgG constant domain, or FcRn binding fragment thereof (preferably a Fc or hinge-Fc domain fragment). See, e.g., International Publication Nos. WO 98/23289; and WO 97/34631; and U.S. Pat. No. 6,277,375, each of which is incorporated herein by reference in its entirety to be used in combination with the heavy chain variants of the invention. Further, antibodies of the invention can be conjugated to albumin in order to make the antibody or antibody fragment more stable in vivo or have a longer half-life in vivo. The techniques well-known in the art, see, e.g., International Publication Nos. WO 93/15199, WO 93/15200, and WO 01/77137, and European Patent No. EP 413,622, all of which are incorporated herein by reference in their entirety.

The variant(s) described herein may be subjected to further modifications, often times depending on the intended use of the variant. Such modifications may involve further alteration of the amino acid sequence (substitution, insertion and/or deletion of amino acid residues), fusion to heterologous polypeptide(s) and/or covalent modifications. Such further modifications may be made prior to, simultaneously with, or following, the amino acid modification(s) disclosed herein which results in altered properties such as an alteration of Fc receptor binding and/or ADCC activity.

Alternatively or additionally, the invention encompasses combining the amino acid modifications disclosed herein with one or more further amino acid modifications that alter C1q binding and/or complement dependent cytoxicity function of the heavy chain as determined in vitro and/or in vivo. Preferably, the starting molecule of particular interest herein is usually one that binds to C1q and displays complement dependent cytotoxicity (CDC). The further amino acid substitutions described herein will generally serve to alter the ability of the starting molecule to bind to C1q and/or modify its complement dependent cytotoxicity function, e.g., to reduce and preferably abolish these effector functions. In other embodiments molecules comprising substitutions at one or more of the described positions with improved C1q binding and/or complement dependent cytotoxicity (CDC) function are contemplated herein. For example, the starting molecule may be unable to bind C1q and/or mediate CDC and may be modified according to the teachings herein such that it acquires these further effector functions. Moreover, molecules with preexisting C1q binding activity, optionally further having the ability to mediate CDC may be modified such that one or both of these activities are altered, e.g., enhanced. In some embodiments, the invention encompasses variant heavy chains having the Fc region of IgG2, IgG3 or IgG4 with altered CDC activity without any alteration in C1q binding. In yet other embodiments, the invention encompasses variant Fc regions with altered CDC activity and altered C1q binding.

To generate an heavy chain with altered C1q binding and/or complement dependent cytotoxicity (CDC) function, the amino acid positions to be modified are generally selected from positions 270, 322, 326, 327, 329, 331, 333, and 334, where the numbering of the residues in an IgG heavy chain is that of the EU index as in Kabat et al., Sequences of Proteins of Immunological Interest, 5th Ed. Public Health Service, National Institutes of Health, Bethesda, Md. (1999). These amino acid modifications may be combined with one or more heavy chain modifications disclosed herein to provide a synergistic or additive effect on C1q binding and/or CDC activity. In other embodiments, the invention encompasses heavy chain variants with altered C1q binding and/or complement dependent cytotoxicity (CDC) function comprising an amino acid substitution at position 396 with leucine and at position 255 with leucine; or an amino acid substitution at position 396 with leucine and at position 419 with histidine; an amino acid substitution at position 396 with leucine and at position 370 with glutamic acid; an amino acid substitution at position 396 with leucine and at position 240 with alanine; an amino acid substitution at position 396 with leucine and at position 392 with threonine; an amino acid substitution at position 247 with leucine and at position 421 with lysine. The invention encompasses any known modification of the Fc region which alters C1q binding and/or complement dependent cytotoxicity (CDC) function such as those disclosed in Idusogie et al., 2001, J. Immunol. 166(4) 2571-5; Idusogie et al., J. Immunol. 2000 164(8): 4178-4184; each of which is incorporated herein by reference in its entirety.

As disclosed above, the invention encompasses a heavy chain region with altered effector function, e.g., modified C1q binding and/or FcR binding and thereby altered CDC activity and/or ADCC activity. In specific embodiments, the invention encompasses variant heavy chains having the Fc region of IgG2, IgG3 or IgG4 which are characterized by improved C1q binding and improved FcγRIII binding; e.g. having both improved ADCC activity and improved CDC activity. In alternative embodiments, the invention encompasses a molecule comprising a variant heavy chain having an Fc regions of IgG2, IgG3 or IgG4 which is characterized by reduced CDC activity and/or reduced ADCC activity. In other embodiments, one may increase only one of these activities, and optionally also reduce the other activity, e.g. to generate a variant heavy chain with improved ADCC activity, but reduced CDC activity and vice versa.

A. Mutants with Enhanced Altered Affinities for FcγRIIIA and/or FcγRIIA

The invention encompasses molecules comprising a variant heavy chain containing an Fc region of IgG2, IgG3 or IgG4, having at least one amino acid modification (e.g., substitutions) relative to a wild type heavy chain containing an Fc region of the same isotype, wherein such modifications alter the affinity of the variant Fc region for an activating FcγR. In some embodiments, molecules of the invention comprise a variant heavy chain which contains an Fc region of IgG2, IgG3 or IgG4, having one or more amino acid modifications (e.g., substitutions) in one or more regions, which modifications increase the affinity of the Fc region of the variant heavy chain for FcγRIIIA and/or FcγRIIA by at least 2-fold, relative to a comparable molecule comprising a wild-type heavy chain having an Fc region of the same isotype. In another specific embodiment, molecules of the invention comprise a variant heavy chain which contains an Fc region of IgG2, IgG3 or IgG4, having one or more amino acid modifications (e.g., substitutions) in one or more regions, which modifications increase the affinity of the Fc region of the variant heavy chain for FcγRIIIA and/or FcγRIIA by greater than 2 fold, relative to a comparable molecule comprising a wild-type heavy chain having an Fc region of the same isotype. In other embodiments of the invention the one or more amino acid modifications increase the affinity of the variant Fc region for FcγRIIIA and/or FcγRIIA by at least 3-fold, 4-fold, 5-fold, 6-fold, 8-fold, or 10-fold relative to a comparable molecule comprising a wild-type heavy chain having an Fc region of the same isotype. In yet other embodiments of the invention the one or more amino acid modifications decrease the affinity of the Fc region of the variant heavy chain for FcγRIIIA and/or FcγRIIA by at least 3-fold, 4-fold, 5-fold, 6-fold, 8-fold, or 10-fold relative to a comparable molecule comprising a wild-type heavy chain having an Fe region of the same isotype. Such fold increases are preferably determined by an ELISA or surface plasmon resonance assays. In a specific embodiment, wherein the Fe region is an IgG2 Fe region, the one or more amino acid modifications do not include or are not solely a substitution at position 233 with glutamic acid; a substitution at position 234 with leucine; a substitution at position 235 with leucine; a substitution or insertion at position 237 with glycine.

In another specific embodiment, the invention encompasses a molecule comprising a variant heavy chain which contains an Fe region of IgG2, IgG3 or IgG4, having at least one amino acid modification relative to a wild type heavy chain containing an Fe region of the same isotype, such that said molecule specifically binds FcγRIIA with a greater affinity than a comparable molecule, i.e., comprising the wild-type heavy chain having an Fe region of the same isotype, binds FcγRIIA. In a specific embodiment, molecules of the invention comprise a variant heavy chain which contains an Fe region of IgG2, IgG3 or IgG4, having one or more amino acid modifications (e.g., substitutions) in one or more regions, which modifications increase the affinity of the Fe region of the variant heavy chain for FcγRIIA by at least 2-fold, relative to a comparable molecule comprising a wild-type heavy chain having an Fe region of the same isotype. In another specific embodiment, molecules of the invention comprise a variant heavy chain which contains an Fe region of IgG2, IgG3 or IgG4, having one or more amino acid modifications (e.g., substitutions) in one or more regions, which modifications increase the affinity of the Fe region of the variant heavy chain for FcγRIIA by greater than 2 fold, relative to a comparable molecule comprising a wild-type heavy chain having an Fe region of the same isotype. In other embodiments of the invention the one or more amino acid modifications increase the affinity of the variant heavy chain for FcγRIIA by at least 3-fold, 4-fold, 5-fold, 6-fold, 8-fold, or 10-fold relative to a comparable molecule comprising a wild-type heavy chain having an Fe region of the same isotype.

In a specific embodiment, the invention encompasses molecules, preferably polypeptides, and more preferably immunoglobulins (e.g., antibodies), comprising a variant heavy chain which contains an Fe region of IgG2, IgG3 or IgG4, having one or more amino acid modifications (e.g., substitutions but also include insertions or deletions), which modifications increase the affinity of the Fe region of the variant heavy chain for FcγRIIIA and/or FcγRIIA by at least 65%, at least 70%, at least 75%, at least 85%, at least 90%, at least 95%, at least 99%, at least 100%, at least 150%, and at least 200%, relative to a comparable molecule comprising a wild-type heavy chain having an Fe region of the same isotype.

In a specific embodiment, the one or more amino acid modifications which increase the affinity of the Fc region of the variant heavy chain comprise a substitution at position 347 with histidine, and at position 339 with valine; or a substitution at position 425 with isoleucine and at position 215 with phenylalanine; or a substitution at position 408 with isoleucine, at position 215 with isoleucine, and at position 125 with leucine; or a substitution at position 385 with glutamic acid and at position 247 with histidine; or a substitution at position 348 with methionine, at position 334 with asparagine, at position 275 with isoleucine, at position 202 with methionine, and at position 147 with threonine; or a substitution at position 275 with isoleucine, at position 334 with asparagine, and at position 348 with methionine; or a substitution at position 279 with leucine and at position 395 with serine; or a substitution at position 246 with threonine and at position 319 with phenylalanine; or a substitution at position 243 with isoleucine and at position 379 with leucine; or a substitution at position 243 with leucine, at position 255 with leucine and at position 318 with lysine; or a substitution at position 334 with glutamic acid, at position 359 with asparagine, and at position 366 with serine; or a substitution at position 288 with methionine and at position 334 with glutamic acid; or a substitution at position 334 with glutamic acid and at position 380 with aspartic acid; or a substitution at position 256 with serine, at position 305 with isoleucine, at position 334 with glutamic acid and at position 390 with serine; or a substitution at position 335 with asparagine, at position 370 with glutamic acid, at position 378 with valine, at position 394 with methionine, and at position 424 with leucine; or a substitution at position 233 with aspartic acid and at position 334 with glutamic acid; or a substitution at position 334 with glutamic acid, at position 359 with asparagine, at position 366 with serine, and at position 386 with arginine; or a substitution at position 246 with threonine and at position 396 with histidine; or a substitution at position 268 with aspartic acid and at position 318 with aspartic acid; or a substitution at position 288 with asparagine, at position 330 with serine, and at position 396 with leucine; or a substitution at position 244 with histidine, at position 358 with methionine, at position 379 with methionine, at position 384 with lysine and at position 397 with methionine; or a substitution at position 217 with serine, at position 378 with valine, and at position 408 with arginine; or a substitution at position 247 with leucine, at position 253 with asparagine, and at position 334 with asparagine; or a substitution at position 246 with isoleucine, and at position 334 with asparagine; or a substitution at position 320 with glutamic acid and at position 326 with glutamic acid; or a substitution at position 375 with cysteine and at position 396 with leucine; or a substitution at position 243 with leucine, at position 292 with proline, at position 300 with leucine, at position 305 with isoleucine, and at position 396 with leucine; or a substitution at position 243 with leucine, at position 292 with proline, at position 300 with leucine, and at position 396 with leucine; or a substitution at position 243 with leucine, at position 292 with proline, and at position 300 with leucine; or a substitution at position 243 with leucine, at position 270 with glutamic acid, at position 392 with asparagine and at position 396 with leucine; or a substitution at position 243 with leucine, at position 255 with leucine, at position 270 with glutamic acid, and at position 396 with leucine. Examples of other amino acid substitutions that results in an enhanced affinity for FcγRIIIA in vitro are disclosed below and summarized in Table 6.

In a specific embodiment, the invention encompasses an isolated polypeptide comprising a variant heavy chain having the Fc region of IgG2, IgG3 or IgG4, wherein said variant heavy chain comprises at least one amino acid modification relative to a wild-type heavy chain having an Fc region of the same isotype, such that said polypeptide specifically binds FcγRIIIA with a greater affinity relative to a comparable polypeptide comprising a wild-type heavy chain having an Fc region of the same isotype, wherein said at least one amino acid modification comprises a substitution at position 396 with histidine; or a substitution at position 248 with methionine; or a substitution at position 396 with leucine; or a substitution at position 379 with methionine; or a substitution at position 219 with tyrosine; or a substitution at position 282 with methionine; or a substitution at position 401 with valine; or a substitution at position 222 with asparagine; or a substitution at position 334 with glutamic acid; or a substitution at position 377 with phenylalanine; or a substitution at position 334 with isoleucine; or a substitution at position 247 with leucine; or a substitution at position 326 with glutamic acid; or a substitution at position 372 with tyrosine; or a substitution at position 224 with leucine; or a substitution at position 243 with leucine; or a substitution at position 292 with proline; or a substitution at position 275 with tyrosine; or a substitution at position 398 with valine; or a substitution at position 334 with asparagine; or a substitution at position 400 with proline; or a substitution at position 407 with isoleucine; or a substitution at position 372 with tyrosine.

In certain embodiments, the invention encompasses an isolated polypeptide comprising a variant heavy chain having the Fc region of IgG2, IgG3 or IgG4, wherein said variant heavy chain comprises at least one amino acid modification relative to a wild-type heavy chain having an Fc region of the same isotype, such that said polypeptide specifically binds FcγRIIIA with a similar affinity relative to a comparable polypeptide comprising a wild-type heavy chain having an Fc region of the same isotype, wherein said at least one amino acid modification comprises substitution at position 392 with arginine; or a substitution at position 315 with isoleucine; or a substitution at position 132 with isoleucine; or a substitution at position 162 with valine; or a substitution at position 366 with asparagine.

In certain embodiments, the invention encompasses an isolated polypeptide comprising a variant heavy chain having the Fe region of IgG2, IgG3 or IgG4, wherein said variant heavy chain comprises at least one amino acid modification relative to a wild-type heavy chain having an Fe region of the same isotype, such that said polypeptide specifically binds FcγRIIIA with a reduced affinity relative to a comparable polypeptide comprising a wild-type heavy chain having an Fe region of the same isotype, wherein said at least one amino acid modification comprises substitution at position 414 with asparagine; or a substitution at position 225 with serine; or a substitution at position 377 with asparagine.

In some embodiments, the molecules of the invention have an altered affinity for FcγRIIIA and/or FcγRIIA as determined using in vitro assays (biochemical or immunological based assays) known in the art for determining heavy chain-antibody receptor interactions, in particular Fc-FcγR interactions, i.e., specific binding of an Fe region to an FcγR including but not limited to ELISA assay, surface plasmon resonance assay, immunoprecipitation assays (See Section 5.2). Preferably, the binding properties of these molecules with altered affinities for activating FcγR receptors are also correlated to their activity as determined by in vitro functional assays for determining one or more FcγR mediator effector cell functions (See Section 5.3), e.g., molecules with variant heavy chains, or regions thereof, with enhanced affinity for FcγRIIIA have an enhanced ADCC activity. In most preferred embodiments, the molecules of the invention that have an altered binding property for an activating Fe receptor, e.g., FcγRIIIA in an in vitro assay also have an altered binding property in in vivo models (such as those described and disclosed herein). However, the present invention does not exclude molecules of the invention that do not exhibit an altered FcγR binding in in vitro based assays but do exhibit the desired phenotype in vivo.

B. Mutants with Enhanced Affinity for FcγRIIIA and Reduced or No Affinity for FcγRIIB

In a specific embodiment, the molecules of the invention comprise a variant heavy chain which contains an Fe region of IgG2, IgG3 or IgG4, having one or more amino acid modifications (i.e., substitutions) in one or more regions, which one or more modifications increase the affinity of the Fe region of the variant heavy chain for FcγRIIIA and decreases the affinity of the Fe region of the variant heavy chain for FcγRIIB, relative to a comparable molecule comprising a wild type heavy chain having an Fe region of the same isotype which binds FcγRIIIA and FcγRIIB with wild-type affinity. In certain embodiments, the one or more amino acid modifications increase the affinity of the Fc region of the varinat heavy chain for FcγRIIIA by at least 65%, at least 70%, at least 75%, at least 85%, at least 90%, at least 95%, at least 99%, at least 100%, at least 200%, at least 300%, at least 400% and decreases the affinity of the Fc region of the variant heavy chain for FcγRIIB by at least 65%, at least 70%, at least 75%, at least 85%, at least 90%, at least 95%, at least 99%, at least 100%, at least 200%, at least 300%, at least 400%.

In a specific embodiment, the molecule of the invention comprising a variant heavy chain that contains an Fc region of IgG2, IgG3 or IgG4, which exhibits an enhanced affinity for FcγRIIIA and a lowered affinity or no affinity for FcγRIIB, as determined based on an ELISA assay and/or an ADCC based assay using ch-4-4-20 antibody, or a surface plasmon resonance assay using a chimeric 4D5 antibody, carrying the variant heavy chain comprises a substitution at position 275 with isoleucine, at position 334 with asparagine, and at position 348 with methionine; or a substitution at position 279 with leucine and at position 395 with serine; or a substitution at position 246 with threonine and at position 319 with phenylalanine; or a substitution at position 243 with leucine, at position 255 with leucine, and at position 318 with lysine; or a substitution at position 334 with glutamic acid, at position 359 with asparagine and at position 366 with serine; or a substitution at position 334 with glutamic acid and at position 380 with aspartic acid; or a substitution at position 256 with serine, at position 305 with isoleucine, at position 334 with glutamic acid, and at position 390 with serine; or a substitution at position 335 with asparagine, at position 370 with glutamic acid, at position 378 with valine, at position 394 with methionine and at position 424 with leucine; or a substitution at position 233 with aspartic acid and at position 334 with glutamic acid; or a substitution at position 334 with glutamic acid, at position 359 with asparagine, at position 366 with serine and at position 386 with arginine; or a substitution at position 312 with glutamic acid, at position 327 with asparagine, and at position 378 with serine; or a substitution at position 288 with asparagine and at position 326 with asparagine; or a substitution at position 247 with leucine and at position 421 with lysine; or a substitution at position 298 with asparagine and at position 381 with arginine; or a substitution at position 280 with glutamic acid, at position 354 with phenylalanine, at position 431 with aspartic acid, and at position 441 with isoleucine; or a substitution at position 255 with glutamine and at position 326 with glutamic acid; or a substitution at position 218 with arginine, at position 281 with aspartic acid and at position 385 with arginine; or a substitution at position 247 with leucine, at position 330 with threonine and at position 440 with glycine; or a substitution at position 284 with alanine and at position 372 with leucine; or a substitution at position 335 with asparagine, as position 387 with serine and at position 435 with glutamine; or a substitution at position 247 with leucine, at position 431 with valine and at position 442 with phenylalanine; or a substitution at position 243 with leucine, at position 292 with proline, at position 305 with isoleucine, and at position 396 with leucine; or a substitution at position 243 leucine, at position 292 with proline, and at position 305 with isoleucine; or a substitution at position 243 leucine, at position 292 with proline, and at position 300 with leucine; or a substitution at position 292 with proline, at position 305 with isoleucine, and at position 396 with leucine; or a substitution at position 243 with leucine, and at position 292 with proline; or a substitution at position 292 with proline.

In a specific embodiment, the molecule of the invention comprising a variant heavy chain that contains an Fc region of IgG2, IgG3 or IgG4, which exhibits an enhanced affinity for FcγRIIIA and a lowered affinity or no affinity for FcγRIIB as determined based on an ELISA assay and/or an ADCC based assay using ch-4-4-20 antibody carrying the variant heavy chain comprises a substitution at position 379 with methionine; at position 219 with tyrosine; at position 282 with methionine; at position 401 with valine; at position 222 with asparagine; at position 334 with isoleucine; at position 334 with glutamic acid; at position 275 with tyrosine; at position 398 with valine. In yet another specific embodiment, the molecule of the invention comprising a variant heavy chain that contains an Fc region of IgG2, IgG3 or IgG4, which exhibits an enhanced affinity for FcγRIIIA and a lowered affinity or no affinity for FcγRIIB as determined based on an ELISA assay and/or an ADCC based assay using ch-4-4-20 antibody, or a surface plasmon resonance assay using a chimeric 4D5 antibody, carrying the variant heavy chain comprises a substitution at position 243 with leucine; at position 292 with proline; and at position 300 with leucine.

C. Mutants with Enhanced Affinity to FcγRIIIA and FcγRIIB

The invention encompasses molecules comprising variant heavy chains that contain Fc regions of IgG2, IgG3 or IgG4, having at least one amino acid modification relative to wild type heavy chains containing Fc regions of the same isotype, which modifications increase the affinity of the variant heavy chain for FcγRIIIA and FcγRIIB by at least 65%, at least 70%, at least 75%, at least 85%, at least 90%, at least 95%, at least 99%, at least 100%, at least 200%, at least 300%, at least 400% and decreases the affinity of the variant Fc region for FcγRIIB by at least 65%, at least 70%, at least 75%, at least 85%, at least 90%, at least 95%, at least 99%, at least 100%, at least 200%, at least 300%, at least 400%. In a specific embodiment, the molecule of the invention comprising a variant heavy chain that contains an Fc region of IgG2, IgG3 or IgG4, having at least one amino acid modification relative to a wild type heavy chain containing an Fc region of the same isotype, exhibits an enhanced affinity for FcγRIIIA and an enhanced affinity for FcγRIIB (as determined based on an ELISA assay and/or an ADCC based assay using ch-4-4-20 antibody, or a surface plasmon resonance assay using a chimeric 4D5 antibody, carrying the variant heavy as described herein) comprises a substitution at position 415 with isoleucine and at position 251 with phenylalanine; or a substitution at position 399 with glutamic acid, at position 292 with leucine, and at position 185 with methionine; or a substitution at position 408 with isoleucine, at position 215 with isoleucine, and at position 125 with leucine; or a substitution at position 385 with glutamic acid and at position 247 with histidine; or a substitution at position 348 with methionine, at position 334 with asparagine, at position 275 with isoleucine, at position 202 with methionine and at position 147 with threonine; or a substitution at position 246 with threonine and at position 396 with histidine; or a substitution at position 268 with aspartic acid and at position 318 with aspartic acid; or a substitution at position 288 with asparagine, at position 330 with serine and at position 396 with leucine; or a substitution at position 244 with histidine, at position 358 with methionine, at position 379 with methionine, at position 384 with lysine and at position 397 with methionine; or a substitution at position 217 with serine, at position 378 with valine, and at position 408 with arginine; or a substitution at position 247 with leucine, at position 253 with asparagine, and at position 334 with asparagine; or a substitution at position 246 with isoleucine and at position 334 with asparagine; or a substitution at position 320 with glutamic acid and at position 326 with glutamic acid; or a substitution at position 375 with cysteine and at position 396 with leucine; or a substitution at position 343 with serine, at position 353 with leucine, at position 375 with isoleucine, at position 383 with asparagine; or a substitution at position 394 with methionine and at position 397 with methionine; or a substitution at position 216 with aspartic acid, at position 345 with lysine and at position 375 with isoleucine; or a substitution at position 288 with asparagine, at position 330 with serine, and at position 396 with leucine; or a substitution at position 247 with leucine and at position 389 with glycine; or a substitution at position 222 with asparagine, at position 335 with asparagine, at position 370 with glutamic acid, at position 378 with valine and at position 394 with methionine; or a substitution at position 316 with aspartic acid, at position 378 with valine and at position 399 with glutamic acid; or a substitution at position 315 with isoleucine, at position 379 with methionine, and at position 394 with methionine; or a substitution at position 290 with threonine and at position 371 with aspartic acid; or a substitution at position 247 with leucine and at position 398 with glutamine; or a substitution at position 326 with glutamine; at position 334 with glutamic acid, at position 359 with asparagine, and at position 366 with serine; or a substitution at position 247 with leucine and at position 377 with phenylalanine; or a substitution at position 378 with valine, at position 390 with isoleucine and at position 422 with isoleucine; or a substitution at position 326 with glutamic acid and at position 385 with glutamic acid; or a substitution at position 282 with glutamic acid, at position 369 with isoleucine and at position 406 with phenylalanine; or a substitution at position 397 with methionine; at position 411 with alanine and at position 415 with asparagine; or a substitution at position 223 with isoleucine, at position 256 with serine and at position 406 with phenylalanine; or a substitution at position 298 with asparagine and at position 407 with arginine; or a substitution at position 246 with arginine, at position 298 with asparagine, and at position 377 with phenylalanine; or a substitution at position 235 with proline, at position 382 with methionine, at position 304 with glycine, at position 305 with isoleucine, and at position 323 with isoleucine; or a substitution at position 247 with leucine, at position 313 with arginine, and at position 388 with glycine; or a substitution at position 221 with tyrosine, at position 252 with isoleucine, at position 330 with glycine, at position 339 with threonine, at position 359 with asparagine, at position 422 with isoleucine, and at position 433 with leucine; or a substitution at position 258 with aspartic acid, and at position 384 with lysine; or a substitution at position 241 with leucine and at position 258 with glycine; or a substitution at position 370 with asparagine and at position 440 with asparagine; or a substitution at position 317 with asparagine and a deletion at position 423; or a substitution at position 243 with isoleucine, at position 379 with leucine and at position 420 with valine; or a substitution at position 227 with serine and at position 290 with glutamic acid; or a substitution at position 231 with valine, at position 386 with histidine, and at position 412 with methionine; or a substitution at position 215 with proline, at position 274 with asparagine, at position 287 with glycine, at position 334 with asparagine, at position 365 with valine and at position 396 with leucine; or a substitution at position 293 with valine, at position 295 with glutamic acid and at position 327 with threonine; or a substitution at position 319 with phenylalanine, at position 352 with leucine, and at position 396 with leucine; or a substitution at position 392 with threonine and at position 396 with leucine; at a substitution at position 268 with asparagine and at position 396 with leucine; or a substitution at position 290 with threonine, at position 390 with isoleucine, and at position 396 with leucine; or a substitution at position 326 with isoleucine and at position 396 with leucine; or a substitution at position 268 with aspartic acid and at position 396 with leucine; or a substitution at position 210 with methionine and at position 396 with leucine; or a substitution at position 358 with proline and at position 396 with leucine; or a substitution at position 288 with arginine, at position 307 with alanine, at position 344 with glutamic acid, and at position 396 with leucine; or a substitution at position 273 with isoleucine, at position 326 with glutamic acid, at position 328 with isoleucine and at position 396 with leucine; or a substitution at position 326 with isoleucine, at position 408 with asparagine and at position 396 with leucine; or a substitution at position 334 with asparagine and at position 396 with leucine; or a substitution at position 379 with methionine and at position 396 with leucine; or a substitution at position 227 with serine and at position 396 with leucine; or a substitution at position 217 with serine and at position 396 with leucine; or a substitution at position 261 with asparagine, at position 210 with methionine and at position 396 with leucine; or a substitution at position 419 with histidine and at position 396 with leucine; or a substitution at position 370 with glutamic acid and at position 396 with leucine; or a substitution at position 242 with phenylalanine and at position 396 with leucine; or a substitution at position 255 with leucine and at position 396 with leucine; or a substitution at position 240 with alanine and at position 396 with leucine; or a substitution at position 250 with serine and at position 396 with leucine; or a substitution at position 247 with serine and at position 396 with leucine; or a substitution at position 410 with histidine and at position 396 with leucine; or a substitution at position 419 with leucine and at position 396 with leucine; or a substitution at position 427 with alanine and at position 396 with leucine; or a substitution at position 258 with aspartic acid and at position 396 with leucine; or a substitution at position 384 with lysine and at position 396 with leucine; or a substitution at position 323 with isoleucine and at position 396 with leucine; or a substitution at position 244 with histidine and at position 396 with leucine; or a substitution at position 305 with leucine and at position 396 with leucine; or a substitution at position 400 with phenylalanine and at position 396 with leucine; or a substitution at position 303 with isoleucine and at position 396 with leucine; or a substitution at position 243 with leucine, at position 305 with isoleucine, at position 378 with aspartic acid, at position 404 with serine and at position 396 with leucine; or a substitution at position 290 with glutamic acid, at position 369 with alanine, at position 393 with alanine and at position 396 with leucine; or a substitution at position 210 with asparagine, at position 222 with isoleucine, at position 320 with methionine and at position 396 with leucine; or a substitution at position 217 with serine, at position 305 with isoleucine, at position 309 with leucine, at position 390 with histidine and at position 396 with leucine; or a substitution at position 246 with asparagine; at position 419 with arginine and at position 396 with leucine; or a substitution at position 217 with alanine, at position 359 with alanine and at position 396 with leucine; or a substitution at position 215 with isoleucine, at position 290 with valine and at position 396 with leucine; or a substitution at position 275 with leucine, at position 362 with histidine, at position 384 with lysine and at position 396 with leucine; or a substitution at position 334 with asparagine; or a substitution at position 400 with proline; or a substitution at position 407 with isoleucine; or a substitution at position 372 with tyrosine; or a substitution at position 366 with asparagine; or a substitution at position 414 with asparagine; or a substitution at position 352 with leucine; or a substitution at position 225 with serine; or a substitution at position 377 with asparagine; or a substitution at position 248 with methionine; or a substitution at position 243 with leucine, at position 292 with proline, at position 300 with leucine, at position 305 with isoleucine, and at position 396 with leucine; or a substitution at position 243 with leucine, at position 292 with proline, and at position 300 with leucine; or a substitution at position 243 with leucine, at position 292 with proline, at position 300 with leucine, and at position 396 with leucine; or a substitution at position 243 with leucine, and at position 396 with leucine; or at position 292 with proline, and at position 305 with isoleucine.

D. Mutants that Do Not Bind Any FcγR

In some embodiments, the invention encompasses molecules comprising variant heavy chains that contain Fc regions of IgG2, IgG3 or IgG4, having at least one amino acid modification in one or more regions, which molecules do not bind any FcγR, as determined by standard assays known in the art and disclosed herein, relative to a comparable molecule comprising the wild type heavy chain having an Fc region of the same isotype. In a specific embodiment, the one or more amino acid modifications which abolish binding to all FcγRs comprise a substitution at position 232 with serine and at position 304 with glycine; or a substitution at position 269 with lysine, at position 290 with asparagine, at position 311 with arginine, and at position 433 with tyrosine; or a substitution at position 252 with leucine; or a substitution at position 216 with aspartic acid, at position 334 with arginine, and at position 375 with isoleucine; or a substitution at position 247 with leucine and at position 406 with phenylalanine, or a substitution at position 335 with asparagine, at position 387 with serine, and at position 435 with glutamine; or a substitution at position 334 with glutamic acid, at position 380 with aspartic acid, and at position 446 with valine; or a substitution at position 303 with isoleucine, at position 369 with phenylalanine, and at position 428 with leucine; or a substitution at position 251 with phenylalanine and at position 372 with leucine; or a substitution at position 246 with glutamic acid, at position 284 with methionine and at position 308 with alanine; or a substitution at position 399 with glutamic acid and at position 402 with aspartic acid; or a substitution at position 399 with glutamic acid and at position 428 with leucine.

D. Mutants with Altered FcγR-mediated effector Functions

The invention encompasses immunoglobulins comprising a variant heavy chain (i.e., a heavy chain having the Fc region of IgG2, IgG3 or IgG4 and one or more amino acid modifications relative to a wild type heavy chain having the Fc region of the same isotype) that exhibit altered or added effector functions, i.e., where the variant exhibits detectable levels of one or more effector functions that are not detectable in the antibody comprising a wild-type heavy chain with an Fc region of the same isotype. In some embodiments, immunoglobulins comprising heavy chain variants mediate effector function more effectively in the presence of effector cells as determined using assays known in the art and exemplified herein. In other embodiments, immunoglobulins comprising heavy chain variants mediate effector function less effectively in the presence of effector cells as determined using assays known in the art and exemplified herein. In specific embodiments, the heavy chain variants of the invention may be combined with other known heavy chain modifications that alter effector function, such that the combination has an additive, synergistic effect. The heavy chain variants of the invention have altered effector function in vitro and/or in vivo.

In a specific embodiment, the immunoglobulins of the invention have an altered or enhanced FcγR-mediated effector function as determined using ADCC activity assays disclosed herein. Examples of effector functions that could be mediated by the molecules of the invention include, but are not limited to, C1q binding, complement-dependent cytotoxicity, antibody-dependent cell mediate cytotoxicity (ADCC), phagocytosis, etc. The effector functions of the molecules of the invention can be assayed using standard methods known in the art, examples of which are disclosed in Section 5.2. In a specific embodiment, the immunoglobulins of the invention comprising a variant heavy chain mediate ADCC 2-fold more effectively, than an immunoglobulin comprising a wild-type heavy chain having an Fc region of the same isotype. In other embodiments, the immunoglobulins of the invention comprising a variant heavy chain mediate ADCC at least 4-fold, at least 8-fold, at least 10-fold, at least 100-fold, at least 1000-fold, at least 10⁴-fold, at least 10⁵-fold more effectively, than an immunoglobulin comprising a wild-type heavy chain having an Fc region of the same isotype. In another specific embodiment, the immunoglobulins of the invention have altered C1q binding activity. In some embodiments, the immunoglobulins of the invention have at least 2-fold, at least 4-fold, at least 8-fold, at least 10-fold, at least 100-fold, at least 1000-fold, at least 10⁴-fold, at least 10⁵-fold higher C1q binding activity than an immunoglobulin comprising a wild-type heavy chain having an Fc region of the same isotype. In yet another specific embodiment, the immunoglobulins of the invention with have altered complement dependent cytotoxicity. In yet another specific embodiment, the immunoglobulins of the invention have an enhanced complement dependent cytotoxicity than an immunoglobulin comprising a wild-type heavy chain having an Fc region of the same isotype. In some embodiments, the immunoglobulins of the invention have at least 2-fold, at least 4-fold, at least 8-fold, at least 10-fold, at least 100-fold, at least 1000-fold, at least 10⁴-fold, at least 10⁵-fold higher complement dependent cytotoxicity than an immunoglobulin comprising a wild-type heavy chain having an Fc region of the same isotype.

In other embodiments, immunoglobulins of the invention have altered or enhanced phagocytosis activity relative to an immunoglobulin comprising a wild-type heavy chain having an Fc region of the same isotype, as determined by standard assays known to one skilled in the art or disclosed herein. In some embodiments, the immunoglobulins of the invention have at least 2-fold, at least 4-fold, at least 8-fold, at least 10-fold higher phagocytosis activity relative to an immunoglobulin comprising a wild-type heavy chain having an Fc region of the same isotype.

In a specific embodiment, the invention encompasses an immunoglobulin comprising a variant heavy chain that contains the Fc region of IgG2, IgG3 or IgG4, having at least one amino acid modification relative to a wild type heavy chain containing an Fc region of the same isotype, such that the immunoglobulin has an enhanced effector function, e.g., antibody dependent cell mediated cytotoxicity, or phagocytosis. In a specific embodiment, the one or more amino acid modifications which increase the ADCC activity of the immunoglobulin comprise a substitution at position 379 with methionine; or a substitution at position 243 with isoleucine and at position 379 with leucine; or a substitution at position 288 with asparagine, at position 330 with serine, and at position 396 with leucine; or a substitution at position 243 leucine and at position 255 with leucine; or a substitution at position 334 with glutamic acid, at position 359 with asparagine, and at position 366 with serine; or a substitution at position 288 with methionine and at position 334 with glutamic acid; or a substitution at position 334 with glutamic acid and at position 292 with leucine; or a substitution at position 316 with aspartic acid, at position 378 with valine, and at position 399 with glutamic acid; or a substitution at position 315 with isoleucine, at position 379 with methionine, and at position 399 with glutamic acid; or a substitution at position 243 with isoleucine, at position 379 with leucine, and at position 420 with valine; or a substitution at position 247 with leucine and at position 421 with lysine; or a substitution at position 248 with methionine; or a substitution at position 392 with threonine and at position 396 with leucine; or a substitution at position 293 with valine, at position 295 with glutamic acid, and at position 327 with threonine; or a substitution at position 268 with asparagine and at position 396 with leucine; or a substitution at position 319 with phenylalanine, at position 352 with leucine, and at position 396 with leucine; or a substitution at position 243 with leucine, at position 292 with proline, at position 300 with leucine, at position 305 with isoleucine, and at position 396 with leucine; or a substitution at position 243 with leucine, at position 292 with proline, at position 300 with leucine, and at position 396 with leucine; or a substitution at position 243 with leucine, at position 292 with proline, and at position 300 with leucine; or a substitution at position 255 with leucine, at position 396 with leucine, at position 270 with glutamic acid, and at position 300 with leucine; or a substitution at position 240 with alanine, at position 396 with leucine, and at position 270 with glutamic acid; or a substitution at position 370 with glutamic acid, at position 396 with leucine, and at position 270 with glutamic acid; or a substitution at position 392 with threonine, at position 396 with leucine, and at position 270 with glutamic acid; or a substitution at position 370 with glutamic acid and at position 396 with leucine; or a substitution at position 419 with histidine and at position 396 with leucine; or a substitution at position 255 with leucine, at position 396 with leucine, at position 270 with glutamic acid, and at position 292 with glycine. In other specific embodiments, the variant heavy chain of the invention has a leucine at position 247, a lysine at position 421 and a glutamic acid at position 270 (MgFc31/60); a threonine at position 392, a leucine at position 396, a glutamic acid at position 270, and a leucine at position 243 (MgFc38/60/F243L); a histidine at position 419, a leucine at position 396, and a glutamic acid at position 270 (MGFc51/60); a histidine at position 419, a leucine at position 396, a glutamic acid at position 270, and a leucine at position 243 (MGFc51/60/F243L); an alanine at position 240, a leucine at position 396, and a glutamic acid at position 270 (MGFc52/60); a lysine at position 255 and a leucine at position 396 (MgFc55); a lysine at position 255, a leucine at position 396, and a glutamic acid at position 270 (MGFc55/60); a lysine at position 255, a leucine at position 396, a glutamic acid at position 270, and a lysine at position 300 (MGFc55/60/Y300L); a lysine at position 255, a leucine at position 396, a glutamic acid at position 270, and a glycine at position 292 (MGFc55/60/R292G); a lysine at position 255, a leucine at position 396, a glutamic acid at position 270, and a leucine at position 243 (MgFc55/60/F243L); a glutamic acid at position 370, a leucine at position 396, and a glutamic acid at position 270 (MGFc59/60); a glutamic acid at position 270, an aspartic acid at position 316, and a glycine at position 416 (MgFc71); a leucine at position 243, a proline at position 292, an isoleucine at position 305, and a leucine at position 396 (MGFc74/P396L); or a leucine at position 243, a glutamic acid at position 270, an asparagine at position 392 and a leucine at position 396; or a leucine at position 243, a leucine at position 255, a glutamic acid at position 270 and a leucine at position 396; or a glutamine at position 297.

In another specific embodiment, the one or more amino acid modifications which increase the ADCC activity of the immunoglobulin is any of the mutations listed below in table 8. The mutations listed in Table 8 were originally identified in the context of an IgG1 Fc region.

TABLE 8
AMINO ACID MODIFICATION WHICH INCREASE ADCC
IN THE CONTEXT OF IgG1 Fc

| | |
|---|---|
| E333A/K334A | K334E, T359N, T366S |
| R292L/K334L | K288M/K334L |
| V379M | K288N/A330S/P396L |
| S219Y | K326E |
| V282M | G316D/A378V/D399E |
| K222N | N315I/V379M/T394M |
| F243L, V379L | F243I/V379L/G420V |
| F243L, R255L, L318K | E293V/Q295E/A327T |
| K334I | Y319F/P352L/P396L |
| Q419H/P396L | K392T/P396L |
| K370E/P396L | K248M |
| L242F/P396L | H268N/P396L |
| F243L/V305I/A378D/F404S/ | K290T/N390I/P396L |
| P396L | |
| R255L/P396L | K326I/P396L |
| V240A/P396L | H268D/P396L |
| T250S/P396L | K210M/P396L |
| P247S/P396L | L358P/P396L |
| K290E/V369A/T393A/P396L | K288R/T307A/K344L/P396L |
| K210N/K222I/K320M/P396L | V273I/K326E/L328I/P396L |
| L410H/P396L | K326I/S408N/P396L |
| Q419L/P396L | K334N/P396L |
| V427A/P396L | V379M/P396L |
| P217S/V305I/I309L/N390H/P396L | P227S/P396L |
| E258D/P396L | P217S/P396L |
| N384K/P396L | K261N/K210M/P396L |
| V323I/P396L | P247L/N421K/D270E |
| K246N/Q419R/P396L | Q419H/P396L/D270E |
| P217A/T359A/P396L | K370E/P396L/D270E |
| P244H/P396L | R255L/P396L/D270E |
| V215I/K290V/P396L | V240A/P396L/D270E |
| F275L/Q362H/N384K/P396L | K392T/P396L/D270E |
| V305L/P396L | F243L/R292P/Y300L/V305I/P396L |
| S400F/P396L | F243L/R292P/Y300L/P396L |
| V303I/P396L | F243L/R292P/Y300L |
| D270E/G316D/R416G | R255L/P396L/D270L/Y300L |
| P247L/N421K | R255L/P396L/D270E/R292G |
| R255L/P396L/D270L/F243L | K392T/P396L/D270E/F243L |
| F243L/D270E/K392N/P396L | Q419H/P396L/D270E/F243L |
| F243L/R255L/D270E/P396L | |

Alternatively or additionally, it may be useful to engineer the molecules of the invention to combine the above amino acid modifications, or any other amino acid modifications disclosed herein, with one or more further amino acid modifications in the context of the non-IgG1 domains or regions of the variant heavy chain such that the molecule exhibits altered or conferred C1q binding and/or complement dependent cytoxicity function. The starting molecule of particular interest herein is usually one that binds to C1q and displays complement dependent cytotoxicity (CDC). The further amino acid substitutions and/or heavy chain modifications, e.g., substitution of the native Fc region with the Fc region of IgG2, IgG3 or IgG4, described herein will generally serve to alter the ability of the starting molecule to bind to C1q and/or modify its complement dependent cytotoxicity function, e.g., to reduce and preferably abolish these effector functions. However, molecules comprising substitutions at one or more of the described positions with conferred or improved C1q binding and/or complement dependent cytotoxicity (CDC) function are contemplated herein. For example, the starting molecule may be unable to bind C1q and/or mediate CDC and may be modified according to the teachings herein such that it acquires these further effector functions. Moreover, molecules with preexisting C1q binding activity, optionally further having the ability to mediate CDC may be modified such that one or both of these activities are enhanced.

As disclosed above, one can design a variant heavy chain with altered effector function, e.g., by substitution of the Fc region thereof and/or amino acid modification, in order to confer C1q binding and/or FcR binding and thereby changing CDC activity and/or ADCC activity. For example, one can generate a variant heavy chain having the Fc region of IgG2, IgG3 or IgG4, having one or more amino acid modifications described herein, which exhibits improved or conferred C1q binding and improved or conferred FcγRIII binding; e.g., having both improved or conferred ADCC activity and improved or conferred CDC activity. Alternatively, where one desires that effector function be reduced or ablated, one may engineer a variant heavy chain comprising the Fc region of IgG2, IgG3 or IgG4, having one or more amino acid modifications described herein, which exhibits reduced CDC activity and/or reduced ADCC activity. In other embodiments, one may increase only one of these activities, and optionally also reduce the other activity, e.g., to generate an heavy chain variant with improved ADCC activity, but reduced CDC activity and vice versa.

The invention encompasses specific amino acid modifications of the heavy chain, in particular the Fc region, that have been previously identified in the context of an IgG1 heavy chain, in particular an IgG1 Fc region, using a yeast library as described in International Application WO04/063351 and U.S. Patent Application Publications 2005/0037000 and 2005/0064514, concurrent applications of the inventors, each of which is incorporated by reference herein in its entirety (Table 9). The IgG1 mutants were assayed using an ELISA assay for determining binding to FcγRIIIA and FcγRIIB. The mutants were also tested in an ADCC assay, by cloning the Fc variants into a ch 4-4-20 antibody using methods disclosed and exemplified herein. Bolded items refer to experiments, in which the ch4-4-20 were purified prior the ADCC assay. The antibody concentration used was standard for ADCC assays, in the range 0.5 μg/mL-1.0 g/mL.

TABLE 9
MUTATIONS IDENTIFIED IN THE Fc REGION OF IgG1

| | | | Binding | |
|---|---|---|---|---|
| | | Binding to | to | 4-4-20 ADCC |
| | | FcγRIIIA | FcγRIIB | (Relative Lysis |
| Mutations | Domain | (ELISA) | (ELISA) | (Mut/Wt) |
| pYD-CH1 library FACS screen with 3A tetramer | | | | |
| Q347H; A339V | CH3 | ↑0.5x | NT | |
| S415I; L251F | CH2, CH3 | ↑0.5x | ↑.75x | 0.82 |
| K392R | CH3 | N/C | NT | |
| D399E; R292L; V185M | CH1, CH2, CH3 | N/C | ↑0.5x | 0.65 |
| | | | | 0.9 |
| K290E; L142P | CH1, CH2 | N/C | NT | |
| R301C; M252L; S192T | CH1, CH2 | ↓.5x | NT | |
| P291S; K288E; H268L: A141V | CH1, CH2 | ↓.5x | NT | |
| N315I | CH2 | N/C | ↑.75x | |
| S132I | CH1 | N/C | NT | |
| S383N; N384K; T256N; V262L; K218E; R214I; K205E; F149Y; K133M | All | ↑0.5x | NT | |
| S408I; V215I; V125L | CH1, CH2, CH3 | ↑0.5x | ↑.75x | 0.62 |
| P396L | CH3 | ↑1x | ↑1x | 0.55 |
| G385E; P247H; | CH2, CH3 | ↑1x | ↑.75x | 0.44 |
| P396H | CH3 | ↑1x | ↑1x | 0.58 |
| A162V | CH1 | N/C | NT | |
| V348M; K334N; F275I; Y202M; K147T | CH1, CH2, CH3 | ↑0.5x | ↑.75x | 0.33 |
| H310Y; T289A; G337E | CH2 | ↑.5x | NT | |
| S119F; G371S; Y407V; E258D | CH1, CH2, CH3 | N/C | N/C | 0.29 |
| K409R; S166N | CH1, CH3 | N/C | NT | |
| in vitro Site Directed mutants | | | | |
| R292L | CH2 | NT | NT | 0.82 |
| T359N | CH3 | NT | NT | 1.06 |
| T366S | CH3 | NT | NT | 0.93 |
| E333A, K334A | CH2 | NT | NT | 1.41 |
| R292L, K334E | CH2 | NT | NT | 1.41; 1.64 |
| R292L, P396L, T359N | CH2, CH3 | NT | NT | 0.89; 1.15 |
| V379L | CH3 | NT | NT | 0.83 |
| K288N | CH2 | NT | NT | 0.78 |
| A330S | CH2 | NT | NT | 0.52 |
| F243L | CH2 | NT | NT | 0.38 |
| E318K | CH2 | NT | NT | 0.86 |
| K288N, A330S | CH2 | NT | NT | 0.08 |
| R255L, E318K | CH2 | NT | NT | 0.82 |
| F243L, E318K | CH2 | NT | NT | 0.07 |
| Mutants in 4-4-20 mini-library | | | | |
| Increased FcγRIIIA binding, decreased or no change to FcγRIIB binding | | | | |
| N/C means no change; N/B means no binding; NT means not tested | | | | |
| V379M | CH3 | ↑2x | N/C | 1.47 |
| S219Y | Hinge | ↑1x | ↓ or N/B | 1.28 |
| V282M | CH2 | ↑1x | ↓ or N/B | 1.25; 1 |
| F275I, K334N, V348M | CH2 | ↑0.5x | N/C | |
| D401V | CH3 | ↑0.5x | N/C | |
| V279L, P395S | CH2 | ↑1x | N/C | |
| K222N | Hinge | ↑1x | ↓ or N/B | 1.33; 0.63 |
| K246T, Y319F | CH2 | ↑1x | N/C | |
| F243I, V379L | CH2, CH3 | ↑1.5x | ↓ or N/B | 1.86; 1.35 |
| F243L, R255L, E318K | CH2 | ↑1x | ↓ or N/B | 1.81; 1.45 |
| K334I | CH2 | ↑1x | N/C | 2.1; 1.97 |
| K334E, T359N, T366S | CH2, CH3 | ↑1.5x | N/C | 1.49; 1.45 |
| K288M, K334E | CH2 | ↑3x | ↓ or N/B | 1.61; 1.69 |
| K334E, E380D | CH2, CH3 | ↑1.5x | N/C | |
| T256S, V305I, K334E, N390S | CH2, CH3 | ↑1.5x | N/C | |
| K334E | CH2 | ↑2.5x | N/C | 1.75; 2.18 |
| T335N, K370E, A378V, T394M, S424L | CH2, CH3 | ↑0.5x | N/C | |
| E233D, K334E | CH2 | ↑1.5x | N/C | 0.94; 1.02 |
| K334E, T359N, T366S, Q386R | CH2 | ↑1x | N/C | |
| Increased Binding to FcγIIIA and FcγRIIB | | | | |
| K246T, P396H | CH2, CH3 | ↑1x | ↑2.5x | |
| H268D, E318D | CH2 | ↑1.5x | ↑5x | |
| K288N, A330S, P396L | CH2, CH3 | ↑5x | ↑3x | 2.34; 1.66; 2.54 |
| I377F | CH3 | ↑1.5x | ↑0.5x | |
| P244H, L358M, V379M, N384K, V397M | CH2, CH3 | ↑1.75x | ↑1.5x | |
| P217S, A378V, S408R | Hinge, CH3 | ↑2x | ↑4.5x | |
| P247L, I253N, K334N | CH2 | ↑3x | ↑2.5x | |
| P247L | CH2 | ↑0.5x | ↑4x | 0.91; 0.84 |
| F372Y | CH3 | ↑0.75x | ↑5.5x | 0.88; 0.59 |
| K326E | CH2 | ↑2x | ↑3.5x | 1.63; 2 |
| K246I, K334N | CH2 | ↑0.5x | ↑4x | 0.66; 0.6 |
| K320E, K326E | CH2 | ↑1x | ↑1x | |
| H224L | Hinge | ↑0.5x | ↑5x | 0.55; |
| | | | | 0.53 |
| S375C, P396L | CH3 | ↑1.5x | ↑4.5x | |
| D312E, K327N, I378S | CH2, CH3 | ↑0.5x | N/C | |
| K288N, K326N | CH2 | ↑1x | N/C | |
| F275Y | CH2 | ↑3x | N/C | 0.64 |
| P247L, N421K | CH2, CH3 | ↑3x | N/C | 2.0 |
| S298N, W381R | CH2, CH3 | ↑2x | N/C | |
| D280E, S354F, A431D, L441I | CH2, CH3 | ↑3x | N/C | 0.62 |
| R255Q, K326E | CH2 | ↑2x | N/C | 0.79 |
| K218R, G281D, G385R | H, CH2, CH3 | ↑3.5x | N/C | 0.67 |
| L398V | CH3 | ↑1.5x | N/C | |
| P247L, A330T, S440G | CH2, CH3 | ↑0.75x | ↓0.25x | |
| V284A, F372L | CH2, CH3 | 1x | N/C | |
| T335N, P387S, H435Q | CH2, CH3 | 1.25x | N/C | |
| P247L, A431V, S442F | CH2, CH3 | 1x | N/C | |
| Increased Binding to FcγRIIIA and FcγRIIB | | | | |
| P343S, P353L, S375I, S383N | CH3 | ↑0.5x | ↑6x | |
| T394M, V397M | CH3 | ↑0.5x | ↑3x | |
| E216D, E345K, S375I | H, CH2, CH3 | ↑0.5x | ↑4x | |
| K334N, | CH2 | ↑0.5x | ↑2x | |
| K288N, A330S, P396L | CH2, CH3 | ↑0.5x | ↑9x | |
| P247L, E389G | CH2, CH3 | ↑1.5x | ↑9x | |
| K222N, T335N, K370E, A378V, T394M | H, CH2, CH3 | ↑1x | ↑7x | |
| G316D, A378V, D399E | CH2, CH3 | ↑1.5x | ↑14x | 2.24 |
| N315I, V379M, T394M | CH2, CH3 | ↑1x | ↑9x | 1.37 |
| K290T, G371D, | CH2, CH3 | ↑0.25x | ↑6x | |
| P247L, L398Q | CH2, CH3 | ↑1.25x | ↑10x | |
| K326Q, K334E, T359N, T366S | CH2, CH3 | ↑1.5x | ↑5x | |
| S400P | CH3 | ↑1x | ↑6x | |
| P247L, I377F | CH2, CH3 | ↑1x | ↑5x | |
| A378V, N390I, V422I | CH3 | ↑0.5x | ↑5x | |
| K326E, G385E | CH2, CH3 | ↑0.5x | ↑15x | |
| V282E, V369I, L406F | CH2, CH3 | ↑0.5x | ↑7x | |
| V397M, T411A, S415N | CH3 | ↑0.25x | ↑5x | |
| T223I, T256S, L406F | H, CH2, CH3 | ↑0.25x | ↑6x | |
| S298N, S407R | CH2, CH3 | ↑0.5x | ↑7x | |
| K246R, S298N, I377F | CH2, CH3 | ↑1x | ↑5x | |
| S407I | CH3 | ↑0.5x | ↑4x | |
| F372Y | CH3 | ↑0.5x | ↑4x | |
| L235P, V382M, S304G, V305I, V323I | CH2, CH3 | ↑2x | ↑2x | |
| P247L, W313R, E388G | CH2, CH3 | ↑1.5x | ↑1x | |
| D221Y, M252I, A330G, A339T, T359N, V422I, H433L | H, CH2, CH3 | ↑2.5x | ↑6x | |
| E258D, N384K | CH2, CH3 | ↑1.25x | ↑4x | |
| F241L, E258G | CH2 | ↑2x | ↑2.5x | −0.08 |
| K370N, S440N | CH3 | ↑1x | ↑3.5x | |
| K317N, F423-deleted | CH2, CH3 | ↑2.5x | ↑7x | 0.18 |
| F243I, V379L, G420V | CH2, CH3 | ↑2.5x | ↑3.5x | 1.35 |
| P227S, K290E | H, CH2 | ↑1x | ↑0.5x | |
| A231V, Q386H, V412M | CH2, CH3 | ↑1.5x | ↑6x | |
| T215P, K274N, A287G, K334N, L365V, P396L | H, CH2, CH3 | ↑2x | ↑4x | |
| Increased Binding to FcγRIIB but not FcγRIIIA | | | | |
| K334E, E380D | CH2, CH3 | N/C | ↑4.5x | |
| T366N | CH3 | N/C | ↑5x | |
| P244A, K326I, C367R, S375I, K447T | CH2, CH3 | N/C | ↑3x | |
| C229Y, A287T, V379M, P396L, L443V | H, CH2, CH3 | ↓0.25x | ↑10x | |
| Decreased binding to FcγRIIIA and FcγRIIB | | | | |
| R301H, K340E, D399E | CH2, CH3 | ↓0.50x | ↓0.25x | |
| K414N | CH3 | ↓0.25x | N/B | |
| P291S, P353Q | CH2, CH3 | ↓0.50x | ↓0.25x | |
| V240I, V281M | CH2 | ↓0.25x | ↓0.25x | |
| P232S, S304G | CH2 | N/B | N/B | |
| E269K, K290N, Q311R, H433Y | CH2, CH3 | N/B | N/B | |
| M352L | CH3 | N/B | N/B | |
| E216D, K334R, S375I | H, CH2, CH3 | N/B | N/B | |
| P247L, L406F | CH2, CH3 | N/B | N/B | |
| T335N, P387S, H435Q | CH2, CH3 | N/B | N/B | |
| T225S | CH2 | ↓0.25x | ↓0.50x | |
| D399E, M428L | CH3 | ↓0.50x | ↓0.50x | |
| K246I, Q362H, K370E | CH2, CH3 | N/B | ↓0.50x | |
| K334E, E380D, G446V | CH2, CH3 | N/B | N/B | |
| I377N | CH3 | ↓0.50x | N/B | |
| V303I, V369F, M428L | CH2, CH3 | N/B | N/B | |
| L251F, F372L | CH2, CH3 | N/B | N/B | |
| K246E, V284M, V308A | CH2, CH3 | N/B | N/B | |
| D399E, G402D | CH3 | N/B | N/B | |
| D399E, M428L | CH3 | N/B | N/B | |
| FcγRIIB depletion/FcγRIIIA selection: Naive Fc library. | | | | |
| E293V, Q295E, A327T | CH2 | ↑0.4x | ↓ or N/B | 4.29 |
| Y319F, P352L, P396L | CH2, CH3 | ↑3.4x | ↑2x | 1.09 |
| K392T, P396L | CH3 | ↑4.5x | ↑2.5x | 3.07 |
| K248M | CH2 | ↑0.4x | ↓ or N/B | 4.03 |
| H268N, P396L | CH2, CH3 | ↑2.2x | ↑4.5x | 2.24 |
| Solution competition 40X FcγRIIB-G2: P396L Library | | | | |
| D221E, D270E, V308A, Q311H, P396L, G402D | | ↑3.6x | ↑0.1x | 3.17 |
| Equilibrium Screen: 0.8 μM FcγRIIIA monomer: P396L library | | | | |
| K290T, N390I, P396L | CH2, CH3 | ↑2.8x | ↑6.1x | 1.93 |
| K326I, P396L | CH2, CH3 | ↑2.9x | ↑5.9x | 1.16 |
| H268D, P396L | CH2, CH3 | ↑3.8x | ↑13.7x | 2.15 |
| K210M, P396L | CH1, CH3 | ↑1.9x | ↑4.6x | 2.02 |
| L358P, P396L | CH3 | ↑1.9x | ↑4.2x | 1.58 |
| K288R, T307A, K344E, P396L | CH2, CH3 | ↑4.1x | ↑2.3x | 3.3 |
| V273I, K326E, L328I, P396L | CH2, CH3 | ↑1.3x | ↑10.8x | 0.78 |
| K326I, S408N, P396L | CH2, CH3 | ↑4x | ↑9.3x | 1.65 |
| K334N, P396L | CH2, CH3 | ↑3.1x | ↑3x | 2.43 |
| V379M, P396L | CH3 | ↑1.9x | ↑5.6x | 2.01 |
| P227S, P396L | CH2, CH3 | ↑1.5x | ↑4x | 2.01 |
| P217S, P396L | H, CH3 | ↑1.6x | ↑4.5x | 2.04 |
| K261N, K210M, P396L | CH2, CH3 | ↑2x | ↑4.2x | 2.06 |
| Kinetic Screen: O.8 μM, 1′ with cold 8 μM FcγRIIIA: P396L Library | | | | |
| term is M, P396L | CH3 | ↑1.9x | ↑7.2x | 3.09 |
| Q419H, P396L | CH3 | ↑2x | ↑6.9x | 2.24 |
| K370E, P396L | CH3 | ↑2x | ↑6.6x | 2.47 |
| L242F, P396L | CH2, CH3 | ↑2.5x | ↑4.1x | 2.4 |
| F243L, V305I, A378D, F404S, P396L | CH2, CH3 | ↑1.6x | ↑5.4x | 3.59 |
| R255L, P396L | CH2, CH3 | ↑1.8x | ↑6x | 2.79 |
| V240A, P396L | CH2, CH3 | ↑1.3x | ↑4.2x | 2.35 |
| T250S, P396L | CH2, CH3 | ↑1.5x | ↑6.8x | 1.60 |
| P247S, P396L | CH2, CH3 | ↑1.2x | ↑4.2x | 2.10 |
| K290E, V369A, T393A, P396L | CH2, CH3 | ↑1.3x | ↑6.7x | 1.55 |
| K210N, K222I, K320M, P396L | H, CH2, CH3 | ↑2.7x | ↑8.7x | 1.88 |
| L410H, P396L | CH3 | ↑1.7x | ↑4.5x | 2.00 |
| Q419L, P396L | CH3 | ↑2.2x | ↑6.1x | 1.70 |
| V427A, P396L | CH3 | ↑1.9x | ↑4.7x | 1.67 |
| P217S, V305I, I309L, N390H, P396L | H, CH2, CH3 | ↑2x | ↑7x | 1.54 |
| E258D, P396L | CH2, CH3 | ↑1.9x | ↑4.9x | 1.54 |
| N384K, P396L | CH3 | ↑2.2x | ↑5.2x | 1.49 |
| V323I, P396L | CH2, CH3 | ↑1.1x | ↑8.2x | 1.29 |
| K246N, Q419R, P396L | CH2, CH3 | ↑1.1x | ↑4.8x | 1.10 |
| P217A, T359A, P396L | H, CH2, CH3 | ↑1.5x | ↑4.8x | 1.17 |
| P244H, P396L | CH2, CH3 | ↑2.5x | ↑4x | 1.40 |
| V215I, K290V, P396L | H, CH2, CH3 | ↑2.2x | ↑4.6x | 1.74 |
| F275L, Q362H, N384K, P396L | CH2, CH3 | ↑2.2x | ↑3.7x | 1.51 |
| V305L, P396L | CH2, CH3 | ↑1.3x | ↑5.5x | 1.50 |
| S400F, P396L | CH3 | ↑1.5x | ↑4.7x | 1.19 |
| V303I, P396L | CH3 | ↑1.1x | ↑4x | 1.01 |
| FcγRIIB depletion FcγRIIIA 158V solid phase selection: Naïve Library | | | | |
| A330V, H433Q, V427M | CH2, CH3 | NT | NT | NT |
| V263Q, E272D, Q419H | CH2, CH3 | NT | NT | NT |
| N276Y, T393N, W417R | CH2, CH3 | NT | NT | NT |
| V282L, A330V, H433Y, T436R | CH2, CH3 | NT | NT | NT |
| A330V, Q419H | CH2, CH3 | NT | NT | NT |
| V284M, S298N, K334E, R355W | CH2, CH3 | NT | NT | NT |
| A330V, G427M, K438R | CH2, CH3 | NT | NT | NT |
| S219T, T225K, D270E, K360R | CH2, CH3 | NT | NT | NT |
| K222E, V263Q, S298N | CH2 | NT | NT | NT |
| V263Q, E272D | CH2 | NT | NT | NT |
| R292G | CH2 | NT | NT | NT |
| S298N | CH2 | NT | NT | NT |
| E233G, P247S, L306P | CH2 | NT | NT | NT |
| D270E | CH2 | NT | NT | NT |
| S219T, T225K, D270E | CH2 | NT | NT | NT |
| K326E, A330T | CH2 | NT | NT | NT |
| E233G | CH2 | NT | NT | NT |
| S254T, A330V, N361D, P243L | CH2, CH3 | NT | NT | NT |
| FcγRIIB depletion FcγRIIIA 158F solid phase selection: Naïve Library | | | | |
| 158F by FACS top 0.2% | | | | |
| V284M, S298N, K334E, R355W R416T | CH2, CH3 | NT | NT | |
| FcγRIIB depletion FcgRIIA 131H solid phase selection: Naïve Library | | | | |
| R292P, V305I | CH2, CH2 | NT | NT | |
| D270E, G316D, R416G | CH2, CH3 | NT | NT | |
| V284M, R292L, K370N | CH2, CH3 | NT | NT | |
| R292P, V305I, F243L | CH2 | NT | NT | |

In certain embodiments, the invention provides modified immunoglobulin molecules (e.g., antibodies) with variant heavy chains containing the Fc region of IgG2, IgG3 or IgG4, having one or more amino acid modifications relative to a wild type heavy chain having an Fc region of the same isotype, which one or more amino acid modifications confer or alter an effector function and/or increase or alter the affinity of the molecule for FcγR. Such immunoglobulins include IgG molecules that naturally contain FcγR binding regions (e.g., FcγRIIIA and/or FcγRIIB binding region), immunoglobulin molecules that do not naturally bind to FcγR, or immunoglobulin derivatives that have been engineered to contain an FcγR binding region (e.g., FcγRIIIA and/or FcγRIIB binding region). The modified immunoglobulins of the invention include any immunoglobulin molecule that binds, preferably, immunospecifically, i.e., competes off non-specific binding as determined by immunoassays well known in the art for assaying specific antigen-antibody binding, an antigen and contains an FcγR binding region (e.g., a FcγRIIIA and/or FcγRIIB binding region). Such antibodies include, but are not limited to, polyclonal, monoclonal, bi-specific, multi-specific, human, humanized, chimeric antibodies, single chain antibodies, Fab fragments, F(ab′)₂ fragments, disulfide-linked Fvs, and fragments containing either a VL or VH domain or even a complementary determining region (CDR) that specifically binds an antigen, in certain cases, engineered to contain or fused to an FcγR binding region.

In some embodiments, the molecules of the invention comprise portions of a heavy chain, in particular comprise an Fc region or portions thereof. As used herein the term “portion of an Fc region” refers to fragments of the Fc region, preferably a portion with effector activity and/or FcγR binding activity (or a comparable region of a mutant lacking such activity). The fragment of an Fc region may range in size from 5 amino acids to the entire Fc region minus one amino acids. The portion of an Fc region may be missing up to 10, up to 20, up to 30 amino acids from the N-terminus or C-terminus.

The IgG molecules of the invention are preferably IgG1 subclass of IgGs, but may also be any other IgG subclasses of given animals, including, but not limited to, rats, mice and primates, e.g., chimpanzee, baboon, and macaque. For example, in humans, the IgG class includes IgG1, IgG2, IgG3, and IgG4; mouse IgG includes IgG1, IgG2a, IgG2b, IgG2c and IgG3; and rat includes IgG1, IgG2a, IgG2b and IgG2c.

The immunoglobulins (and other polypeptides used herein) may be from any animal origin including birds and mammals. Preferably, the antibodies are human, rodent (e.g., mouse and rat), donkey, sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example, in U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide or may be specific for heterologous epitopes, such as a heterologous polypeptide or solid support material. See, e.g., PCT publications WO 93/17715; WO 92/08802; WO 91/00360; WO 92/05793; Tutt, et al., J. Immunol, 147:60-69, 1991; U.S. Pat. Nos. 4,474,893; 4,714,681; 4,925,648; 5,573,920; 5,601,819; Kostelny et al., J. Immunol, 148:1547-1553, 1992.

Multispecific antibodies have binding specificities for at least two different antigens. While such molecules normally will only bind two antigens (i.e. bispecific antibodies, BsAbs), antibodies with additional specificities such as trispecific antibodies are encompassed by the instant invention. Examples of BsAbs include without limitation those with one arm directed against a tumor cell antigen and the other arm directed against a cytotoxic molecule.

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983); which is incorporated herein by reference in its entirety). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions that are independently selected from IgG2, IgG3 or IgG4. It is preferred to have the first heavy-chain constant region (CH1) containing the site necessary for light chain binding, present in at least one of the fusions. In some embodiments, the CH1 region of the molecule of the invention is from IgG1. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when, the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986). According to another approach described in WO96/27011, a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Antibodies with more than two valencies are contemplated. For example, trispecific antibodies can be prepared. See, e.g., Tutt et al., 1991, J. Immunol. 147:60, which is incorporated herein by reference.

The antibodies of the invention include derivatives that are otherwise modified, i.e., by the covalent attachment of any type of molecule to the antibody such that covalent attachment does not prevent the antibody from binding antigen and/or generating an anti-idiotypic response. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a constant region derived from a human immunoglobulin. Methods for producing chimeric antibodies are known in the art. See e.g., Morrison, Science, 229:1202, 1985; Oi et al., BioTechniques, 4:214 1986; Gillies et al., J. Immunol Methods, 125:191-202, 1989; U.S. Pat. Nos. 5,807,715; 4,816,567; and 4,816,397, which are incorporated herein by reference in their entireties. Humanized antibodies are antibody molecules from non-human species that bind the desired antigen having one or more complementarity determining regions (CDRs) from the non-human species and framework regions and constant domains from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. No. 5,585,089; Riechmann et al., Nature, 332:323, 1988, which are incorporated herein by reference in their entireties. Antibodies can be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, Molecular Immunology, 28(4/5):489-498, 1991; Studnicka et al., Protein Engineering, 7(6):805-814, 1994; Roguska et al., Proc Natl. Acad. Sci. USA, 91:969-973, 1994), and chain shuffling (U.S. Pat. No. 5,565,332), all of which are hereby incorporated by reference in their entireties. Humanized antibodies may be generated using any of the methods disclosed in U.S. Pat. Nos. 5,693,762 (Protein Design Labs), 5,693,761, (Protein Design Labs) 5,585,089 (Protein Design Labs), 6,180,370 (Protein Design Labs), and U.S. Publication Nos. 20040049014, 200300229208, each of which is incorporated herein by reference in its entirety.

Completely human antibodies are particularly desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741, each of which is incorporated herein by reference in its entirety.

Human antibodies can also be produced using transgenic mice which are incapable of expressing functional endogenous immunoglobulins, but which can express human immunoglobulin genes. For an overview of this technology for producing human antibodies, see Lonberg and Huszar, Int. Rev. Immunol., 13:65-93, 1995. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. In addition, companies such as Abgenix, Inc. (Freemont, Calif.), Medarex (J) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., Bio/technology, 12:899-903, 1988).

The invention encompasses engineering human or humanized therapeutic antibodies (e.g., tumor specific monoclonal antibodies) in the heavy, both by substitution or replacement of a native region or domain with the corresponding region or domain of a heterologous isotype and by modification (e.g., substitution, insertion, deletion) of at least one amino acid residue, which modifications alters or increases the affinity of the Fc region of the variant heavy chain for FcγR, e.g., FcγRIIIA and/or FcγRIIA and/or confers or alters an effector function activity, e.g., ADCC activity, complement activation, phagocytosis activity, etc., as determined by standard assays known to those skilled in the art relative to an antibody comprising a wild-type heavy chain having an Fc region of the same isotype. In other embodiments, the engineered therapeutic antibodies may exhibit oligomerization activity mediated by the Fc region of the variant heavy chain. In another embodiment, the invention relates to engineering human or humanized therapeutic antibodies (e.g., tumor specific monoclonal antibodies) in the heavy chain, both by substitution or replacement of a native region or domain with the corresponding region or domain of a heterologous isotype and by modification (e.g., substitution, insertion, deletion) of at least one amino acid residue, which modifications increase the affinity of the Fc region for FcγRIIIA and/or FcγRIIA and further decreases the affinity of the Fc region for FcγRIIB.

In a specific embodiment, the invention encompasses engineering a humanized monoclonal antibody specific for Her2/neu protooncogene (e.g., Ab4D5 humanized antibody as disclosed in Carter et al., 1992, Proc. Natl. Acad. Sci. USA 89:4285-9) both by substitution or replacement of the native Fc region with the Fc region of IgG2, IgG3 or IgG4 and by modification (e.g., substitution, insertion, deletion) of at least one amino acid residue, which modifications increase the affinity of the Fc region for FcγRIIIA and/or FcγRIIA. In another specific embodiment, modification of the humanized Her2/neu monoclonal antibody may also further decrease the affinity of the Fc region of the variant heavy chain for FcγRIIB. In yet another specific embodiment, the humanized monoclonal antibodies specific for Her2/neu engineered in accordance with the invention may further have an enhanced effector function as determined by standard assays known in the art and disclosed and exemplified herein.

In another embodiment, the invention encompasses engineering a mouse human chimeric anti-CD20 monoclonal antibody, 2H7 both by substitution or replacement of a native region or domain with the corresponding region or domain of a heterologous isotype and by modification (e.g., substitution, insertion, deletion) of at least one amino acid residue, which modifications increase the affinity of the Fc region for FcγRIIIA and/or FcγRIIA. In another specific embodiment, modification of the anti-CD20 monoclonal antibody, 2H7 may also further decrease the affinity of the Fc region for FcγRIIB. In yet another specific embodiment, the engineered anti-CD20 monoclonal antibody, 2H7 may further have an enhanced effector function as determined by standard assays known in the art and disclosed and exemplified herein.

In a specific embodiment, the invention encompasses engineering a humanized antibody comprising the CDRs of 2B6 or of 3H7. In particular, an antibody comprising the heavy chain variable domain having the amino acid sequence of SEQ ID NO: 1 and the light chain variable domain having the amino acid sequence of SEQ ID NO: 2, SEQ ID NO: 3, or SEQ ID NO: 4. In a specific embodiment, the invention encompasses engineering a humanized antibody comprising the heavy chain variable domain having the amino acid sequence of SEQ ID NO: 5 and the light chain variable domain having the amino acid sequence of SEQ ID NO: 6.

In another specific embodiment, the invention encompasses engineering an anti-FcγRIIB antibody including but not limited to any of the antibodies disclosed in U.S. Provisional Application No. 60/403,266 filed on Aug. 12, 2002, U.S. application Ser. No. 10/643,857 filed on Aug. 14, 2003, U.S. Provisional Application No. 60/562,804 filed on Apr. 16, 2004, U.S. Provisional Application No. 60/582,044 filed on Jun. 21, 2004, U.S. Provisional Application No. 60/582,045 filed on Jun. 21, 2004, U.S. Provisional Application No. 60/636,663 filed on Dec. 15, 2004 and U.S. application Ser. No. 10/524,134 filed Feb. 11, 2005 both by substitution or replacement of a native region or domain with the corresponding region or domain of a heterologous isotype and by modification (e.g., substitution, insertion, deletion) of at least one amino acid residue, which modifications increase the affinity of the Fc region for FcγRIIIA and/or FcγRIIA. In another specific embodiment, the invention encompasses engineering a humanized anti-FcγRIIB antibody including but not limited to any of the antibodies disclosed in U.S. Provisional Application No. 60/569,882 filed on May 10, 2004, U.S. Provisional Application No. 60/582, 043 filed on Jun. 21, 2004 and U.S. application Ser. No. 11/126,978, filed on May 10, 2005 both by substitution or replacement of a native region or domain with the corresponding region or domain of a heterologous isotype and by modification (e.g., substitution, insertion, deletion) of at least one amino acid residue, which modifications increase the affinity of the Fc region for FcγRIIIA and/or FcγRIIA. Each of the above mentioned applications is incorporated herein by reference in its entirety. Examples of anti-FcγRIIB antibodies, which may or may not be humanized, that may be engineered in accordance with the methods of the invention are 2B6 monoclonal antibody having ATCC accession number PTA-4591 and 3H7 having ATCC accession number PTA-4592, ID5 monoclonal antibody having ATCC accession number PTA-5958, 1F2 monoclonal antibody having ATCC accession number PTA-5959, 2D11 monoclonal antibody having ATCC accession number PTA-5960, 2E1 monoclonal antibody having ATCC accession number PTA-5961 and 2H9 monoclonal antibody having ATCC accession number PTA-5962 (all deposited at 10801 University Boulevard, Manassas, Va. 02209-2011), which are incorporated herein by reference. In another specific embodiment, modification of the anti-FcγRIIB antibody may also further decrease the affinity of the Fe region for FcγRIIB. In yet another specific embodiment, the engineered anti-FcγRIIB antibody may further have an enhanced effector function as determined by standard assays known in the art and disclosed and exemplified herein. In a specific embodiment, the 2B6 monoclonal antibody comprises a modification at position 334 with glutamic acid, at position 359 with asparagine, and at position 366 with serine (MgFc13); or a substitution at position 316 with aspartic acid, at position 378 with valine, and at position 399 with glutamic acid (MgFc27); or a substitution at position 243 with isoleucine, at position 379 with leucine, and at position 420 with valine (MgFc29); or a substitution at position 392 with threonine and at position 396 with leucine (MgFc38); or a substitution at position 221 with glutamic acid, at position 270 with glutamic acid, at position 308 with alanine, at position 311 with histidine, at position 396 with leucine, and at position 402 with aspartic (MgFc42); or a substitution at position 410 with histidine, and at position 396 with leucine (MgFc53); or a substitution at position 243 with leucine, at position 305 with isoleucine, at position 378 with aspartic acid, at position 404 with serine, and at position 396 with leucine (MgFc54); or a substitution at position 255 with isoleucine, and at position 396 with leucine (MgFc55); or a substitution at position 370 with glutamic acid, and at position 396 with leucine (MgFc59); or a substitution at position 243 with leucine, at position 292 with proline, at position 300 with leucine, at position 305 with isoleucine, and at position 396 with leucine (MgFc88); or a substitution at position 243 with leucine, at position 292 with proline, at position 300 with leucine, and at position 396 with leucine (MgFc88A); or a substitution at position 243 with leucine, at position 292 with proline, and at position 300 with leucine (MgFc155) (See Tables 6 & 7).

In a specific embodiment, the invention encompasses a modified molecule comprising a heavy chain with a substitution at position 255 with leucine, at position 396 with leucine, at position 270 with glutamic acid, and at position 300 with leucine; or a substitution at position 419 with histidine, at position 396 with leucine, and at position 270 with glutamic acid; or a substitution at position 240 with alanine, at position 396 with leucine, and at position 270 with glutamic acid; or a substitution at position 370 with glutamic acid, at position 396 with leucine, and at position 270 with glutamic acid; or a substitution at position 392 with threonine, at position 396 with leucine, and at position 270 with glutamic acid; or a substitution at position 370 with glutamic acid and at position 396 with leucine; or a substitution at position 419 with histidine and at position 396 with leucine; or a substitution at position 247 with leucine, at position 421 with lysine, and at position 270 with glutamic acid; or a substitution at position 255 with leucine, at position 396 with leucine, at position 270 with glutamic acid, and at position 292 with glycine. In other specific embodiments, the variant Fe region has a leucine at position 247, a lysine at position 421 and a glutamic acid at position 270 (MgFc31/60); a threonine at position 392, a leucine at position 396, a glutamic acid at position 270, and a leucine at position 243 (MgFc38/60/F243L); a histidine at position 419, a leucine at position 396, and a glutamic acid at position 270 (MGFc51/60); a histidine at position 419, a leucine at position 396, a glutamic acid at position 270, and a leucine at position 243 (MGFc51/60/F243L); an alanine at position 240, a leucine at position 396, and a glutamic acid at position 270 (MGFc52/60); a lysine at position 255 and a leucine at position 396 (MgFc55); a lysine at position 255, a leucine at position 396, and a glutamic acid at position 270 (MGFc55/60); a lysine at position 255, a leucine at position 396, a glutamic acid at position 270, and a lysine at position 300 (MGFc55/60/Y300L); a lysine at position 255, a leucine at position 396, a glutamic acid at position 270, and a glycine at position 292 (MGFc55/60/R292G); a lysine at position 255, a leucine at position 396, a glutamic acid at position 270, and a leucine at position 243 (MgFc55/60/F243L); a glutamic acid at position 370, a leucine at position 396, and a glutamic acid at position 270 (MGFc59/60); a glutamic acid at position 270, an aspartic acid at position 316, and a glycine at position 416 (MgFc71); a leucine at position 243, a proline at position 292, an isoleucine at position 305, and a leucine at position 396 (MGFc74/P396L); or a leucine at position 243, a glutamic acid at position 270, an asparagine at position 392 and a leucine at position 396; or a leucine at position 243, a leucine at position 255, a glutamic acid at position 270 and a leucine at position 396; or a glutamine at position 297.

In preferred embodiments, the invention encompasses molecules comprising a variant heavy chain having the Fe region of IgG2, IgG3 or IgG4 and having one or more amino acid modifications relative to a wild type heavy chain having an Fe region of the same isotype, wherein said one or more amino acid modifications does not comprise or does not solely comprise modification at the interface between the variant heavy chain, in particular the Fe region thereof, and the Fe ligand. Fe ligands include but are not limited to FcγRs, C1q, FcRn, C3, mannose receptor, protein A, protein G, mannose receptor, and undiscovered molecules that bind to the immunoglobulin heavy chain, and, in particular, the Fe region. Amino acids at the interface between an Fe region and an Fe ligand are defined as those amino acids that make a direct and/or indirect contact between the Fe region or the heavy chain and the ligand, play a structural role in determining the conformation of the interface, or are within at least 3 angstroms, preferably at least 2 angstroms of each other as determined by structural analysis, such as x-ray crystallography and molecular modeling The amino acids at the interface between an Fe region and an Fe ligand include those amino acids that make a direct contact with an FcγR based on crystallographic and structural analysis of Fc-FcγR interactions such as those disclosed by Sondermann et al., (2000, Nature, 406: 267-273; which is incorporated herein by reference in its entirety). Examples of positions within the Fc region that make a direct contact with FcγR are amino acids 234-239 (hinge region), amino acids 265-269 (B/C loop), amino acids 297-299 (C′/E loop), and amino acids 327-332 (F/G) loop. In some embodiments, the molecules of the invention comprising variant Fc regions comprise modification of at least one residue that does not make a direct contact with an FcγR based on structural and crystallographic analysis, e.g., is not within the Fc-FcγR binding site.

Preferably, the one or more amino acid modifications encompassed by the invention do not solely modify any of the amino acids as identified by Shields et al., which correspond to the amino acids in the IgG1 CH2 domain of an Fc region proximal to the hinge region, e.g., Leu234-Pro238; Ala327, Pro329, and affect binding of an Fc region to all human FcγRs.

In other embodiments, the invention encompasses heavy chain variants having the Fc regions of IgG2, IgG3 or IgG4, and having one or more amino acid modifications relative to a wild type heavy chain having the Fc region of the same isotype, which heavy chains exhibit altered FcγR affinities and/or altered effector functions, such that the heavy chain variant does not have or does not solely have an amino acid modification at a position at the interface between the Fc region of the variant heavy chain and the Fc ligand. Preferably, heavy chain variants of the invention in combination with one or more other amino acid modifications which are at the interface between the Fc region and the Fc ligand have a further impact on the particular property to be engineered, e.g. altered FcγR affinity. Modifying amino acids at the interface between the Fc region of the variant heavy chain and an Fc ligand may be done using methods known in the art, for example based on structural analysis of Fc-ligand complexes. For example, but not by way of limitation, by exploring energetically favorable substitutions at positions within the heavy chain Fc that impact the binding interface, variants can be engineered that sample new interface conformations, some of which may improve binding to the Fc ligand, some of which may reduce Fc ligand binding, and some of which may have other favorable properties. Such new interface conformations could be the result of, for example, direct interaction with Fc ligand residues that form the interface, or indirect effects caused by the amino acid modifications such as perturbation of side chain or backbone conformations

The invention encompasses molecules comprising heavy chain variants comprising any of the amino acid modifications disclosed herein in combination with other modifications in which the conformation of the carbohydrate at position 297, which is within the Fc region, is altered. The invention encompasses conformational and compositional changes in the N297 carbohydrate that result in a desired property, for example increased or reduced affinity for an FcγR. Such modifications may further enhance the phenotype of the original amino acid modification of the heavy chain variants of the invention. Although not intending to be bound by a particular mechanism of actions such a strategy is supported by the observation that the carbohydrate structure and conformation dramatically affect Fc-FcγR and Fc/C1q binding (Umaha et al., 1999, Nat Biotechnol 17:176-180; Davies et al., 2001, Biotechnol Bioeng 74:288-294; Mimura et al., 2001, J Biol Chem 276:45539; Radaev et al., 2001, J Biol Chem 276:16478-16483; Shields et al. 2002, Biol Chem 277:26733-26740; Shinkawa et al., 2003, J Biol Chem 278:3466-3473).

In certain embodiments, the invention encompasses molecules comprising a variant heavy chain having the Fc region of IgG2, IgG3 or IgG4, and having one or more amino acid modifications relative to a wild type heavy chain having an Fc region of the same isotype, wherein said one or more modifications eliminates the structural and functional dependence of the Fc region of said variant heavy chain on glycosylation. This strategy involves the optimization of heavy chain and/or Fc structure, stability, solubility, and function (for example affinity of Fc of the variant heavy chain for one or more Fc ligands) in the absence of the N297 carbohydrate. In one approach, positions that are exposed to solvent in the absence of glycosylation are modified such that they are stable, structurally consistent with wild type Fc structure, and have no tendency to aggregate. Approaches for optimizing heavy chains engineered according to the invention which are aglycosylated in the Fc region may involve but are not limited to designing amino acid modifications that enhance aglycoslated Fc region stability and/or solubility by incorporating polar and/or charged residues that face inward towards the Cg2-Cg2 dimer axis, and by designing amino acid modifications that directly enhance the aglycosylated Fc-FcγR interface or the interface of aglycosylated Fc with some other Fc ligand.

The heavy chain variants of the present invention may be combined with other heavy chain modifications, including but not limited to modifications that alter effector function. The invention encompasses combining an heavy chain variant of the invention with other heavy chain modifications to provide additive, synergistic, or novel properties in antibodies or Fc fusions. Such modifications may be in the CH1, CH2, hinge or CH3 domains or a combination thereof. Preferably the heavy chain variants of the invention enhance the property of the modification with which they are combined. For example, if a heavy chain variant of the invention is combined with a mutant known to bind FcγRIIIA with a higher affinity than a comparable molecule comprising a wild type Fc region having an Fc region of the same isotype; the combination with a mutant of the invention results in a greater fold enhancement in FcγRIIIA affinity.

In one embodiment, the heavy chain variants of the present invention, e.g., a heavy chain having the Fc region of IgG2, IgG3 or IgG4 and comprising one or more amino acid modifications (e.g., substitutions) relative to a wild-type heavy chain having the Fc region of the same isotype, may be combined with other known heavy chain variants such as those disclosed in Duncan et al., 1988, Nature 332:563-564; Lund et al., 1991, J. Immunol. 147:2657-2662; Lund et al., 1992, Mol Immunol 29:53-59; Alegre et al., 1994, Transplantation 57:1537-1543; Hutchins et al., 1995, Proc Natl. Acad Sci USA 92:11980-11984; Jefferis et al., 1995, Immunol Lett. 44:111-117; Lund et al., 1995, Faseb J 9:115-119; Jefferis et al., 1996, Immunol Lett 54:101-104; Lund et al., 1996, J Immunol 157:49634969; Armour et al., 1999, Eur J Immunol 29:2613-2624; Idusogie et al., 2000, J Immunol 164:41784184; Reddy et al., 2000, J Immunol 164:1925-1933; Xu et al., 2000, Cell Immunol 200:16-26; Idusogie et al., 2001, J Immunol 166:2571-2575; Shields et al., 2001, J Biol Chem 276:6591-6604; Jefferis et al., 2002, Immunol Lett 82:57-65; Presta et al., 2002, Biochem Soc Trans 30:487-490); U.S. Pat. No. 5,624,821; U.S. Pat. No. 5,885,573; U.S. Pat. No. 6,194,551; PCT WO 00/42072; PCT WO 99/58572; each of which is incorporated herein by reference in its entirety.

6.1.1 Polypeptide and Antibody Conjugates

Molecules of the invention (i.e., polypeptides, antibodies) comprising variant heavy chains may be recombinantly fused or chemically conjugated (including both covalently and non-covalently conjugations) to heterologous polypeptides (i.e., an unrelated polypeptide; or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences.

Further, molecules of the invention (i.e., polypeptides, antibodies) comprising variant heavy chains may be conjugated to a therapeutic agent or a drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin (i.e., PE-40), or diphtheria toxin, ricin, gelonin, and pokeweed antiviral protein, a protein such as tumor necrosis factor, interferons including, but not limited to, α-interferon (IFN-α), β-interferon (IFN-β), nerve growth factor (NGF), platelet derived growth factor (PDGF), tissue plasminogen activator (TPA), an apoptotic agent (e.g., TNF-α, TNF-β, AIM I as disclosed in PCT Publication No. WO 97/33899), AIM II (see, PCT Publication No. WO 97/34911), Fas Ligand (Takahashi et al., J. Immunol., 6:1567-1574, 1994), and VEGI (PCT Publication No. WO 99/23105), a thrombotic agent or an anti-angiogenic agent (e.g., angiostatin or endostatin), or a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”), macrophage colony stimulating factor, (“M-CSF”), or a growth factor (e.g., growth hormone (“GH”); proteases, or ribonucleases.

Molecules of the invention (i.e., polypeptides, antibodies) can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA, 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., Cell, 37:767 1984) and the “flag” tag (Knappik et al., Biotechniques, 17(4):754-761, 1994).

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of molecules of the invention (e.g., antibodies with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety). Molecules of the invention comprising variant Fc regions, or the nucleic acids encoding the molecules of the invention, may be further altered by being subjected to random mutagenesis by error-prone PCR, random nucleotide insertion or other methods prior to recombination. One or more portions of a polynucleotide encoding a molecule of the invention, may be recombined with one or more components, motifs, sections, parts, domains, fragments, etc. of one or more heterologous molecules.

The present invention also encompasses molecules of the invention comprising variant heavy chains (i.e., antibodies, polypeptides) conjugated to a diagnostic or therapeutic agent or any other molecule for which serum half-life is desired to be increased and/or targeted to a particular subset of cells. The molecules of the invention can be used diagnostically to, for example, monitor the development or progression of a disease, disorder or infection as part of a clinical testing procedure to, e.g., determine the efficacy of a given treatment regimen. Detection can be facilitated by coupling the molecules of the invention to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance may be coupled or conjugated either directly to the molecules of the invention or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Such diagnosis and detection can be accomplished by coupling the molecules of the invention to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²¹I, ¹²³I, ¹²¹I), lanthanium (¹⁴⁰La), lutetium (¹⁷⁷Lu), manganese (⁵⁴Mn), molybdenum (⁹⁹Mo), palladium (103Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

Molecules of the invention (i.e., antibodies, polypeptides) comprising a variant heavy chain may be conjugated to a therapeutic moiety such as a cytotoxin (e.g., a cytostatic or cytocidal agent), a therapeutic agent or a radioactive element (e.g., alpha-emitters, gamma-emitters, etc.). Cytotoxins or cytotoxic agents include any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC), and anti-mitotic agents (e.g., vincristine and vinblastine).

Moreover, a molecule of the invention can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol 26:943-50 each of which is incorporated herein by reference in their entireties.

Techniques for conjugating such therapeutic moieties to antibodies are well known; see, e.g., Arnon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al (eds.), 1985, pp. 303-16, Academic Press; and Thorpe et al., Immunol Rev., 62:119-58, 1982.

In one embodiment, where the molecule of the invention is an antibody comprising a variant heavy chains, it can be administered with or without a therapeutic moiety conjugated to it, administered alone, or in combination with cytotoxic factor(s) and/or cytokine(s) for use as a therapeutic treatment. Alternatively, an antibody of the invention can be conjugated to a second antibody to form an antibody heteroconjugate as described by Segal in U.S. Pat. No. 4,676,980, which is incorporated herein by reference in its entirety. Antibodies of the invention may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

6.2 Screening of Molecules with Variant Heavy Chains for Enhanced FcγRIII Binding and Characterization of Same

The affinities and binding properties of the molecules of the invention for an FcγR are initially determined using in vitro assays (biochemical or immunological based assays) known in the art for determining heavy chain-antibody receptor, and in particular, Fc-FcγR, interactions, i.e., specific binding of an Fc region to an FcγR including but not limited to ELISA assay, surface plasmon resonance assay, immunoprecipitation assays. Preferably, the binding properties of the molecules of the invention are also characterized by in vitro functional assays for determining one or more FcγR mediator effector cell functions. In most preferred embodiments, the antibodies of the invention have similar binding properties in in vivo models (such as those described and disclosed herein) as those in in vitro based assays. However, the present invention does not exclude molecules of the invention that do not exhibit the desired phenotype in in vitro based assays but do exhibit the desired phenotype in vivo.

In some embodiments, screening and identifying molecules comprising variant heavy chains with altered FcγR affinities (e.g., enhanced FcγRIIIA affinity) are done functional based assays, preferably in a high throughput manner. The functional based assays can be any assay known in the art for characterizing one or more FcγR mediated effector cell functions such as those described herein in Section 5.3. Non-limiting examples of effector cell functions that can be used in accordance with the methods of the invention, include but are not limited to, antibody-dependent cell mediated cytotoxicity (ADCC), antibody-dependent phagocytosis, phagocytosis, opsonization, opsonophagocytosis, cell binding, rosetting, C1q binding, and complement dependent cell mediated cytotoxicity.

The term “specific binding” of an Fc region to an FcγR refers to an interaction of the Fc region and a particular FcγR which has an affinity constant of at least about 150 nM, in the case of monomeric FcγRIIIA and at least about 60 nM in the case of dimeric FcγRIIB as determined using, for example, an ELISA or surface plasmon resonance assay (e.g., a BIAcore™). The affinity constant of an Fc region for monomeric FcγRIIIA may be 150 nM, 200 nM or 300 nM. The affinity constant of an Fc region for dimeric FcγRIIB may be 60 nM, 80 nM, 90 nM, or 100 nM. Dimeric FcγRIIB for use in the methods of the invention may be generated using methods known to one skilled in the art. Typically, the extracellular region of FcγRIIB is covalently linked to a heterologous polypeptide which is capable of dimerization, so that the resulting fusion protein is a dimer, e.g., see, U.S. Application No. 60/439,709 filed on Jan. 13, 2003 (Attorney Docket No. 11183-005-888), which is incorporated herein by reference in its entirety. A specific interaction generally is stable under physiological conditions, including, for example, conditions that occur in a living individual such as a human or other vertebrate or invertebrate, as well as conditions that occur in a cell culture such conditions as used for maintaining and culturing mammalian cells or cells from another vertebrate organism or an invertebrate organism.

In a specific embodiment, characterizing the binding of the molecule of the invention comprising the variant heavy chain to an FcγR (one or more) is done using a biochemical assay for determining Fc-FcγR interaction, preferably, an ELISA based assay. Once the molecule comprising a variant heavy chain has been characterized for its interaction with one or more FcγRs and determined to have an altered affinity for one or more FcγRs, by at least one biochemical based assay, e.g., an ELISA assay, the molecule maybe engineered into a complete immunoglobulin, using standard recombinant DNA technology methods known in the art, and the immunoglobulin comprising the variant heavy chain expressed in mammalian cells for further biochemical characterization. The immunoglobulin into which a variant heavy chain of the invention is introduced (e.g., replacing the Fc region of the immunoglobulin) can be any immunoglobulin including, but not limited to, polyclonal antibodies, monoclonal antibodies, bispecific antibodies, multi-specific antibodies, humanized antibodies, and chimeric antibodies. In preferred embodiments, a variant heavy chain is introduced into an immunoglobulin specific for a cell surface receptor, a tumor antigen, or a cancer antigen. The immunoglobulin into which a variant heavy chain of the invention is introduced may specifically bind a cancer or tumor antigen for example, including, but not limited to, KS 1/4 pan-carcinoma antigen (Perez and Walker, 1990, J. Immunol. 142: 3662-3667; Bumal, 1988, Hybridoma 7(4): 407-415), ovarian carcinoma antigen (CA125) (Yu et al., 1991, Cancer Res. 51(2): 468-475), prostatic acid phosphate (Tailor et al., 1990, Nucl. Acids Res. 18(16): 4928), prostate specific antigen (Henttu and Vihko, 1989, Biochem. Biophys. Res. Comm. 160(2): 903-910; Israeli et al., 1993, Cancer Res. 53: 227-230), melanoma-associated antigen p97 (Estin et al., 1989, J. Natl. Cancer Instit. 81(6): 445-446), melanoma antigen gp75 (Vijayasardahl et al., 1990, J. Exp. Med. 171(4): 1375-1380), high molecular weight melanoma antigen (HMW-MAA) (Natali et al., 1987, Cancer 59: 55-63; Mittelman et al., 1990, J. Clin. Invest. 86: 2136-2144), prostate specific membrane antigen, carcinoembryonic antigen (CEA) (Foon et al., 1994, Proc. Am. Soc. Clin. Oncol. 13: 294), polymorphic epithelial mucin antigen, human milk fat globule antigen, colorectal tumor-associated antigens such as: CEA, TAG-72 (Yokata et al., 1992, Cancer Res. 52: 3402-3408), C017-1A (Ragnhammar et al., 1993, Int. J. Cancer 53: 751-758); GICA 19-9 (Herlyn et al., 1982, J. Clin. Immunol. 2: 135), CTA-1 and LEA, Burkitt's lymphoma antigen-38.13, CD19 (Ghetie et al., 1994, Blood 83: 1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shiara et al., 1993, Cancer Immunol. Immunother. 36:373-380), ganglioside GM2 (Livingston et al., 1994, J. Clin. Oncol. 12: 1036-1044), ganglioside GM3 (Hoon et al., 1993, Cancer Res. 53: 5244-5250), tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46: 3917-3923), antigens of fibrosarcoma, human leukemia T cell antigen-Gp37 (Bhattacharya-Chatterjee et al., 1988, J. of Immun. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245: 301-304), differentiation antigen (Feizi, 1985, Nature 314: 53-57) such as I antigen found in fetal erythrocytes, primary endoderm I antigen found in adult erythrocytes, preimplantation embryos, I(Ma) found in gastric adenocarcinomas, M18, M39 found in breast epithelium, SSEA-1 found in myeloid cells, VEP8, VEP9, My1, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, GD3, D1.1, OFA-1, G_{M2}, OFA-2, G_{D2}, and M1:22:25:8 found in embryonal carcinoma cells, and SSEA-3 and SSEA-4 found in 4 to 8-cell stage embryos. In one embodiment, the antigen is a T cell receptor derived peptide from a Cutaneous Tcell Lymphoma (see, Edelson, 1998, The Cancer Journal 4:62).

In some embodiments, a variant heavy chain of the invention is introduced into an anti-fluoresceine monoclonal antibody, 4-4-20 (Kranz et al., 1982 J. Biol. Chem. 257(12): 6987-6995; which is incorporated herein by reference in its entirety). In yet other embodiments, a variant heavy chain of the invention is introduced into a mouse-human chimeric anti-CD20 monoclonal antibody 2H7, which recognizes the CD20 cell surface phosphoprotein on B cells (Liu et al., 1987, Journal of Immunology, 139: 3521-6; which is incorporated herein by reference in its entirety). In yet other embodiments, a variant heavy chain of the invention is introduced into a humanized antibody (Ab4D5) against the human epidermal growth factor receptor 2 (p185 HER2) as described by Carter et al. (1992, Proc. Natl. Acad. Sci. USA 89: 4285-9; which is incorporated herein by reference in its entirety). In yet other embodiments, a variant heavy chain of the invention is introduced into a humanized anti-TAG72 antibody (CC49) (Sha et al., 1994 Cancer Biother. 9(4): 341-9, which is incorporated by reference herein in its entirety). In other embodiments, a variant heavy chains of the invention is introduced into RITUXAN® (humanized anti-CD20 antibody; rituximab) (International Patent Publication No. WO 02/096948; which is incorporated herein by reference in its entirety) which is used for treating lymphomas.

In another specific embodiment, the invention encompasses engineering an anti-FcγRIIB antibody including but not limited to any of the antibodies disclosed in U.S. Provisional Application No. 60/403,266 filed on Aug. 12, 2002; U.S. application Ser. No. 10/643,857 filed on Aug. 14, 2003; U.S. Provisional Application No. 60/562,804 filed on Apr. 16, 2004; U.S. Provisional Application Nos. 60/582,044, 60/582,045, and 60/582,043, each of which was filed on Jun. 21, 2004; U.S. Provisional Application No. 60/636,663 filed on Dec. 15, 2004 and U.S. application Ser. No. 10/524,134 filed Feb. 11, 2005 by modification (e.g., substitution, insertion, deletion) of at least one amino acid residue which modification increases the affinity of the Fc region for FcγRIIIA and/or FcγRIIA. In another specific embodiment, the invention encompasses engineering a humanized anti-FcγRIIB antibody including but not limited to any of the antibodies disclosed in U.S. Provisional Application No. 60/569,882 filed on May 10, 2004 and U.S. application Ser. No. 11/126,978, filed on May 10, 2005; by modification (e.g., substitution, insertion, deletion) of at least one amino acid residue which modification increases the affinity of the Fe region for FcγRIIIA and/or FcγRIIA. Each of the above mentioned applications is incorporated herein by reference in its entirety. Examples of anti-FcγRIIB antibodies, which may or may not be humanized, that may be engineered in accordance with the methods of the invention are 2B6 monoclonal antibody having ATCC accession number PTA-4591 and 3H7 having ATCC accession number PTA-4592, 1D5 monoclonal antibody having ATCC accession number PTA-5958, 1F2 monoclonal antibody having ATCC accession number PTA-5959, 2D11 monoclonal antibody having ATCC accession number PTA-5960, 2E1 monoclonal antibody having ATCC accession number PTA-5961 and 2H9 monoclonal antibody having ATCC accession number PTA-5962 (all deposited at 10801 University Boulevard, Manassas, Va. 02209-2011), which are incorporated herein by reference. In another specific embodiment, modification of the anti-FcγRIIB antibody may also further decrease the affinity of the Fe region for FcγRIIB. In yet another specific embodiment, the engineered anti-FcγRIIB antibody may further have an enhanced effector function as determined by standard assays known in the art and disclosed and exemplified herein. In some embodiments, a variant heavy chain of the invention having the Fe region of IgG2, IgG3 or IgG4 is introduced into a therapeutic monoclonal antibody specific for a cancer antigen or cell surface receptor including but not limited to, Erbitux™ (also known as IMC-C225) (ImClone Systems Inc.), a chimerized monoclonal antibody against EGFR; HERCEPTIN® (Trastuzumab) (Genentech, CA) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection. Other examples are a humanized anti-CD18 F(ab′)₂ (Genentech); CDP860 which is a humanized anti-CD18 F(ab′)₂ (Celltech, UK); PRO542 which is an anti-HIV gp120 antibody fused with CD4 (Progenics/Genzyme Transgenics); C14 which is an anti-CD14 antibody (ICOS Pharm); a humanized anti-VEGF IgG1 antibody (Genentech); OVAREX™ which is a murine anti-CA 125 antibody (Altarex); PANOREX™ which is a murine anti-17-IA cell surface antigen IgG2a antibody (Glaxo Wellcome/Centocor); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ (rituximab) which is a chimeric anti-CD20 IgG1 antibody (IDEC Pharm/Genentech, Roche/Zettyaku); LYMPHOCIDE™ which is a humanized anti-CD22 IgG antibody (Immunomedics); Smart ID10 which is a humanized anti-HLA antibody (Protein Design Lab); ONCOLYM™ (Lym-1) is a radiolabelled murine anti-HLA DR antibody (Techniclone); anti-CD11a is a humanized IgG1 antibody (Genetech/Xoma); ICM3 is a humanized anti-ICAM3 antibody (ICOS Pharm); IDEC-114 is a primatized anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); MDX-33 is a human anti-CD64 (FcγR) antibody (Medarex/Centeon); rhuMab-E25 is a humanized anti-IgE IgG1 antibody (Genentech/Norvartis/Tanox Biosystems); IDEC-152 is a primatized anti-CD23 antibody (IDEC Pharm); ABX-CBL is a murine anti CD-147 IgM antibody (Abgenix); BTI-322 is a rat anti-CD2 IgG antibody (Medimmune/Bio Transplant); Orthoclone/OKT3 is a murine anti-CD3 IgG2a antibody (ortho Biotech); SIMULECT™ is a chimeric anti-CD25 IgG1 antibody (Novartis Pharm); LDP-01 is a humanized anti-β₂-integrin IgG antibody (LeukoSite); Anti-LFA-1 is a murine anti CD18 F(ab′)₂ (Pasteur-Merieux/Immunotech); CAT-152 is a human anti-TGF-β₂ antibody (Cambridge Ab Tech); and Corsevin M is a chimeric anti-Factor VII antibody (Centocor).

The variant heavy chains of the invention, preferably in the context of an immunoglobulin, can be further characterized using one or more biochemical assays and/or one or more functional assays, preferably in a high throughput manner. In some alternate embodiments, the variant heavy chains of the inventions are not introduced into an immunoglobulin and are further characterized using one or more biochemical based assays and/or one or more functional assays, preferably in a high throughput manner. The one or more biochemical assays can be any assay known in the art for identifying immunoglobulin-antigen, heavy chain-antibody receptor, or Fc-FcγR interactions, including, but not limited to, an ELISA assay, and surface plasmon resonance-based assay, e.g., BIAcore assay, for determining the kinetic parameters of Fc-FcγR or immunoglobulin-antigen interaction. Characterization of target antigen binding affinity or assessment of target antigen density on a cell surface may be assessed by methods well known in the art such as Scatchard analysis or by the use of kits as per manufacturer's instructions, such as Quantum™ Simply Cellular® (Bangs Laboratories, Inc., Fishers, Ind.). The one or more functional assays can be any assay known in the art for characterizing one or more FcγR mediated effector cell function as known to one skilled in the art or described herein. In specific embodiments, the immunoglobulins comprising the variant Fc regions are assayed in an ELISA assay for binding to one or more FcγRs, e.g., FcγRIIIA, FcγRIIA, FcγRIIA; followed by one or more ADCC assays. In some embodiments, the immunoglobulins comprising the variant Fc regions are assayed further using a surface plasmon resonance-based assay, e.g., BIAcore. Surface plasmon resonance-based assays are well known in the art, and are further discussed in Section 5.2, and exemplified herein, e.g., in Example 6.1.

An exemplary high throughput assay for characterizing immunoglobulins comprising variant heavy chains may comprise: introducing a variant heavy chain of the invention, e.g., by standard recombinant DNA technology methods, in a 4-4-20 antibody; characterizing the specific binding of the 4-4-20 antibody comprising the variant heavy chain to an FcγR (e.g., FcγRIIIA, FcγRIIB) in an ELISA assay; characterizing the 4-4-20 antibody comprising the variant heavy chain in an ADCC assay (using methods disclosed herein) wherein the target cells are opsonized with the 4-4-20 antibody comprising the variant heavy chain; the variant heavy chain may then be cloned into a second immunoglobulin, e.g., 4D5, 2H7, and that second immunoglobulin characterized in an ADCC assay, wherein the target cells are opsonized with the second antibody comprising the variant heavy chain. The second antibody comprising the variant heavy chain is then further analyzed using an ELISA-based assay to confirm the specific binding to an FcγR.

Preferably, a variant heavy chain of the invention binds FcγRIIIA and/or FcγRIIA with a higher affinity than a wild type heavy chain having an Fc region of the same isotype as determined in an ELISA assay. Most preferably, a variant heavy chain of the invention binds FcγRIIIA and/or FcγRIIA with a higher affinity and binds FcγRIIB with a lower affinity than a wild type heavy chain having an Fc region of the same isotype as determined in an ELISA assay. In some embodiments, the variant heavy chain binds FcγRIIIA and/or FcγRIIA with at least 2-fold higher, at least 4-fold higher, more preferably at least 6-fold higher, most preferably at least 8 to 10-fold higher affinity than a wild type heavy chain having an Fc region of the same isotype binds FcγRIIIA and/or FcγRIIA and binds FcγRIIB with at least 2-fold lower, at least 4-fold lower, more preferably at least 6-fold lower, most preferably at least 8 to 10-fold lower affinity than a wild type heavy chain having an Fe region of the same isotype binds FcγRIIB as determined in an ELISA assay.

The immunoglobulin comprising the variant heavy chains of the invention may be analyzed at any point using a surface plasmon based resonance based assay, e.g., BIAcore, for defining the kinetic parameters of the Fc-FcγR interaction, using methods disclosed herein and known to those of skill in the art. Preferably, the Kd of the molecules of the invention for binding to a monomeric FcγRIIIA and/or FcγRIIA as determined by BIAcore analysis are about 100 nM, preferably about 70 nM, most preferably about 40 nM.; and the Kd of the molecules of the invention for binding a dimeric FcγRIIB is about 80 nM, about 100 nM, more preferably about 200 nM.

In most preferred embodiments, the immunoglobulin comprising the variant heavy chain of the invention (i.e., a heavy chain containing the Fe region of IgG2, IgG3, or IgG4, having at least one amino acid modification relative to a wild type chain having an Fe region of the same isotype) is further characterized in an animal model for interaction with an FcγR. Preferred animal models for use in the methods of the invention are, for example, transgenic mice expressing human FcγRs, e.g., any mouse model described in U.S. Pat. Nos. 5,877,397, and 6,676,927 which are incorporated herein by reference in their entirety. Transgenic mice for use in the methods of the invention include, but are not limited to, nude knockout FcγRIIIA mice carrying human FcγRIIIA; nude knockout FcγRIIIA mice carrying human FcγRIIA; nude knockout FcγRIIIA mice carrying human FcγRIIB and human FcγRIIIA; nude knockout FcγRIIIA mice carrying human FcγRIIB and human FcγRIIA; nude knockout FcγRIIIA and FcγRIIA mice carrying human FcγRIIIA and FcγRIIA and nude knockout FcγRIIIA, FcγRIIA and FcγRIIB mice carrying human FcγRIIIA, FcγRIIA and FcγRIIB.

6.2.1 FcγR-Fc Binding Assay

An FcγR-Fc binding assay was developed for determining the binding of the molecules of the invention to FcγR, which allowed detection and quantitation of the interaction, despite the inherently weak affinity of the receptor for its ligand, e.g., in the micromolar range for FcγRIIB and FcγRIIIA. The method is described in detail in International Application WO04/063351 and U.S. Patent Application Publications 2005/0037000 and 2005/0064514. Briefly, the method involves the formation of an FcγR complex that has an improved avidity for an Fe region of the variant heavy chain, relative to an uncomplexed FcγR. According to the invention, the preferred molecular complex is a tetrameric immune complex, comprising: (a) the soluble region of FcγR (e.g., the soluble region of FcγRIIIA, FcγRIIA or FcγRIIB); (b) a biotinylated 15 amino acid AVITAG sequence (AVITAG) operably linked to the C-terminus of the soluble region of FcγR (e.g., the soluble region of FcγRIIIA, FcγRIIA or FcγRIIB); and (c) streptavidin-phycoerythrin (SA-PE); in a molar ratio to form a tetrameric FcγR complex (preferably in a 5:1 molar ratio). According to a preferred embodiment of the invention, the fusion protein is biotinylated enzymatically, using for example, the E. coli Bir A enzyme, a biotin ligase which specifically biotinylates a lysine residue in the 15 amino acid AVITAG sequence. In a specific embodiment of the invention, 85% of the fusion protein is biotinylated, as determined by standard methods known to those skilled in the art, including but not limited to streptavidin shift assay. According to preferred embodiments of the invention, the biotinylated soluble FcγR proteins are mixed with SA-PE in a 1×SA-PE:5× biotinylated soluble FcγR molar ratio to form a tetrameric FcγR complex.

In a preferred embodiment of the invention, polypeptides comprising Fc regions bind the tetrameric FcγR complexes, with at least an 8-fold higher affinity than the monomeric uncomplexed FcγR. The binding of polypeptides comprising Fc regions to the tetrameric FcγR complexes may be determined using standard techniques known to those skilled in the art, such as for example, fluorescence activated cell sorting (FACS), radioimmunoassays, ELISA assays, etc.

The invention encompasses the use of the immune complexes comprising molecules of the invention, and formed according to the methods described above, for determining the functionality of molecules comprising an Fc region in cell-based or cell-free assays.

As a matter of convenience, the reagents may be provided in an assay kit, i.e., a packaged combination of reagents for assaying the ability of molecules comprising an Fc regions i to bind FcγR tetrameric complexes. Other forms of molecular complexes for use in determining Fc-FcγR interactions are also contemplated for use in the methods of the invention, e.g., fusion proteins formed as described in U.S. Provisional Application 60/439,709, filed on Jan. 13, 2003; which is incorporated herein by reference in its entirety.

6.2.2 Use of Yeast Display Libraries

Molecular interactions between the Fc regions of IgG heavy chains and Fc receptors have been previously studied by both structural and genetic techniques. These studies identified amino acid residues that are critical for functional binding of Fc to different FcγR. None of these changes have been shown to improve human FcγR mediated efficacy of therapeutic antibodies in animal models. A complete analysis of all potential amino acid changes at these residues or other potentially important residues has not been reported.

The instant invention encompasses the use of heavy chain and/or Fc mutations disclosed in International Application WO04/063351 and U.S. Patent Application Publications 2005/0037000 and 2005/0064514, concurrent applications of the inventions, each of which is incorporated herein by reference in its entirety. The amino acid modifications (i.e., mutations) were identified using a library of randomly mutagenized IgG1 Fc and the screening assays described in detail in the applications. In addition regions for modification may be chosen based on available information, e.g., crystal structure data, Mouse/Human isotype FcγR binding differences, genetic data, and additional sites identified by mutagenesis. It will be appreciated by one of skill in the art, that once molecules of the invention with desired binding properties (e.g., molecules with variant Fc regions with at least one amino acid modification, which modification enhances the affinity of the variant Fc region for FcγRIIIA relative to a comparable molecule, comprising a wild-type Fc region) have been identified (See Section 5.1 and, e.g., Tables 3 and 9), other molecules (i.e, therapeutic antibodies) may be engineered using standard recombinant DNA techniques and any known mutagenesis techniques, as described herein or known in the art to produce engineered molecules carrying the identified mutation sites.

The following tables, adapted from International Application WO04/063351 and U.S. Patent Application Publications 2005/0037000 and 2005/0064514, referenced supra, summarize those mutations that were found to alter Fc-FcγR interaction, specifically the Fc interaction of IgG1 with FcγRIIIA and FcγRIIB. Table 10 and Table 11 summarize mutations that improved affinity and decreased the K_{off} of the variant IgG1 Fc-FcγRIIIA interaction, respectively, which mutations were identified by the Inventors using sequential equilibrium or kinetic FACS screening. Table 12 and Table 13 summarize mutations that allowed IgG1 Fc binding to FcγRIIIA but eliminated IgG1 Fc-FcγRIIB binding, which mutations were identified by the Inventors using sequential solid-phase separation screening.

TABLE 10
IgG1 Mutants selected by FACS using an Equilibrium screen
with concentrations of FcγRIIIA of approximately 7 nM.

| Mutant | Amino Acid changes |
|---|---|
| MgFc43b | K288R, T307A, K344E, P396L |
| MgFc44 | K334N, P396L |
| MgFc46 | P217S, P396L |
| MgFc47 | K210M, P396L |
| MgFc48 | V379M, P396L |
| MgFc49 | K261N, K210M, P396L |
| MgFc60 | P217S, P396L |

TABLE 11
IgG1 Mutants selected by FACS using a Kinetic screen using
equimolar amounts of unlabeled FcγRIIIA for 1 minute.

| Mutants | Amino Acid changes |
|---|---|
| MgFc50 | P247S, P396L |
| MgFc51 | Q419H, P396L |
| MgFc52 | V240A, P396L |
| MgFc53 | L410H, P396L |
| MgFc54 | F243L, V305I, A378D, F404S, P396L |
| MgFc55 | R255l, P396L |
| MgFc57 | L242F, P396L |
| MgFc59 | K370E, P396L |

TABLE 12
IgG1 Mutants selected by sequential solid phase depletion and
selection using Magnetic beads coated with FcγRIIB followed by
selection with magnetic beads coated with FcγRIIIA.

| Mutant | Amino Acid changes |
|---|---|
| MgFc37 | K248M |
| MgFc38 | K392T, P396L |
| MgFc39 | E293V, Q295E, A327T |
| MgFc41 | H268N, P396LN |
| MgFc43 | Y319F, P352L, P396L |
| MgFc42 | D221E, D270E, V308A, Q311H, P396L, G402D |

TABLE 13
IgG1 Mutants selected by magnetic bead depletion using beads
coated with CD32B and final selection by FACS using FcγRIIIA
158Valine or 158Phenylalanine

| Mutants | Amino Acid Changes |
|---|---|
| MgFc61 | A330V |
| MgFc62 | R292G |
| MgFc63 | S298N, K360R, N361D |
| MgFc64 | E233G |
| MgFc65 | N276Y |
| MgFc66 | A330V, V427M |
| MgFc67 | V284M, S298N, K334E, R355W, R416T |

Table 14 summarizes mutations and their FcγR binding characteristics previously determined by the Inventors using both yeast display based assays and ELISA. In Table 14, the symbols represent the following: • corresponds to a 1-fold increase in affinity; + corresponds to a 50% increase in affinity; − corresponds to a 1-fold decrease in affinity; corresponds to no change in affinity compared to a comparable molecule comprising a wild-type Fc region.

TABLE 14
IgG1 Fc Mutations Identified and Binding Characteristics by ELISA

| Clone | | | IIIA | |
|---|---|---|---|---|
| # | Mutation Sites | Domain | binding | IIB binding |
| 4 | A339V, Q347H | CH2, CH3 | + | + |
| 5 | L251P, S415I | CH2, CH3 | + | + |
| 7 | Aga2p-T43I | Note: This is a | | Aga2p-T43I |
| | | mutation in | | |
| | | Aga2P that | | |
| | | enhances | | |
| | | display. | | |
| 8 | V185M, K218N, | CH1, hinge, CH2, | no | − |
| | R292L, D399E | CH3 | change | |
| 12 | K290E, L142P | CH1, CH2 | + | not tested |
| 16 | A141V, H268L, | CH1, CH2 | − | not tested |
| | K288E, P291S | | | |
| 19 | L133M, P150Y, | CH1, CH2, CH3 | − | not tested |
| | K205E, S383N, | | | |
| | N384K | | | |
| 21 | P396L | CH3 | • | •+ |
| 25 | P396H | CH3 | ••• | •• |
| 6 | K392R | CH3 | no | no change |
| | | | change | |
| 15 | R301C, M252L, | CH1, CH2 | − | not tested |
| | S192T | | | |
| 17 | N315I | CH2 | no | not tested |
| | | | change | |
| 18 | S132I | CH1 | no | not tested |
| | | | change | |
| 26 | A162V | CH1 | no | not tested |
| | | | change | |
| 27 | V348M, K334N, | CH1, Ch2 | + | + |
| | F275I, Y202M, | | | |
| | K147T | | | |
| 29 | H310Y, T289A, | CH2 | − | not tested |
| | G337E | | | |
| 30 | S119F, G371S, | CH1, CH2, CH3 | + | no change |
| | Y407N, E258D | | | |
| 31 | K409R, S166N | CH1, CH3 | no | not tested |
| | | | change | |
| 20 | S408I, V215I, V125I | CH1, hinge, CH3 | + | no change |
| 24 | G385E, P247H | CH2, CH3 | ••• | + |
| 16 | V379M | CH3 | •• | no change |
| 17 | S219Y | Hinge | • | − |
| 18 | V282M | CH2 | • | − |
| 31 | F275I, K334N, | CH2 | + | no change |
| | V348M | | | |
| 35 | D401V | CH3 | + | no change |
| 37 | V280L, P395S | CH2 | + | − |
| 40 | K222N | Hinge | • | no change |
| 41 | K246T, Y319F | CH2 | • | no change |
| 42 | F243I, V379L | CH2, CH3 | •+ | − |
| 43 | K334E | CH2 | •+ | − |
| 44 | K246T, P396H | CH2, CH3 | • | ••+ |
| 45 | H268D, E318D | CH2 | •+ | ••••• |
| 49 | K288N, A330S, | CH2, CH3 | ••••• | ••• |
| | P396L | | | |
| 50 | F243L, R255L, | CH2 | • | − |
| | E318K | | | |
| 53 | K334E, T359N, | CH2, CH3 | • | no change |
| | T366S | | | |
| 54 | I377F | CH3 | •+ | + |
| 57 | K334I | CH2 | • | no change |
| 58 | P244H, L358M, | CH2, CH3 | •+ | •+ |
| | V379M, N384K, | | | |
| | V397M | | | |
| 59 | K334E, T359N, | CH2, CH3 | •+ | no change |
| | T366S | | | |
| | (independent isolate) | | | |
| 61 | I377F (independent | CH3 | ••• | ••+ |
| | isolate) | | | |
| 62 | P247L | CH2 | •• | ••+ |
| 64 | P217S, A378V, | Hinge, CH3 | •• | ••••+ |
| | S408R | | | |
| 65 | P247L, I253N, | CH2 | ••• | ••+ |
| | K334N | | | |
| 66 | K288M, K334E | CH2 | ••• | − |
| 67 | K334E, E380D | CH2, CH3 | •+ | − |
| 68 | P247L (independent | CH2 | + | •••• |
| | isolate) | | | |
| 69 | T256S, V305I, | CH2, CH3 | •+ | no change |
| | K334E, N390S | | | |
| 70 | K326E | CH2 | •+ | ••+ |
| 71 | F372Y | CH3 | + | •••••+ |
| 72 | K326E (independent | CH2 | + | •• |
| | isolate) | | | |
| 74 | K334E, T359N, | CH2, CH3 | •• | no change |
| | T366S | | | |
| | (independent isolate) | | | |
| 75 | K334E (independent | CH2 | ••+ | no change |
| | isolate) | | | |
| 76 | P396L (independent | CH3 | •+ | no change |
| | isolate) | | | |
| 78 | K326E (independent | CH2 | •• | •••+ |
| | isolate) | | | |
| 79 | K246I, K334N | CH2 | • | •••• |
| 80 | K334E (independent | CH2 | • | no change |
| | isolate) | | | |
| 81 | T335N, K370E, | CH2, CH3 | • | no change |
| | A378, T394M, | | | |
| | S424L | | | |
| 82 | K320E, K326E | CH2 | • | • |
| 84 | H224L | Hinge | • | ••••• |
| 87 | S375C, P396L | CH3 | •+ | ••••+ |
| 89 | E233D, K334E | CH2 | •+ | no change |
| 91 | K334E (independent | CH2 | • | no change |
| | isolate) | | | |
| 92 | K334E (independent | CH2 | • | no change |
| | isolate) | | | |
| 94 | K334E, T359N, | CH2 | • | no change |
| | T366S, Q386R | | | |

6.3 FACS Assays; Solid Phased Assays and Immunological Based Assays

FcγR Molecules of the present invention (e.g., antibodies, fusion proteins, conjugated molecules) may be characterized in a variety of ways. In particular, molecules of the invention comprising modified heavy chains may be assayed for the ability to immunospecifically bind to a ligand, e.g., FcγRIIIA tetrameric complex. Such an assay may be performed in solution (e.g., Houghten, Bio/Techniques, 13:412-421, 1992), on beads (Lam, Nature, 354:82-84, 1991, on chips (Fodor, Nature, 364:555-556, 1993), on bacteria (U.S. Pat. No. 5,223,409), on spores (U.S. Pat. Nos. 5,571,698; 5,403,484; and 5,223,409), on plasmids (Cull et al., Proc. Natl. Acad. Sci. USA, 89:1865-1869, 1992) or on phage (Scott and Smith, Science, 249:386-390, 1990; Devlin, Science, 249:404-406, 1990; Cwirla et al., Proc. Natl. Acad. Sci. USA, 87:6378-6382, 1990; and Felici, J. Mol. Biol., 222:301-310, 1991) (each of these references is incorporated by reference herein in its entirety). Molecules that have been identified to immunospecifically bind to an ligand, e.g., FcγRIIIA can then be assayed for their specificity and affinity for the ligand.

Molecules of the invention that have been engineered to comprise modified heavy chains (e.g., therapeutic antibodies) may be assayed for immunospecific binding to an antigen (e.g., cancer antigen and cross-reactivity with other antigens (e.g., FcγR) by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety).

One exemplary system for characterizing the molecules of the invention comprises a mammalian expression vector containing the heavy chain of the anti-fluorescein monoclonal antibody 4-4-20, into which the nucleic acids encoding the molecules of the invention with variant heavy chains are cloned. The resulting recombinant clone is expressed in a mammalian host cell line (i.e., human kidney cell line 293H), and the resulting recombinant immunoglobulin is analyzed for binding to FcγR using any standard assay known to those in the art, including but not limited to ELISA and FACS.

The binding affinity of the molecules of the present invention comprising modified heavy chains to a ligand, e.g., FcγR tetrameric complex and the off-rate of the interaction can be determined by competitive binding assays. One example of a competitive binding assay is a radioimmunoassay comprising the incubation of labeled ligand, such as tetrameric FcγR (e.g., ³H or ¹²⁵I) with a molecule of interest (e.g., molecules of the present invention comprising variant heavy chains (e.g., a heavy chain having the Fc region of IgG2, IgG3 or IgG4 and comprising one or more amino acid modifications relative to a wild-type heavy chain comprising an Fc region of the same isotype)) in the presence of increasing amounts of unlabeled ligand, such as tetrameric FcγR, and the detection of the molecule bound to the labeled ligand. The affinity of the molecule of the present invention for the ligand and the binding off-rates can be determined from the saturation data by scatchard analysis.

In a preferred embodiment, BIAcore kinetic analysis is used to determine the binding on and off rates of molecules of the present invention to a ligand such as FcγR. BIAcore kinetic analysis comprises analyzing the binding and dissociation of a ligand from chips with immobilized molecules (e.g., molecules comprising modified Fc regions) on their surface.

Characterization of binding to FcγR by molecules comprising the variant heavy chains of the invention of the invention may be done using any FcγR, including but not limited to polymorphic variants of FcγR. In some embodiments, selection of the Fc variants is done using a polymorphic variant of FcγRIIIA which contains a phenylalanine at position 158. In other embodiments, characterization is done using a polymorphic variant of FcγRIIIA which contains a valine at position 158. FcγRIIIA 158V displays a higher affinity for IgG1 than 158F and an increased ADCC activity (see, e.g., Koene et al., 1997, Blood, 90:1109-14; Wu et al., 1997, J. Clin. Invest. 100: 1059-70, both of which are incorporated herein by reference in their entireties); this residue in fact directly interacts with the lower hinge region of IgG1 as recently shown by IgG1-FcγRIIIA co-crystallization studies, see, e.g., Sonderman et al., 2000, Nature, 100: 1059-70, which is incorporated herein by reference in its entirety. Studies have shown that in some cases therapeutic antibodies have improved efficacy in FcγRIIIA-158V homozygous patients. For example, humanized anti-CD20 monoclonal antibody Rituximab was therapeutically more effective in FcγRIIIA158V homozygous patients compared to FcγRIIIA 158F homozygous patients (See, e.g., Cartron et al., 2002 Blood, 99(3): 754-8). In other embodiments, therapeutic antibodies may also be more effective on patients heterozygous for FcγRIIIA-158V and FcγRIIIA-158F, and in patients with FcγRIIA-131H. Although not intending to be bound by a particular mechanism of action, selection of molecules of the invention with alternate allotypes may provide for variants that once engineered into therapeutic antibodies will be clinically more efficacious for patients homozygous for said allotype.

The invention encompasses screening molecules comprising the variant heavy chain of the invention according to the methods described in Sections 5.2 and 5.3. One aspect of the invention provides a method of screening for molecules exhibiting a desirable binding property, specifically, the ability of the variant heavy chain, or portion thereof, to bind FcγRIIIA and/or FcγRIIA with a greater affinity than a comparable polypeptide comprising a wild-type heavy chain having an Fc region of the same isotype binds FcγRIIIA and/or FcγRIIA. In another embodiment, the invention provides a method for selecting those variant heavy chains, or portions thereof, that exhibit a desirable binding property, specifically, the ability of the variant heavy chain, or portion thereof, to bind FcγRIIIA and/or FcγRIIA with a greater affinity than a comparable polypeptide comprising a wild-type heavy chain having an Fc region of the same isotype binds FcγRIIIA and/or FcγRIIA, and further the ability of the variant heavy chain, or portion thereof, to bind FcγRIIB with a lower affinity than a comparable polypeptide comprising a wild-type heavy chain having an Fc region of the same isotype binds FcγRIIB. It will be appreciated by one skilled in the art, that the methods of the invention can be used for screening any mutations in the heavy chains of the invention, for any desired binding characteristic.

Preferably, fluorescence activated cell sorting (FACS), using any of the techniques known to those skilled in the art, is used for immunological or functional based assay to characterize molecules of the invention. Flow sorters are capable of rapidly examining a large number of individual cells that have been bound, e.g., opsonized, by molecules of the invention (e.g., 10-100 million cells per hour) (Shapiro et al., Practical Flow Cytometry, 1995). Additionally, specific parameters used for optimization of antibody behaviour, include but are not limited to, ligand concentration (i.e., FcγRIIIA tetrameric complex), kinetic competition time, or FACS stringency, each of which may be varied in order to select for the antibodies comprising colecules of the invention which exhibit specific binding properties, e.g., higher affinity for FcγRIIIA compared to a comparable polypeptide comprising a wild-type heavy chain having an Fc region of the same isotype. Flow cytometers for sorting and examining biological cells are well known in the art. Known flow cytometers are described, for example, in U.S. Pat. Nos. 4,347,935; 5,464,581; 5,483,469; 5,602,039; 5,643,796; and 6,211,477; the entire contents of which are incorporated by reference herein. Other known flow cytometers are the FACS Vantage™ system manufactured by Becton Dickinson and Company, and the COPAS™ system manufactured by Union Biometrica.

6.3.1 Functional Assays of Molecules with Variant Heavy Chains

The invention encompasses characterization of the molecules of the invention (e.g., an antibody comprising a variant heavy chain having the Fc region of IgG2, IgG3 or IgG4, and comprising mutations identified by the yeast display technology/analysis of IgG1 Fc regions; or therapeutic monoclonal antibodies engineered according to the methods of the invention) using assays known to those skilled in the art for identifying the effector cell function of the molecules. In particular, the invention encompasses characterizing the molecules of the invention for FcγR-mediated effector cell function. Examples of effector cell functions that can be assayed in accordance with the invention, include but are not limited to, antibody-dependent cell mediated cytotoxicity, phagocytosis, opsonization, opsonophagocytosis, C1q binding, and complement dependent cell mediated cytotoxicity. Any cell-based or cell free assay known to those skilled in the art for determining effector cell function activity can be used (For effector cell assays, see Perussia et al., 2000, Methods Mol. Biol. 121: 179-92; Baggiolini et al., 1998 Experientia, 44(10): 841-8; Lehmann et al., 2000 J. Immunol. Methods, 243(1-2): 229-42; Brown E J. 1994, Methods Cell Biol., 45: 147-64; Munn et al., 1990 J. Exp. Med., 172: 231-237, Abdul-Majid et al., 2002 Scand. J. Immunol. 55: 70-81; Ding et al., 1998, Immunity 8:403-411, each of which is incorporated by reference herein in its entirety).

In one embodiment, the molecules of the invention can be assayed for FcγR-mediated phagocytosis in human monocytes. Alternatively, the FcγR-mediated phagocytosis of the molecules of the invention may be assayed in other phagocytes, e.g., neutrophils (polymorphonuclear leuckocytes; PMN); human peripheral blood monocytes, monocyte-derived macrophages, which can be obtained using standard procedures known to those skilled in the art (e.g., see Brown E J. 1994, Methods Cell Biol, 45: 147-164). In one embodiment, the function of the molecules of the invention is characterized by measuring the ability of THP-1 cells to phagocytose fluoresceinated IgG-opsonized sheep red blood cells (SRBC) by methods previously described (Tridandapani et al., 2000, J. Biol. Chem. 275: 20480-7). For example, an exemplary assay for measuring phagocytosis of the molecules of the invention comprising variant heavy chains with enhanced affinities for FcγRIIIA, comprises of: treating THP-1 cells with a molecule of the invention or with a control antibody that does not bind to FcγRIIIA, comparing the activity levels of said cells, wherein a difference in the activities of the cells (e.g., rosetting activity (the number of THP-1 cells binding IgG-coated SRBC), adherence activity (the total number of SRBC bound to THP-1 cells), and phagocytic rate) would indicate the functionality of the molecule of the invention. It can be appreciated by one skilled in the art that this exemplary assay can be used to assay any of the molecules identified by the methods of the invention.

Another exemplary assay for determining the phagocytosis of the molecules of the invention is an antibody-dependent opsonophagocytosis assay (ADCP) which can comprise the following: coating a target bioparticle such as Escherichia coli-labeled FITC (Molecular Probes) or Staphylococcus aureus-FITC with (i) wild-type 4-4-20 antibody, an antibody to fluorescein (See Bedzyk et al., 1989, J. Biol. Chem., 264(3): 1565-1569, which is incorporated herein by reference in its entirety), as the control antibody for FcγR-dependent ADCP; or (ii) 4-4-20 antibody harboring the D265A mutation that knocks out binding to FcγRIII, as a background control for FcγR-dependent ADCP (iii) 4-4-20 antibody carrying variant Fc regions identified by the methods of the invention and produced as exemplified in Example 6.6; and forming the opsonized particle; adding any of the osponized particles described (i-iii) to THP-1 effector cells (a monocytic cell line available from ATCC) at a 1:1, 10:1, 30:1, 60:1, 75:1 or a 100:1 ratio to allow FcγR-mediated phagocytosis to occur; preferably incubating the cells and E. coli-FITC/antibody at 37° C. for 1.5 hour; adding trypan blue after incubation (preferably at room temperature for 2-3 min.) to the cells to quench the fluorescence of the bacteria that are adhered to the outside of the cell surface without being internalized; transferring cells into a FACS buffer (e.g., 0.1%, BSA in PBS, 0.1%, sodium azide), analyzing the fluorescence of the THP1 cells using FACS (e.g., BD FACS Calibur). Preferably, the THP-1 cells used in the assay are analyzed by FACS for expression of FcγR on the cell surface. THP-1 cells express both CD32A and CD64. CD64 is a high affinity FcγR that is blocked in conducting the ADCP assay in accordance with the methods of the invention. The THP-1 cells are preferably blocked with 100 μg/mL soluble IgG1 or 10% human serum. To analyze the extent of ADCP, the gate is preferably set on THP-1 cells and median fluorescence intensity is measured. The ADCP activity for individual mutants is calculated and reported as a normalized value to the wild type chMab 4-4-20 obtained. The opsonized particles are added to THP-1 cells such that the ratio of the opsonized particles to THP-1 cells is 30:1 or 60:1. In most preferred embodiments, the ADCP assay is conducted with controls, such as E. coli-FITC in medium, E. coli-FITC and THP-1 cells (to serve as FcγR-independent ADCP activity), E. coli-FITC, THP-1 cells and wild-type 4-4-20 antibody (to serve as FcγR-dependent ADCP activity), E. coli-FITC, THP-1 cells, 4-4-20 D265A (to serve as the background control for FcγR-dependent ADCP activity).

In another embodiment, the molecules of the invention can be assayed for FcγR-mediated ADCC activity in effector cells, e.g., natural killer cells, using any of the standard methods known to those skilled in the art (See e.g., Perussia et al., 2000, Methods Mol Biol 121: 179-92; Weng et al., 2003, J. Clin. Oncol. 21:3940-3947; Ding et al., Immunity, 1998, 8:403-11). An exemplary assay for determining ADCC activity of the molecules of the invention is based on a ⁵¹Cr release assay comprising of: labeling target cells with [⁵¹Cr]Na₂CrO₄ (this cell-membrane permeable molecule is commonly used for labeling since it binds cytoplasmic proteins and although spontaneously released from the cells with slow kinetics, it is released massively following target cell necrosis); opsonizing the target cells with the molecules of the invention comprising variant heavy chains; combining the opsonized radiolabeled target cells with effector cells in a microtitre plate at an appropriate ratio of target cells to effector cells; incubating the mixture of cells for 16-18 hours at 37° C.; collecting supernatants; and analyzing radioactivity. The cytotoxicity of the molecules of the invention can then be determined, for example using the following formula: % lysis=(experimental cpm−target leak cpm)/(detergent lysis cpm−target leak cpm)×100%. Alternatively, % lysis=(ADCC−AICC)/(maximum release-spontaneous release). Specific lysis can be calculated using the formula: specific lysis=% lysis with the molecules of the invention−% lysis in the absence of the molecules of the invention. A graph can be generated by varying either the target: effector cell ratio or antibody concentration.

Preferably, the effector cells used in the ADCC assays of the invention are peripheral blood mononuclear cells (PBMC) that are preferably purified from normal human blood, using standard methods known to one skilled in the art, e.g., using Ficoll-Paque density gradient centrifugation. Preferred effector cells for use in the methods of the invention express different FcγR activating receptors. The invention encompasses, effector cells, THP-1, expressing FcγRI, FcγRIIA and FcγRIIB, and monocyte derived primary macrophages derived from whole human blood expressing both FcγRIIIA and FcγRIIB, to determine if heavy chain antibody mutants show increased ADCC activity and phagocytosis relative to wild type IgG1 antibodies.

The human monocyte cell line, THP-1, activates phagocytosis through expression of the high affinity receptor FcγRI and the low affinity receptor FcγRIIA (Fleit et al., 1991, J. Leuk. Biol. 49: 556). THP-1 cells do not constitutively express FcγRIIA or FcγRIIB. Stimulation of these cells with cytokines effects the FcR expression pattern (Pricop et al., 2000 J. Immunol. 166: 531-7). Growth of THP-1 cells in the presence of the cytokine IL4 induces FcγRIIB expression and causes a reduction in FcγRIIA and FcγRI expression. FcγRIIB expression can also be enhanced by increased cell density (Tridandapani et al., 2002, J. Biol. Chem. 277: 5082-9). In contrast, it has been reported that IFNγ can lead to expression of FcγRIIIA (Pearse et al., 1993 PNAS USA 90: 4314-8). The presence or absence of receptors on the cell surface can be determined by FACS using common methods known to one skilled in the art. Cytokine induced expression of FcγR on the cell surface provides a system to test both activation and inhibition in the presence of FcγRIIB. If THP-1 cells are unable to express the FcγRIIB the invention also encompasses another human monocyte cell line, U937. These cells have been shown to terminally differentiate into macrophages in the presence of IFNγ and TNF (Koren et al., 1979, Nature 279: 328-331).

FcγR dependent tumor cell killing is mediated by macrophage and NK cells in mouse tumor models (Clynes et al., 1998, PNAS USA 95: 652-656). The invention encompasses the use of elutriated monocytes from donors as effector cells to analyze the efficiency Fc mutants to trigger cell cytotoxicity of target cells in both phagocytosis and ADCC assays. Expression patterns of FcγRI, FcγRIIIA, and FcγRIIB are affected by different growth conditions. FcγR expression from frozen elutriated monocytes, fresh elutriated monocytes, monocytes maintained in 10% FBS, and monocytes cultured in FBS+GM-CSF and or in human serum may be determined using common methods known to those skilled in the art. For example, cells can be stained with FcγR specific antibodies and analyzed by FACS to determine FcR profiles. Conditions that best mimic macrophage in vivo FcγR expression is then used for the methods of the invention.

In some embodiments, the invention encompasses the use of mouse cells especially when human cells with the right FcγR profiles are unable to be obtained. In some embodiments, the invention encompasses the mouse macrophage cell line RAW264.7(ATCC) which can be transfected with human FcγRIIIA and stable transfectants isolated using methods known in the art, see, e.g., Ralph et al., J. Immunol. 119: 950-4). Transfectants can be quantitated for FcγRIIIA expression by FACS analysis using routine experimentation and high expressors can be used in the ADCC assays of the invention. In other embodiments, the invention encompasses isolation of spleen peritoneal macrophage expressing human FcγR from knockout transgenic mice such as those disclosed herein.

Lymphocytes may be harvested from peripheral blood of donors (PBM) using a Ficoll-Paque gradient (Pharmacia). Within the isolated mononuclear population of cells the majority of the ADCC activity occurs via the natural killer cells (NK) containing FcγRIIIA but not FcγRIIB on their surface. Results with these cells indicate the efficacy of the mutants on triggering NK cell ADCC and establish the reagents to test with elutriated monocytes.

Target cells used in the ADCC assays of the invention include, but are not limited to, breast cancer cell lines, e.g., SK-BR-3 with ATCC accession number HTB-30 (see, e.g., Tremp et al., 1976, Cancer Res. 33-41); B-lymphocytes; cells derived from Burkitts lymphoma, e.g., Raji cells with ATCC accession number CCL-86 (see, e.g., Epstein et al., 1965, J. Natl. Cancer Inst. 34: 231-240), and Daudi cells with ATCC accession number CCL-213 (see, e.g., Klein et al., 1968, Cancer Res. 28: 1300-10). The target cells must be recognized by the antigen binding site of the immunoglobulin to be assayed.

The ADCC assay is based on the ability of NK cells to mediate cell death via an apoptotic pathway. NK cells mediate cell death in part by FcγRIIIA's recognition of IgG bound to an antigen on a cell surface. The ADCC assays used in accordance with the methods of the invention may be radioactive based assays or fluorescence based assays. The ADCC assay used to characterize the molecules of the invention comprising variant Fc regions comprises labeling target cells, e.g., SK-BR-3, MCF-7, OVCAR3, Raji, Daudi cells, opsonizing target cells with an antibody that recognizes a cell surface receptor on the target cell via its antigen binding site; combining the labeled opsonized target cells and the effector cells at an appropriate ratio, which can be determined by routine experimentation; harvesting the cells; detecting the label in the supernatant of the lysed target cells, using an appropriate detection scheme based on the label used. The target cells may be labeled either with a radioactive label or a fluorescent label, using standard methods known in the art. For example the labels include, but are not limited to, [⁵¹Cr]Na₂CrO₄; and the acetoxymethyl ester of the fluorescence enhancing ligand, 2,2′:6′,2″-terpyridine-6-6″-dicarboxylate (TDA).

In a specific preferred embodiment, a time resolved fluorimetric assay is used for measuring ADCC activity against target cells that have been labeled with the acetoxymethyl ester of the fluorescence enhancing ligand, 2,2′:6′,2″-terpyridine-6-6″-dicarboxylate (TDA). Such fluorimetric assays are known in the art, e.g., see, Blomberg et al., 1996, Journal of Immunological Methods, 193: 199-206; which is incorporated herein by reference in its entirety. Briefly, target cells are labeled with the membrane permeable acetoxymethyl diester of TDA (bis(acetoxymethyl) 2,2′:6′,2″-terpyridine-6-6″-dicarboxylate, (BATDA), which rapidly diffuses across the cell membrane of viable cells. Intracellular esterases split off the ester groups and the regenerated membrane impermeable TDA molecule is trapped inside the cell. After incubation of effector and target cells, e.g., for at least two hours, up to 3.5 hours, at 37° C., under 5% CO₂, the TDA released from the lysed target cells is chelated with Eu3+ and the fluorescence of the Europium-TDA chelates formed is quantitated in a time-resolved fluorometer (e.g., Victor 1420, Perkin Elmer/Wallac).

In another specific embodiment, the ADCC assay used to characterize the molecules of the invention comprising variant heavy chains comprises the following steps: Preferably 4-5×10⁶ target cells (e.g., SK-BR-3, MCF-7, OVCAR3, Raji cells) are labeled with bis(acetoxymethyl) 2,2′:6′,2″-terpyridine-t-6″-dicarboxylate (DELFIA BATDA Reagent, Perkin Elmer/Wallac). For optimal labeling efficiency, the number of target cells used in the ADCC assay should preferably not exceed 5×10⁶. BATDA reagent is added to the cells and the mixture is incubated at 37° C. preferably under 5% CO₂, for at least 30 minutes. The cells are then washed with a physiological buffer, e.g., PBS with 0.125 mM sulfinpyrazole, and media containing 0.125 mM sulfinpyrazole. The labeled target cells are then opsonized (coated) with a molecule of the invention comprising a variant heavy chain, i.e., an immunoglobulin comprising a variant heavy chain of the invention, including, but not limited to, a polyclonal antibody, a monoclonal antibody, a bispecific antibody, a multi-specific antibody, a humanized antibody, or a chimeric antibody. In preferred embodiments, the immunoglobulin comprising a variant heavy chain used in the ADCC assay is specific for a cell surface receptor, a tumor antigen, or a cancer antigen. The immunoglobulin into which a variant heavy chain of the invention is introduced may specifically bind any cancer or tumor antigen, such as those listed in section 5.2 and 5.5.1. Additionally, the immunoglobulin into which a variant Fc region of the invention is introduced may be any therapeutic antibody specific for a cancer antigen, such as those listed in section 5.5.1.2. In some embodiments, the immunoglobulin comprising a variant Fc region used in the ADCC assay is an anti-fluoresceine monoclonal antibody, 4-4-20 (Kranz et al., 1982 J. Biol Chem. 257(12): 6987-6995) a mouse-human chimeric anti-CD20 monoclonal antibody 2H7 (Liu et al., 1987, Journal of Immunology, 139: 3521-6); or a humanized antibody (Ab4D5) against the human epidermal growth factor receptor 2 (p185 HER2) (Carter et al. (1992, Proc. Natl. Acad. Sci. USA 89: 4285-9). The target cells in the ADCC assay are chosen according to the immunoglobulin into which a variant heavy chain of the invention has been introduced so that the immunoglobulin binds a cell surface receptor of the target cell specifically. Preferably, the ADCC assays of the invention are performed using more than one engineered antibody, e.g., anti Her2/neu, 4-4-20, 2B6, RITUXAN®, and 2H7, harboring the variant heavy chains of the invention.

Target cells are added to effector cells, e.g., PBMC, to produce effector:target ratios of approximately 1:1, 10:1, 30:1, 50:1, 75:1, or 100:1. In a specific embodiment, when the immunoglobulin comprising a variant heavy chain has the variable domain of the antifluoresceine antibody 4-4-20, (Kranz et al., 1982, J. Biol. Chem., 257:6987-6995), the effector:target is 75:1. The effector and target cells are incubated for at least two hours, up to 3.5 hours, at 37° C., under 5% CO₂. Cell supernatants are harvested and added to an acidic europium solution (e.g., DELFIA Europium Solution, Perkin Elmer/Wallac). The fluorescence of the Europium-TDA chelates formed is quantitated in a time-resolved fluorometer (e.g., Victor 1420, Perkin Elmer/Wallac). Maximal release (MR) and spontaneous release (SR) are determined by incubation of target cells with 1% TX-100 and media alone, respectively. Antibody independent cellular cytotoxicity (AICC) is measured by incubation of target and effector cells in the absence of antibody. Each assay is preferably performed in triplicate. The mean percentage specific lysis is calculated as: Experimental release (ADCC)−AICC)/(MR−SR)×100.

The invention encompasses characterization of molecules comprising heavy chain variants of the invention (i.e., a heavy chain having the Fc region of IgG2, IgG3 or IgG4 and comprising at least one amino acid modification (e.g. substitution) relative to a wild-type heavy chain having an Fc region of the same isotype) in both NK-dependent and macrophage dependent ADCC assays. Heavy chain variants of the invention have altered phenotypes such as an altered effector function as assayed in an NK dependent or macrophage dependent assay. Heavy chain variants identified as altering effector function are disclosed both in the instant application, e.g., in Table 9, and as disclosed in International Application WO04/063351 and U.S. Patent Application Publications 2005/0037000 and 2005/0064514, cocurrent applications of the Inventors, each of which is incorporated by reference in its entirety. For example, five IgG1 mutants summarized in table 9 had an enhanced ADCC activity relative to wild type Fc region: MGFc-27 (G316D, A378V, D399E); MGFc-31 (P247L, N421K); MGFc-10 (K288N, A330S, P396L); MGFc-28 (N315I, V379M, T394M); MGFc-29 (F243I, V379L, G420V). Additional mutants that altered ADCC activity relative to wild type Fc region were disclosed in International Application WO04/063351. In WO04/063351, the mutants were identified by cloning variant Fc regions into the humanized antibody Ab4D5 (specific for the human epidermal growth factor receptor (HER2/neu)) or the anti CD-20 monoclonal antibody, 2H7. Relative to antibodies comprising wild type Fc, ten IgG1 mutants had enhanced ADCC activity in the context of 4D5 or 2H7 (MgFc42 (G402D), MgFc44 (K344N, P396L), MgFc45 (H268D, E318D), MgFc49 (K261N, K210M, P396L), MgFc51 (Q419H, P396L), MgFc52 (V240A, P396L), MgFc53 (L410H, P396L), MgFc54 (F243L, V305I, A378D, F404S, P396L), MgFc55 (R2551, P396L) and MgFc59 (K370E, P396L)) and four IgG1 mutants had increased ADCC activity in the context of 4D5 but only equivalent or decreased ADCC activity in the context of 2H7 (MgFc46 (P217S, P396L), MgFc47 (K210M, P396L), MgFc48 (V379M, P396L) and MgFc50(P247S, P396L)). MgFc38 (K392T, P396L) and MgFc43b (K288R, T307A, K344E, P396L) were only tested in the context of 4D5 and showed an increase in ADCC activity relative to 4D5 comprising a wild type Fc. MgFc27 (G316D, A378V, D399E), MgFc29 (F2431, V379L, G420V) and MgFc57 (L242F, P396L) were only tested in the context of 2H7 and showed ambigous (MgFc27) or increased (MgFc 29 and MgFc57) ADCC activity relative to 2H7 comprising wild type Fc. Further mutants identified by the Inventors and analyzed in the context of 4-4-20 (an IgG1) are summarized in Table 15, adapted from U.S. Patent Application Publication 2005/0037000. In Table 14, the ADCC activity of antibodies containing the variant IgG1 Fc is presented relative to the activity of the wild-type antibody (Wt).

TABLE 25
Analysis of ADCC mediated by 4-4-20 anti-Fluorescein IgG1
antibody on SKBR3 cells coated with fluorescein.

| | | Relative rate of lysis | |
|---|---|---|---|
| | | (mutant/Wt) | |
| Mutant | Amino Acid Variation | (1 μg · ml) | (0.5 μg/ml) |
| MgFc39 | E293V, Q295E, A327T | | 4.29 |
| MgFc37 | K248M | | 3.83 |
| MgFc54 | F243L, V305I, A378D, F404S, | 3.59 | |
| | P396L | | |
| MgFc42 | D221E, D270E,V308A, Q311H, | 3.17 | |
| | P396L, G402D | | |
| MgFc43b | K288R, T307A, K344E, P396L | 3.3 | |
| MgFc55 | R2551, P396L | 2.79 | |
| MgFc59 | K370E, P396L | 2.47 | |
| MgFc44 | K334N, P396L | 2.43 | |
| MgFc57 | L242F, P396L | 2.4 | |
| MgFc52 | V240A, P396L | 2.35 | |
| MgFc27 | G316D, A378V, D399E | 2.24 | 3.60 |
| MgFc51 | Q419H, P396L | 2.24 | |
| MgFc38 | K392T, P396L | | 3.07 |
| MgFc50 | P247S, P396L | 2.10 | |
| MgFc49 | K261N, K210M, P396L | 2.06 | |
| MgFc31 | P247L, N421K | 2.05 | 2.90 |
| MgFc46 | P217S, P396L | 2.04 | |
| MgFc41 | H268N, P396LN | | 2.24 |
| MgFc47 | K210M, P396L | 2.02 | |
| MgFc48 | V379M, P396L | 2.01 | |
| MgFc53 | L410H, P396L | 2 | |
| MgFc10 | K288N, A330S, P396L | 1.66 | 1.67 |
| MgFc60 | P217S, P396L | 1.44 | |
| MgFc28 | N315I, V379M, T394M | 1.37 | 1.69 |
| MgFc29 | F243I, V379L, G420V | 1.35 | 1.17 |
| MgFc43 | Y319F, P352L, P396L | | 1.09 |
| Wt | None | 1 | 1 |
| MgFc35 | R255Q, K326E | 0.79 | 0.53 |
| MgFc36 | K218R, G281D, G385R | 0.67 | 0.78 |
| MgFc30 | F275Y | 0.64 | 0.37 |
| MgFc32 | D280E, S354F, A431D, L441I | 0.62 | 0.75 |
| MgFc33 | K317N, F423deleted | 0.18 | −0.22 |
| MgFc34 | F241L, E258G | −0.08 | −0.71 |
| MgFc26 | D265A | 0.08 | −0.45 |

The invention encompasses assays known in the art, and exemplified herein, to characterize the binding of C1q and mediation of complement dependent cytotoxicity (CDC) by molecules of the invention. To determine C1q binding, a C1q binding ELISA may be performed. An exemplary assay may comprise the following: assay plates may be coated overnight at 4 C with polypeptide comprising a molecule of the invention or starting polypeptide (control) in coating buffer. The plates may then be washed and blocked. Following washing, an aliquot of human C1q may be added to each well and incubated for 2 hrs at room temperature. Following a further wash, 100 uL of a sheep anti-complement C1q peroxidase conjugated antibody may be added to each well and incubated for 1 hour at room temperature. The plate may again be washed with wash buffer and 100 ul of substrate buffer containing OPD (O-phenylenediamine dihydrochloride (Sigma)) may be added to each well. The oxidation reaction, observed by the appearance of a yellow color, may be allowed to proceed for 30 minutes and stopped by the addition of 100 ul of 4.5 NH2 SO4. The absorbance may then read at (492-405) nm.

A preferred molecule in accordance with the invention is one that displays a significant reduction in C1q binding, as detected and measured in this assay or a similar assay. Preferably the molecule comprising a variant heavy chain displays about 50 fold reduction, about 60 fold, about 80 fold, or about 90 fold reduction in C1q binding compared to a control antibody comprising a variant heavy chain having an Fc region of the same isotype. In the most preferred embodiment, the molecule comprising an Fc variant does not bind C1q, i.e. the variant displays about 100 fold or more reduction in C1q binding compared to the control antibody.

Another exemplary molecule of the invention is one which comprises greater binding affinity for human C1q than a comparable, control molecule (e.g., a molecule comprising a wild type heavy chain having an Fc region of the same isotype). Such a molecule may display, for example, about two-fold or more, and preferably about five-fold or more, improvement in human C1q binding compared to the parent molecule comprising wild type heavy chain having an Fc region of the same isotype. For example, human C1q binding may be about two-fold to about 500-fold, and preferably from about two-fold or from about five-fold to about 1000-fold improved compared to the molecule comprising wild type Fc region.

To assess complement activation, a complement dependent cytotoxicity (CDC) assay may be performed, e.g. as described in Gazzano-Santoro et al., J. Immunol. Methods 202:163 (1996), which is incorporated herein by reference in its entirety. Briefly, various concentrations of the molecule comprising a variant heavy chain and human complement may be diluted with buffer. Cells which express the antigen to which the molecule comprising a variant heavy chain binds may be diluted to a density of about 1×10⁶ cells/ml. Mixtures of the molecule comprising a variant heavy chain, diluted human complement and cells expressing the antigen may be added to a flat bottom tissue culture 96 well plate and allowed to incubate for 2 hrs at 37° C. and 5% CO₂ to facilitate complement mediated cell lysis. 50 μL of alamar blue (Accumed International) may then be added to each well and incubated overnight at 37° C. The absorbance is measured using a 96-well fluorometer with excitation at 530 nm and emission at 590 nm. The results may be expressed in relative fluorescence units (RFU). The sample concentrations may be computed from a standard curve and the percent activity as compared to nonvariant molecule, i.e., a molecule comprising wild type heavy chain, is reported for the variant of interest.

In some embodiments, an heavy chain variant of the invention does not activate complement Preferably the variant does not appear to have any CDC activity in the above CDC assay. The invention also pertains to a variant with enhanced CDC compared to a control molecule (a molecule comprising wild type heavy chain), e.g., displaying about two-fold to about 100-fold improvement in CDC activity in vitro or in vivo (e.g., at the IC50 values for each molecule being compared). Complement assays may be performed with guinea pig, rabbit or human serum. Complement lysis of target cells may be detected by monitoring the release of intracellular enzymes such as lactate dehydrogenase (LDH), as described in Korzeniewski et al., 1983 J. Immunol. Methods 64(3): 313-20; and Decker et al., 1988 J. Immunol Methods 115(1): 61-9, each of which is incorporated herein by reference in its entirety; or the release of an intracellular label such as europium, chromium 51 or indium 111 in which target cells are labeled as described herein.

6.3.2 Other Assays

The molecules of the invention comprising variant Fc regions may also be assayed using any surface plasmon resonance based assays known in the art for characterizing the kinetic parameters of Fc-FcγR interaction binding. Any SPR instrument commercially available including, but not limited to, BIAcore Instruments, available from Biacore AB (Uppsala, Sweden); IAsys instruments available from Affinity Sensors (Franklin, Mass.); IBIS system available from Windsor Scientific Limited (Berks, UK), SPR-CELLIA systems available from Nippon Laser and Electronics Lab (Hokkaido, Japan), and SPR Detector Spreeta available from Texas Instruments (Dallas, Tex.) can be used in the instant invention. For a review of SPR-based technology see Mullet et al., 2000, Methods 22: 77-91; Dong et al., 2002, Review in Mol. Biotech., 82: 303-23; Fivash et al., 1998, Current Opinion in Biotechnology 9: 97-101; Rich et al., 2000, Current Opinion in Biotechnology 111: 54-61; all of which are incorporated herein by reference in their entirety. Additionally, any of the SPR instruments and SPR based methods for measuring protein-protein interactions described in U.S. Pat. Nos. 6,373,577; 6,289,286; 5,322,798; 5,341,215; 6,268,125 are contemplated in the methods of the invention, all of which are incorporated herein by reference in their entirety.

Briefly, SPR based assays involve immobilizing a member of a binding pair on a surface, and monitoring its interaction with the other member of the binding pair in solution in real time. SPR is based on measuring the change in refractive index of the solvent near the surface that occurs upon complex formation or dissociation. The surface onto which the immobilization occur is the sensor chip, which is at the heart of the SPR technology; it consists of a glass surface coated with a thin layer of gold and forms the basis for a range of specialized surfaces designed to optimize the binding of a molecule to the surface. A variety of sensor chips are commercially available especially from the companies listed supra, all of which may be used in the methods of the invention. Examples of sensor chips include those available from BIAcore AB, Inc., e.g., Sensor Chip CM5, SA, NTA, and HPA. A molecule of the invention may be immobilized onto the surface of a sensor chip using any of the immobilization methods and chemistries known in the art, including but not limited to, direct covalent coupling via amine groups, direct covalent coupling via sulfhydryl groups, biotin attachment to avidin coated surface, aldehyde coupling to carbohydrate groups, and attachment through the histidine tag with NTA chips.

In some embodiments, the kinetic parameters of the binding of molecules of the invention comprising variant heavy chains, e.g., immunoglobulins comprising an Fc region, to an FcγR may be determined using a BIAcore instrument (e.g., BIAcore instrument 1000, BIAcore Inc., Piscataway, N.J.). Any FcγR can be used to assess the interaction with the molecules of the invention comprising variant Fc regions. In a specific embodiment the FcγR is FcγRIIIA, preferably a soluble monomeric FcγRIIIA. For example, in one embodiment, the soluble monomeric FcγRIIIA is the extracellular region of FcγRIIIA joined to the linker-AVITAG sequence (see, U.S. Provisional Application No. 60/439,498, filed on Jan. 9, 2003 (Attorney Docket No. 11183-004-888) and U.S. Provisional Application No. 60/456,041 filed on Mar. 19, 2003, which are incorporated herein by reference in their entireties). In another specific embodiment, the FcγR is FcγRIIB, preferably a soluble dimeric FcγRIIB. For example in one embodiment, the soluble dimeric FcγRIIB protein is prepared in accordance with the methodology described in U.S. Provisional application No. 60/439,709 filed on Jan. 13, 2003, which is incorporated herein by reference in its entirety.

An exemplary assay for determining the kinetic parameters of a molecule comprising a variant heavy chain, in particular comprising an Fc region, wherein the molecule is the 4-4-20 antibody, to an FcγR using a BIAcore instrument comprises the following: BSA-FITC is immobilized on one of the four flow cells of a sensor chip surface, preferably through amine coupling chemistry such that about 5000 response units (RU) of BSA-FITC is immobilized on the surface. Once a suitable surface is prepared, 4-4-20 antibodies carrying the heavy chain variants of the invention are passed over the surface, preferably by one minute injections of a 20 μg/mL solution at a 5 μL/mL flow rate. The level of 4-4-20 antibodies bound to the surface ranges between 400 and 700 RU. Next, dilution series of the receptor (FcγRIIA and FcγRIIB-Fc fusion protein) in HBS-P buffer (20 mM HEPES, 150 mM NaCl, 3 mM EDTA, pH 7.5) are injected onto the surface at 100 μL/min Antibody regeneration between different receptor dilutions is carried out preferably by single 5 second injections of 100 mM NaHCO₃ pH 9.4; 3M NaCl. Any regeneration technique known in the art is contemplated in the method of the invention.

Once an entire data set is collected, the resulting binding curves are globally fitted using computer algorithms supplied by the SPR instrument manufacturer, e.g., BIAcore, Inc. (Piscataway, N.J.). These algorithms calculate both the Kₒₙ and K_{off}, from which the apparent equilibrium binding constant, K_{d} is deduced as the ratio of the two rate constants (i.e., K_{off}/Kₒₙ). More detailed treatments of how the individual rate constants are derived can be found in the BIAevaluaion Software Handbook (BIAcore, Inc., Piscataway, N.J.). The analysis of the generated data may be done using any method known in the art. For a review of the various methods of interpretation of the kinetic data generated see Myszka, 1997, Current Opinion in Biotechnology 8: 50-7; Fisher et al., 1994, Current Opinion in Biotechnology 5: 389-95; O'Shannessy, 1994, Current Opinion in Biotechnology, 5:65-71; Chaiken et al., 1992, Analytical Biochemistry, 201: 197-210; Morton et al., 1995, Analytical Biochemistry 227: 176-85; O'Shannessy et al., 1996, Analytical Biochemistry 236: 275-83; all of which are incorporated herein by reference in their entirety.

In preferred embodiments, the kinetic parameters determined using an SPR analysis, e.g., BIAcore, may be used as a predictive measure of how a molecule of the invention will function in a functional assay, e.g., ADCC. An exemplary method for predicting the efficacy of a molecule of the invention based on kinetic parameters obtained from an SPR analysis may comprise the following: determining the K_{off} values for binding of a molecule of the invention to FcγRIIIA and FcγRIIB; plotting (1) K_{off} (wt)/K_{off} (mut) for FcγRIIIA; (2) K_{off} (mut)/K_{off} (wt) for FcγRIIB against the ADCC data. Numbers higher than one show a decreased dissociation rate for FcγRIIIA and an increased dissociation rate for FcγRIIB relative to wild type; and possess and enhanced ADCC function.

The invention encompasses antibodies with specific variants of the heavy chain that have been identified using BIAcore kinetic analyses as described herein or as disclosed in International Application WO04/063351 and U.S. Patent Application Publications 2005/0037000 and 2005/0064514, concurrent applications of the Inventors, each of which is incorporated by reference in its entirety. Tables 26-22 summarize various mutants that were characterized in the context of an IgG1 using BIAcore analysis as disclosed herein and as described in said applications. Those mutants listed in Tables 16-18 were also tested using an ELISA assay, for determining binding to FcγRIIIA and FcγRIIB, and an ADCC assay. The antibody concentration used was standard for ADCC assays, in the range of 0.5 μg/ml-1.0 μg/ml. Those mutants listed in Tables 20-22 were characterized in the context of an IgG1 using BIAcore analysis for the binding to multiple allotypes of FcγRIIIA and FcγRIIA. Mutants listed in Table 21 were characterized using BIAcore analysis for binding to C1q. For either the BIAcore or ADCC assays, Fc mutations were cloned into a ch4-4-20, 2B6 or 4D5 antibody (each an IgG1) as indicated.

TABLE 16
SUMMARY OF MUTANTS

| | | | | ELISA | ELISA | | 4-4-20 | 4D5 |
|---|---|---|---|---|---|---|---|---|
| Fc | Amino Acid | FcRIIIA, | FcRIIB, | IIIA | IIB | Phagocytosis | ADCC | ADCC |
| Variant | changes | K_{D}/Koff | K_{D}/K_{off} | binding | binding | (mutant/Wt) | (mutant/wt) | (mutant/wt) |
| Wt | none | 198/0.170 | 94/.094 | 1 | 1 | 1 | 1 | 1 |
| MGFc 5 | V379M | 160/0.167 | 70/0.10 | 2X | N/C | 0.86 | 2.09 | 1.77 |
| MGFc 9 | P243I, V379L | 99.7/0.105 | 120/0.113 | 1.5X | reduced | ? | 2.25 | 2.04 |
| MGFc 10 | K288N, A330S, P396L | 128/0.115 | 33.4/0.050 | 5X | 3X | 1.2 | 2.96 | 2.50 |
| MGFc 11 | F243L, R255L | 90/0.075 | 74.7/0.09 | 1x | reduced | 0.8 | 2.38 | 1.00 |
| MGFc13 | K334E, T359N, T366S | 55.20.128 | 72/0.11 | 1.5X | N/C | [ | 1.57 | 3.67 |
| MGFc 14 | K288M, K334E | 75.4/0.1 | 95.6/0.089 | 3X | reduced | [ | 1.74 | |
| MGFc 23 | K334E, R292L | 70.2/0.105 | 108/0.107 | | | [ | 2.09 | 1.6 |
| MGFc 27 | G316D, A378V, | 72/0.117 | 46/0.06 | 1.5X | 14X | 1.4 | 3.60 | 6.88 |
| | D399E | | | | | | | |
| MGFc 28 | N315I, A379M, | | | 1X | 9X | 1.37 | 1.69 | 1.00 |
| | D399E | | | | | | | |
| MGFc 29 | P243I, V379L, G420V | 108/0.082 | 93.4/.101 | 2.5X | 7X | 0.93 | 1.17 | 1.00 |
| MGFc 31 | P247L, N421K | 62/0.108 | 66/0.065 | 3X | N/C | 1.35 | 2.90 | 1.00 |
| MGFc 37 | K248M | 154/0.175 | 100/0.091 | 1.4X | reduced | 0.98 | 3.83 | 0.67 |
| MGFc 38 | K392T, P396L | 84/0.104 | 50/0.041 | 4.5X | 2.5X | 1.4 | 3.07 | 2.50 |
| MGFc 39 | E293V, Q295E, | 195/0.198 | 86/0.074 | 1.4X | reduced | 1.5 | 4.29 | 0.50 |
| | A327T | | | | | | | |
| MGFc 40 | K248M | 180/0.186 | 110/0.09 | 1.4X | reduced | 1.14 | 4.03 | |
| MGFc 41 | H268N, P396L | 178/0.159 | 46.6/0.036 | 2.2X | 4.5X | 1.96 | 2.24 | 0.67 |
| MGFc 43 | Y319F, P352L, P396L | 125/0.139 | 55.7/0.041 | 3.5X | 2X | 1.58 | 1.09 | |

TABLE 17
Kinetic parameters of FcRIIIA binding to ch4-4-20Ab obtained
by “separate fit”of 200 nM and 800 nM binding curves

| | BIAcore | Kₒₙ | K_{off} | ELISA | |
|---|---|---|---|---|---|
| Ch4-4-20Ab | Kd, nM | 1/Ms | 1/s | OD | ADCC % |
| Wt(0225) | 319 | 6.0 × 10⁵ | 0.170 | 0.5 | 17.5 |
| MgFc11(0225) | 90 | 8.22 × 10⁵ | 0.075 | 0.37 | 32 |
| Mut5(0225) | 214 | 8.2 × 10⁵ | 0.172 | 0.75 | 26 |
| Mut6(0225) | 264 | 6.67 × 10⁵ | 0.175 | 0.6 | 23 |
| Mut8(0225) | 234 | 8.3 × 10⁵ | 0.196 | 0.5 | 22 |
| Mut10(0225) | 128 | 9.04 × 10⁵ | 0.115 | 1.0 | 41 |
| Mut12(0225) | 111 | 1.04 × 10⁶ | 0.115 | 1.0 | 37 |
| Mut15(0225) | 67.9 | 1.97 × 10⁶ | 0.133 | 1.0 | 15 |
| Mut16(0225) | 84.8 | 1.60 × 10⁶ | 0.133 | 1.0 | 15 |
| Mut18(0225) | 92 | 1.23 × 10⁶ | 0.112 | 1.0 | 28 |
| Mut25(0225) | 48.6 | 2.05 × 10⁶ | 0.1 | 1.0 | 41 |
| Mut14(0225) | 75.4 | 1.37 × 10⁶ | 0.1 | 1.1 | 28 |
| Mut17(0225) | 70.5 | 1.42 × 10⁶ | 0.1 | 1.25 | 30 |
| Mut19(0225) | 100 | 1.20 × 10⁶ | 0.120 | 0.75 | 11 |
| Mut20(0225) | 71.5 | 1.75 × 10⁶ | 0.126 | 0.5 | 10 |
| Mut23(0225) | 70.2 | 1.43 × 10⁶ | 0.105 | 1.25 | 25 |

TABLE 18
Kinetic parameters of FcRIIB-Fc binding to wild type and
mutant ch4-4-20Ab obtained by “separate fit” of 200 nM and 800 nM
binding curves.

| | BIAcore | Kₒₙ | K_{off} | ELISA | |
|---|---|---|---|---|---|
| Ch4-4-20Ab | Kd, nM | 1/Ms | 1/s | OD | ADCC % |
| Wt(0225) | 61.4 | | 0.085 | 0.4 | 17.5 |
| Mut11(0225) | 82.3 | | 0.1 | 0.08 | 32 |
| Mut5(0225) | 50 | | 0.057 | 0.6 | 26 |
| Mut6(0225) | 66.5 | | 0.060 | 0.35 | 23 |
| Mut8(0225) | 44.2 | | 0.068 | 0.25 | 22 |
| Mut10(0225) | 41.3 | | 0.05 | 1.2 | 41 |
| Mut12(0225) | 40.1 | | 0.051 | 0.4 | 37 |
| Mut15(0225) | 37.8 | | 0.040 | 1.55 | 15 |
| Mut16(0225) | 40 | | 0.043 | 1.55 | 15 |
| Mut18(0225) | 51.7 | | 0.043 | 1.25 | 28 |
| Mut25(0225) | | | 0.112 | 0.08 | 41 |
| Mut14(0225) | 95.6 | | 0.089 | 0.13 | 28 |
| Mut17(0225) | 55.3 | | 0.056 | 0.38 | 30 |
| Mut19(0225) | 45.3 | | 0.046 | 1.0 | 11 |
| Mut20(0225) | 24.1 | | 0.028 | 0.8 | 10 |
| Mut23(0225) | 108 | | 0.107 | 0.1 | 25 |

TABLE 19
Kinetic parameters of FcγRIIIA (158V) and FcγRIIB binding to
ch4-4-20 obtained by “separate fit” of 200 nM and 800 nM
binding curves

| Fc | | FcγRIIIA158V | FcγRIIB |
|---|---|---|---|
| mutant | AA residues | (Koff WT/Mut) | (Koff WT/Mut) |
| MgFc37 | K248M | 0.977 | 1.03 |
| MgFc38 | K392T, P396L | 1.64 | 2.3 |
| MgFc39 | E293V, Q295E, A327T | 0.86 | 1.3 |
| MgFc41 | H268N, P396LN | 0.92 | 1.04 |
| MgFc43 | Y319F, P352L, P396L | 1.23 | 2.29 |
| MgFc42 | D221E, D270E, | 1.38 | |
| | V308A, Q311H, | | |
| | P396L, G402D | | |
| MgFc43b | K288R, T307A, | 1.27 | 0.89 |
| | K344E, P396L | | |
| MgFc44 | K334N, P396L | 1.27 | 1.33 |
| MgFc46 | P217S, P396L | 1.17 | 0.95 |
| MgFc49 | K261N, K210M, P396L | 1.29 | 0.85 |
| MgFc61 | A330V | 1 | 0.61 |
| MgFc62 | R292G | 1 | 0.67 |
| MgFc63 | S298N, K360R, N361D | 1 | 0.67 |
| MgFc64 | E233G | 1 | 0.54 |
| MgFc65 | N276Y | 1 | 0.64 |
| MgFc66 | A330V, G427M, | 1 | 0.62 |

TABLE 20
Kinetic parameters of binding to 4D5. Parameters of FcγRIIIA
(158V) and FcγRIIIA (158F) obtained by “separate fit” of 400 nM
and 800 nM binding curves; parameters of FcγRIIB and FcγRIIA
(131H) obtained by “separate fit” of 100 nM and 200 nM binding
curves.

| | | | | | | FcγR Receptor | | | |
|---|---|---|---|---|---|---|---|---|---|
| | Amino Acid at Position | | | | | FcγRIIIA | FcγRIIIA | | FcγRIIA |
| 4D5 Mutant | 243 | 292 | 300 | 305 | 396 | 158V | 158F | FcγRIIB | 131H |
| Wild Type | F | R | Y | V | P | 0.186 | 0.294 | 0.096 | 0.073 |
| MgFc0088 | L | P | L | I | L | 0.016 | 0.064 | 0.058 | 0.035 |
| MGFc0143 | I | P | L | I | L | 0.017 | 0.094 | 0.091 | 0.049 |
| Quadruple | | | | | | | | | |
| MGFc0088A | L | P | L | | L | 0.016 | 0.094 | 0.075 | 0.044 |
| MGFc0084 | L | P | | I | L | 0.048 | 0.133 | 0.278 | 0.083 |
| MGFc0142 | L | | L | I | L | | | | |
| Triple | | | | | | | | | |
| MGFc0155 | L | P | L | | | 0.029 | 0.135 | 0.155 | 0.057 |
| MGFc0074 | L | P | | I | | 0.063 | 0.37 | NB | 0.166 |
| MGFc0093 | | P | | I | L | 0.080 | 0.197 | 0.125 | 0.190 |
| Double | | | | | | | | | |
| MGFc0162 | L | P | | | | 0.041 | 0.515 | 0.900 | 0.18 |
| MGFc0091 | L | | | | L | 0.108 | 0.330 | 0.036 | 0.026 |
| MGFc0070 | | P | | I | | 0.101 | 0.250 | 0.030 | 0.025 |
| Single | | | | | | | | | |
| SV12/F243L | L | | | | | 0.048 | 0.255 | 0.112 | 0.100 |
| MGFc0161 | | P | | | | 0.067 | 0.485 | 0.421 | 0.117 |
| | | G | | | | 0.124 | NT | 0.384 | NT |
| MGFc0092 | | | L | | | 0.211 | NT | 0.058 | 0.02 |
| MGFc0089 | | | | | L | 0.127 | 0.306 | 0.031 | 0.039 |

TABLE 21
Kinetic parameters of FcγRIIIA (158V), FcγRIIIA (158F),
FcγRIIB, FcγRIIA (131R), FcγRIIA (131H) and C1q binding to
2B6 obtained by “separate fit” of 200 nM and 800 nM binding
curves. Addition of D270E mutation (“/60”)enhances FcγRIIIA
and FcγRIIB (131H) binding and reduces FcγRIIB binding

| | | FcγRIIIA | | FcγRIIA | | |
|---|---|---|---|---|---|---|
| 2B6Mutants | FcγRIIIA 158V | 158F | FcγRIIB | 131R | FcγRIIA 131H | C1q |
| WT | 0.192 | 0.434 | 0.056 | 0.070 | 0.053 | 0.124 |
| MgFc38 | 0.114 | 0.243 | 0.024 | 0.028 | 0.024 | 0.096 |
| MgFc38/60 | 0.084 | 0.238 | 0.094 | 0.127 | 0.034 | 0.210 |
| MgFc51 | 0.142 | 0.310 | 0.030 | 0.036 | 0.028 | 0.074 |
| MgFc51/60 | 0.112 | 0.293 | 0.077 | 0.089 | 0.028 | 0.079 |
| MgFc55 | 0.146 | 0.330 | 0.030 | 0.034 | 0.028 | 0.080 |
| MgFc55/60 | 0.113 | 0.288 | 0.078 | 0.099 | 0.025 | 0.108 |
| MgFc59 | 0.149 | 0.338 | 0.028 | 0.033 | 0.028 | 0.078 |
| MgFc59/60 | 0.105 | 0.296 | 0.078 | 0.095 | 0.024 | 0.107 |

TABLE 22
Kinetic parameters of FcγRIIIA (158V), FcγRIIIA (158F),
FcγRIIB, FcγRIIA (131R) and FcγRIIA (131H) binding to 4D5
obtained by “separate fit” of 200 nM and 800 nM binding curves.
Addition of D270E mutation enhances FcγRIIIA and FcγRIIB
(131H) binding and reduces FcγRIIB binding

| | FcγRIIIA | FcγRIIIA | | FcγRIIA | FcγRIIA |
|---|---|---|---|---|---|
| 2B6Mutants | 158V | 158F | FcγRIIB | 131R | 131H |
| Wt pure | 0.175 | 0.408 | 0.078 | 0.067 | 0.046 |
| MgFc55 | 0.148 | 0.381 | 0.036 | 0.033 | 0.029 |
| MgFc55/60 | 0.120 | 0.320 | 0.092 | 0.087 | 0.013 |
| MgFc55/60 + R292G | 0.116 | 0.405 | 0.124 | 0.112 | 0.037 |
| MgFc55/60 + Y300L | 0.106 | 0.304 | 0.092 | 0.087 | 0.015 |
| MgFc52 | 0.140 | 0.359 | 0.038 | 0.040 | 0.026 |
| MgFc52/60 | 0.122 | 0.315 | 0.094 | 0.087 | 0.013 |
| MgFc59 | 0.145 | 0.378 | 0.039 | 0.047 | 0.033 |
| MgFc59/60 | 0.117 | 0.273 | 0.088 | 0.082 | 0.012 |
| MgFc31 | 0.125 | 0.305 | 0.040 | 0.043 | 0.030 |
| MgFc31/60 | 0.085 | 0.215 | 0.139 | 0.132 | 0.020 |
| MgFc51 | 0.135 | 0.442 | 0.060 | 0.047 | 0.062 |
| MgFc51/60 | 0.098 | 0.264 | 0.118 | 0.106 | 0.023 |
| MgFc38 | 0.108 | 0.292 | 0.034 | 0.025 | 0.032 |
| MgFc38/60 | 0.089 | 0.232 | 0.101 | 0.093 | 0.021 |
| MgFc70 | 0.101 | 0.250 | 0.030 | 0.025 | 0.025 |
| (R292P, V305I) | | | | | |
| MgFc71 | 0.074 | 0.212 | 0.102 | 0.094 | 0.020 |
| (G316D, R416G, D270E) | | | | | |
| MgFc73 | 0.132 | 0.306 | 0.190 | — | 0.024 |
| (V284M, R292L, | | | | | |
| K370N) | | | | | |
| MgFc74 | 0.063 | 0.370 | n.b. | 0.311 | 0.166 |
| (F243L, R292P, V305I) | | | | | |

6.4 Methods of Recombinantly Producing Molecules of the Invention

6.4.1 Polynucleotides Encoding Molecules of the Invention

The present invention also includes polynucleotides that encode the molecules of the invention, including the polypeptides and antibodies. The polynucleotides encoding the molecules of the invention may be obtained, and the nucleotide sequence of the polynucleotides determined, by any method known in the art.

Once the nucleotide sequence of the molecules (e.g., antibodies) that are identified by the methods of the invention is determined, the nucleotide sequence may be manipulated using methods well known in the art, e.g., recombinant DNA techniques, site directed mutagenesis, PCR, etc. (see, for example, the techniques described in Sambrook et al., 2001, Molecular Cloning, A Laboratory Manual., 3rd Ed., Cold Spring Harbor Laboratory, Cold Spring Harbor, N.Y.; and Ausubel et al., eds., 1998, Current Protocols in Molecular Biology, John Wiley & Sons, NY, which are both incorporated by reference herein in their entireties), to generate, for example, antibodies having a different amino acid sequence, for example by generating amino acid substitutions, deletions, and/or insertions.

In a specific embodiment, when the nucleic acids encode antibodies, one or more of the CDRs are inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions).

In another embodiment, human libraries or any other libraries available in the art, can be screened by standard techniques known in the art, to clone the nucleic acids encoding the molecules of the invention.

6.4.2 Recombinant Expression of Molecules of the Invention

Once a nucleic acid sequence encoding molecules of the invention (i.e., antibodies) has been obtained, the vector for the production of the molecules may be produced by recombinant DNA technology using techniques well known in the art. Methods which are well known to those skilled in the art can be used to construct expression vectors containing the coding sequences for the molecules of the invention and appropriate transcriptional and translational control signals. These methods include, for example, in vitro recombinant DNA techniques, synthetic techniques, and in vivo genetic recombination. (See, for example, the techniques described in Sambrook et al., 1990, Molecular Cloning, A Laboratory Manual, 2d Ed., Cold Spring Harbor Laboratory, Cold Spring Harbor, N.Y. and Ausubel et al. eds., 1998, Current Protocols in Molecular Biology, John Wiley & Sons, NY).

An expression vector comprising the nucleotide sequence of a molecule identified by the methods of the invention (i.e., an antibody) can be transferred to a host cell by conventional techniques (e.g., electroporation, liposomal transfection, and calcium phosphate precipitation) and the transfected cells are then cultured by conventional techniques to produce the molecules of the invention. In specific embodiments, the expression of the molecules of the invention is regulated by a constitutive, an inducible or a tissue, specific promoter.

The host cells used to express the molecules identified by the methods of the invention may be either bacterial cells such as Escherichia coli, or, preferably, eukaryotic cells, especially for the expression of whole recombinant immunoglobulin molecule. In particular, mammalian cells such as Chinese hamster ovary cells (CHO), in conjunction with a vector such as the major intermediate early gene promoter element from human cytomegalovirus is an effective expression system for immunoglobulins (Foecking et al., 1998, Gene 45:101; Cockett et al., 1990, Bio/Technology 8:2).

A variety of host-expression vector systems may be utilized to express the molecules identified by the methods of the invention. Such host-expression systems represent vehicles by which the coding sequences of the molecules of the invention may be produced and subsequently purified, but also represent cells which may, when transformed or transfected with the appropriate nucleotide coding sequences, express the molecules of the invention in situ. These include, but are not limited to, microorganisms such as bacteria (e.g., E. coli and B. subtilis) transformed with recombinant bacteriophage DNA, plasmid DNA or cosmid DNA expression vectors containing coding sequences for the molecules identified by the methods of the invention; yeast (e.g., Saccharomyces Pichia) transformed with recombinant yeast expression vectors containing sequences encoding the molecules identified by the methods of the invention; insect cell systems infected with recombinant virus expression vectors (e.g., baculovirus) containing the sequences encoding the molecules identified by the methods of the invention; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the molecules identified by the methods of the invention; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the molecule being expressed. For example, when a large quantity of such a protein is to be produced, for the generation of pharmaceutical compositions of an antibody, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 293T, 3T3, W138, BT483, Hs578T, HTB2, BT20 and T47D, CRL7030 and Hs578Bst.

For long-term, high-yield production of recombinant proteins, stable expression is preferred. For example, cell lines which stably express an antibody of the invention may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibodies of the invention. Such engineered cell lines may be particularly useful in screening and evaluation of compounds that interact directly or indirectly with the antibodies of the invention.

A number of selection systems may be used, including but not limited to the herpes simplex virus thymidine kinase (Wigler et al., 1977, Cell 11: 223), hypoxanthine-guanine phosphoribosyltransferase (Szybalska & Szybalski, 1992, Proc. Natl. Acad. Sci. USA 48: 202), and adenine phosphoribosyltransferase (Lowy et al., 1980, Cell 22: 817) genes can be employed in tk−, hgprt− or aprt− cells, respectively. Also, antimetabolite resistance can be used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78: 1527); gpt, which confers resistance to mycophenolic acid (Mulligan & Berg, 1981, Proc. Natl. Acad. Sci. USA 78: 2072); neo, which confers resistance to the aminoglycoside G-418 Clinical Pharmacy 12: 488-505; Wu and Wu, 1991, 3:87-95; Tolstoshev, 1993, Ann. Rev. Pharmacol Toxicol. 32:573-596; Mulligan, 1993, Science 260:926-932; and Morgan and Anderson, 1993, Ann. Rev. Biochem. 62:191-217; May, 1993, TIB TECH 11(5):155-215). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol 150:1; and hygro, which confers resistance to hygromycin (Santerre et al., 1984, Gene 30:147).

The expression levels of an antibody of the invention can be increased by vector amplification (for a review, see Bebbington and Hentschel, The use of vectors based on gene amplification for the expression of cloned genes in mammalian cells in DNA cloning, Vol. 3 (Academic Press, New York, 1987). When a marker in the vector system expressing an antibody is amplifiable, increase in the level of inhibitor present in culture of host cell will increase the number of copies of the marker gene. Since the amplified region is associated with the nucleotide sequence of the antibody, production of the antibody will also increase (Crouse et al., 1983, Mol Cell Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once a molecule of the invention (i.e., antibodies) has been recombinantly expressed, it may be purified by any method known in the art for purification of polypeptides or antibodies, for example, by chromatography (e.g., ion exchange, affinity, particularly by affinity for the specific antigen after Protein A, and sizing column chromatography), centrifugation, differential solubility, or by any other standard technique for the purification of polypeptides or antibodies.

6.5 Prophylactic and Therapeutic Methods

The molecules of the invention with conferred and/or modified effector function activity are particularly useful for the treatment and/or prevention of a disease, disorder or infection where an enhanced efficacy of effector cell function (e.g., ADCC) mediated by FcγR is desired (e.g., cancer, infectious disease), and in enhancing the therapeutic efficacy of therapeutic antibodies, the effect of which is mediated by an effector function activity, e.g., ADCC.

The invention encompasses methods and compositions for treatment, prevention or management of a cancer in a subject, comprising administering to the subject a therapeutically effective amount of one or more molecules comprising a variant heavy chain engineered in accordance with the invention, which molecule further binds a cancer antigen. Molecules of the invention comprising the variant heavy chains are particularly useful for the prevention, inhibition, reduction of growth or regression of primary tumors, metastasis of cancer cells, and infectious diseases. Although not intending to be bound by a particular mechanism of action, molecules of the invention enhance the efficacy of cancer therapeutics by enhancing antibody mediated effector function resulting in an enhanced rate of tumor clearance or an enhanced rated of tumor reduction or a combination thereof. In alternate embodiments, the modified antibodies of the invention enhance the efficacy of cancer therapeutics by conferring oligomerization activity to the Fc region of the variant heavy chains of the invention, resulting in cross-linking of cell surface antigens and/or receptors and enhanced apoptosis or negative growth regulatory signaling.

According to an aspect of the invention, immunotherapeutics may be enhanced by modifying the heavy chain in accordance with the invention to confer or increase the potency of an antibody effector function activity, e.g., ADCC, CDC, phagocytosis, opsonization, etc., of the immunotherapeutic. In a specific embodiment, antibody dependent cellular toxicity and/or phagocytosis of tumor cells or infected cells is enhanced by modifying immunotherapeutics with variant heavy chains of the invention. Molecules of the invention may enhance the efficacy of immunotherapy treatment by enhancing at least one antibody-mediated effector function activity. In one particular embodiment, the efficacy of immunotherapy treatment is enhanced by enhancing the complement dependent cascade. In another embodiment of the invention, the efficacy of immunotherapy treatment is enhanced by enhancing the phagocytosis and/or opsonization of the targeted cells, e.g., tumor cells. In another embodiment of the invention, the efficacy of treatment is enhanced by enhancing antibody-dependent cell-mediated cytotoxicity (“ADCC”) in destruction of the targeted cells, e.g., tumor cells. The molecules of the invention may make an antibody that does not have a therapeutic effect in patients or in a subpopulation of patients have a therapeutic effect.

Although not intending to be bound by a particular mechanism of action, therapeutic antibodies engineered in accordance with the invention have enhanced therapeutic efficacy, in part, due to the ability of the Fc portion of the variant heavy chain to bind a target cell which expresses the particular FcγRs at reduced levels, for example, by virtue of the ability of the antibody to remain on the target cell longer due to an improved off rate for FcγR interaction.

The antibodies of the invention with enhanced affinity and avidity for FcγRs are particularly useful for the treatment, prevention or management of a cancer, or another disease or disorder, in a subject, wherein the FcγRs are expressed at low levels in the target cell populations. As used herein, FcγR expression in cells is defined in terms of the density of such molecules per cell as measured using common methods known to those skilled in the art. The molecules of the invention comprising variant heavy chains preferably also have a conferred or an enhanced avidity and affinity and/or effector function in cells which express a target antigen, e.g., a cancer antigen, at a density of 30,000 to 20,000 molecules/cell, at a density of 20,000 to 10,000 molecules/cell, at a density of 10,000 molecules/cell or less, at a density of 5000 molecules/cell or less, or at a density of 1000 molecules/cell or less. The molecules of the invention have particular utility in treatment, prevention or management of a disease or disorder, such as cancer, in a sub-population, wherein the target antigen is expressed at low levels in the target cell population.

The molecules of the invention may also be advantageously utilized in combination with other therapeutic agents known in the art for the treatment or prevention of diseases, such as cancer, autoimmune disease, inflammatory disorders, and infectious diseases. In a specific embodiment, molecules of the invention may be used in combination with monoclonal or chimeric antibodies, lymphokines, or hematopoietic growth factors (such as, e.g., IL-2, IL-3 and IL-7), which, for example, serve to increase the number or activity of effector cells which interact with the molecules and, increase immune response. The molecules of the invention may also be advantageously utilized in combination with one or more drugs used to treat a disease, disorder, or infection such as, for example anti-cancer agents, anti-inflammatory agents or anti-viral agents, e.g., as detailed in sections 5.4.1.2 and 5.4.2.1 below.

6.5.1 Cancers

The invention encompasses methods and compositions for treatment or prevention of cancer in a subject comprising administering to the subject a therapeutically effective amount of one or more molecules comprising a variant Fc region. In some embodiments, the invention encompasses methods and compositions for the treatment or prevention of cancer in a subject with FcγR polymorphisms such as those homozygous for the FγRIIIA-158V or FcγRIIIA-158F alleles. In some embodiments, the invention encompasses engineering therapeutic antibodies, e.g., tumor specific monoclonal antibodies in accordance with the methods of the invention such that the engineered antibodies have enhanced efficacy in patients homozygous for the low affinity allele of FcγRIIIA (158F). In other embodiments, the invention encompasses engineering therapeutic antibodies, e.g., tumor specific monoclonal antibodies in accordance with the methods of the invention such that the engineered antibodies have enhanced efficacy in patients homozygous for the high affinity allele of FcγRIIIA (158V).

The efficacy of monoclonal antibodies may depend on the FcγR polymorphism of the subject (Carton et al., 2002 Blood, 99: 754-8; Weng et al., 2003 J Clin Oncol. 21(21):3940-7 both of which are incorporated herein by reference in their entireties). These receptors are expressed on the surface of the effector cells and mediate ADCC. High affinity alleles, of the low affinity activating receptors, improve the effector cells' ability to mediate ADCC. The methods of the invention allow engineering molecules harboring Fc mutations to enhance their affinity to FcγR on effector cells via their altered Fc domains. The engineered antibodies of the invention provide better immunotherapy reagents for patients regardless of their FcγR polymorphism.

Molecules harboring the variant heavy chains engineered in accordance with the invention are tested by ADCC using either a cultured cell line or patient derived PMBC cells to determine the ability of the Fc mutations to enhance ADCC. Standard ADCC is performed using methods disclosed herein. Lymphocytes are harvested from peripheral blood using a Ficoll-Paque gradient (Pharmacia). Target cells, i.e., cultured cell lines or patient derived cells, are loaded with Europium (PerkinElmer) and incubated with effectors for 4 hrs at 37° C. Released Europium is detected using a fluorescent plate reader (Wallac). The resulting ADCC data indicates the efficacy of the Fc variants to trigger NK cell mediated cytotoxicity and establish which Fc variants can be tested with both patient samples and elutriated monocytes. Fc variants showing the greatest potential for enhancing the efficacy of the molecule are then tested in an ADCC assay using PBMCs from patients. PBMC from healthy donors are used as effector cells.

According to an aspect of the invention, molecules of the invention comprising variant heavy chains enhance the efficacy of immunotherapy by conferring or increasing the potency of an antibody effector function relative to a molecule containing the wild-type Fc region, e.g., ADCC, CDC, phagocytosis, opsonization, etc. In a specific embodiment, antibody dependent cellular toxicity and/or phagocytosis of tumor cells is conferred or enhanced using the molecules of the invention with variant heavy chains. Molecules of the invention may enhance the efficacy of immunotherapy cancer treatment by conferring or enhancing at least one antibody-mediated effector function. In one particular embodiment, a molecule of the invention comprising a variant heavy chain confers or enhances the efficacy of immunotherapy treatment by enhancing the complement dependent cascade. In another embodiment of the invention, the molecule of the invention comprising a variant heavy chain enhances the efficacy of immunotherapy treatment by conferring or enhancing the phagocytosis and/or opsonization of the targeted tumor cells. In another embodiment of the invention, the molecule of the invention comprising a variant heavy chain enhances the efficacy of treatment by conferring or enhancing antibody-dependent cell-mediated cytotoxicity (“ADCC”) in destruction of the targeted tumor cells.

The invention further contemplates engineering therapeutic antibodies (e.g., tumor specific monoclonal antibodies) for enhancing the therapeutic efficacy of the therapeutic antibody, for example, by enhancing the effector function of the therapeutic antibody (e.g., ADCC), or conferring effector function to a therapeutic antibody which doesn't have that effector function (at least detectable in an in vitro or in vivo assay). Preferably the therapeutic antibody is a cytotoxic and/or opsonizing antibody. It will be appreciated by one of skill in the art, that once molecules of the invention with desired binding properties (e.g., molecules comprising a variant heavy chain containing the Fc region of IgG2, IgG3 or IgG4 and having at least one amino acid modification relative to a wild-type heavy chain having an Fc region of the same isotype, which modification enhances the affinity of the Fc region of the variant heavy chain for FcγRIIIA and/or FcγRIIA relative to a comparable molecule (i.e., relative to a wild-type heavy chain having an Fc region of the same isotype)) have been identified (See Section 5.2 and Table 9) according to the methods of the invention, therapeutic antibodies may be engineered using standard recombinant DNA techniques and any known mutagenesis techniques, as described in Section 5.1 to produce engineered therapeutic carrying the identified mutation sites with the desired binding properties. Any of the therapeutic antibodies listed in Table 23 that have demonstrated therapeutic utility in cancer treatment, may be engineered according to the methods of the invention, for example, by modifying domains or regions of the variant heavy chain to confer an effector function or have an enhanced affinity for FcγRIIIA and/or FcγRIIA compared to a therapeutic antibody having a wild-type heavy chain containing an Fc region of the same isotype, and used for the treatment and or prevention of a cancer characterized by a cancer antigen. Other therapeutic antibodies include those against pathogenic agents such as those against Streptococcus pneumoniae Serotype 6B, see, e.g., Sun et al., 1999, Infection and Immunity, 67(3): 1172-9.

The heavy chain variants of the invention may be incorporated into therapeutic antibodies such as those disclosed herein or other polypeptide clinical candidates, i.e., a molecule comprising a heavy chain or portion thereof (e.g., an Fc region), which has been approved for us in clinical trials or any other molecule that may benefit from the heavy chain variants of the instant invention, and humanized, affinity matured, modified or engineered versions thereof.

The invention also encompasses engineering any other polypeptide comprising a heavy chain or region thereof which has therapeutic utility, including but not limited to ENBREL, according to the methods of the invention, in order to enhance the therapeutic efficacy of such polypeptides, for example, by enhancing the effector function of the polypeptide comprising a heavy chain or portion thereof necessary for eliciting effector function (e.g., Fc region).

TABLE 23
Therapeutic antibodies that can be engineered according to the
methods of the invention

| Company | Product | Disease | Target |
|---|---|---|---|
| Abgenix | ABX-EGF | Cancer | EGF receptor |
| AltaRex | OvaRex | ovarian cancer | tumor antigen CA125 |
| | BravaRex | metastatic cancers | tumor antigen MUC1 |
| Antisoma | Theragyn | ovarian cancer | PEM antigen |
| | (pemtumomabytrrium-90) | | |
| | Therex | breast cancer | PEM antigen |
| Boehringer | Blvatuzumab | head &neck cancer | CD44 |
| Ingelheim | | | |
| Centocor/J&J | Panorex | Colorectal cancer | 17-1A |
| | ReoPro | PTCA | gp IIIb/IIIa |
| | ReoPro | Acute MI | gp IIIb/IIIa |
| | ReoPro | Ischemic stroke | gp IIIb/IIIa |
| Corixa | Bexocar | NHL | CD20 |
| CRC | MAb, idiotypic 105AD7 | colorectal cancer | gp72 |
| Technology | | vaccine | |
| Crucell | Anti-EpCAM | cancer | Ep-CAM |
| Cytoclonal | MAb, lung cancer | non-small cell lung | NA |
| | | cancer | |
| Genentech | HERCEPTIN ® | metastatic breast | HER-2 |
| | | cancer | |
| | HERCEPTIN ® | early stage breast | HER-2 |
| | | cancer | |
| | RITUXAN ® | Relapsed/refractory | CD20 |
| | | low-grade or | |
| | | follicular NHL | |
| | RITUXAN ® | intermediate & | CD20 |
| | | high-grade NHL | |
| | MAb-VEGF | NSCLC, metastatic | VEGF |
| | MAb-VEGF | Colorectal cancer, | VEGF |
| | | metastatic | |
| | AMD Fab | age-related macular | CD18 |
| | | degeneration | |
| | E-26 (2^{nd} gen. IgE) | allergic asthma & | IgE |
| | | rhinitis | |
| IDEC | Zevalin (RITUXAN ®) + | low grade of | CD20 |
| | yttrium-90) | follicular, relapsed | |
| | | or refractory, CD20- | |
| | | positive, B-cell | |
| | | NHL and | |
| | | Rituximab- | |
| | | refractory NHL | |
| ImClone | Cetuximab + innotecan | refractory colorectal | EGF receptor |
| | | carcinoma | |
| | Cetuximab + cisplatin & | newly diagnosed or | EGF receptor |
| | radiation | recurrent head & | |
| | | neck cancer | |
| | Cetuximab + | newly diagnosed | EGF receptor |
| | gemcitabine | metastatic | |
| | | pancreatic | |
| | | carcinoma | |
| | Cetuximab + cisplatin + | recurrent or | EGF receptor |
| | 5FU or Taxol | metastatic head & | |
| | | neck cancer | |
| | Cetuximab + carboplatin + | newly diagnosed | EGF receptor |
| | paclitaxel | non-small cell lung | |
| | | carcinoma | |
| | Cetuximab + cisplatin | head &neck cancer | EGF receptor |
| | | (extensive incurable | |
| | | local-regional | |
| | | disease &distant | |
| | | metasteses) | |
| | Cetuximab + radiation | locally advanced | EGF receptor |
| | | head &neck | |
| | | carcinoma | |
| | BEC2 + Bacillus | small cell lung | mimics ganglioside |
| | Calmette Guerin | carcinoma | GD3 |
| | BEC2 + Bacillus | melanoma | mimics ganglioside |
| | Calmette Guerin | | GD3 |
| | IMC-1C11 | colorectal cancer | VEGF-receptor |
| | | with liver | |
| | | metasteses | |
| ImmonoGen | nuC242-DM1 | Colorectal, gastric, | nuC242 |
| | | and pancreatic | |
| | | cancer | |
| ImmunoMedics | LymphoCide | Non-Hodgkins | CD22 |
| | | lymphoma | |
| | LymphoCide Y-90 | Non-Hodgkins | CD22 |
| | | lymphoma | |
| | CEA-Cide | metastatic solid | CEA |
| | | tumors | |
| | CEA-Cide Y-90 | metastatic solid | CEA |
| | | tumors | |
| | CEA-Scan (Tc-99m- | colorectal cancer | CEA |
| | labeled arcitumomab) | (radioimaging) | |
| | CEA-Scan (Tc-99m- | Breast cancer | CEA |
| | labeled arcitumomab) | (radioimaging) | |
| | CEA-Scan (Tc-99m- | lung cancer | CEA |
| | labeled arcitumomab) | (radioimaging) | |
| | CEA-Scan (Tc-99m- | intraoperative | CEA |
| | labeled arcitumomab) | tumors (radio | |
| | | imaging) | |
| | LeukoScan (Tc-99m- | soft tissue infection | CEA |
| | labeled sulesomab) | (radioimaging) | |
| | LymphoScan (Tc-99m- | lymphomas | CD22 |
| | labeled) | (radioimaging) | |
| | AFP-Scan (Tc-99m- | liver 7 gem-cell | AFP |
| | labeled) | cancers | |
| | | (radioimaging) | |
| Intracel | HumaRAD-HN | head &neck cancer | NA |
| | (+yttrium-90) | | |
| | HumaSPECT | colorectal imaging | NA |
| Medarex | MDX-101 (CTLA-4) | Prostate and other | CTLA-4 |
| | | cancers | |
| | MDX-210 (her-2 | Prostate cancer | HER-2 |
| | overexpression) | | |
| | MDX-210/MAK | Cancer | HER-2 |
| MedImmune | Vitaxin | Cancer | αvβ₃ |
| Merck KGaA | MAb 425 | Various cancers | EGF receptor |
| | IS-IL-2 | Various cancers | Ep-CAM |
| Millennium | Campath (alemtuzumab) | chronic lymphocytic | CD52 |
| | | leukemia | |
| NeoRx | CD20-streptavidin | Non-Hodgkins | CD20 |
| | (+biotin-yttrium 90) | lymphoma | |
| | Avidicin (albumin + | metastatic cancer | NA |
| | NRLU13) | | |
| Peregrine | Oncolym (+iodine-131) | Non-Hodgkins | HLA-DR 10 beta |
| | | lymphoma | |
| | Cotara (+iodine-131) | unresectable | DNA-associated |
| | | malignant glioma | proteins |
| Pharmacia | C215 (+staphylococcal | pancreatic cancer | NA |
| Corporation | enterotoxin) | | |
| | MAb, lung/kidney | lung &kidney | NA |
| | cancer | cancer | |
| | nacolomab tafenatox | colon &pancreatic | NA |
| | (C242 + staphylococcal | cancer | |
| | enterotoxin) | | |
| Protein Design | Nuvion | T cell malignancies | CD3 |
| Labs | SMART M195 | AML | CD33 |
| | SMART 1D10 | NHL | HLA-DR antigen |
| Titan | CEAVac | colorectal cancer, | CEA |
| | | advanced | |
| | TriGem | metastatic | GD2-ganglioside |
| | | melanoma &small | |
| | | cell lung cancer | |
| | TriAb | metastatic breast | MUC-1 |
| | | cancer | |
| Trilex | CEAVac | colorectal cancer, | CEA |
| | | advanced | |
| | TriGem | metastatic | GD2-ganglioside |
| | | melanoma &small | |
| | | cell lung cancer | |
| | TriAb | metastatic breast | MUC-1 |
| | | cancer | |
| Viventia | NovoMAb-G2 | Non-Hodgkins | NA |
| Biotech | radiolabeled | lymphoma | |
| | Monopharm C | colorectal & | SK-1 antigen |
| | | pancreatic | |
| | | carcinoma | |
| | GlioMAb-H (+gelonin | gliorna, melanoma | NA |
| | toxin) | &neuroblastoma | |
| Xoma | RITUXAN ® | Relapsed/refractory | CD20 |
| | | low-grade or | |
| | | follicular NHL | |
| | RITUXAN ® | intermediate & | CD20 |
| | | high-grade NHL | |
| | ING-1 | adenomcarcinoma | Ep-CAM |

Accordingly, the invention provides methods of preventing or treating cancer characterized by a cancer antigen, using a therapeutic antibody that binds a cancer antigen and is cytotoxic and has been modified at one or more sites in the Fc region, according to the invention, to bind FcγRIIIA and/or FcγRIIA with a higher affinity than the parent therapeutic antibody, and/or mediates one or more effector function (e.g., ADCC, phagocytosis) either not detectably mediated by the parent antibody or more effectively than the parent antibody. In another embodiment, the invention provides methods of preventing or treating cancer characterized by a cancer antigen, using a therapeutic antibody that binds a cancer antigen and is cytotoxic, and has been engineered according to the invention to bind FcγRIIIA and/or FcγRIIA with a higher affinity and bind FcγRIIB with a lower affinity than the parent therapeutic antibody, and/or mediates one or more effector function (e.g., ADCC, phagocytosis) either not detectably mediated by the parent antibody or more effectively than the parent antibody. The therapeutic antibodies that have been engineered according to the invention are useful for prevention or treatment of cancer, since they have an enhanced cytotoxic activity (e.g., enhanced tumor cell killing and/or enhanced for example, ADCC activity or CDC activity).

Cancers associated with a cancer antigen may be treated or prevented by administration of a therapeutic antibody that binds a cancer antigen and is cytotoxic, and has been engineered according to the methods of the invention to have, for example, an enhanced effector function. In one particular embodiment, the therapeutic antibodies engineered according to the methods of the invention enhance the antibody-mediated cytotoxic effect of the antibody directed at the particular cancer antigen. For example, but not by way of limitation, cancers associated with the following cancer antigens may be treated or prevented by the methods and compositions of the invention: KS1/4 pan-carcinoma antigen (Perez and Walker, 1990, J. Immunol. 142:32-37; Bumal, 1988, Hybridoma 7(4):407-415), ovarian carcinoma antigen (CA125) (Yu et al., 1991, Cancer Res. 51(2):48-475), prostatic acid phosphate (Tailor et al., 1990, Nucl Acids Res. 18(1):4928), prostate specific antigen (Henttu and Vihko, 1989, Biochem. Biophys. Res. Comm. 10(2):903-910; Israeli et al., 1993, Cancer Res. 53:227-230), melanoma-associated antigen p97 (Estin et al., 1989, J. Natl. Cancer Instit. 81(6):445-44), melanoma antigen gp75 (Vijayasardahl et al., 1990, J. Exp. Med. 171(4):1375-1380), high molecular weight melanoma antigen (HMW-MAA) (Natali et al., 1987, Cancer 59:55-3; Mittelman et al., 1990, J. Clin. Invest. 86:2136-2144)), prostate specific membrane antigen, carcinoembryonic antigen (CEA) (Foon et al., 1994, Proc. Am. Soc. Clin. Oncol 13:294), polymorphic epithelial mucin antigen, human milk fat globule antigen, Colorectal tumor-associated antigens such as: CEA, TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shiara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immun. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erthrocytes and primary endoderm, I(Ma) found in gastric adencarcinomas, M18 and M39 found in breast epithelium, SSEA-1 found in myeloid cells, VEP8, VEP9, My1, VIM-D5, and D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC 10.2 found in embryonal carcinoma cells, gastric adenocarcinoma, CO-514 (blood group Le^{a}) found in adenocarcinoma, NS-10 found in adenocarcinomas, CO-43 (blood group Le^{b}), G49, EGF receptor, (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, M1:22:25:8 found in embryonal carcinoma cells and SSEA-3, SSEA-4 found in 4-8-cell stage embryos. In another embodiment, the antigen is a T cell receptor derived peptide from a cutaneous T cell lymphoma (see Edelson, 1998, The Cancer Journal 4:62).

Cancers and related disorders that can be treated or prevented by methods and compositions of the present invention include, but are not limited to, the following: Leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including but not limited to, ocular melanoma such as iris melanoma, choroidal melanoma, and cilliary body melanoma, and retinoblastoma; vaginal cancers, including but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including but not limited to, squamous cell carcinoma, and adenocarcinoma; uterine cancers including but not limited to, endometrial carcinoma and uterine sarcoma; ovarian cancers including but not limited to, ovarian epithelial carcinoma, borderline tumor, germ cell tumor, and stromal tumor; esophageal cancers including but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including but not limited to, adenocarcinoma, fungaling (polypoid), ulcerating, superficial spreading, diffusely spreading, malignant lymphoma, liposarcoma, fibrosarcoma, and carcinosarcoma; colon cancers; rectal cancers; liver cancers including but not limited to hepatocellular carcinoma and hepatoblastoma, gallbladder cancers including but not limited to, adenocarcinoma; cholangiocarcinomas including but not limited to, pappillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including but not limited to, adenocarcinoma, leiomyosarcoma, and rhabdomyosarcoma; penal cancers; oral cancers including but not limited to, squamous cell carcinoma; basal cancers; salivary gland cancers including but not limited to, adenocarcinoma, mucoepidermoid carcinoma, and adenoidcystic carcinoma; pharynx cancers including but not limited to, squamous cell cancer, and verrucous; skin cancers including but not limited to, basal cell carcinoma, squamous cell carcinoma and melanoma, superficial spreading melanoma, nodular melanoma, lentigo malignant melanoma, acral lentiginous melanoma; kidney cancers including but not limited to, renal cell cancer, adenocarcinoma, hypemephroma, fibrosarcoma, transitional cell cancer (renal pelvis and/or uterer); Wilms' tumor; bladder cancers including but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, prostate, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions of the invention in the ovary, bladder, breast, colon, lung, skin, pancreas, or uterus. In other specific embodiments, sarcoma, melanoma, or leukemia is treated or prevented by the methods and compositions of the invention.

In a specific embodiment, a molecule of the invention (e.g., an antibody comprising a variant heavy chain, or a therapeutic monoclonal antibody engineered according to the methods of the invention) inhibits or reduces the growth of cancer cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth of cancer cells in the absence of said molecule of the invention.

In a specific embodiment, a molecule of the invention (e.g., an antibody comprising a variant heavy chain, or a therapeutic monoclonal antibody engineered according to the methods of the invention) kills cells or inhibits or reduces the growth of cancer cells at least 5%, at least 10%, at least 20%, at least 25%, at least 30%, at least 35%, at least 40%, at least 45%, at least 50%, at least 60%, at least 70%, at least 75%, at least 80%, at least 85%, at least 90%, at least 95%, or at least 100% better than the parent molecule.

6.5.1.1 Combination Therapy

The invention further encompasses administering the molecules of the invention in combination with other therapies known to those skilled in the art for the treatment or prevention of cancer or infectious disease, including but not limited to, current standard and experimental chemotherapies, hormonal therapies, biological therapies, immunotherapies, radiation therapies, or surgery. In some embodiments, the molecules of the invention may be administered in combination with a therapeutically or prophylactically effective amount of one or more anti-cancer agents, therapeutic antibodies or other agents known to those skilled in the art for the treatment and/or prevention of cancer (See Section 5.5.1.2).

In certain embodiments, one or more molecule of the invention is administered to a mammal, preferably a human, concurrently with one or more other therapeutic agents useful for the treatment of cancer. The term “concurrently” is not limited to the administration of prophylactic or therapeutic agents at exactly the same time, but rather it is meant that a molecule of the invention and the other agent are administered to a mammal in a sequence and within a time interval such that the molecule of the invention can act together with the other agent to provide an increased benefit than if they were administered otherwise. For example, each prophylactic or therapeutic agent (e.g., chemotherapy, radiation therapy, hormonal therapy or biological therapy) may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route. In various embodiments, the prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

In other embodiments, the prophylactic or therapeutic agents are administered at about 2 to 4 days apart, at about 4 to 6 days apart, at about 1 week part, at about 1 to 2 weeks apart, or more than 2 weeks apart. In preferred embodiments, the prophylactic or therapeutic agents are administered in a time frame where both agents are still active. One skilled in the art would be able to determine such a time frame by determining the half life of the administered agents.

In certain embodiments, the prophylactic or therapeutic agents of the invention are cyclically administered to a subject. Cycling therapy involves the administration of a first agent for a period of time, followed by the administration of a second agent and/or third agent for a period of time and repeating this sequential administration. Cycling therapy can reduce the development of resistance to one or more of the therapies, avoid or reduce the side effects of one of the therapies, and/or improves the efficacy of the treatment.

In certain embodiments, prophylactic or therapeutic agents are administered in a cycle of less than about 3 weeks, about once every two weeks, about once every 10 days or about once every week. One cycle can comprise the administration of a therapeutic or prophylactic agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In yet other embodiments, the therapeutic and prophylactic agents of the invention are administered in metronomic dosing regimens, either by continuous infusion or frequent administration without extended rest periods. Such metronomic administration can involve dosing at constant intervals without rest periods. Typically the therapeutic agents, in particular cytotoxic agents, are used at lower doses. Such dosing regimens encompass the chronic daily administration of relatively low doses for extended periods of time. In preferred embodiments, the use of lower doses can minimize toxic side effects and eliminate rest periods. In certain embodiments, the therapeutic and prophylactic agents are delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months. The scheduling of such dose regimens can be optimized by the skilled oncologist.

In other embodiments, courses of treatment are administered concurrently to a mammal, i.e., individual doses of the therapeutics are administered separately yet within a time interval such that molecules of the invention can work together with the other agent or agents. For example, one component may be administered one time per week in combination with the other components that may be administered one time every two weeks or one time every three weeks. In other words, the dosing regimens for the therapeutics are carried out concurrently even if the therapeutics are not administered simultaneously or within the same patient visit.

When used in combination with other prophylactic and/or therapeutic agents, the molecules of the invention and the prophylactic and/or therapeutic agent can act additively or, more preferably, synergistically. In one embodiment, a molecule of the invention is administered concurrently with one or more therapeutic agents in the same pharmaceutical composition. In another embodiment, a molecule of the invention is administered concurrently with one or more other therapeutic agents in separate pharmaceutical compositions. In still another embodiment, a molecule of the invention is administered prior to or subsequent to administration of another prophylactic or therapeutic agent. The invention contemplates administration of a molecule of the invention in combination with other prophylactic or therapeutic agents by the same or different routes of administration, e.g., oral and parenteral. In certain embodiments, when a molecule of the invention is administered concurrently with another prophylactic or therapeutic agent that potentially produces adverse side effects including, but not limited to, toxicity, the prophylactic or therapeutic agent can advantageously be administered at a dose that falls below the threshold that the adverse side effect is elicited.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity and type of cancer, the route of administration, as well as age, body weight, response, and the past medical history of the patient. Suitable regimens can be selected by one skilled in the art by considering such factors and by following, for example, dosages reported in the literature and recommended in the Physician's Desk Reference (56^{th} ed., 2002).

6.5.1.2 Other Therapeutic/Prophylactic Agents

In a specific embodiment, the methods of the invention encompass the administration of one or more molecules of the invention with one or more therapeutic agents used for the treatment and/or prevention of cancer. In one embodiment, angiogenesis inhibitors may be administered in combination with the molecules of the invention. Angiogenesis inhibitors that can be used in the methods and compositions of the invention include but are not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; farnesyl transferase inhibitors (FTI); and bisphosphonates.

Anti-cancer agents that can be used in combination with the molecules of the invention in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, micro algal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

Examples of therapeutic antibodies that can be used in methods of the invention include but are not limited to ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Smart M195 which is a humanized anti-CD33 IgG antibody (Protein Design Lab/Kanebo); LYMPHOCIDE™ which is a humanized anti-CD22 IgG antibody (Immunomedics); ICM3 is a humanized anti-ICAM3 antibody (ICOS Pharm); IDEC-114 is a primatied anti-CD80 antibody (IDEC Pharm/Mitsubishi); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α. IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech). Other examples of therapeutic antibodies that can be used in accordance with the invention are presented in Table 10.

6.5.2 Autoimmune Disease and Inflammatory Diseases

In some embodiments, molecules of the invention comprise a variant heavy chain containing the Fc region of IgG2, IgG3 or IgG4, and have one or more amino acid modifications in one or more regions relative to a wild type heavy chain having an Fc region of the same isotype, which modification increases the affinity of the variant Fc region for FcγRIIB but decreases the affinity of the variant Fc region for FcγRIIIA and/or FcγRIIA. Molecules of the invention with such binding characteristics are useful in regulating the immune response, e.g., in inhibiting the immune response in connection with autoimmune diseases or inflammatory diseases. Although not intending to be bound by any mechanism of action, molecules of the invention with an enhanced affinity for FcγRIIB and a decreased affinity for FcγRIIIA and/or FcγRIIA may lead to dampening of the activating response to FcγR and inhibition of cellular responsiveness.

In some embodiments, a molecule of the invention comprising a variant heavy chain is not an immunoglobulin, and comprises at least one amino acid modification which modification increases the affinity of the variant heavy chain for FcγRIIB relative to a molecule comprising a wild-type heavy chain having an Fc region of the same isotype. In other embodiments, said molecule further comprises one or more amino acid modifications, which modifications decreases the affinity of the molecule for an activating FcγR. In some embodiments, the molecule is a soluble (i.e., not membrane bound) variant heavy chain or portion thereof (e.g., Fc region). The invention contemplates other amino acid modifications within the soluble variant heavy chain, or region thereof, which modulate its affinity for various Fc receptors, including those known to one skilled in the art as described herein. In other embodiments, the molecule (e.g., variant heavy chain containing an Fc region of IgG2, IgG3 or IgG4 and having one or more amino acid modification relative to a wild type heavy chain having an Fc region of the same isotype) is modified using techniques known to one skilled in the art and as described herein to increase the in vivo half life of the molecule. Such molecules have therapeutic utility in treating and/or preventing an autoimmune disorder. Although not intending to be bound by any mechanism of actions, such molecules with enhanced affinity for FcγRIIB will lead to a dampening of the activating receptors and thus a dampening of the immune response and have therapeutic efficacy for treating and/or preventing an autoimmune disorder.

In certain embodiments, the one or more amino acid modifications, which increase the affinity of the Fc region of the variant heavy chain for FcγRIIB but decrease the affinity of the Fc region of the variant heavy chain for FcγRIIIA comprise a substitution at position 246 with threonine and at position 396 with histidine; or a substitution at position 268 with aspartic acid and at position 318 with aspartic acid; or a substitution at position 217 with serine, at position 378 with valine, and at position 408 with arginine; or a substitution at position 375 with cysteine and at position 396 with leucine; or a substitution at position 246 with isolcucine and at position 334 with asparagine; or a substitution at position 247 with leucine; or a substitution at position 372 with tyrosine; or a substitution at position 326 with glutamic acid; or a substitution at position 224 with leucine.

The variant heavy chains of the invention that have an enhanced affinity for FcγRIIB and a decreased affinity for FcγRIIIA and/or FcγRIIA relative to a comparable molecule comprising a wild-type heavy chain having an Fc region of the same isotype, may be used to treat or prevent autoimmune diseases or inflammatory diseases. The present invention provides methods of preventing, treating, or managing one or more symptoms associated with an autoimmune or inflammatory disorder in a subject, comprising administering to said subject a therapeutically or prophylactically effective amount of one or more molecules of the invention with variant heavy chains that have an enhanced affinity for FcγRIIB and a decreased affinity for FcγRIIIA and or FcγRIIA relative to a comparable molecule comprising a wild type heavy chain having an Fc region of the same isotype.

The invention also provides methods for preventing, treating, or managing one or more symptoms associated with an inflammatory disorder in a subject further comprising, administering to said subject a therapeutically or prophylactically effective amount of one or more anti-inflammatory agents. The invention also provides methods for preventing, treating, or managing one or more symptoms associated with an autoimmune disease further comprising, administering to said subject a therapeutically or prophylactically effective amount of one or more immunomodulatory agents. Section 5.4.2.1 provides non-limiting examples of anti-inflammatory agents and immunomodulatory agents.

Examples of autoimmune disorders that may be treated by administering the molecules of the present invention include, but are not limited to, alopecia greata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Ménière's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis. Examples of inflammatory disorders include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections. As described herein in Section 2.2.2, some autoimmune disorders are associated with an inflammatory condition. Thus, there is overlap between what is considered an autoimmune disorder and an inflammatory disorder. Therefore, some autoimmune disorders may also be characterized as inflammatory disorders. Examples of inflammatory disorders which can be prevented, treated or managed in accordance with the methods of the invention include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections.

Molecules of the invention with variant heavy chains that have an enhanced affinity for FcγRIIB and a decreased affinity for FcγRIIIA relative to a comparable molecule comprising a wild-type heavy chain having an Fc region of the same isotype can also be used to reduce the inflammation experienced by animals, particularly mammals, with inflammatory disorders. In a specific embodiment, a molecule of the invention reduces the inflammation in an animal by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the inflammation in an animal, which is not administered the said molecule or which is administered the parent molecule.

Molecules of the invention with variant heavy chains that have an enhanced affinity for FcγRIIB and a decreased affinity for FcγRIIIA relative to a comparable molecule comprising a wild-type heavy chain having an Fc region of the same isotype can also be used to prevent the rejection of transplants.

The invention further contemplates engineering any of the antibodies known in the art for the treatment and/or prevention of autoimmune disease or inflammatory disease, so that the antibodies comprise a variant heavy chain of the invention comprising one or more amino acid modifications relative to a wild-type heavy chain having an Fc region of the same isotype, which have been identified to have a conferred effector function and/or enhanced affinity for FcγRIIB and a decreased affinity for FcγRIIIA relative to a comparable molecule comprising a wild type heavy chain having an Fc region of the same isotype. A non-limiting example of the antibodies that are used for the treatment or prevention of inflammatory disorders which can be engineered according to the invention is presented in Table 24A, and a non-limiting example of the antibodies that are used for the treatment or prevention of autoimmune disorder is presented in Table 24B.

TABLE 24A
Antibodies for inflammitory diseases and autoimmune diseases
that can be engineered in accordance with the invention

| Antibody | Target | Product | | | |
|---|---|---|---|---|---|
| Name | Antigen | Type | Isotype | Sponsors | Indication |
| 5G1.1 | Complement | Humanized | IgG | Alexion Pharm Inc | Rheumatoid |
| | (C5) | | | | Arthritis |
| 5G1.1 | Complement | Humanized | IgG | Alexion Pharm Inc | SLE |
| | (C5) | | | | |
| 5G1.1 | Complement | Humanized | IgG | Alexion Pharm Inc | Nephritis |
| | (C5) | | | | |
| 5G1.1-SC | Complement | Humanized | ScFv | Alexion Pharm Inc | Cardiopulmonary |
| | (C5) | | | | Bypass |
| 5G1.1-SC | Complement | Humanized | ScFv | Alexion Pharm Inc | Myocardial |
| | (C5) | | | | Infarction |
| 5G1.1-SC | Complement | Humanized | ScFv | Alexion Pharm Inc | Angioplasty |
| | (C5) | | | | |
| ABX-CBL | CBL | Human | | Abgenix Inc | GvHD |
| ABX-CBL | CD147 | Murine | IgG | Abgenix Inc | Allograft |
| | | | | | rejection |
| ABX-IL8 | IL-8 | Human | IgG2 | Abgenix Inc | Psoriasis |
| Antegren | VLA-4 | Humanized | IgG | Athena/Elan | Multiple |
| | | | | | Sclerosis |
| Anti-CD11a | CD11a | Humanized | IgG1 | Genentech | Psoriasis |
| | | | | Inc/Xoma | |
| Anti-CD18 | CD18 | Humanized | Fab′2 | Genentech Inc | Myocardial |
| | | | | | infarction |
| Anti-LFA1 | CD18 | Murine | Fab′2 | Pasteur-Merieux/ | Allograft |
| | | | | Immunotech | rejection |
| Antova | CD40L | Humanized | IgG | Biogen | Allograft |
| | | | | | rejection |
| Antova | CD40L | Humanized | IgG | Biogen | SLE |
| BTI-322 | CD2 | Rat | IgG | Medimmune Inc | GvHD, |
| | | | | | Psoriasis |
| CDP571 | TNF-alpha | Humanized | IgG4 | Celltech | Crohn's |
| | | | | | Disease |
| CDP571 | TNF-alpha | Humanized | IgG4 | Celltech | Rheumatoid |
| | | | | | Arthritis |
| CDP850 | E-selectin | Humanized | | Celltech | Psoriasis |
| Corsevin M | Fact VII | Chimeric | | Centocor | Anticoagulant |
| D2E7 | TNF-alpha | Human | | CAT/BASF | Rheumatoid |
| | | | | | Arthritis |
| Hu23F2G | CD11/18 | Humanized | | ICOS Pharm Inc | Multiple |
| | | | | | Sclerosis |
| Hu23F2G | CD11/18 | Humanized | IgG | ICOS Pharm Inc | Stroke |
| IC14 | CD14 | | | ICOS Pharm Inc | Toxic shock |
| ICM3 | ICAM-3 | Humanized | | ICOS Pharm Inc | Psoriasis |
| IDEC-114 | CD80 | Primatised | | IDEC | Psoriasis |
| | | | | Pharm/Mitsubishi | |
| IDEC-131 | CD40L | Humanized | | IDEC Pharm/Eisai | SLE |
| IDEC-131 | CD40L | Humanized | | IDEC Pharm/Eisai | Multiple |
| | | | | | Sclerosis |
| IDEC-151 | CD4 | Primatised | IgG1 | IDEC | Rheumatoid |
| | | | | Pharm/GlaxoSmith | Arthritis |
| | | | | Kline | |
| IDEC-152 | CD23 | Primatised | | IDEC Pharm | Asthma/ |
| | | | | | Allergy |
| Infliximab | TNF-alpha | Chimeric | IgG1 | Centocor | Rheumatoid |
| | | | | | Arthritis |
| Infliximab | TNF-alpha | Chimeric | IgG1 | Centocor | Crohn's |
| LDP-01 | beta2-integrin | Humanized | IgG | Millennium Inc | Stroke |
| | | | | (LeukoSite Inc.) | |
| LDP-01 | beta2-integrin | Humanized | IgG | Millennium Inc | Allograft |
| | | | | (LeukoSite Inc.) | rejection |
| LDP-02 | alpha4beta7 | Humanized | | Millennium Inc | Ulcerative |
| | | | | (LeukoSite Inc.) | Colitis |
| MAK-195F | TNF alpha | Murine | Fab′2 | Knoll Pharm, BASF | Toxic shock |
| MDX-33 | CD64 (FcR) | Human | | Medarex/Centeon | Autoimmune |
| | | | | | haematogical |
| | | | | | disorders |
| MDX-CD4 | CD4 | Human | IgG | Medarex/Eisai/ | Rheumatoid |
| | | | | Genmab | Arthritis |
| MEDI-507 | CD2 | Humanized | | Medimmune Inc | Psoriasis |
| MEDI-507 | CD2 | Humanized | | Medimmune Inc | GvHD |
| OKT4A | CD4 | Humanized | IgG | Ortho Biotech | Allograft |
| | | | | | rejection |
| OrthoClone | CD4 | Humanized | IgG | Ortho Biotech | Autoimmune |
| OKT4A | | | | | disease |
| Orthoclone/ | CD3 | Murine | mIgG2a | Ortho Biotech | Allograft |
| anti-CD3 | | | | | rejection |
| OKT3 | | | | | |
| RepPro/ | gpIIbIIIa | Chimeric | Fab | Centocor/Lilly | Complications |
| Abciximab | | | | | of coronary |
| | | | | | angioplasty |
| rhuMab- | IgE | Humanized | IgG1 | Genentech/Novartis/ | Asthma/ |
| E25 | | | | Tanox Biosystems | Allergy |
| SB-240563 | IL5 | Humanized | | GlaxoSmithKline | Asthma/ |
| | | | | | Allergy |
| SB-240683 | IL-4 | Humanized | | GlaxoSmithKline | Asthma/ |
| | | | | | Allergy |
| SCH55700 | IL-5 | Humanized | | Celltech/Schering | Asthma/ |
| | | | | | Allergy |
| Simulect | CD25 | Chimeric | IgG1 | Novartis Pharm | Allograft |
| | | | | | rejection |
| SMART | CD3 | Humanized | | Protein Design Lab | Autoimmune |
| a-CD3 | | | | | disease |
| SMART | CD3 | Humanized | | Protein Design Lab | Allograft |
| a-CD3 | | | | | rejection |
| SMART | CD3 | Humanized | IgG | Protein Design Lab | Psoriasis |
| a-CD3 | | | | | |
| Zenapax | CD25 | Humanized | IgG1 | Protein Design | Allograft |
| | | | | Lab/Hoffman- | rejection |
| | | | | La Roche | |

TABLE 24B
Antibodies for autoimmune disorders that can be engineered in
accordance with the invnetion

| Antibody | Indication | Target Antigen |
|---|---|---|
| ABX-RB2 | | antibody to CBL antigen on T cells, |
| | | B cells and NK cells |
| | | fully human antibody from the |
| | | Xenomouse |
| 5c8 (Anti CD-40 | Phase II trials were halted in Oct. | CD-40 |
| ligand antibody) | 99 examine “adverse events” | |
| IDEC 131 | systemic lupus erythyematous | anti CD40 |
| | (SLE) | humanized |
| IDEC 151 | rheumatoid arthritis | primatized; anti-CD4 |
| IDEC 152 | Asthma | primatized; anti-CD23 |
| IDEC 114 | Psoriasis | primatized anti-CD80 |
| MEDI-507 | rheumatoid arthritis; multiple | anti-CD2 |
| | sclerosis | |
| | Crohn's disease | |
| | Psoriasis | |
| LDP-02 (anti-b7 | inflammatory bowel disease | a4b7 integrin receptor on white |
| mAb) | Chron's disease | blood cells (leukocytes) |
| | ulcerative colitis | |
| SMART Anti- | autoimmune disorders | Anti-Gamma Interferon |
| Gamma Interferon | | |
| antibody | | |
| Verteportin | rheumatoid arthritis | |
| MDX-33 | blood disorders caused by | monoclonal antibody against FcRI |
| | autoimmune reactions | receptors |
| | Idiopathic Thrombocytopenia | |
| | Purpurea (ITP) | |
| | autoimmune hemolytic anemia | |
| MDX-CD4 | treat rheumatoid arthritis and | monoclonal antibody against CD4 |
| | other autoimmunity | receptor molecule |
| VX-497 | autoimmune disorders | inhibitor of inosine monophosphate |
| | multiple sclerosis | dehydrogenase |
| | rheumatoid arthritis | (enzyme needed to make new RNA |
| | inflammatory bowel disease | and DNA |
| | lupus | used in production of nucleotides |
| | psoriasis | needed for lymphocyte |
| | | proliferation) |
| VX-740 | rheumatoid arthritis | inhibitor of ICE |
| | | interleukin-1 beta (converting |
| | | enzyme |
| | | controls pathways leading to |
| | | aggressive immune response) |
| VX-745 | specific to inflammation | inhibitor of P38MAP kinase |
| | involved in chemical signalling of | mitogen activated protein kinase |
| | immune response | |
| | onset and progression of | |
| | inflammation | |
| Enbrel (etanercept) | | targets TNF (tumor necrosis factor) |
| IL-8 | | fully human monoclonal antibody |
| | | against IL-8 (interleukin 8) |
| Apogen MP4 | | recombinant antigen |
| | | selectively destroys disease |
| | | associated T-cells |
| | | induces apoptosis |
| | | T-cells eliminated by programmed |
| | | cell death |
| | | no longer attack body's own cells |
| | | specific apogens target specific T- |
| | | cells |

6.5.2.1 Immunomodulatory Agents and Anti-Inflammatory Agents

The present invention provides methods of treatment for autoimmune diseases and inflammatory diseases comprising administration of the molecules with variant heavy chain having an enhanced affinity for FcγRIIB and a decreased affinity for FcγRIIIA and/or FcγRIIA in conjunction with other treatment agents. Examples of immunomodulatory agents include, but are not limited to, methothrexate, ENBREL, REMICADE™, leflunomide, cyclophosphamide, cyclosporine A, and macrolide antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators.

Anti-inflammatory agents have exhibited success in treatment of inflammatory and autoimmune disorders and are now a common and a standard treatment for such disorders. Any anti-inflammatory agent well-known to one of skill in the art can be used in the methods of the invention. Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (ALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

6.5.3 Infectious Disease

The invention also encompasses methods for treating or preventing an infectious disease in a subject comprising administering a therapeutically or prophylatically effective amount of one or more molecules of the invention. Infectious diseases that can be treated or prevented by the molecules of the invention are caused by infectious agents including but not limited to viruses, bacteria, fungi, protozae, and viruses.

Viral diseases that can be treated or prevented using the molecules of the invention in conjunction with the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, small pox, Epstein Barr virus, human immunodeficiency virus type I (HIV-I), human immunodeficiency virus type II (HIV-II), and agents of viral diseases such as viral miningitis, encephalitis, dengue or small pox.

Bacterial diseases that can be treated or prevented using the molecules of the invention in conjunction with the methods of the present invention, that are caused by bacteria include, but are not limited to, mycobacteria rickettsia, mycoplasma, neisseria, S. pneumonia, Borrelia burgdorferi (Lyme disease), Bacillus antracis (anthrax), tetanus, streptococcus, staphylococcus, mycobacterium, tetanus, pertissus, cholera, plague, diptheria, chlamydia, S. aureus and legionella.

Protozoal diseases that can be treated or prevented using the molecules of the invention in conjunction with the methods of the present invention, that are caused by protozoa include, but are not limited to, leishmania, kokzidioa, trypanosoma or malaria.

Parasitic diseases that can be treated or prevented using the molecules of the invention in conjunction with the methods of the present invention, that are caused by parasites include, but are not limited to, chlamydia and rickettsia.

According to one aspect of the invention, molecules of the invention comprising variant heavy chains have an enhanced antibody effector function towards an infectious agent, e.g., a pathogenic protein, relative to a comparable molecule comprising a wild-type Fc region. Examples of infectious agents include but are not limited to bacteria (e.g., Escherichia coli, Klebsiella pneumoniae, Staphylococcus aureus, Enterococcus faecials, Candida albicans, Proteus vulgaris, Staphylococcus viridans, and Pseudomonas aeruginosa), a pathogen (e.g., B-lymphotropic papovavirus (LPV); Bordatella pertussis; Boma Disease virus (BDV); Bovine coronavirus; Choriomeningitis virus; Dengue virus; a virus, E. coli; Ebola; Echovirus 1; Echovirus-11 (EV); Endotoxin (LPS); Enteric bacteria; Enteric Orphan virus; Enteroviruses; Feline leukemia virus; Foot and mouth disease virus; Gibbon ape leukemia virus (GALV); Gram-negative bacteria; Heliobacter pylori; Hepatitis B virus (HBV); Herpes Simplex Virus; HIV-1; Human cytomegalovirus; Human coronovirus; Influenza A, B & C; Legionella; Leishmania mexicana; Listeria monocytogenes; Measles virus; Meningococcus; Morbilliviruses; Mouse hepatitis virus; Murine leukemia virus; Murine gamma herpes virus; Murine retrovirus; Murine coronavirus mouse hepatitis virus; Mycobacterium avium-M; Neisseria gonorrhoeae; Newcastle disease virus; Parvovirus B 19; Plasmodium falciparum; Pox Virus; Pseudomonas; Rotavirus; Samonella typhiurium; Shigella; Streptococci; T-cell lymphotropic virus 1; Vaccinia virus).

In a specific embodiment, molecules of the invention enhance the efficacy of treatment of an infectious disease by enhancing phagocytosis and/or opsonization of the infectious agent causing the infectious disease. In another specific embodiment, molecules of the invention enhance the efficacy of treatment of an infectious disease by enhancing ADCC of infected cells causing the infectious disease.

In some embodiments, the molecules of the invention may be administered in combination with a therapeutically or prophylactically effective amount of one or additional therapeutic agents known to those skilled in the art for the treatment and/or prevention of an infectious disease. The invention contemplates the use of the molecules of the invention in combination with antibiotics known to those skilled in the art for the treatment and or prevention of an infectious disease. Antibiotics that can be used in combination with the molecules of the invention include, but are not limited to, macrolide (e.g., tobramycin (Tobi®), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

In certain embodiments, the molecules of the invention can be administered in combination with a therapeutically or prophylactically effective amount of one or more antifungal agents. Antifungal agents that can be used in combination with the molecules of the invention include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofuldin.

In some embodiments, the molecules of the invention can be administered in combination with a therapeutically or prophylactically effective amount of one or more anti-viral agent. Useful anti-viral agents that can be used in combination with the molecules of the invention include, but are not limited to, protease inhibitors, nucleoside reverse transcriptase inhibitors, non-nucleoside reverse transcriptase inhibitors and nucleoside analogs. Examples of antiviral agents include but are not limited to zidovudine, acyclovir, gangcyclovir, vidarabine, idoxuridine, trifluridine, and ribavirin, as well as foscamet, amantadine, rimantadine, saquinavir, indinavir, amprenavir, lopinavir, ritonavir, the alpha-interferons; adefovir, clevadine, entecavir, pleconaril.

6.6 Vaccine Therapy

The invention further encompasses using a composition of the invention to induce an immune response against an antigenic or immunogenic agent, including but not limited to cancer antigens and infectious disease antigens (examples of which are disclosed infra). The vaccine compositions of the invention comprise one or more antigenic or immunogenic agents to which an immune response is desired, wherein the one or more antigenic or immunogenic agents is coated with a variant antibody of the invention that has an enhanced affinity to FcγRIIIA. Although not intending to be bound by a particular mechanism of action, coating an antigenic or immunogenic agent with a variant antibody of the invention that has an enhanced affinity to FcγRIIIA, enhances the immune response to the desired antigenic or immunogenic agent by inducing humoral and cell-mediated responses. The vaccine compositions of the invention are particularly effective in eliciting an immune response, preferably a protective immune response against the antigenic or immunogenic agent.

In some embodiments, the antigenic or immunogenic agent in the vaccine compositions of the invention comprise a virus against which an immune response is desired. The viruses may be recombinant or chimeric, and are preferably attenuated. Production of recombinant, chimeric, and attenuated viruses may be performed using standard methods known to one skilled in the art. The invention encompasses a live recombinant viral vaccine or an inactivated recombinant viral vaccine to be formulated in accordance with the invention. A live vaccine may be preferred because multiplication in the host leads to a prolonged stimulus of similar kind and magnitude to that occurring in natural infections, and therefore, confers substantial, long-lasting immunity. Production of such live recombinant virus vaccine formulations may be accomplished using conventional methods involving propagation of the virus in cell culture or in the allantois of the chick embryo followed by purification.

In a specific embodiment, the recombinant virus is non-pathogenic to the subject to which it is administered. In this regard, the use of genetically engineered viruses for vaccine purposes may require the presence of attenuation characteristics in these strains. The introduction of appropriate mutations (e.g., deletions) into the templates used for transfection may provide the novel viruses with attenuation characteristics. For example, specific missense mutations which are associated with temperature sensitivity or cold adaption can be made into deletion mutations. These mutations should be more stable than the point mutations associated with cold or temperature sensitive mutants and reversion frequencies should be extremely low. Recombinant DNA technologies for engineering recombinant viruses are known in the art and encompassed in the invention. For example, techniques for modifying negative strand RNA viruses are known in the art, see, e.g., U.S. Pat. No. 5,166,057, which is incorporated herein by reference in its entirety.

Alternatively, chimeric viruses with “suicide” characteristics may be constructed for use in the intradermal vaccine formulations of the invention. Such viruses would go through only one or a few rounds of replication within the host. When used as a vaccine, the recombinant virus would go through limited replication cycle(s) and induce a sufficient level of immune response but it would not go further in the human host and cause disease. Alternatively, inactivated (killed) virus may be formulated in accordance with the invention. Inactivated vaccine formulations may be prepared using conventional techniques to “kill” the chimeric viruses. Inactivated vaccines are “dead” in the sense that their infectivity has been destroyed. Ideally, the infectivity of the virus is destroyed without affecting its immunogenicity. In order to prepare inactivated vaccines, the chimeric virus may be grown in cell culture or in the allantois of the chick embryo, purified by zonal ultracentrifugation, inactivated by formaldehyde or β-propiolactone, and pooled.

In certain embodiments, completely foreign epitopes, including antigens derived from other viral or non-viral pathogens can be engineered into the virus for use in the intradermal vaccine formulations of the invention. For example, antigens of non-related viruses such as HIV (gp160, gp120, gp41) parasite antigens (e.g., malaria), bacterial or fungal antigens or tumor antigens can be engineered into the attenuated strain.

Virtually any heterologous gene sequence may be constructed into the chimeric viruses of the invention for use in the intradermal vaccine formulations. Preferably, heterologous gene sequences are moieties and peptides that act as biological response modifiers. Preferably, epitopes that induce a protective immune response to any of a variety of pathogens, or antigens that bind neutralizing antibodies may be expressed by or as part of the chimeric viruses. For example, heterologous gene sequences that can be constructed into the chimeric viruses of the invention include, but are not limited to, influenza and parainfluenza hemagglutinin neuraminidase and fusion glycoproteins such as the HN and F genes of human PIV3. In yet another embodiment, heterologous gene sequences that can be engineered into the chimeric viruses include those that encode proteins with immuno-modulating activities. Examples of immuno-modulating proteins include, but are not limited to, cytokines, interferon type 1, gamma interferon, colony stimulating factors, interleukin-1, -2, -4, -5, -6, -12, and antagonists of these agents.

In yet other embodiments, the invention encompasses pathogenic cells or viruses, preferably attenuated viruses, which express the variant antibody on their surface.

In alternative embodiments, the vaccine compositions of the invention comprise a fusion polypeptide wherein an antigenic or immunogenic agent is operatively linked to a variant antibody of the invention that has an enhanced affinity for FcγRIIIA. Engineering fusion polypeptides for use in the vaccine compositions of the invention is performed using routine recombinant DNA technology methods and is within the level of ordinary skill.

The invention further encompasses methods to induce tolerance in a subject by administering a composition of the invention. Preferably a composition suitable for inducing tolerance in a subject, comprises an antigenic or immunogenic agent coated with a variant antibody of the invention, wherein the variant antibody has a higher affinity to FcγRIIB. Although not intending to be bound by a particular mechanism of action, such compositions are effective in inducing tolerance by activating the FcγRIIB meditated inhibitory pathway.

6.7 Compositions and Methods of Administering

The invention provides methods and pharmaceutical compositions comprising molecules of the invention (i.e., antibodies, polypeptides) comprising variant heavy chains having the Fc region of IgG2, IgG3 or IgG4. The invention also provides methods of treatment, prophylaxis, and amelioration of one or more symptoms associated with a disease, disorder or infection by administering to a subject an effective amount of a fusion protein or a conjugated molecule of the invention, or a pharmaceutical composition comprising a fusion protein or a conjugated molecule of the invention. In a preferred aspect, an antibody, a fusion protein, or a conjugated molecule, is substantially purified (i.e., substantially free from substances that limit its effect or produce undesired side-effects). In a specific embodiment, the subject is an animal, preferably a mammal such as non-primate (e.g., cows, pigs, horses, cats, dogs, rats etc.) and a primate (e.g., monkey such as, a cynomolgous monkey and a human). In a preferred embodiment, the subject is a human. In yet another preferred embodiment, the antibody of the invention is from the same species as the subject.

Various delivery systems are known and can be used to administer a composition comprising molecules of the invention (i.e., antibodies, polypeptides), comprising variant heavy chain having an Fc region of IgG2, IgG3 or IgG4, e.g., encapsulation in liposomes, microparticles, microcapsules, recombinant cells capable of expressing the antibody or fusion protein, receptor-mediated endocytosis (See, e.g., Wu and Wu, 1987, J. Biol. Chem. 262:4429-4432), construction of a nucleic acid as part of a retroviral or other vector, etc. Methods of administering a molecule of the invention include, but are not limited to, parenteral administration (e.g., intradermal, intramuscular, intraperitoneal, intravenous and subcutaneous), epidural, and mucosal (e.g., intranasal and oral routes). In a specific embodiment, the molecules of the invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

The invention also provides that the molecules of the invention (i.e., antibodies, polypeptides) comprising variant heavy chains having the Fc region of IgG2, IgG3 or IgG4, are packaged in a hermetically sealed container such as an ampoule or sachette indicating the quantity of antibody. In one embodiment, the molecules of the invention are supplied as a dry sterilized lyophilized powder or water free concentrate in a hermetically sealed container and can be reconstituted, e.g., with water or saline to the appropriate concentration for administration to a subject. Preferably, the molecules of the invention are supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, or at least 75 mg. The lyophilized molecules of the invention should be stored at between 2 and 8° C. in their original container and the molecules should be administered within 12 hours, preferably within 6 hours, within 5 hours, within 3 hours, or within 1 hour after being reconstituted. In an alternative embodiment, molecules of the invention are supplied in liquid form in a hermetically sealed container indicating the quantity and concentration of the molecule, fusion protein, or conjugated molecule. Preferably, the liquid form of the molecules of the invention are supplied in a hermetically sealed container at least 1 mg/ml, more preferably at least 2.5 mg/ml, at least 5 mg/ml, at least 8 mg/ml, at least 10 mg/ml, at least 15 mg/kg, at least 25 mg/ml, at least 50 mg/ml, at least 100 mg/ml, at least 150 mg/ml, at least 200 mg/ml of the molecules.

The amount of the composition of the invention which will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

For antibodies encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In one embodiment, the dosage of the molecules of the invention administered to a patient are 0.01 mg to 1000 mg/day, when used as single agent therapy. In another embodiment the molecules of the invention are used in combination with other therapeutic compositions and the dosage administered to a patient are lower than when said molecules are used as a single agent therapy.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a molecule of the invention, care must be taken to use materials to which the molecule does not absorb.

In another embodiment, the compositions can be delivered in a vesicle, in particular a liposome (See Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compositions can be delivered in a controlled release or sustained release system. Any technique known to one of skill in the art can be used to produce sustained release formulations comprising one or more molecules of the invention. See, e.g., U.S. Pat. No. 4,526,938; PCT publication WO 91/05548; PCT publication WO 96/20698; Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397; Cleek et al., 1997, “Biodegradable Polymeric Carriers for a bFGF Antibody for Cardiovascular Application,” Pro. Int'l. Symp. Control. Rel. Bioact. Mater. 24:853-854; and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in its entirety. In one embodiment, a pump may be used in a controlled release system (See Langer, supra; Sefton, 1987, CRC Crit. Ref. Biomed. Eng. 14:20; Buchwald et al., 1980, Surgery 88:507; and Saudek et al., 1989, N. Engl. J. Med. 321:574). In another embodiment, polymeric materials can be used to achieve controlled release of antibodies (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; See also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly-vinyl pyrrolidone), poly(vinyl alcohol), polyacrylamide, poly(ethylene glycol), polylactides (PLA), poly(lactide-co-glycolides) (PLGA), and polyorthoesters. In yet another embodiment, a controlled release system can be placed in proximity of the therapeutic target (e.g., the lungs), thus requiring only a fraction of the systemic dose (see, e.g., Goodson, in Medical Applications of Controlled Release, supra, vol. 2, pp. 115-138 (1984)). In another embodiment, polymeric compositions useful as controlled release implants are used according to Dunn et al. (See U.S. Pat. No. 5,945,155). This particular method is based upon the therapeutic effect of the in situ controlled release of the bioactive material from the polymer system. The implantation can generally occur anywhere within the body of the patient in need of therapeutic treatment. In another embodiment, a non-polymeric sustained delivery system is used, whereby a non-polymeric implant in the body of the subject is used as a drug delivery system. Upon implantation in the body, the organic solvent of the implant will dissipate, disperse, or leach from the composition into surrounding tissue fluid, and the non-polymeric material will gradually coagulate or precipitate to form a solid, microporous matrix (See U.S. Pat. No. 5,888,533).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control Rel. Bioact. Mater. 24:853-854; and Lam et al., 1997, Proc. Int'l. Symp. Control Rel. Bioact. Mater. 24:759-760, each of which is incorporated herein by reference in its entirety.

In a specific embodiment where the composition of the invention is a nucleic acid encoding an antibody, the nucleic acid can be administered in vivo to promote expression of its encoded antibody, by constructing it as part of an appropriate nucleic acid expression vector and administering it so that it becomes intracellular, e.g., by use of a retroviral vector (See U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (See e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

For antibodies, the therapeutically or prophylactically effective dosage administered to a subject is typically 0.1 mg/kg to 200 mg/kg of the subject's body weight. Preferably, the dosage administered to a subject is between 0.1 mg/kg and 20 mg/kg of the subject's body weight and more preferably the dosage administered to a subject is between 1 mg/kg to 10 mg/kg of the subject's body weight. The dosage and frequency of administration of antibodies of the invention may be reduced also by enhancing uptake and tissue penetration (e.g., into the lung) of the antibodies or fusion proteins by modifications such as, for example, lipidation.

Treatment of a subject with a therapeutically or prophylactically effective amount of molecules of the invention can include a single treatment or, preferably, can include a series of treatments. In a preferred example, a subject is treated with molecules of the invention in the range of between about 0.1 to 30 mg/kg body weight, one time per week for between about 1 to 10 weeks, preferably between 2 to 8 weeks, more preferably between about 3 to 7 weeks, and even more preferably for about 4, 5, or 6 weeks. In other embodiments, the pharmaceutical compositions of the invention are administered once a day, twice a day, or three times a day. In other embodiments, the pharmaceutical compositions are administered once a week, twice a week, once every two weeks, once a month, once every six weeks, once every two months, twice a year or once per year. It will also be appreciated that the effective dosage of the molecules used for treatment may increase or decrease over the course of a particular treatment.

6.7.1 Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of one or more molecules of the invention and a pharmaceutically acceptable carrier.

In one particular embodiment, the pharmaceutical composition comprises a therapeutically effective amount of one or more molecules of the invention comprising a variant heavy chain having the Fc region of IgG2, IgG3 or IgG4, wherein Fc region of said variant heavy chain binds FcγRIIIA and/or FcγRIIA with a greater affinity than a comparable molecule comprising a wild-type heavy chain having the Fc region of the same isotype binds FcγRIIIA and/or FcγRIIA and/or said variant heavy chain confers an effector function or mediates an effector function at least 2-fold more effectively than a comparable molecule comprising a wild-type heavy chain having an Fc region of the same isotype, and a pharmaceutically acceptable carrier. In another embodiment, the pharmaceutical composition comprises a therapeutically effective amount of one or more molecules of the invention comprising a variant heavy chain, wherein the Fc region of said variant heavy chain binds FcγRIIIA with a greater affinity than a comparable molecule comprising a wild-type heavy chain having an Fc region of the same isotype binds FcγRIIIA, and said variant heavy chain binds FcγRIIB with a lower affinity than a comparable molecule comprising a wild-type heavy chain having an Fc region of the same isotype binds FcγRIIB, and/or said variant heavy chain mediates an effector function at least 2-fold more effectively than a comparable molecule comprising a wild-type heavy chain having an Fc region of the same isotype, and a pharmaceutically acceptable carrier. In another embodiment, said pharmaceutical compositions further comprise one or more anti-cancer agents.

The invention also encompasses pharmaceutical compositions comprising a therapeutic antibody (e.g., tumor specific monoclonal antibody) that is specific for a particular cancer antigen, comprising one or more amino acid modifications in the heavy chain in accordance with the instant invention, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

6.7.2 Gene Therapy

In a specific embodiment, nucleic acids comprising sequences encoding molecules of the invention, are administered to treat, prevent or ameliorate one or more symptoms associated with a disease, disorder, or infection, by way of gene therapy. Gene therapy refers to therapy performed by the administration to a subject of an expressed or expressible nucleic acid. In this embodiment of the invention, the nucleic acids produce their encoded antibody or fusion protein that mediates a therapeutic or prophylactic effect.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression A Laboratory Manual, Stockton Press, NY (1990).

In a preferred aspect, a composition of the invention comprises nucleic acids encoding an antibody, said nucleic acids being part of an expression vector that expresses the antibody in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; and Zijlstra et al., 1989, Nature 342:435-438).

In another preferred aspect, a composition of the invention comprises nucleic acids encoding a fusion protein, said nucleic acids being a part of an expression vector that expresses the fusion protein in a suitable host. In particular, such nucleic acids have promoters, preferably heterologous promoters, operably linked to the coding region of a fusion protein, said promoter being inducible or constitutive, and optionally, tissue-specific. In another particular embodiment, nucleic acid molecules are used in which the coding sequence of the fusion protein and any other desired sequences are flanked by regions that promote homologous recombination at a desired site in the genome, thus providing for intrachromosomal expression of the fusion protein.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (See, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (See, e.g., PCT Publications WO 92/06180; WO 92/22635; WO92/20316; WO93/14188; WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contain nucleic acid sequences encoding a molecule of the invention (e.g., an antibody or a fusion protein) are used. For example, a retroviral vector can be used (See Miller et al., 1993, Meth. Enzymol. 217:581-599). These retroviral vectors contain the components necessary for the correct packaging of the viral genome and integration into the host cell DNA. The nucleic acid sequences encoding the antibody or a fusion protein to be used in gene therapy are cloned into one or more vectors, which facilitates delivery of the nucleotide sequence into a subject. More detail about retroviral vectors can be found in Boesen et al., (1994, Biotherapy 6:291-302), which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (Current Opinion in Genetics and Development 3:499-503, 1993, present a review of adenovirus-based gene therapy. Bout et al., (Human Gene Therapy, 5:3-10, 1994) demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a preferred embodiment, adenovirus vectors are used.

Adeno-associated virus (AAV) has also been proposed for use in gene therapy (see, e.g., Walsh et al., 1993, Proc. Soc. Exp. Biol. Med. 204:289-300 and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to, transfection, electroporation, microinjection, infection with a viral or bacteriophage vector, containing the nucleic acid sequences, cell fusion, chromosome-mediated gene transfer, microcellmediated gene transfer, spheroplast fusion, etc. Numerous techniques are known in the art for the introduction of foreign genes into cells (See, e.g., Loeffler and Behr, 1993, Meth. Enzymol. 217:599-618, Cohen et al., 1993, Meth. Enzymol. 217:618-644; and Clin. Pharma. Ther. 29:69-92, 1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the subject.

In an embodiment in which recombinant cells are used in gene therapy, nucleic acid sequences encoding an antibody or a fusion protein are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (See e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

6.7.3 Kits

The invention provides a pharmaceutical pack or kit comprising one or more containers filled with the molecules of the invention (i.e., antibodies, polypeptides comprising variant heavy chain containing the Fc region of IgG2, IgG3 or IgG4 and having at least one amino acid modification relative to a wodl type heavy chain having an Fc region of the same isotype). Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises one or more molecules of the invention. In another embodiment, a kit further comprises one or more other prophylactic or therapeutic agents useful for the treatment of cancer, in one or more containers. In another embodiment, a kit further comprises one or more cytotoxic antibodies that bind one or more cancer antigens associated with cancer. In certain embodiments, the other prophylactic or therapeutic agent is a chemotherapeutic. In other embodiments, the prophylactic or therapeutic agent is a biological or hormonal therapeutic.

6.8 Characterization and Demonstration of Therapeutic Utility

Several aspects of the pharmaceutical compositions, prophylactic, or therapeutic agents of the invention are preferably tested in vitro, in a cell culture system, and in an animal model organism, such as a rodent animal model system, for the desired therapeutic activity prior to use in humans. For example, assays which can be used to determine whether administration of a specific pharmaceutical composition is desired, include cell culture assays in which a patient tissue sample is grown in culture, and exposed to or otherwise contacted with a pharmaceutical composition of the invention, and the effect of such composition upon the tissue sample is observed. The tissue sample can be obtained by biopsy from the patient. This test allows the identification of the therapeutically most effective prophylactic or therapeutic molecule(s) for each individual patient. In various specific embodiments, in vitro assays can be carried out with representative cells of cell types involved in an autoimmune or inflammatory disorder (e.g., T cells), to determine if a pharmaceutical composition of the invention has a desired effect upon such cell types.

Combinations of prophylactic and/or therapeutic agents can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, combinations of prophylactic and/or therapeutic agents are tested in a mouse model system. Such model systems are widely used and well-known to the skilled artisan. Prophylactic and/or therapeutic agents can be administered repeatedly. Several aspects of the procedure may vary. Said aspects include the temporal regime of administering the prophylactic and/or therapeutic agents, and whether such agents are administered separately or as an admixture.

Preferred animal models for use in the methods of the invention are, for example, transgenic mice expressing human FcγRs on mouse effector cells, e.g., any mouse model described in U.S. Pat. No. 5,877,396 (which is incorporated herein by reference in its entirety) can be used in the present invention. Transgenic mice for use in the methods of the invention include, but are not limited to, mice carrying human FcγRIIIA; mice carrying human FcγRIIA; mice carrying human FcγRIIB and human FcγRIIIA; mice carrying human FcγRIIB and human FcγRIIA.

Preferably, mutations showing the highest levels of activity in the functional assays described above will be tested for use in animal model studies prior to use in humans. Antibodies harboring the Fc mutants identified using the methods of the invention and tested in ADCC assays, including ch4D5 and ch520C9, two anti-Erb-B2 antibodies, and chCC49, an anti-TAG72 antibody, are preferred for use in animal models since they have been used previously in xenograft mouse model (Hudsiak et al., 1989, Mol Cell Biol. 9: 1165-72; Lewis et al., 1993, Cancer Immunol. Immunother. 37: 255-63; Bergman et al., 2001 Clin. Cancer Res. 7: 2050-6; Johnson et al., 1995, Anticancer Res. 1387-93). Sufficient quantities of antibodies may be prepared for use in animal models using methods described supra, for example using mammalian expression systems and IgG purification methods disclosed and exemplified herein.

Mouse xenograft models may be used for examining efficacy of mouse antibodies generated against a tumor specific target based on the affinity and specificity of the CDR regions of the antibody molecule and the ability of the Fc region of the antibody to elicit an immune response (Wu et al., 2001, Trends Cell Biol. 11: S2-9). Transgenic mice expressing human FcγRs on mouse effector cells are unique and are tailor-made animal models to test the efficacy of human Fc-FcγR interactions. Pairs of FcγRIIIA, FcγRIIIB and FcγRIIA transgenic mouse lines generated in the lab of Dr. Jeffrey Ravetch (Through a licensing agreement with Rockefeller U. and Sloan Kettering Cancer center) can be used such as those listed in the Table 25 below.

TABLE 25
Mice Strains

| Strain Background | Human FcR |
|---|---|
| Nude/CD16A KO | None |
| Nude/CD16A KO | FcγRIIIA |
| Nude/CD16A KO | FcγR IIA |
| Nude/CD16A KO | FcγR IIA and IIIA |
| Nude/CD32B KO | None |
| Nude/CD32B KO | FcγR IIB |

Preferably molecules of the invention showing both enhanced binding to FcγRIIIA and reduced binding to FcγRIIB, increased activity in ADCC and phagocytosis assays are tested in animal model experiments. The animal model experiments examine the increase in efficacy of variant heavy chain bearing antibodies in FcγRIIIA transgenic, nude mCD16A knockout mice compared to a control which has been administered native antibody. Preferably, groups of 8-10 mice are examined using a standard protocol. An exemplary animal model experiment may comprise the following steps: in a breast cancer model, ˜2×10⁶ SK-BR-3 cells are injected subcutaneously on day 1 with 0.1 mL PBS mixed with Matrigel (Becton Dickinson). Initially a wild type chimeric antibody and isotype control are administered to establish a curve for the predetermined therapeutic dose, intravenous injection of 4D5 on day 1 with an initial dose of 4 μg/g followed by weekly injections of 2 μg/g. Tumor volume is monitored for 6-8 weeks to measure progress of the disease. Tumor volume should increase linearly with time in animals injected with the isotype control. In contrast very little tumor growth should occur in the group injected with 4D5. Results from the standard dose study are used to set an upper limit for experiments testing the Fc mutants. These studies are done using subtherapeutic doses of the Fc mutant containing antibodies. A one tenth dose was used on xenograft models in experiments done in FcγRIIB knockout mice, see, Clynes et al., 2000, Nat. Med. 6: 443-6, with a resultant block in tumor cell growth. Since the mutants of the invention preferrably show an increase in FcγRIIIA activation and reduction in FcγRIIB binding the mutants are examined at one tenth therapeutic dose. Examination of tumor size at different intervals indicates the efficacy of the antibodies at the lower dose. Statistical analysis of the data using t test provides a way of determining if the data is significant. Fe mutants that show increased efficacy are tested at incrementally lower doses to determine the smallest possible dose as a measure of their efficacy.

The anti-inflammatory activity of the combination therapies of invention can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993). Experimental and spontaneous animal models of inflammatory arthritis and autoimmune rheumatic diseases can also be used to assess the anti-inflammatory activity of the combination therapies of invention. The following are some assays provided as examples, and not by limitation.

The principle animal models for arthritis or inflammatory disease known in the art and widely used include: adjuvant-induced arthritis rat models, collagen-induced arthritis rat and mouse models and antigen-induced arthritis rat, rabbit and hamster models, all described in Crofford L. J. and Wilder R. L., “Arthritis and Autoimmunity in Animals”, in Arthritis and Allied Conditions: A Textbook of Rheumatology, McCarty et al., (eds.), Chapter 30 (Lee and Febiger, 1993), incorporated herein by reference in its entirety.

The anti-inflammatory activity of the combination therapies of invention can be assessed using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hansra P. et al., “Carrageenan-Induced Arthritis in the Rat,” Inflammation, 24(2): 141-155, (2000). Also commonly used are zymosan-induced inflammation animal models as known and described in the art.

The anti-inflammatory activity of the combination therapies of invention can also be assessed by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547, (1962). This assay has been used as a primary in vivo screen for the anti-inflammatory activity of most NSAIDs, and is considered predictive of human efficacy. The anti-inflammatory activity of the test prophylactic or therapeutic agents is expressed as the percent inhibition of the increase in hind paw weight of the test group relative to the vehicle dosed control group.

Additionally, animal models for inflammatory bowel disease can also be used to assess the efficacy of the combination therapies of invention (Kim et al., 1992, Scand. J. Gastroentrol. 27:529-537; Strober, 1985, Dig. Dis. Sci. 30(12 Suppl):3S-10S). Ulcerative cholitis and Crohn's disease are human inflammatory bowel diseases that can be induced in animals. Sulfated polysaccharides including, but not limited to amylopectin, carrageen, amylopectin sulfate, and dextran sulfate or chemical irritants including but not limited to trinitrobenzenesulphonic acid (TNBS) and acetic acid can be administered to animals orally to induce inflammatory bowel diseases.

Animal models for autoimmune disorders can also be used to assess the efficacy of the combination therapies of invention. Animal models for autoimmune disorders such as type 1 diabetes, thyroid autoimmunity, sytemic lupus eruthematosus, and glomerulonephritis have been developed (Flanders et al., 1999, Autoimmunity 29:235-246; Krogh et al., 1999, Biochimie 81:511-515; Foster, 1999, Semin. Nephrol. 19:12-24).

Further, any assays known to those skilled in the art can be used to evaluate the prophylactic and/or therapeutic utility of the combinatorial therapies disclosed herein for autoimmune and/or inflammatory diseases.

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The anti-cancer activity of the therapies used in accordance with the present invention also can be determined by using various experimental animal models for the study of cancer such as the SCID mouse model or transgenic mice or nude mice with human xenografts, animal models, such as hamsters, rabbits, etc. known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties.

Preferred animal models for determining the therapeutic efficacy of the molecules of the invention are mouse xenograft models. Tumor cell lines that can be used as a source for xenograft tumors include but are not limited to, SKBR3 and MCF7 cells, which can be derived from patients with breast adenocarcinoma. These cells have both erbB2 and prolactin receptors. SKBR3 cells have been used routinely in the art as ADCC and xenograft tumor models. Alternatively, OVCAR3 cells derived from a human ovarian adenocarcinoma can be used as a source for xenograft tumors.

The protocols and compositions of the invention are preferably tested in vitro, and then in vivo, for the desired therapeutic or prophylactic activity, prior to use in humans. Therapeutic agents and methods may be screened using cells of a tumor or malignant cell line. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, decreased growth and/or colony formation in soft agar or tubular network formation in three-dimensional basement membrane or extracellular matrix preparation, etc.

Compounds for use in therapy can be tested in suitable animal model systems prior to testing in humans, including but not limited to in rats, mice, chicken, cows, monkeys, rabbits, hamsters, etc., for example, the animal models described above. The compounds can then be used in the appropriate clinical trials.

Further, any assays known to those skilled in the art can be used to evaluate the prophylactic and/or therapeutic utility of the combinatorial therapies disclosed herein for treatment or prevention of cancer, inflammatory disorder, or autoimmune disease.

6.9 Diagnostic Assays

The invention encompasses molecules, e.g., antibodies, with altered affinities and avidities for one or more FcγRs. The antibodies of the invention with enhanced affinity and avidity for one or more FcγRs are particularly useful in cellular systems (for example for research or diagnostic purposes) where the FcγRs are expressed at low levels. Although not intending to be bound by a particular mechanism of action, the molecules of the invention with enhanced affinity and avidity for a particular FcγR are valuable as research and diagnostic tools by enhancing the sensitivity of detection of FcγRs which are normally undetectable due to a low level of expression.

### 7. EXAMPLES

### 7.1 Correlation Binding to Activating FcγR and Enhanced Effector Function

Heavy chain mutations which enhance FcγRIIIA and FcγRIIA binding and reduce binding to FcγRIIB have been suggested to positively correlate with the appearance or improvement of both ADCC and complement function, see e.g., WO 04/063351. This hypothesis was tested by cloning promising mutations into the heavy chain of the chimeric anti-FITC antibody ch4-420 for BIAcore assays and antitumor monoclonal antibody 4D5 (anti-HER2/neu), chimeric anti-CD32B monoclonal antibody ch2B6 and the anti-CD20 antibody Rituxin™ for effector function assays. Fc modifications which improved or conferred binding to activating Fc receptors as determined by BIAcore assay, were shown to improve or confer effector function to the variant antibodies as determined by standard ADCC assays. The increase in binding and/or effector function activity was further shown to be a function of the Fc modification and not the target antigen.

Materials and Methods

Preparation of Antibodies: Fc Mutations which Improved or Conferred Binding to activating FcγRs were cloned into the heavy chains of the antibodies using standard techniques. The chimeric antibodies were expressed by transient transfection into 293H cells and purified over a protein G column.

BIAcore Assay: The binding of ch4-420 antibodies comprising variant Fc regions to FcγRs was analyzed for alterations in kinetic parameters using a BIAcore assay (BIAcore instrument 1000, BIAcore Inc., Piscataway, N.J.) and associated software as described in Section 5.2.1 and 5.3.2). The FcγRIIIA and FcγRIIA used in this assay were soluble monomeric proteins, the extracellular region of the receptors joined to the linker-AVITAG sequence as described in Section 5.2.1, supra. The FcγRIIB used in this assay was a soluble dimeric protein prepared in accordance with the methodology described in U.S. Provisional Application No. 60/439,709 filed on Jan. 13, 2003, which is incorporated herein by reference. Briefly, the FcγRIIB used was the extracellular domain of FcγRIIB fused to the hinge-CH2-CH3 domain of human IgG2.

BSA-FITC (36 μg/mL in 10 mM Acetate Buffer at pH 5.0) was immobilized on one of the four flow cells (flow cell 2) of a sensor chip surface through amine coupling chemistry (by modification of carboxymethyl groups with mixture of NHS/EDC) such that about 5000 response units (RU) of BSA-FITC was immobilized on the surface. Following this, the unreacted active esters were “capped off” with an injection of 1M Et-NH2. Once a suitable surface was prepared, ch 4-4-20 antibodies carrying the Fc mutations were passed over the surface by one minute injections of a 20 μg/mL solution at a 5 μL/mL flow rate. The level of ch-4-4-20 antibodies bound to the surface ranged between 400 and 700 RU. Next, dilution series of the receptor (FcγRIIIA and FcγRIIB-Fc fusion protein) in HBS-P buffer (10 mM HEPES, 150 mM NaCl, 0.005% Surfactant P20, 3 mM EDTA, pH 7.4) were injected onto the surface at 100 μL/min Antibody regeneration between different receptor dilutions was carried out by single 5 second injections of 100 mM NaHCO₃ pH 9.4; 3M NaCl.

The same dilutions of the receptor were also injected over a BSA-FITC surface without any ch-4-4-20 antibody at the beginning and at the end of the assay as reference injections.

Once an entire data set was collected, the resulting binding curves were globally fitted using computer algorithms supplied by the manufacturer, BIAcore, Inc. (Piscataway, N.J.). These algorithms calculate both the Kₒₙ and K_{off}, from which the apparent equilibrium binding constant, K_{D} is deduced as the ratio of the two rate constants (i.e., K_{off}/Kₒₙ). More detailed treatments of how the individual rate constants are derived can be found in the BIAevaluaion Software Handbook (BIAcore, Inc., Piscataway, N.J.).

Binding curves for two different concentrations (200 nM and 800 nM for activating FcγRs and 200 nM and 400 nM for FcγRIIB fusion protein) were aligned and responses adjusted to the same level of captured antibodies, and the reference curves were subtracted from the experimental curves. Association and dissociation phases were fitted separately. Dissociation rate constant was obtained for interval 32-34 sec of the dissociation phase; association phase fit was obtained by a 1:1 Langmuir model and base fit was selected on the basis Rₘₐₓ and chi² criteria.

Binding ability of antibodies comprising the Fc variant regions was characterized by cloning the mutations into appropriate antibodies, e.g., 4D5 or 2B6, and immunostaining target cells with either FITC conjugated variant antibody or the variant antibodies and a PE-conjugated polyclonal F(ab)₂ goat anti-human Fc antibody (Jackson Immunoresearch Laboratories, Inc.). FACS analysis was used to quantitate the staining.

ADCC assay: The chimeric variant antibodies were tested in an ADCC or CDC assay as described supra (Section 5.3).

Effector cell preparation: Peripheral blood mononuclear cells (PBMC) were purified by Ficoll-Paque (Pharmacia, 17-1440-02) Ficoll-Paque density gradient centrifugation from normal peripheral human blood (Biowhittaker/Poietics, 1W-406). Blood was shipped the same day at ambient temperature, and diluted 1:1 in PBS and glucose (1 g/1 L) and layered onto Ficoll in 15 mL conical tubes (3 mL Ficoll; 4 mL PBS/blood) or 50 mL conical tubes (15 mL: Ficoll; 20 mL PBS/blood). Centrifugation was done at 1500 rpm (400 rcf) for 40 minutes at room temperature. The PBMC layer was removed (approximately 4-6 mL from 50 mL conical tube) and diluted 1:10 in PBS (which contains no Ca²⁺ or Mg²⁺) in a 50 mL conical tube, and spun for an additional ten minutes at 1200 rpm (250 rcf) at room temperature. The supernatant was removed and the pellets were resuspended in 10-12 mL PBS (which contains no Ca²⁺ or Mg²⁺), transferred to 15 mL conical tubes, and spun for another 10 minutes at 1200 rpm at room temperature. The supernatant was removed and the pellets were resuspended in a minimum volume (1-2 mL) of media (Isocove's media (IMDM)+10% fetal bovine serum (FBS), 4 mM Gln, Penicillin/Streptomycin (P/S)). The resuspended PBMC were diluted to the appropriate volume for the ADCC assay; two fold dilutions were done in an ELISA 96 well plate (Nunc F96 MaxiSorp Immunoplate). The yield of PBMC was approximately 3-5×10⁷ cells per 40-50 mL of whole blood.

Target cell preparation: Target cells used in the assay were: for 4D5 antibodies, SK-BR-3 cells (high Her2/neu expression, ATCC Accession number HTB-30; Trempe et al., 1976, Cancer Res. 33-41) and HT29 cells (low Her2/neu expression, ATCC Accession number HTB-38); for ch2B6 antibodies, Daudi cells (ATCC Accession number CCL-213; Klein et al., 1968, Cancer Res. 28: 1300-10) or BK41 cells (both high CD32B expression) and Ramos cells (low CD32B expression, ATCC Accession number CRL-1596); for the Rituxin™ antibody, CHO cells that were engineered to express both CD32B and CD20 using standard techniques; K562 cells (ATCC Accession number CCL-243) were used as control cells for NK activity. Target cells were labeled with europium chelate bis(acetoxymethyl) 2,2″:6′,2″ terpyridine 6,6′ dicarboxylate (BATDA reagent; Perkin Elmer DELFIA reagent; C136-100). Suspension cells, e.g., Daudi cells, were spun down; the attachment dependent cells, e.g., SK-BR-3 cells, were trypsinized for 2-5 minutes at 37° C., 5% CO₂ and the media was neutralized prior to being spun down at 200-350 G. The number of target cells used in the assays was about 4-5×10⁶ cells and it did not exceed 5×10⁶ since labeling efficiency was best with as few as 2×10⁶ cells. Once the cells were spun down, the media was aspirated to 0.5 mL in 15 mL Falcon tubes. 2.5 μl of BATDA reagent was added and the mixture was incubated at 37° C., 5% CO₂ for 30 minutes. Cells were washed twice in 10 mL PBS and 0.125 mM sulfinpyrazole (“SP”; SIGMA S-9509); and twice in 10 mL assay media (cell media+0.125 mM sulfinpyrazole). Cells were resuspended in 1 mL assay media, counted and diluted.

When SK-BR-3 cells were used as target cells after the first PBS/SP wash, the PBS/SP was aspirated and 500 μg/mL of FITC was added (PIERCE 461110) in IMDM media containing SP, Gln, and P/S and incubated for 30 minutes at 37° C., 5% CO₂. Cells were washed twice with assay media; resuspended in 1 mL assay media, counted and diluted.

Antibody Opsonization: Once target cells were prepared as described supra, they were opsonized with the appropriate antibodies. In the case of Fc variants, 50 μL of 1×10⁵ cells/mL were added to 2× concentration of the antibody harboring the Fc variant. Final concentrations of antibodies were standard in the art for ADCC assays, e.g., 1-100 ng/mL, and may be routinely determined by a skilled worker.

Opsonized target cells were added to effector cells to produce an effector:target ratio of 75:1 in the case of the 4-4-20 antibodies with Fc variants. In the case of the Ch4D5 or 2B6 antibodies with Fc variants, effector: target ratio of 50:1 or 75:1 were achieved. Effective PBMC gradient for the assay ranges from 100:1 to 1:1. Spontaneous release (SR) was measured by adding 100 μL of assay media to the cells; maximal release (MR) was measured by adding 4% TX-100. Cells were spun down at 200 rpm in a Beckman centrifuge for 1 minute at room temperature at 57 G. Cells were incubated for 3-3.5 hours at 37° C., 5% CO₂. After incubation, the cells were spun at 1000 rpm in a Beckman centrifuge (about 220×g) for five minutes at 110° C. 20 μl of supernatant was collected; 200 μL of Eu solution was added and the mixture was shaken for 15 minutes at room temperature at 120 rpm on a rotary shaker. The fluorescence was quantitated in a time resolved fluorometer (Victor 1420, Perkin Elmer)

For all antibodies, the effects of antigen density on binding or on cell lysis by ADCC/CDC were tested by using cells with high or low expression of antigen. Antigen density was determined using Quantum™ Simply Cellular® kit from Bangs Laboratories, Inc. (Fishers, Ind.) according to the manufacturer's instructions.

Results

FIGS. 3 and 4 show the capture of 4D5 antibodies with mutant Fe regions on the BSA-FITC-immobilized sensor chip. BIAcore data was analyzed as described in Section 6.1. Either triple mutants (FIG. 3) or quadruple mutants (FIG. 4) showed reduced K_{d} to the activating Fe receptors and increased K_{d} to the inhibitory Fe receptor.

Although the Fc mutant 31/60 (P247L; N421K; D270E) did not enhance 4D5 binding to cells expressing low levels of Her2/neu (FIG. 5), this modification, as well as variants 71 (D270E; G316D; R416G), 59/60 (K370E; P396L; D270E), 55/60 (R255L; P396L; D270E), 51/60 (Q419H; P396L; D270E), 55/60/F243L (R255L; P396L; D270E; F243L), and 74/P396L (F243L; R292P; V305I; P396L) improved the wild-type ADCC mediated lysis of cells expressing low levels of antigen (FIGS. 6 and 7).

When similar Fc mutations, variants 31/60, 59/60, and 71, are introduced into an antibody with only limited binding to cells expressing low levels of antigen and no native effector function on the same cells, the results are more dramatic. FIG. 8 demonstrates that wild-type ch2B6 binding to Ramos cells can be substantially improved by the introduction the Fe mutations of variant 31/60 and 59/60. Similarly, effector function can be introduced by Fe mutations. Where the wild-type antibody has no detectable effector function, Fe mutations can result in a gain-of-function phenotype. Mutations which improved the binding of ch2B6 to Ramos cells also enabled the mutated antibody to mediate ADCC, variant 31/60, or CDC, variants 31/60 and 71 (FIGS. 9 and 10, respectively). FIGS. 11 and 12 also show the spectrum of response available, dependent on the specific mutation. Where the wild type ch2B6 antibody is capable of mediating at least some effector function, e.g. in cells with high expression of CD32B, Daudi cells, the same Fe mutations, variant 31/60 and 71, improve the effect (FIG. 11).

The increase in ADCC activity was shown to be a function of the Fe modification and not the target antigen. The mutation variant 55/60, previously identified as improving ADCC activity in 4D5 antibody, conferred effector function to the anti-CD20 antibody, Rituxin™. FIGS. 12 A and B show that the engineered CHO cell line expressed similar levels of CD32B and CD20 when tested with FITC-conjugated 2B6 or Rituxin™, respectively. Although the cells were sensitive to ADCC mediated by wild-type 2B6, ADCC activity was completely undetectable using wild-type Rituxin™ (FIG. 13 A). The introduction of the mutation variant 55/60 into Rituxin™, as in 4D5, was, however, able to confer effector function to the modified antibody (FIG. 13 B).

Possible mechanisms by which the mutated antibodies were able to improve both binding and effector function were observed when the binding affinities of variant ch2B6 antibodies to FcγRIIB were correlated with their ability to bind Ramos cells (FIG. 14 A-B). For example, variant 55/60 had both the highest k_{off} and binding affinity to Ramos cells. It is theorized the limited ability of the wild-type antibody to bind FcγRIIB is due to Fc-FcγRIIB interaction, effectively withdrawing the additional cell surface receptors from further antibody binding. The theory was investigated by challenging opsonized Ramos cells with CD16A, an activating FcγR. In accord with the theory, at low antigen density, Fc-engineered ch2B6, but not wild type Fe, was able bind the activating receptor (FIG. 15).

### 7.2 Variant Antibody Mediated Tumor Growth Control in an In Vivo Tumor Model

Heavy chain mutations identified as comprising enhanced affinity for FcγIIIA and/or FcγIIA were further analyzed for relative efficacy of tumor control using an in vivo tumor model system.

Materials and Methods

Antibodies harboring heavy chain mutants were tested for anti-tumor activity in a murine xenograft system. Balbc/nude mice injected subcutaneously with 5×10⁶ Daudi cells in approximately 0.10 ml of HBSS and subsequently monitored for general signs of illness, e.g. weight gain/loss and alteration in grooming activity. Without treatment, this model system resulted in 100% mortality with an average survival time of approximately 2 weeks post tumor cell inoculation. Treatment consisted of doses of wild-type antibody or antibody comprising a variant heavy chain administered at weekly intervals. Animals administered buffer alone according to the same schedule served as control. Tumor weight was calculated based on estimated volume of the subcutaneous tumor as determined by caliper measurement according to the formula (width²×length)/2.

### Results

At weekly intervals, mice inoculated with Daudi cells received wild-type humanized 2B6 (“h2B6”), humanized 2B6 comprising mutant FcMG0088 (F243L, R292P, Y300L, V305I P396L) (“h2B6 0088”) or buffer alone. Wild-type and Fe mutant h2B6 antibody showed similar levels of tumor suppression at the highest dose schedule tested, weekly doses of 25 μg (FIGS. 16 A and B). However, significant differences in antibody efficacy were observed when dosages were reduced. 100 and 10 fold reduction in wild-type h2B6 dosages provided no greater tumor control than administration of buffer alone (FIG. 16 A). In contrast, h2B60088 provided significant protection at weekly doses of 2.5 μg and at least limited protection at weekly doses of 0.25 μg (FIG. 18 B).

The protection conferred by even the lowest dose of Fe mutant antibody was confirmed in survival comparisons. At 11 weeks, 4 out of 7 mice remained alive in the group treated with 0.25 μg doses of h2B6 0088 compared to only 1 out of 7 in the group treated with the same dose of wild-type h2B6 (FIGS. 17 A & B).

### 7.3 Effect of Mutations Identified as Enhancing ADCC Function in ADCC Assays Using Tumor Cells Isolated from RITUXAN Treated Patients

Heavy cahin mutations which enhance FcγRIIIA and FcγRIIA binding, reduce binding to FcγRIIB and enhance ADCC and/or complement function (Section 6.1) were cloned into the anti-CD20 antibody Rituxin™ using standard techniques. These chimeric antibodies were expressed by transient transfection into 293H cells and purified over a protein G column. The variant antibodies were tested in an ADCC or CDC assay as described, supra, in cells isolated from RITUXAN® treated patients.

During the course of phase I and phase II clinical trials of Rituximab, lymphoma cells from biopsy specimens obtained from patients with B cell lymphoma prior to receiving the antibody were collected. Participating patients underwent surgical removal of a lymph node near the surface of the body. This was done using a local anesthetic. A portion of the tissue was analyzed by routine histopathology in the pathology lab. A portion of the lymph node was used to make a cell suspension for the in vitro studies.

Additionally, pre- and post-treatment PBMC via leukapheresis in some of the patients were collected to study the effector cells and T cell immune response after Rituximab treatment. Peripheral blood T cells and effector cells were collected via leukapheresis from patients treated with Rituximab. Participating patients underwent leukapheresis before the Rituximab treatment and one month after completion of the treatment to collect the T lymphocytes and effector cells. The collected blood components were mixed with an anti-coagulant (ACD-A) as it was drawn to prevent clotting. The effector cells collected via leukapheresis were used to determine if effector cells of different FcγR genotypes mediate ADCC differently.

Results

The results of the ADCC assays for the different Fc Engineered rituximab antibodies in six of the patients are shown in FIGS. 18A-F. Tables 26 and 27 provide a ranking of the effectiveness of the antibodies in six patients with 1 being the most effective for that patient and 11 being the least effective for that patient. A normal donor provided PBMC for this experiment. The genotype of the normal donor was heterozygous for the FcRIIIA 158V and FcRIIA 131R alleles. In most patients, the Fc engineered rituximab antibodies showed an improvement over rituximab in ADCC activity.

TABLE 26
(10:1 Effector:Target Ratio)

| | | | Fc Mutant | | | | | | | | |
|---|---|---|---|---|---|---|---|---|---|---|---|
| | IgG1 | Rituximab | 55/60/300L | 51/60 | 52/60 | 59/60 | 38/60 | 59 | 51 | 31/60 | 55/60/292G |
| Patient 1 | 11 | 10 | 5 | 3 | 4 | 1 | 9 | 8 | 7 | 6 | 2 |
| Patient 2 | 11 | 9 | 2 | 10 | 4 | 1 | 7 | 3 | 6 | 5 | 8 |
| Patient 3 | 11 | 10 | 3 | 4 | 8 | 2 | 9 | 5 | 7 | 6 | 1 |
| Patient 4 | 11 | 9 | 1 | 6 | 8 | 5 | 10 | 7 | 3 | 4 | 2 |
| Patient 5 | 11 | 7 | 8 | 10 | 2 | 1 | 9 | 3 | 6 | 5 | 4 |
| Patient 6 | 11 | 10 | 8 | 4 | 1 | 2 | 6 | 5 | 9 | 7 | 2 |

TABLE 27
(30:1 Effector:Target Ratio)

| | | | Fc Mutant | | | | | | | | |
|---|---|---|---|---|---|---|---|---|---|---|---|
| | IgG1 | Rituximab | 55/60/300L | 51/60 | 52/60 | 59/60 | 38/60 | 59 | 51 | 31/60 | 55/60/292G |
| Patient 1 | 11 | 10 | 6 | 7 | 8 | 2 | 9 | 4 | 5 | 3 | 1 |
| Patient 2 | 11 | 8 | 1 | 4 | 5 | 2 | 6 | 3 | 10 | 7 | 9 |
| Patient 3 | 11 | 8 | 2 | 1 | 3 | 6 | 7 | 5 | 10 | 4 | 10 |
| Patient 4 | 11 | 5 | 1 | 2 | 9 | 3 | 8 | 6 | 10 | 4 | 7 |
| Patient 5 | 11 | 9 | 2 | 5 | 6 | 1 | 10 | 4 | 8 | 3 | 7 |
| Patient 6 | 11 | 10 | 6 | 8 | 4 | 1 | 2 | 3 | 9 | 5 | 7 |

As shown in FIG. 18 A, rituximab has minimal ADCC killing activity as compared to the other engineered rituximab antibodies tested. Patient 1 fits our definition of a non-responder (i.e., is refractory) to rituximab treatment (FIG. 18 A). In contrast, in patient 2, wild-type rituximab shows some ADCC activity; however all tested variants except 59/60 and 52/60 exhibited improved ADCC activity.

### 7.4 Characterization of Non-IgG1 Antibodies Comprising Heavy Chain Variants

Heavy chain mutations originally identified in the context of the IgG1 isotype were cloned into antibodies comprising an IgG2 or IgG3 Fc region to test whether the identified mutations influenced the functional characteristics of the antibody, i.e., binding or effector function activity, independent of the IgG isotype. Antibodies comprising non-IgG1 Fc regions and selected heavy chain mutations were compared to antibodies comprising IgG1 fc regions harboring the same heavy chain mutations. BIAcore and ADCC analysis indicated that the effects of heavy chain mutations were heavily influenced by isotype selection.

Construction of Antibodies: Antibodies were constructed to compare the effects of Fc mutations in the context of varying IgG isotypes. Using standard techniques, the Fc domain of ch4D5 antibody (IgG1) was replaced with that of an IgG2 or IgG3 antibody. The tested antibodies thus comprised the CH1 and hinge region of IgG1 and the Fc region of IgG2 or IgG3 (FIG. 19). The wild-type Fc region of IgG2, however, binds only FcγRIIA 131H, severely limiting comparisons to antibodies comprising the Fc regions of other isotypes. Mutations which expand the binding repitoire and effector function activity of IgG2 Fc (originally identified in Chappel et al., 1991, Proc. Natl. Acad. Sci. USA 88:9036-9040, which is hereby incorporated by reference in its entirety) were therefore introduced by site directed mutatgenesis into the IgG2 Fc region used for this experiment, creating ch4D5 MgFc2006. MgFc2006 served as the backbone for all further IgG2 mutations analyzed. The alignment of wild type IgG1 Fc region, wild type IgG2 Fc region (MgFc2010) and IgG2 Fc region comprising the muations of Chappel et al. (a substitution at position 233 with glutamic acid, at position 234 with leucine, at position 235 with leucine and an insertion at position 237 with glycine) (MgFc2006) are provided in FIG. 20.

SPR Analysis: Kinetic parameters of ch4D5 antibodies comprising variant heavy chains were determined by surface plasmon resonance analysis (“SPR” or “BIAcore”), described in Sections 5.3.2 and 6.1. Antibodies were injected at a flow rate of 5 μl/min for 240 sec. over the surface of a recombinant human ErbB2/Fcaglycosyl chimera immobilized at high density on a CM-5 chip. Soluble receptors FcγRIIIA (158V) and FcγRIIIA (158F) were injected in duplicates at a flow rate of 50 μl/min for 120 sec. at concentrations of 400 and 800 nM, respectively. Soluble receptors FcγRIIB and FcγRIIB (131H) were injected at a concentration of 200 nM (binding site concentration). Real time binding curves for soluble receptors were normalized by the level of captured antibody at the moment of injection. Steady state response units and dissociation rate constant, K_{off}, were calculated by BIAevaluation software.

ADCC Assay: ADCC activity of antibodies was determined by ADCC assays, described in Section 5.3 and 6.1. Target cells were SKBR lymphoma cells. Target cells at a concentration of 1×10⁷ cells/mL were labeled with 100 μCi Indium-111 oxine (Amersham Health) at room temperature for 15-30 minutes. Unicorporated Indium-111 was removed by 4 sequential washes with cell media. Target cells were opsonized with antibodies of the invention and combined with PMBC in U-bottom 96 well plates at an effector to target ratio of 75:1. Approximately 500o Target cells were used per well. Following an 18 hr incubation in an incubator at 37° C., 5% CO₂, cell supernatants were harvested (Skatron Supernatant Collection System, Molecular Devices) and released Indium-111 was quantified using a gamma counter (Wallac 1470, Perkin Elmer). Maximal release (MR) and spontaneous release (SR) were determined by incubation of target cells with 2% Triton X-100 in cell media and cell media, respectively. Antibody independent cellular cytotoxicity (AICC) was measured by incubation of target and effector cells in the absence of antibody. All assays were performed in triplicate and the mean percentage specific lysis was calculated as (Experimental-AICC)/(MR-SR)×100.

Results

Functional Characteristisation of MgFc2006: SPR analysis of ch4D5 (IgG1) and ch4D5 MgFc2006 binding to FcγRIIA (158V) and FcγRIIA (158F) revealed that the two antibodies bound with similar affinity to both alleles CD16, demonstrated by similar steady state dissociation constants (FIG. 21). Both antibodies also demonstrated equivalent effector function activity when tested in an ADCC assay against SKBR target cells (FIG. 22). FIG. 22 also contrasts the effector function activity of MgFc2006 to that of ch4D5 antibody comprising a wild type IgG2 Fc region (MgFc2010). The results demonstrate that, although the mutations used in MgFc2006 were identified by Chappel et al. to convey enhanced affinity to CD64, the mutations also enhance CD 16A-associated functionality as well. Further, the similar binding characteristics of wt4D5 (IgG1) and MgFc2006 (comprising a variant IgG2 Fc region) allow a direct comparison of the effect of isotype background on heavy chain mutations.

Comparison of mutations MgFc0088 and MgFc0155 in the context of IgG1 and IgG2: Heavy chain mutations previously identified by the Inventors to enhance both binding affinity to activating FcγRs and effector function activity in the context of IgG1 were cloned into the MgFc2006. Mutations corresponding to the IgG1 MgFc0155 (243L, 292P, Y300L) and IgG1 MgFc088 (243L, 292P, 300L, 305I, 396L) were introduced into MgFc2006 to generate MgFc2012 and MgFc2016, respectively. The alignment of the Fc regions of wild-type IgG1, MgFc2012 and MgFc2016 is presented in FIG. 23.

SPR analysis of IgG1 variants, MgFc0088 and MgFc0155, and the IgG2 counterparts, MgFc2016 and MgFc2012, respectively, demonstrated that the effect of heavy chain mutations was isotype sensitive. While the mutations corresponding to MgFc0088/MgFc2016 and MgFc0155/MgFc2012 resulted in the same pattern of antibody binding to CD16A (FcγRIIIA) regardless of isotype (FIG. 24 A-B and FIG. 25 A-B, respectively) the binding of this variant to CD32A 131H (FcγRIIA 131H) or CD32B (FcγRIIB) exhibited distinct isotype differences. In the context of IgG1, MgFc0088 increased binding to both CD32A 131H and CD32B (FIGS. 24 C and D, respectively). However, in the context of IgG2, the same mutation (MgFc2016) had no effect on binding to CD32A 131H and decreased binding to CD32B (FIGS. 24 C and D, respectively). In the context of IgG1, MgFc0155 had no effect on the binding to CD32A 131H and decreased binding to CD32B (FIGS. 25 C and D, respectively); in the context of IgG2, the same mutation, MgFc2012, increased the binding of the antibody to both CD32A 131H and CD32B (FIGS. 25 C and D, respectively). The data suggest that the mutations corresponding to MgFc0088 and MgFc0155 behave differently in the context of IgG1 and IgG2, rendering the choice of antibody isotype critical in the design of an antibody comprising a variant heavy chain.

Mutation MgFc0088 in the context of IgG1 and IgG3: Mutations corresponding to MgFc0088 were introduced into a ch4D5 antibody comprising an IgG3 Fc region by site directed mutagenesis to produce MgFc3013. The same mutations were also introduced into the wild type IgG3 Fc region further comprising the mutation R345H, which mediated binding to protein A, to produce MgFc3014. The alignment of the Fc regions of wild-type IgG3, MgFc3013 and MgFc3014 is presented in FIG. 26.

SPR analysis of MgFc3013 and MgFc3014 demonstrated that the mutations corresponding to MgFc088 exhibited similar effects with regard to antibody binding regardless of isotype (FIGS. 27 and 24). MgFc3013 and MgFc3014 exhibited increased binding to CD16A 158V or 158F, relative to that of an antibody comprising the wild type IgG1 Fc region, and failed to affect binding to either CD32A 131H or CD32B (FIG. 27). The same patterns were observed when the binding of MgFc088 and MgFc2016 were tested (FIG. 24).

Mutation MgFc0155 in the context of IgG1 and IgG3: The wild type IgG3 Fc region was based on the amino acid sequence of the Fc region of the IgG3 heavy chain provided at Genbank Accession No. X03604. Mutations corresponding to MgFc0155 were introduced into a ch4D5 antibody comprising and IgG3 Fc region by site directed mutagenesis to produce MgFc3012. The alignment of the Fc regions of wild-type IgG3 and MgFc3012 is presented in FIG. 28. Note that FIG. 28 also contains the alignment of IgG3 mutant MgFc3011, which was used as the “wild-type” control for the IgG3 Fc region. MgFc3011 contains modifications to IgG1 hinge corresponding to a wild type IgG3 allotype wild type IgG3 Fc region. Note that MgFc3012 contains an IgG1 hinge region.

SPR analysis of wild type IgG3, MgFc3011, and the IgG3 variant, MgFc3012, revealed that, as in the context of IgG1 or IgG2 (see FIG. 25), the mutations corresponding to MgFc0155 increased binding of the antibody to CD16A 158V or 158F relative to that of an antibody comprising the wild type IgG3 Fe region (FIGS. 29 A and B). However, in the context of IgG3, the same mutation failed to affect binding of the antibody to either CD32A 131H or CD32B (FIGS. 29 C and D). This contrasts markedly with the effect of the mutation in the context of either IgG1 or IgG2, wherein antibodies comprising the mutation MgFc0155 were altered in their binding to one or both of CD32A or CD32B, depending on isotype (FIGS. 25 C and D).

Mutations corresponding to MgFc0155 were also introduced into an IgG3 Fc alloype. The amino acid sequence of the Fe region of the allotype was the same as that of Genbank Accession number X03604 and used in MgFc3011, but contained the mutation Y296F. Introduction of mutations corresponding to MgFc0155 into this second IgG3 allotype produced MgFc3002. An additional P396L mutation was introduced into MgFc3002 to produce MgFc3003 to allow direct comparison to MgFc0088a (243L, R292P, F300L, P396L). MgFc0088a and MgFc3003 therefore comprise the same set of mutations but in an IgG1 and IgG3 background, respectively. The alignment of the Fe regions of X03604, MgFc3002 and MgFc3003 is provided in FIG. 30.

Similar to the results in the context of the first allotype of IgG3 tested, SPR analysis of MgFc3002, revealed that, in the context of this IgG3 allotype, MgFc0155 increased binding of the antibody to CD16A 158V or 158F, but failed to affect the binding of the antibody to CD32A 131H or CD32B (FIG. 31). As discussed supra, this contrasts sharply with the effects of the mutation in the context of IgG1 or IgG2 (FIGS. 25 C and D).

The isotype-dependent effects on mutation effects are yet more pronounced when the behaviour of MgFc3003 is considered. Unlike the other mutations considered, in the context of IgG3, MgFc3002 failed to alter the binding of antibody to any receptor tested. This is in great contrast to the effects of the mutation in the context of IgG1 (MgFc0088A), wherein the IgG1 variant results in increased binding to all receptors (data not shown).

SPR analysis of receptor binding to Fc mutants identified in IgG1 context indicated that alteration of the isotype or allotype context can either have no affect on mutant behavior relative to wild-type context or dramatically alter it. Regardless of isotype, mutations corresponding to MgFc0088 maintained a similar pattern of binding to the receptors tested. Similarly, the pattern of variant binding to CD16 was apparently predictable across isotype context. However, the binding properties of antibodies comprising similar mutations in the context of differing isotypes of Fe to CD32A or CD32B was variable. Strategies for antibody therapy originally developed in a single IgG context can therefore not simply be applied in another IgG context, but must be independently evaluated considering the desired properties before implementation.

The invention described and claimed herein is not to be limited in scope by the specific embodiments herein disclosed since these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout this application various publications are cited. Their contents are hereby incorporated by reference into the present application in their entireties for all purposes.

What is claimed is:

1. An antibody comprising:
(1) a CH1 domain of a human IgG1;
(2) a hinge domain of a human IgG1; and
(3) a variant Fe region
wherein said variant Fe region is an Fe region of a human IgG selected from the group consisting of: IgG2, IgG3 and IgG4, which comprises at least one amino acid modification relative to the corresponding amino acid sequence of a wild type Fe region of said IgG, such that said antibody binds an FcγR with altered affinity relative to an antibody comprising said wild-type Fe region, and wherein when said variant Fe region is a variant Fe region of IgG2 said at least one amino acid modification does not solely comprise:
(a) a substitution at position 233 with glutamic acid, at position 234 with leucine, at position 235 with leucine and an insertion at position 237 with glycine; or
(b) a substitution at position 234 with leucine, at position 235 with leucine, and an insertion at position 237 with glycine.

2. The antibody of claim 1, wherein said variant Fe region binds FcγRIIIA with a greater affinity than a comparable antibody comprising the wild-type Fe region binds FcγRIIIA.

3. The antibody of claim 1, wherein said variant Fe region binds FcγRIIA with a greater affinity than a comparable antibody comprising the wild-type Fe region binds FcγRIIA.

4. The antibody of claim 1 wherein said variant Fe region binds FcγRIIB with a lower affinity than a comparable antibody comprising the corresponding wild-type Fe region binds FcγRIIB.

5. The antibody of claim 1 wherein said antibody comprises a variable domain which binds to CD16A.

6. The antibody of claim 1, wherein said antibody comprises a variable domain which binds to CD32B.

7. A nucleic acid comprising a nucleotide sequence encoding the heavy chain of the antibody of claim 1.

8. A therapeutic antibody that specifically binds a cancer antigen, said therapeutic antibody comprising:
(1) a CH1 domain of a human IgG1;
(2) a hinge domain of a human IgG1; and
(3) a variant Fc region
wherein said variant Fc region is an Fc region of a human IgG selected from the group consisting of: IgG2, IgG3 and IgG4, which comprises at least one amino acid modification relative to the corresponding amino acid sequence of a wild type Fc region of said IgG, such that said antibody binds an FcγR with altered affinity relative to an antibody comprising said wild-type Fc region, and wherein when said variant Fc region is a variant Fc region of IgG2 said at least one amino acid modification does not solely comprise:
(a) a substitution at position 233 with glutamic acid, at position 234 with leucine, at position 235 with leucine and an insertion at position 237 with glycine; or
(b) a substitution at position 234 with leucine, at position 235 with leucine, and an insertion at position 237 with glycine.

9. The antibody of claim 8, wherein said variant Fc region binds FcγRIIIA with a greater affinity than a comparable antibody comprising the wild-type Fc region binds FcγRIIIA.

10. The antibody of claim 8, wherein said variant Fc region binds FcγRIIA with a greater affinity than a comparable antibody comprising the wild-type Fc region binds FcγRIIA.

11. The antibody of claim 8, wherein said Fc region binds FcγRIIB with a lower affinity than a comparable antibody comprising the wild-type Fc region binds FcγRIIB.

12. The therapeutic antibody of claim 8, wherein said therapeutic antibody mediates enhanced antibody dependent cell mediated cytotoxicity relative to that of an antibody comprising:
(1) said CH1 domain of said human IgG1;
(2) said hinge domain of said human IgG1; and
(3) said wild type Fc region.

13. The therapeutic antibody of claim 8, wherein said cancer antigen is MAGE-1, MAGE-3, BAGE, GAGE-1, GAGE-2, N-acetylglucosaminyltransferase, p15, beta-catenin, MUM-1, CDK4, HER-2/neu, human papillomavirus-E6, human papillomavirus-E7, MUC-1, CD20 or CD32B.

14. The therapeutic antibody of claim 13, wherein said antibody is 4D5.

15. The therapeutic antibody of claim 13, wherein said antibody is humanized 4D5.

16. A method of treating cancer in a patient having a cancer characterized by a cancer antigen, said method comprising administering to said patient a therapeutically effective amount of the therapeutic antibody of claim 17 that binds said cancer antigen.

17. The method of claim 16, wherein said cancer antigen is MAGE-1, MAGE-3, BAGE, GAGE-1, GAGE-2, N-acetylglucosaminyltransferase, p15, beta-catenin, MUM-1, CDK4, HER-2/neu, human papillomavirus-E6, human papillomavirus-E7, MUC-1, CD20 or CD32B.

18. The method of claim 16, wherein said cancer antigen is a breast, ovarian, prostate, cervical, or pancreatic carcinoma antigen.

19. A pharmaceutical composition comprising a therapeutically effective amount of the antibody of claim 1, and a pharmaceutically acceptable carrier.

20. The pharmaceutical composition of claim 19, further comprising one or more additional anti-cancer agents selected from the group consisting of a chemotherapeutic agent, a radiation therapeutic agent, a hormonal therapeutic agent, or an immunotherapeutic agent.
